(19) United States
(12) Patent Application Publication
(10) Pub. No.: US 2024/0344077 A1
(43) Pub. Date: Oct. 17, 2024

(54) GENETICALLY ALTERED NFR1 RECEPTOR KINASES

(71) Applicant: Aarhus Universitet, Aarhus (DK)

(72) Inventors: Elena Simona RADUTOIU, Aarhus (DK); Kasper Røjkjær ANDERSEN, Aarhus (DK); Magdalini TSITSIKLI, Aarhus (DK); Simon Boje HANSEN, Aarhus (DK); Camilla Gottlieb ANDERSEN, Aarhus (DK); Damiano LIRONI, Aarhus (DK); Christina KRÖNAUER, Aarhus (DK); Maria Meisner LARSEN, Aarhus (DK); Kira Dorothea GYSEL, Aarhus (DK); Marie Vogel KOLTE, Aarhus (DK); Jens Stougaard JENSEN, Aarhus (DK); Bine Wissendorf SIMONSEN, Aarhus (DK)

(73) Assignee: Aarhus Universitet, Aarhus (DK)

(21) Appl. No.: 18/605,796

(22) Filed: Mar. 14, 2024

Related U.S. Application Data
(60) Provisional application No. 63/490,219, filed on Mar. 14, 2023.

(51) Int. Cl. C12N 15/82; C07K 14/415; C12N 9/22; C12N 15/11

(52) U.S. Cl. CPC C12N 15/8271; C07K 14/415; C12N 9/22; C12N 15/11; C12N 15/8205; C12N 15/8227; C12N 2310/20

(57) ABSTRACT

The present disclosure relates to genetically modified plant LysM receptors and methods of producing the same. In particular, the present disclosure relates to modified plant LysM receptors including a modified juxtamembrane (JM) zone 4, and optionally further including a modified JM zone 2, a modified JM zone 3, a modified extracellular domain, and/or a modified kinase C-terminus region or a modified kinase N-terminus region. The modified LysM receptors of the present disclosure are either able to able to initiate NFR1-mediated root nodule symbiosis signaling or able to initiate ROS signaling. In addition, the present disclosure relates to genetically modified plants or parts thereof including the genetically modified plant LysM receptors and methods of producing the same. The present disclosure further relates to expression vectors, isolated DNA molecules, or recombinant nucleic acids encoding the genetically modified plant LysM receptors.

### CROSS-REFERENCE TO RELATED APPLICATIONS

This application claims the benefit of priority to U.S. Provisional 63/490,219, filed Mar. 14, 2023, which is hereby incorporated by reference in its entirety.

### REFERENCE TO AN ELECTRONIC SEQUENCE LISTING

The contents of the electronic sequence listing (794542002300seglist.xml; Size: 207,448 bytes; and Date of Creation: Mar. 8, 2024) are herein incorporated by reference in its entirety.

### REFERENCE TO LARGE TABLES

The contents of the large table CERK6 (CERK6Z4_refine_60.txt; Size: 1,432,541 bytes; and Date of Creation: Mar. 1, 2023) and the large table LYK3 (LYK3Z4_refine_67.txt; Size: 727,916 bytes; and Date of Creation: Mar. 1, 2023) are herein incorporated by reference in their entireties.

### TECHNICAL FIELD

The present disclosure relates to genetically modified plant LysM receptors and methods of producing the same. In particular, the present disclosure relates to modified plant LysM receptors including a modified juxtamembrane (JM) zone 4, and optionally further including a modified JM zone 2, a modified JM zone 3, a modified extracellular domain, and/or a modified kinase C-terminus region or a modified kinase N-terminus region. The modified LysM receptors of the present disclosure are either able to able to initiate NFR1-mediated root nodule symbiosis signaling or able to initiate ROS signaling. In addition, the present disclosure relates to genetically modified plants or parts thereof including the genetically modified plant LysM receptors and methods of producing the same. The present disclosure further relates to expression vectors, isolated DNA molecules, or recombinant nucleic acids encoding the genetically modified plant LysM receptors.

### BACKGROUND

Receptor kinases (RKs) are located on the cell surface of eukaryotic cells, and act as signal sensors and transducers to regulate crucial processes. Plant RKs are known as receptor-like kinases (RLKs, which have intracellular domains) or receptor-like proteins (RLPs, which lack intracellular domains). Most RKs and RLKs are characterized by similar protein domains: an extracellular domain (EC), a single pass transmembrane domain (TM), a cytoplasmic flexible region known as juxtamembrane domain (JM), and a kinase domain (K) followed by a C-terminal tail.

All plants use lysin motif (LysM) RLKs called chitin elicitor kinases (CERKs) to recognize chitin oligomers (CO6-8; chitooligosaccharides) produced by pathogenic fungi and to activate an immune response. Legume plants have evolved highly similar LysM RLKs called nod factor receptors (NFRs) with increased sensitivity for decorated chitin oligomers (LCOs; lipochitooligosaccharides) produced by nitrogen-fixing soil bacteria. These receptors provide legumes with an ecological advantage as they are able to initiate symbiosis signaling leading to nodule organogenesis, bacterial infection, and symbiotic nitrogen fixation in nutrient-poor soils. Both CERKs and NFRs contain three tandem LysM domains in their extracellular region, a single-pass transmembrane (TM) domain, a juxtamembrane (JM) domain, and an intracellular kinase domain. The kinase domains of CERKs and NFRs phosphorylate specific substrates and initiate distinct signaling pathways: immunity for the CERKs or symbiosis for the NFRs.

The endosymbiotic relationship between plants and nitrogen-fixing bacteria is known as root nodule symbiosis (RNS), and there are different types of RNS. The most common type of RNS is rhizobia-legume symbiosis, which occurs between legume plants (of the Fabaceae or Leguminosae family), and nitrogen-fixing bacteria known as rhizobia (Beringer et al. (1979) Proceedings of the Royal Society of London, 204(1155):219-33). Plants that engage in RNS are found only within the orders Fabales, Fagales, Cucurbitales, and Rosales, which together constitute the FaFaCuRo or nitrogen-fixing clade (NFC) (Kistner and Parniske (2002) Trends in Plant Science, 7(11):511-18; Soltis et al. (1995) PNAS, 92(7):2647-51). However, only 10 out of 28 families of the NFC contain plant species able to establish RNS (nodulating species) (Doyle (2011) MPMI, 24(11):1289-95).

Rhizobia-legume RNS requires the plant LysM receptor kinase pair NFR1 and NFR5 for the perception of Nod factors (Radutoiu et al. (2003) Nature 425, 585-592) and the subsequent initiation of the symbiosis pathway. It has been shown that receptor signaling requires the catalytic activity of the NFR1 protein kinase, while the mechanistic role for the catalytically inactive NFR5 pseudokinase is less well understood. LjNFR1 and MtLYK3, both of which are NFRs with kinase domains, are thought to be key receptors for symbiotic signaling in the nodulating model legume species Lotus japonicus and Medicago truncatula, respectively (Arrighi et al. (2006) Plant Phys., 142(1):265-79; Madsen et al. (2011) Plant J., 65(3):404-17.; Mbengue et al. (2010) Plant Cell, 22(10):3474). In Lotus japonicus, NFR1 is indispensable for signaling, since its absence results in the lack of either physiological change (e.g., root hair deformation in response to NF) or molecular changes (e.g., no calcium signaling or expression of key nodulation genes, such as the transcription factor NIN) (Madsen et al. (2003) Nature, 425(6958):637-40.; Miwa et al. (2006) MPMI, 9(8):914-23; Radutoiu et al. (2003) Nature, 425(6958):585-92). Despite the importance of NFRs with kinase domains, the structural domains of the receptors that are responsible for determining the downstream symbiosis pathway have remained unidentified.

There exists a need to understand the specific domains of the NFR1 kinase involved in the initiation of the symbiosis pathway. More broadly, there is a need to identify the structural domains of LysM receptors that determine whether the downstream symbiosis pathway is initiated or the downstream immunity pathway. The identification of these domains will allow the engineering of existing LysM receptor kinases involved in immunity signaling (CERKs) into LysM receptor kinases involved in symbiosis signaling (NFRs) and vice versa. Further identifying these domains will allow targeted modification of existing LysM receptor kinases into kinases able to initiate specific downstream signaling pathways. Identification of these domains is a key in LysM receptor kinase engineering, and represents an important step for engineering NFR1-mediated root nodule symbiosis signaling in non-legume species.

### BRIEF SUMMARY

The present disclosure identifies a zone in the NFR1 juxtamembrane (JM) domain as being critical for NFR1-mediated root nodule symbiosis signaling, namely JM zone 4. Additional JM domains identified in the present disclosure, JM zone 2 and JM zone 3, are thought to be essential for receptor dimerization. In addition, the present disclosure establishes that the C-terminus of the NFR1 kinase domain is required for symbiosis signaling (i.e., NFR1-mediated root nodule symbiosis signaling), and that the N-terminus of the CERK6 kinase domain is required for immunity signaling (i.e., ROS signaling). In combination with previously identified ectodomain regions of LysM receptors, which determine receptor specificity, the transmembrane/juxtamembrane and intracellular residues and regions of the present disclosure provide essential components needed for engineering NFR1-mediated root nodule symbiosis signaling in other plant LysM receptor proteins and in non-legume species. Further, the present disclosure provides transmembrane/juxtamembrane and intracellular residues and regions required for engineering immunity signaling in plant LysM receptors.

An aspect of the disclosure includes a modified plant LysM receptor polypeptide including a first JM zone 4, wherein the first JM zone 4 has been modified by insertion, deletion, or substitution of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 4 with the corresponding amino acids from a second JM zone 4 of a second plant LysM receptor polypeptide or wherein the plant LysM receptor polypeptide lacks a first JM zone 4 and the plant LysM receptor polypeptide has been modified by insertion of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 4 with the corresponding amino acids from the second JM zone 4 into the corresponding site when aligning the plant LysM receptor and the second plant LysM receptor. In a further embodiment of this aspect, the first JM zone 4, the second JM zone 4, or both correspond to amino acids 305 to 327 when aligned to SEQ ID NO: 1 or correspond to amino acids 303 to 325 when aligned to SEQ ID NO: 8. In an additional embodiment of this aspect, which may be combined with any of the preceding embodiments, the first JM zone 4 is modified by substituting at least two or at least three amino acid residues in the first JM zone 4 with corresponding amino acid residues that are different in the second JM zone 4, or the first JM zone 4 is modified by substituting three amino acid residues in the first JM zone 4 with corresponding amino acid residues that are different in the second JM zone 4. In yet another embodiment of this aspect, substitution includes deletion of an amino acid not found in the second JM zone 4 and insertion of an amino acid found in the second JM zone 4 but not in the first JM zone 4. In still another embodiment of this aspect, which may be combined with any of the preceding embodiments, the second JM zone 4 is able to initiate NFR1-mediated root nodule symbiosis signaling, ROS signaling, or different signaling than the first JM zone 4. In another embodiment of this aspect, the second JM zone 4 is able to initiate NFR1-mediated root nodule symbiosis signaling. In an additional embodiment of this aspect, the first JM zone 4 includes SEQ ID NO: 13, is selected from the group of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40, or conservative substitutions thereof, or optionally any of the preceding amino acid sequences with one, two, or three amino acid substitutions, insertions and/or deletions, or includes SEQ ID NO: 60; and/or wherein the second JM zone 4 includes SEQ ID NO: 6, or is selected from the group of SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and SEQ ID NO: 52, or conservative substitutions thereof, or optionally any of the preceding amino acid sequences with one, two, or three amino acid substitutions, insertions and/or deletions, or optionally any of the preceding amino acid sequences with one, two, or three amino acid substitutions, insertions and/or deletions. In a further embodiment of this aspect, the second JM zone 4 is able to initiate ROS signaling. In still another embodiment of this aspect, the first JM zone 4 includes SEQ ID NO: 6, or is selected from the group of SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and SEQ ID NO: 52, or conservative substitutions thereof, or optionally any of the preceding amino acid sequences with one, two, or three amino acid substitutions, insertions and/or deletions, and/or wherein the second JM zone 4 includes SEQ ID NO: 13, or is selected from the group of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40, or optionally any of the preceding amino acid sequences with one, two, or three amino acid substitutions, insertions and/or deletions. In yet another embodiment of this aspect, which may be combined with any of the preceding embodiments, the substituted amino acid residues are selected from amino acid residues corresponding to M306, A308, and K320 of SEQ ID NO: 1 or from amino acid residues corresponding to T304, D306, and T318 of SEQ ID NO: 8.

A further aspect of the disclosure includes a modified plant non-NFR1 LysM receptor polypeptide engineered for NFR1-mediated root nodule symbiosis signaling including a first JM zone 4, wherein the first JM zone 4 was modified by substitution of one or more amino acids in the first JM zone 4 with the corresponding amino acids from a second JM zone 4 from an NFR1 LysM receptor polypeptide with NFR1-mediated root nodule symbiosis signaling. In an additional embodiment of this aspect, substitution includes deletion of an amino acid not found in the second JM zone 4 and insertion of an amino acid found in the second JM zone 4 but not in the first JM zone 4. In a further embodiment of this aspect, which may be combined with any of the preceding embodiments, the first JM zone 4 is modified by substituting at least two or at least three amino acid residues in the first JM zone 4 with corresponding amino acid residues that are different in the second JM zone 4. In yet another embodiment of this aspect, the first JM zone 4 is modified by substituting three amino acid residues in the first JM zone 4 with corresponding amino acid residues that are different in the second JM zone 4. In still another embodiment of this aspect, which may be combined with any of the preceding embodiments, the first JM zone 4 includes SEQ ID NO: 13, is selected from the group of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40, or optionally any of the preceding amino acid sequences with one, two, or three amino acid substitutions, insertions and/or deletions, or includes SEQ ID NO: 60, and/or wherein the second JM zone 4 includes SEQ ID NO: 6, or is selected from the group of SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and SEQ ID NO: 52, or conservative substitutions thereof, or optionally any of the preceding amino acid sequences with one, two, or three amino acid substitutions, insertions and/or deletions. In a further embodiment of this aspect, which may be combined with any of the preceding embodiments, the substituted amino acid residues are selected from amino acid residues corresponding to M306, A308, and K320 of SEQ ID NO: 1 or from amino acid residues corresponding to T304, D306, and T318 of SEQ ID NO: 8. In an additional embodiment of this aspect, which may be combined with any of the preceding embodiments, the modified plant non-NFR1 LysM receptor polypeptide further includes a first kinase C-terminus region, wherein the first kinase C-terminus region has been modified as compared to the amino acid sequence of the corresponding unmodified plant LysM receptor polypeptide by substitution of one or more amino acids in the first kinase C-terminus region with the corresponding amino acids from an NFR1 LysM receptor polypeptide.

An additional aspect of the disclosure includes a modified plant LysM receptor polypeptide with enhanced ROS signaling including a first JM zone 4, wherein the first JM zone 4 was modified by substitution of one or more amino acids in the first JM zone 4 with the corresponding amino acids from a second JM zone 4 from a LysM receptor polypeptide with ROS signaling. In a further embodiment of this aspect, the first JM zone 4 is modified by substituting at least two or at least three amino acid residues in the first JM zone 4 with corresponding amino acid residues that are different in the second JM zone 4, or the first JM zone 4 is modified by substituting three amino acid residues in the first JM zone 4 with corresponding amino acid residues that are different in the second JM zone 4. In an additional embodiment of this aspect, substitution includes deletion of an amino acid not found in the second JM zone 4 and insertion of an amino acid found in the second JM zone 4 but not in the first JM zone 4. In yet another embodiment of this aspect, which may be combined with any of the preceding embodiments, the first JM zone 4 includes SEQ ID NO: 6, or is selected from the group of SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and SEQ ID NO: 52, or conservative substitutions thereof, or optionally any of the preceding amino acid sequences with one, two, or three amino acid substitutions, insertions and/or deletions, and/or wherein the second JM zone 4 includes SEQ ID NO: 13, or is selected from the group of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40, or optionally any of the preceding amino acid sequences with one, two, or three amino acid substitutions, insertions and/or deletions. In still another embodiment of this aspect, which may be combined with any of the preceding embodiments, the substituted amino acid residues are selected from amino acid residues corresponding to M306, A308, and K320 of SEQ ID NO: 1 or from amino acid residues corresponding to T304, D306, and T318 of SEQ ID NO: 8.

In a further embodiment of the preceding aspects, which may be combined with any of the preceding embodiments, the modified plant LysM receptor polypeptide further includes a first JM zone 2, wherein the first JM zone 2 has been modified by insertion, deletion, or substitution of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 2 with the corresponding amino acids from a second JM zone 2 of a second plant LysM receptor polypeptide or wherein the plant LysM receptor polypeptide lacks a first JM zone 2 and the plant LysM receptor polypeptide has been modified by insertion of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 2 with the corresponding amino acids from the second JM zone 2 into the corresponding site when aligning the plant LysM receptor and the second plant LysM receptor, wherein substitution optionally includes deletion of an amino acid not found in the second JM zone 2 and insertion of an amino acid found in the second JM zone 2 but not in the first JM zone 2. In an additional embodiment of this aspect, the first JM zone 2, the second JM zone 2, or both correspond to amino acids 256 to 280 when aligned to SEQ ID NO: 1 or correspond to amino acids 256 to 281 when aligned to SEQ ID NO: 8. In yet another embodiment of this aspect, the first JM zone 2 and/or the second JM zone 2 includes SEQ ID NO: 11, SEQ ID NO: 58, or SEQ ID NO: 4.

In an additional embodiment of the preceding aspects, which may be combined with any of the preceding embodiments, the modified plant LysM receptor polypeptide further includes a first JM zone 3, wherein the first JM zone 3 has been modified by insertion, deletion, or substitution of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 3 with the corresponding amino acids from a second JM zone 3 of a second plant LysM receptor polypeptide or wherein the plant LysM receptor polypeptide lacks a first JM zone 3 and the plant LysM receptor polypeptide has been modified by insertion of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids from the second JM zone 3 into the corresponding site when aligning the plant LysM receptor and the second plant LysM receptor, wherein substitution optionally includes deletion of an amino acid not found in the second JM zone 3 and insertion of an amino acid found in the second JM zone 2 but not in the first JM zone 3. In an additional embodiment of this aspect, the first JM zone 3, the second JM zone 3, or both correspond to amino acids 281 to 304 when aligned to SEQ ID NO: 1 or correspond to amino acids 282 to 302 when aligned to SEQ ID NO: 8. In yet another embodiment of this aspect, the first JM zone 3 and/or the second JM zone 3 includes SEQ ID NO: 12, SEQ ID NO: 59, or SEQ ID NO: 5.

Yet another aspect of the disclosure includes a modified plant non-NFR1 LysM receptor polypeptide engineered for NFR1-mediated root nodule symbiosis signaling including the first kinase C-terminus region has been modified by insertion, deletion, or substitution of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the first kinase C-terminus region with the corresponding amino acids from a C-terminus region of a NFR1 LysM receptor polypeptide or wherein the plant non-NFR1 LysM receptor polypeptide lacks a first C-terminus region and the plant non-NFR1 LysM receptor polypeptide has been modified by insertion of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the second C-terminus region into the corresponding site when aligning the plant non-NFR1 LysM receptor and the plant NFR1 LysM receptor. In a further embodiment of this aspect, the first kinase C-terminus region corresponds to amino acids T467, S471, S473, P475, G477, V496, V500, Y510, D517, 1519, V520, 5523, 5525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, and G621 when aligned to SEQ ID NO: 8, and/or wherein the second kinase C-terminus region corresponds to amino acids 1469, N473, T475, Q477, 1497, 1501, F511, N518, V520, L521, G524, L526, V527, A528, E529, E539, A540, N542, K543, S544, C547, D548, A549, P556, E560, E564, L568, 1570, G574, R575, R579, L583, L591, L601, E603, C605, D607, E608, S610, S613, T615, 1617, L620, and V622 when aligned to SEQ ID NO: 1. In an additional embodiment of this aspect, which may be combined with any of the preceding embodiments, the first kinase C-terminus region includes amino acids T467, S471, S473, P475, G477, V496, V500, Y510, D517, 1519, V520, S523, S525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, and G621 of SEQ ID NO: 8. In a further embodiment of this aspect, the first kinase C-terminus region is modified by substituting one or more amino acids of T467, S471, S473, P475, G477, V496, V500, Y510, D517, 1519, V520, S523, S525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, or G621 of SEQ ID NO: 8 with one or more amino acids of 1469, N473, T475, Q477, 1497, 1501, F511, N518, V520, L521, G524, L526, V527, A528, E529, E539, A540, N542, K543, S544, C547, D548, A549, P556, E560, E564, L568, 1570, G574, R575, R579, L583, L591, L601, E603, C605, D607, E608, S610, S613, T615, 1617, L620, or V622 of SEQ ID NO: 1. In yet another embodiment of this aspect, which may be combined with any of the preceding embodiments, the modified non-NFR1 LysM receptor polypeptide is able to initiate NFR1-mediated root nodule symbiosis signaling.

Still another aspect of the disclosure includes a modified plant non-CERK6 LysM receptor polypeptide engineered for immune signaling including a first kinase N-terminus region, wherein the first kinase N-terminus region has been modified by insertion, deletion, or substitution of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the first kinase N-terminus region with the corresponding amino acids from a second N-terminus region of a CERK6 LysM receptor polypeptide or wherein the non-CERK6 plant LysM receptor polypeptide lacks the first N-terminus region and the non-CERK6 plant LysM receptor polypeptide has been modified by insertion of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the second N-terminus region into the corresponding site when aligning the non-CERK6 plant LysM receptor and the CERK6 LysM receptor. In an additional embodiment of this aspect, the first kinase N-terminus region corresponds to D328, A338, K347, K348, T349, V356, T360, C364, H371, C384, V385, H395, D397, G402, Y404, H406, K410, E411, S416, S417, A425, V445, and L456 when aligned to SEQ ID NO: 1, and the second kinase N-terminus region corresponds to A326, S336, E345, R346, A347, M354, K358, A362, R369, S382, 1383, F393, E395, S400, H402, R404, R408, D409, A414, T415, S423, 1443, and Y454 when aligned to SEQ ID NO: 8. In a further embodiment of this aspect, which may be combined with any of the preceding embodiments, the first kinase N-terminus region includes D328, A338, K347, K348, T349, V356, T360, C364, H371, C384, V385, H395, D397, G402, Y404, H406, K410, E411, S416, S417, A425, V445, and L456 of SEQ ID NO: 1. In yet another embodiment of this aspect, the first kinase N-terminus region is modified by substituting one or more amino acids of D328, A338, K347, K348, T349, V356, T360, C364, H371, C384, V385, H395, D397, G402, Y404, H406, K410, E411, S416, S417, A425, V445, or L456 of SEQ ID NO: 1 with one or more amino acids of A326, S336, E345, R346, A347, M354, K358, A362, R369, S382, 1383, F393, E395, S400, H402, R404, R408, D409, A414, T415, S423, 1443, or Y454 of SEQ ID NO: 8. In still another embodiment of this aspect, which may be combined with any of the preceding embodiments, the modified plant non-CERK6 LysM receptor polypeptide is able to initiate ROS signaling.

In a further embodiment of this aspect, which may be combined with any of the preceding embodiments that has a modified plant LysM receptor polypeptide including a first JM zone 4 modified by insertion, deletion, or substitution of one or more amino acids in the first JM zone 4 with the corresponding amino acids from a second JM zone 4 able to initiate NFR1-mediated root nodule symbiosis signaling, the modified plant LysM receptor polypeptide further includes the first kinase C-terminus region has been modified by insertion, deletion, or substitution of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the first kinase C-terminus region with the corresponding amino acids from a C-terminus region of a NFR1 LysM receptor polypeptide or wherein plant non-NFR1 LysM receptor polypeptide lacks a first C-terminus region and the plant non-NFR1 LysM receptor polypeptide has been modified by insertion of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the second C-terminus region into the corresponding site when aligning the plant non-NFR1 LysM receptor and the plant NFR1 LysM receptor. In an additional embodiment of this aspect, the first kinase C-terminus region corresponds to amino acids T467, S471, S473, P475, G477, V496, V500, Y510, D517, 1519, V520, 5523, S525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, and G621 when aligned to SEQ ID NO: 8, and the second kinase C-terminus region corresponds to amino acids 1469, N473, T475, Q477, 1497, 1501, F511, N518, V520, L521, G524, L526, V527, A528, E529, E539, A540, N542, K543, S544, C547, D548, A549, P556, E560, E564, L568, 1570, G574, R575, R579, L583, L591, L601, E603, C605, D607, E608, S610, S613, T615, 1617, L620, and V622 when aligned to SEQ ID NO: 1. In yet another embodiment of this aspect, which may be combined with any of the preceding embodiments, the first kinase C-terminus region includes amino acids T467, S471, S473, P475, G477, V496, V500, Y510, D517, 1519, V520, S523, S525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, and G621 of SEQ ID NO: 8. In still another embodiment of this aspect, the first kinase C-terminus region is modified by substituting one or more amino acids of T467, S471, S473, P475, G477, V496, V500, Y510, D517, 1519, V520, S523, S525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, or G621 of SEQ ID NO: 8 with one or more amino acids of 1469, N473, T475, Q477, 1497, 1501, F511, N518, V520, L521, G524, L526, V527, A528, E529, E539, A540, N542, K543, S544, C547, D548, A549, P556, E560, E564, L568, 1570, G574, R575, R579, L583, L591, L601, E603, C605, D607, E608, S610, S613, T615, 1617, L620, or V622 of SEQ ID NO: 1. In a further embodiment of this aspect, which may be combined with any of the preceding embodiments, the modified plant LysM receptor polypeptide is able to initiate NFR1-mediated root nodule symbiosis signaling.

In an additional embodiment of this aspect, which may be combined with any of the preceding embodiments that has a modified plant LysM receptor polypeptide including a first JM zone 4 modified by insertion, deletion, or substitution of one or more amino acids in the first JM zone 4 with the corresponding amino acids from a second JM zone 4 able to ROS signaling, the modified plant LysM receptor polypeptide further includes a first kinase N-terminus region, wherein the first kinase N-terminus region has been modified by insertion, deletion, or substitution of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the first kinase N-terminus region with the corresponding amino acids from a second N-terminus region of a CERK6 LysM receptor polypeptide or wherein the non-CERK6 plant LysM receptor polypeptide lacks the first N-terminus region and the non-CERK6 plant LysM receptor polypeptide has been modified by insertion of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the second N-terminus region into the corresponding site when aligning the non-CERK6 plant LysM receptor and the CERK6 LysM receptor. In a further embodiment of this aspect, the first kinase N-terminus region corresponds to D328, A338, K347, K348, T349, V356, T360, C364, H371, C384, V385, H395, D397, G402, Y404, H406, K410, E411, S416, S417, A425, V445, and L456 when aligned to SEQ ID NO: 1, and the second kinase N-terminus region corresponds to A326, S336, E345, R346, A347, M354, K358, A362, R369, S382, 1383, F393, E395, S400, H402, R404, R408, D409, A414, T415, S423, 1443, and Y454 when aligned to SEQ ID NO: 8. In yet another embodiment of this aspect, which may be combined with any one of the preceding embodiments, the first kinase N-terminus region includes D328, A338, K347, K348, T349, V356, T360, C364, H371, C384, V385, H395, D397, G402, Y404, H406, K410, E411, S416, S417, A425, V445, and L456 of SEQ ID NO: 1. In still another embodiment of this aspect, the first kinase N-terminus region is modified by substituting one or more amino acids of D328, A338, K347, K348, T349, V356, T360, C364, H371, C384, V385, H395, D397, G402, Y404, H406, K410, E411, S416, S417, A425, V445, or L456 of SEQ ID NO: 1 with one or more amino acids of A326, S336, E345, R346, A347, M354, K358, A362, R369, S382, 1383, F393, E395, S400, H402, R404, R408, D409, A414, T415, S423, 1443, or Y454 of SEQ ID NO: 8. In a further embodiment of this aspect, which may be combined with any one of the preceding embodiments, the modified LysM receptor polypeptide is able to initiate ROS signaling.

In still another embodiment of this aspect, which may be combined with any of the preceding embodiments that has a modified plant non-NFR1 LysM receptor polypeptide, the modified plant non-NFR1 LysM receptor polypeptide further includes the first kinase C-terminus region has been modified by insertion, deletion, or substitution of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the first kinase C-terminus region with the corresponding amino acids from a C-terminus region of a NFR1 LysM receptor polypeptide or wherein plant non-NFR1 LysM receptor polypeptide lacks a first C-terminus region and the plant non-NFR1 LysM receptor polypeptide has been modified by insertion of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the second C-terminus region into the corresponding site when aligning the plant non-NFR1 LysM receptor and the plant NFR1 LysM receptor. In a further embodiment of this aspect, the first kinase C-terminus region corresponds to amino acids T467, S471, S473, P475, G477, V496, V500, Y510, D517, 1519, V520, 5523, S525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, and G621 when aligned to SEQ ID NO: 8, and the second kinase C-terminus region corresponds to amino acids 1469, N473, T475, Q477, 1497, I501, F511, N518, V520, L521, G524, L526, V527, A528, E529, E539, A540, N542, K543, S544, C547, D548, A549, P556, E560, E564, L568, 1570, G574, R575, R579, L583, L591, L601, E603, C605, D607, E608, S610, S613, T615, 1617, L620, and V622 when aligned to SEQ ID NO: 1. In an additional embodiment of this aspect, which may be combined with any one of the preceding embodiments, the first kinase C-terminus region includes amino acids T467, S471, S473, P475, G477, V496, V500, Y510, D517, 1519, V520, S523, S525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, and G621 of SEQ ID NO: 8. In yet another embodiment of this aspect, the first kinase C-terminus region is modified by substituting one or more amino acids of T467, S471, S473, P475, G477, V496, V500, Y510, D517, 1519, V520, S523, S525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, or G621 of SEQ ID NO: 8 with one or more amino acids of 1469, N473, T475, Q477, 1497, I501, F511, N518, V520, L521, G524, L526, V527, A528, E529, E539, A540, N542, K543, S544, C547, D548, A549, P556, E560, E564, L568, 1570, G574, R575, R579, L583, L591, L601, E603, C605, D607, E608, S610, S613, T615, 1617, L620, or V622 of SEQ ID NO: 1. In still another embodiment of this aspect, which may be combined with any one of the preceding embodiments, the modified plant non-NFR1 LysM receptor polypeptide is able to initiate NFR1-mediated root nodule symbiosis signaling.

In yet another embodiment of this aspect, which may be combined with any of the preceding embodiments that has a modified plant LysM receptor polypeptide with enhanced ROS signaling, the modified plant LysM receptor polypeptide further includes a first kinase N-terminus region, wherein the first kinase N-terminus region has been modified by insertion, deletion, or substitution of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the first kinase N-terminus region with the corresponding amino acids from a second N-terminus region of a CERK6 LysM receptor polypeptide or wherein the non-CERK6 plant LysM receptor polypeptide lacks the first N-terminus region and the non-CERK6 plant LysM receptor polypeptide has been modified by insertion of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the second N-terminus region into the corresponding site when aligning the non-CERK6 plant LysM receptor and the CERK6 LysM receptor. In an additional embodiment of this aspect, the first kinase N-terminus region corresponds to D328, A338, K347, K348, T349, V356, T360, C364, H371, C384, V385, H395, D397, G402, Y404, H406, K410, E411, S416, S417, A425, V445, and L456 when aligned to SEQ ID NO: 1, and the second kinase N-terminus region corresponds to A326, S336, E345, R346, A347, M354, K358, A362, R369, S382, 1383, F393, E395, S400, H402, R404, R408, D409, A414, T415, S423, 1443, and Y454 when aligned to SEQ ID NO: 8. In a further embodiment of this aspect, which may be combined with any one of the preceding embodiments, the first kinase N-terminus region includes D328, A338, K347, K348, T349, V356, T360, C364, H371, C384, V385, H395, D397, G402, Y404, H406, K410, E411, S416, S417, A425, V445, and L456 of SEQ ID NO: 1. In yet another embodiment of this aspect, the first kinase N-terminus region is modified by substituting one or more amino acids of D328, A338, K347, K348, T349, V356, T360, C364, H371, C384, V385, H395, D397, G402, Y404, H406, K410, E411, S416, S417, A425, V445, or L456 of SEQ ID NO: 1 with one or more amino acids of A326, S336, E345, R346, A347, M354, K358, A362, R369, S382, 1383, F393, E395, S400, H402, R404, R408, D409, A414, T415, S423, 1443, or Y454 of SEQ ID NO: 8. In still another embodiment of this aspect, which may be combined with any one of the preceding embodiments, the modified LysM receptor polypeptide is able to initiate ROS signaling.

In an additional embodiment of the preceding aspects, which may be combined with any of the preceding embodiments, the modified plant LysM receptor polypeptide further includes a first extracellular domain, wherein the first extracellular domain is modified as compared to the amino acid sequence of the corresponding unmodified plant LysM receptor polypeptide. In a further embodiment of this aspect, the first extracellular domain is modified by substituting one or more amino acids of the first extracellular domain with one or more amino acids of a second extracellular domain, and wherein the second extracellular domain has a different affinity, selectivity, and/or specificity for oligosaccharides than the first extracellular domain.

Some aspects of the disclosure include a genetically modified plant or part thereof including the modified plant LysM receptor polypeptide of any one of the preceding embodiments that has a modified plant LysM receptor polypeptide including a first JM zone 4 modified by insertion, deletion, or substitution of one or more amino acids in the first JM zone 4 with the corresponding amino acids from a second JM zone 4 able to initiate NFR1-mediated root nodule symbiosis signaling. In a further embodiment of this aspect, the modified plant LysM receptor polypeptide includes a first JM zone 4 modified by insertion, deletion, or substitution of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 4 with the corresponding amino acids from a second JM zone 4 of a second plant LysM receptor polypeptide or wherein the plant LysM receptor polypeptide lacks a first JM zone 4 and the plant LysM receptor polypeptide has been modified by insertion of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 4 with the corresponding amino acids from the second JM zone 4 into the corresponding site when aligning the plant LysM receptor and the second plant LysM receptor, wherein the first JM zone 4 includes SEQ ID NO: 13, is selected from the group of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40, or optionally any of the preceding amino acid sequences with one, two, or three amino acid substitutions, insertions and/or deletions, or includes SEQ ID NO: 60, and/or wherein the second JM zone 4 includes SEQ ID NO: 6, or is selected from the group of SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and SEQ ID NO: 52, or conservative substitutions thereof, or optionally any of the preceding amino acid sequences with one, two, or three amino acid substitutions, insertions and/or deletions; and optionally further includes: (i) a first JM zone 2, wherein the first JM zone 2 has been modified by insertion, deletion, or substitution of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 2 with the corresponding amino acids from a second JM zone 2 of a second plant LysM receptor polypeptide or wherein the plant LysM receptor polypeptide lacks a first JM zone 2 and the plant LysM receptor polypeptide has been modified by insertion of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 2 with the corresponding amino acids from the second JM zone 2 into the corresponding site when aligning the plant LysM receptor and the second plant LysM receptor, and wherein the first JM zone 2, the second JM zone 2, or both correspond to amino acids 256 to 280 when aligned to SEQ ID NO: 1 or correspond to amino acids 256 to 281 when aligned to SEQ ID NO: 8; (ii) a first JM zone 3, wherein the first JM zone 3 has been modified by insertion, deletion, or substitution of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 3 with the corresponding amino acids from a second JM zone 3 of a second plant LysM receptor polypeptide or wherein the plant LysM receptor polypeptide lacks a first JM zone 3 and the plant LysM receptor polypeptide has been modified by insertion of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids from the second JM zone 3 into the corresponding site when aligning the plant LysM receptor and the second plant LysM receptor, and wherein the first JM zone 3, the second JM zone 3, or both correspond to amino acids 281 to 304 when aligned to SEQ ID NO: 1 or correspond to amino acids 282 to 302 when aligned to SEQ ID NO: 8; (iii) the first kinase C-terminus region has been modified by insertion, deletion, or substitution of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the first kinase C-terminus region with the corresponding amino acids from a C-terminus region of a NFR1 LysM receptor polypeptide or wherein plant non-NFR1 LysM receptor polypeptide lacks a first C-terminus region and the plant non-NFR1 LysM receptor polypeptide has been modified by insertion of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the second C-terminus region into the corresponding site when aligning the plant non-NFR1 LysM receptor and the plant NFR1 LysM receptor, wherein the first kinase C-terminus region corresponds to amino acids T467, S471, S473, P475, G477, V496, V500, Y510, D517, 1519, V520, S523, S525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, and G621 when aligned to SEQ ID NO: 8, and/or wherein the second kinase C-terminus region corresponds to amino acids 1469, N473, T475, Q477, 1497, 1501, F511, N518, V520, L521, G524, L526, V527, A528, E529, E539, A540, N542, K543, S544, C547, D548, A549, P556, E560, E564, L568, 1570, G574, R575, R579, L583, L591, L601, E603, C605, D607, E608, S610, S613, T615, 1617, L620, and V622 when aligned to SEQ ID NO: 1; and/or (iv) a first extracellular domain modified by substituting one or more amino acids of the first extracellular domain with one or more amino acids of a second extracellular domain, wherein the second extracellular domain has a different affinity, selectivity, and/or specificity for oligosaccharides than the first extracellular domain. In an additional embodiment of this aspect, which may be combined with any of the preceding embodiments, the modified plant LysM receptor polypeptide is able to initiate NFR1-mediated root nodule symbiosis signaling.

Some aspects of the disclosure include a genetically modified plant or part thereof including the modified plant LysM receptor polypeptide of any one of the preceding embodiments that has a modified plant LysM receptor polypeptide including a first JM zone 4 modified by insertion, deletion, or substitution of one or more amino acids in the first JM zone 4 with the corresponding amino acids from a second JM zone 4 able to ROS signaling. In a further embodiment of this aspect, the modified plant LysM receptor polypeptide includes a first JM zone 4 modified by insertion, deletion, or substitution of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 4 with the corresponding amino acids from a second JM zone 4 of a second plant LysM receptor polypeptide or wherein the plant LysM receptor polypeptide lacks a first JM zone 4 and the plant LysM receptor polypeptide has been modified by insertion of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 4 with the corresponding amino acids from the second JM zone 4 into the corresponding site when aligning the plant LysM receptor and the second plant LysM receptor, wherein the first JM zone 4 includes SEQ ID NO: 6, or is selected from the group of SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and SEQ ID NO: 52, or conservative substitutions thereof, or optionally any of the preceding amino acid sequences with one, two, or three amino acid substitutions, insertions and/or deletions, and/or wherein the second JM zone 4 includes SEQ ID NO: 13, or is selected from the group of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40, or optionally any of the preceding amino acid sequences with one, two, or three amino acid substitutions, insertions and/or deletions; and optionally further includes: (i) a first JM zone 2, wherein the first JM zone 2 has been modified by insertion, deletion, or substitution of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 2 with the corresponding amino acids from a second JM zone 2 of a second plant LysM receptor polypeptide or wherein the plant LysM receptor polypeptide lacks a first JM zone 2 and the plant LysM receptor polypeptide has been modified by insertion of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 2 with the corresponding amino acids from the second JM zone 2 into the corresponding site when aligning the plant LysM receptor and the second plant LysM receptor, and wherein the first JM zone 2, the second JM zone 2, or both correspond to amino acids 256 to 280 when aligned to SEQ ID NO: 1 or correspond to amino acids 256 to 281 when aligned to SEQ ID NO: 8; (ii) a first JM zone 3, wherein the first JM zone 3 has been modified by insertion, deletion, or substitution of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 3 with the corresponding amino acids from a second JM zone 3 of a second plant LysM receptor polypeptide or wherein the plant LysM receptor polypeptide lacks a first JM zone 3 and the plant LysM receptor polypeptide has been modified by insertion of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids from the second JM zone 3 into the corresponding site when aligning the plant LysM receptor and the second plant LysM receptor, and wherein the first JM zone 3, the second JM zone 3, or both correspond to amino acids 281 to 304 when aligned to SEQ ID NO: 1 or correspond to amino acids 282 to 302 when aligned to SEQ ID NO: 8; (iii) a first kinase N-terminus region, wherein the first kinase N-terminus region has been modified by insertion, deletion, or substitution of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the first kinase N-terminus region with the corresponding amino acids from a second N-terminus region of a CERK6 LysM receptor polypeptide or wherein the non-CERK6 plant LysM receptor polypeptide lacks the first N-terminus region and the non-CERK6 plant LysM receptor polypeptide has been modified by insertion of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the second N-terminus region into the corresponding site when aligning the non-CERK6 plant LysM receptor and the CERK6 LysM receptor, wherein the first kinase N-terminus region corresponds to D328, A338, K347, K348, T349, V356, T360, C364, H371, C384, V385, H395, D397, G402, Y404, H406, K410, E411, S416, S417, A425, V445, and L456 when aligned to SEQ ID NO: 1, and/or wherein the second kinase N-terminus region corresponds to A326, S336, E345, R346, A347, M354, K358, A362, R369, S382, 1383, F393, E395, S400, H402, R404, R408, D409, A414, T415, S423, 1443, and Y454 when aligned to SEQ ID NO: 8; and/or (iv) a first extracellular domain modified by substituting one or more amino acids of the first extracellular domain with one or more amino acids of a second extracellular domain, wherein the second extracellular domain has a different affinity, selectivity, and/or specificity for oligosaccharides than the first extracellular domain. In an additional embodiment of this aspect, which may be combined with any one of the preceding embodiments, the modified plant LysM receptor polypeptide is able to initiate ROS signaling.

Some aspects of the disclosure include a genetically modified plant or part thereof including the modified plant LysM receptor polypeptide of any one of the preceding embodiments that has a modified plant non-NFR1 LysM receptor polypeptide. In a further embodiment of this aspect, the modified plant non-NFR1 LysM receptor polypeptide includes a first JM zone 4, wherein the first JM zone 4 was modified by substitution of one or more amino acids in the first JM zone 4 with the corresponding amino acids from a second JM zone 4 from an NFR1 LysM receptor polypeptide with NFR1-mediated root nodule symbiosis signaling, wherein the first JM zone 4 includes SEQ ID NO: 13, is selected from the group of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40, or optionally any of the preceding amino acid sequences with one, two, or three amino acid substitutions, insertions and/or deletions, or includes SEQ ID NO: 60, and/or wherein the second JM zone 4 includes SEQ ID NO: 6, or is selected from the group of SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and SEQ ID NO: 52, or conservative substitutions thereof, or optionally any of the preceding amino acid sequences with one, two, or three amino acid substitutions, insertions and/or deletions; and optionally further includes: (i) a first JM zone 2, wherein the first JM zone 2 has been modified by insertion, deletion, or substitution of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 2 with the corresponding amino acids from a second JM zone 2 of a second plant LysM receptor polypeptide or wherein the plant LysM receptor polypeptide lacks a first JM zone 2 and the plant LysM receptor polypeptide has been modified by insertion of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 2 with the corresponding amino acids from the second JM zone 2 into the corresponding site when aligning the plant LysM receptor and the second plant LysM receptor, and wherein the first JM zone 2, the second JM zone 2, or both correspond to amino acids 256 to 280 when aligned to SEQ ID NO: 1 or correspond to amino acids 256 to 281 when aligned to SEQ ID NO: 8; (ii) a first JM zone 3, wherein the first JM zone 3 has been modified by insertion, deletion, or substitution of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 3 with the corresponding amino acids from a second JM zone 3 of a second plant LysM receptor polypeptide or wherein the plant LysM receptor polypeptide lacks a first JM zone 3 and the plant LysM receptor polypeptide has been modified by insertion of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids from the second JM zone 3 into the corresponding site when aligning the plant LysM receptor and the second plant LysM receptor, and wherein the first JM zone 3, the second JM zone 3, or both correspond to amino acids 281 to 304 when aligned to SEQ ID NO: 1 or correspond to amino acids 282 to 302 when aligned to SEQ ID NO: 8; (iii) the first kinase C-terminus region has been modified by insertion, deletion, or substitution of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the first kinase C-terminus region with the corresponding amino acids from a C-terminus region of a NFR1 LysM receptor polypeptide or wherein plant non-NFR1 LysM receptor polypeptide lacks a first C-terminus region and the plant non-NFR1 LysM receptor polypeptide has been modified by insertion of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the second C-terminus region into the corresponding site when aligning the plant non-NFR1 LysM receptor and the plant NFR1 LysM receptor, wherein the first kinase C-terminus region corresponds to amino acids T467, S471, S473, P475, G477, V496, V500, Y510, D517, 1519, V520, S523, S525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, and G621 when aligned to SEQ ID NO: 8, and/or wherein the second kinase C-terminus region corresponds to amino acids 1469, N473, T475, Q477, 1497, 1501, F511, N518, V520, L521, G524, L526, V527, A528, E529, E539, A540, N542, K543, S544, C547, D548, A549, P556, E560, E564, L568, 1570, G574, R575, R579, L583, L591, L601, E603, C605, D607, E608, S610, S613, T615, 1617, L620, and V622 when aligned to SEQ ID NO: 1; and/or (iv) a first extracellular domain modified by substituting one or more amino acids of the first extracellular domain with one or more amino acids of a second extracellular domain, wherein the second extracellular domain has a different affinity, selectivity, and/or specificity for oligosaccharides than the first extracellular domain. In an additional embodiment of this aspect, which may be combined with any one of the preceding embodiments, the modified plant non-NFR1 LysM receptor polypeptide is able to initiate NFR1-mediated root nodule symbiosis signaling.

Some aspects of the disclosure include a genetically modified plant or part thereof including the modified plant LysM receptor polypeptide of any one of the preceding embodiments that has a modified plant LysM receptor polypeptide with enhanced ROS signaling. In a further embodiment of this aspect, the modified plant LysM receptor polypeptide includes a first JM zone 4, wherein the first JM zone 4 was modified by substitution of one or more amino acids in the first JM zone 4 with the corresponding amino acids from a second JM zone 4 from a LysM receptor polypeptide with ROS signaling, and wherein the first JM zone 4 includes SEQ ID NO: 6, or is selected from the group of SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and SEQ ID NO: 52, or conservative substitutions thereof, or optionally any of the preceding amino acid sequences with one, two, or three amino acid substitutions, insertions and/or deletions, and/or wherein the second JM zone 4 includes SEQ ID NO: 13, or is selected from the group of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40, or optionally any of the preceding amino acid sequences with one, two, or three amino acid substitutions, insertions and/or deletions; and optionally further includes: (i) a first JM zone 2, wherein the first JM zone 2 has been modified by insertion, deletion, or substitution of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 2 with the corresponding amino acids from a second JM zone 2 of a second plant LysM receptor polypeptide or wherein the plant LysM receptor polypeptide lacks a first JM zone 2 and the plant LysM receptor polypeptide has been modified by insertion of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 2 with the corresponding amino acids from the second JM zone 2 into the corresponding site when aligning the plant LysM receptor and the second plant LysM receptor, and wherein the first JM zone 2, the second JM zone 2, or both correspond to amino acids 256 to 280 when aligned to SEQ ID NO: 1 or correspond to amino acids 256 to 281 when aligned to SEQ ID NO: 8; (ii) a first JM zone 3, wherein the first JM zone 3 has been modified by insertion, deletion, or substitution of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 3 with the corresponding amino acids from a second JM zone 3 of a second plant LysM receptor polypeptide or wherein the plant LysM receptor polypeptide lacks a first JM zone 3 and the plant LysM receptor polypeptide has been modified by insertion of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids from the second JM zone 3 into the corresponding site when aligning the plant LysM receptor and the second plant LysM receptor, and wherein the first JM zone 3, the second JM zone 3, or both correspond to amino acids 281 to 304 when aligned to SEQ ID NO: 1 or correspond to amino acids 282 to 302 when aligned to SEQ ID NO: 8; (iii) a first kinase N-terminus region, wherein the first kinase N-terminus region has been modified by insertion, deletion, or substitution of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the first kinase N-terminus region with the corresponding amino acids from a second N-terminus region of a CERK6 LysM receptor polypeptide or wherein the non-CERK6 plant LysM receptor polypeptide lacks the first N-terminus region and the non-CERK6 plant LysM receptor polypeptide has been modified by insertion of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the second N-terminus region into the corresponding site when aligning the non-CERK6 plant LysM receptor and the CERK6 LysM receptor, wherein the first kinase N-terminus region corresponds to D328, A338, K347, K348, T349, V356, T360, C364, H371, C384, V385, H395, D397, G402, Y404, H406, K410, E411, 5416, 5417, A425, V445, and L456 when aligned to SEQ ID NO: 1, and/or wherein the second kinase N-terminus region corresponds to A326, 5336, E345, R346, A347, M354, K358, A362, R369, S382, 1383, F393, E395, 5400, H402, R404, R408, D409, A414, T415, S423, 1443, and Y454 when aligned to SEQ ID NO: 8; and/or (iv) a first extracellular domain modified by substituting one or more amino acids of the first extracellular domain with one or more amino acids of a second extracellular domain, wherein the second extracellular domain has a different affinity, selectivity, and/or specificity for oligosaccharides than the first extracellular domain. In an additional embodiment of this aspect, which may be combined with any one of the preceding embodiments, the modified plant LysM receptor polypeptide is able to initiate ROS signaling.

In a further embodiment of the preceding aspects, which may be combined with any of the preceding embodiments that has a genetically modified plant or part thereof, the plant part is a leaf, a stem, a root, a root primordia, a flower, a seed, a fruit, a kernel, a grain, a cell, or a portion thereof. In yet another embodiment of the preceding aspects, which may be combined with any of the preceding embodiments, the plant is selected from the group of cassava, yam, sweet potato, corn, cowpea, rice, barley, wheat, Trema spp., apple, pear, plum, apricot, peach, almond, walnut, strawberry, raspberry, blackberry, red currant, black currant, melon, cucumber, pumpkin, squash, grape, bean, soybean, pea, chickpea, pigeon pea, lentil, Bambara groundnut, lupin, pulses, Medicago spp., Lotus spp., forage legumes, indigo, legume trees, or hemp.

Further aspects of the present disclosure relate to methods of producing the genetically modified plant or part thereof of any one of the preceding embodiments that has a modified plant LysM receptor polypeptide including a first JM zone 4 modified by insertion, deletion, or substitution of one or more amino acids in the first JM zone 4 with the corresponding amino acids from a second JM zone 4 able to initiate NFR1-mediated root nodule symbiosis signaling or that has a modified plant non-NFR1 LysM receptor polypeptide, including introducing a genetic alteration to the plant including a first nucleic acid sequence encoding the modified plant LysM receptor polypeptide or the modified plant non-NFR1 LysM receptor polypeptide. In an additional embodiment of this aspect, the nucleic acid sequence is operably linked to a promoter, wherein the promoter is a root specific promoter, an inducible promoter, a constitutive promoter, or a combination thereof. In a further embodiment of this aspect, the promoter is selected from the group of a NFR1 promoter, a NFR5 promoter, a LYK3 promoter, a CERK6 promoter, a NFP promoter, a Lotus japonicus NFR5 promoter (SEQ ID NO: 109), a Lotus japonicus NFR1 promoter (SEQ ID NO: 151), a Lotus japonicus CERK6 promoter (SEQ ID NO: 111), a Medicago truncatula NFP promoter (SEQ ID NO: 110), a Medicago truncatula LYK3 promoter (SEQ ID NO: 112), a maize metallothioneine promoter, a chitinase promoter, a maize ZRP2 promoter, a tomato LeExt1 promoter, a glutamine synthetase soybean root promoter, a RCC3 promoter, a rice antiquitin promoter, a LRR receptor kinase promoter, or an Arabidopsis pCO2 promoter. In yet another embodiment of this aspect, the promoter is selected from the group of a CaMV35S promoter, a derivative of the CaMV35S promoter, a maize ubiquitin promoter, a polyubiquitin promoter, a vein mosaic cassava virus promoter, or an Arabidopsis UBQ10 promoter. In yet another embodiment of this aspect, the nucleic acid sequence is inserted into the genome of the plant so that the nucleic acid sequence is operably linked to an endogenous promoter, and wherein the endogenous promoter is a root specific promoter.

Additional aspects of the present disclosure relate to methods of producing the genetically modified plant or part thereof of any one of the preceding embodiments that has a modified plant LysM receptor polypeptide including a first JM zone 4 modified by insertion, deletion, or substitution of one or more amino acids in the first JM zone 4 with the corresponding amino acids from a second JM zone 4 able to initiate NFR1-mediated root nodule symbiosis signaling or that has a modified plant non-NFR1 LysM receptor polypeptide, including genetically modifying the plant or part thereof by transforming the plant or part thereof with one or more gene editing components that target an endogenous nuclear genome sequence encoding an endogenous plant LysM receptor polypeptide or plant non-NFR1 LysM receptor polypeptide to genetically modify a first JM zone 4 by insertion, deletion, or substitution of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 4 with the corresponding amino acids from a second JM zone 4 of a second plant LysM receptor polypeptide or wherein the plant LysM receptor polypeptide lacks a first JM zone 4 and the plant LysM receptor polypeptide has been modified by insertion of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 4 with the corresponding amino acids from the second JM zone 4 into the corresponding site when aligning the plant LysM receptor and the second plant LysM receptor, and optionally further including: (i) genetically modifying a first JM zone 2 by insertion, deletion, or substitution of one or more amino acids in the first JM zone 2 with the corresponding amino acids from a second JM zone 2 of a second plant LysM receptor polypeptide; (ii) genetically modifying a first JM zone 3 by insertion, deletion, or substitution of one or more amino acids in the first JM zone 3 with the corresponding amino acids from a second JM zone 3 of a second plant LysM receptor polypeptide; (iii) genetically modifying a first kinase C-terminus region by substitution of one or more amino acids in the first kinase C-terminus region with the corresponding amino acids from a second kinase C-terminus region from a NFR1 LysM receptor polypeptide; and/or (iv) genetically modifying a first extracellular domain by substituting one or more amino acids of the first extracellular domain with one or more amino acids of a second extracellular domain, wherein the second extracellular domain has a different affinity, selectivity, and/or specificity for oligosaccharides than the first extracellular domain. In a further embodiment of this aspect, the one or more gene editing components include a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence.

Further aspects of the present disclosure relate to methods of producing the genetically modified plant or part thereof of any one of the preceding embodiments that has a modified plant LysM receptor polypeptide including a first JM zone 4 modified by insertion, deletion, or substitution of one or more amino acids in the first JM zone 4 with the corresponding amino acids from a second JM zone 4 able to ROS signaling or a modified plant LysM receptor polypeptide with enhanced ROS signaling, including introducing a genetic alteration to the plant including a first nucleic acid sequence encoding the modified plant LysM receptor polypeptide or the modified plant LysM receptor polypeptide with enhanced ROS signaling. In an additional embodiment of this aspect, the nucleic acid sequence is operably linked to a promoter, wherein the promoter is a root specific promoter, an inducible promoter, a constitutive promoter, or a combination thereof. In a further embodiment of this aspect, the promoter is selected from the group of a NFR1 promoter, a NFR5 promoter, a LYK3 promoter, a CERK6 promoter, a NFP promoter, a Lotus japonicus NFR5 promoter (SEQ ID NO: 109), a Lotus japonicus NFR1 promoter (SEQ ID NO: 151), a Lotus japonicus CERK6 promoter (SEQ ID NO: 111), a Medicago truncatula NFP promoter (SEQ ID NO: 110), a Medicago truncatula LYK3 promoter (SEQ ID NO: 112), a maize metallothioneine promoter, a chitinase promoter, a maize ZRP2 promoter, a tomato LeExt1 promoter, a glutamine synthetase soybean root promoter, a RCC3 promoter, a rice antiquitin promoter, a LRR receptor kinase promoter, or an Arabidopsis pCO2 promoter. In yet another embodiment of this aspect, the promoter is selected from the group of a CaMV35S promoter, a derivative of the CaMV35S promoter, a maize ubiquitin promoter, a polyubiquitin promoter, a vein mosaic cassava virus promoter, or an Arabidopsis UBQ10 promoter. In yet another embodiment of this aspect, the nucleic acid sequence is inserted into the genome of the plant so that the nucleic acid sequence is operably linked to an endogenous promoter, and wherein the endogenous promoter is a root specific promoter.

Additional aspects of the present disclosure relate to methods of producing the genetically modified plant or part thereof of any one of the preceding embodiments that has a modified plant LysM receptor polypeptide including a first JM zone 4 modified by insertion, deletion, or substitution of one or more amino acids in the first JM zone 4 with the corresponding amino acids from a second JM zone 4 able to ROS signaling or a modified plant LysM receptor polypeptide with enhanced ROS signaling, including genetically modifying the plant or part thereof by transforming the plant or part thereof with one or more gene editing components that target an endogenous nuclear genome sequence encoding an endogenous plant LysM receptor polypeptide to genetically modify a first JM zone 4 by insertion, deletion, or substitution of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 4 with the corresponding amino acids from a second JM zone 4 of a second plant LysM receptor polypeptide or wherein the plant LysM receptor polypeptide lacks a first JM zone 4 and the plant LysM receptor polypeptide has been modified by insertion of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 4 with the corresponding amino acids from the second JM zone 4 into the corresponding site when aligning the plant LysM receptor and the second plant LysM receptor, and optionally to further including: (i) genetically modifying a first JM zone 2 by insertion, deletion, or substitution of one or more amino acids in the first JM zone 2 with the corresponding amino acids from a second JM zone 2 of a second plant LysM receptor polypeptide; (ii) genetically modifying a first JM zone 3 by insertion, deletion, or substitution of one or more amino acids in the first JM zone 3 with the corresponding amino acids from a second JM zone 3 of a second plant LysM receptor polypeptide; (iii) genetically modifying a first kinase N-terminus region by substitution of one or more amino acids in the first kinase N-terminus region with the corresponding amino acids from a second kinase N-terminus region from a NFR1 LysM receptor polypeptide; and/or (iv) genetically modifying a first extracellular domain by substituting one or more amino acids of the first extracellular domain with one or more amino acids of a second extracellular domain, wherein the second extracellular domain has a different affinity, selectivity, and/or specificity for oligosaccharides than the first extracellular domain. In a further embodiment of this aspect, the one or more gene editing components include a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence.

A further aspect of the disclosure includes an expression vector, isolated DNA molecule, or recombinant nucleic acid including a modified plant LysM receptor polypeptide including a modified JM zone 4 domain, a modified JM zone 3 domain, a modified JM zone 2 domain, a modified kinase C-terminus region, and/or a modified extracellular domain operably linked to at least one expression control sequence. In an additional embodiment of this aspect, (i) the modified JM zone 4 was modified by substitution of one or more amino acids in a first JM zone 4 with the corresponding amino acids from a second JM zone 4 from an NFR1 LysM receptor polypeptide with NFR1-mediated root nodule symbiosis signaling, wherein the first JM zone 4 includes SEQ ID NO: 13, is selected from the group of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40, or optionally any of the preceding amino acid sequences with one, two, or three amino acid substitutions, insertions and/or deletions, or includes SEQ ID NO: 60, and/or wherein the second JM zone 4 includes SEQ ID NO: 6, or is selected from the group of SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and SEQ ID NO: 52, or conservative substitutions thereof, or optionally any of the preceding amino acid sequences with one, two, or three amino acid substitutions, insertions and/or deletions; (ii) the modified JM zone 2 was modified by insertion, deletion, or substitution of one or more amino acids in a first JM zone 2 with the corresponding amino acids from a second JM zone 2 of a second plant LysM receptor polypeptide, and wherein the first JM zone 2, the second JM zone 2, or both correspond to amino acids 256 to 280 when aligned to SEQ ID NO: 1 or correspond to amino acids 256 to 281 when aligned to SEQ ID NO: 8; (iii) the modified JM zone 3 was modified by insertion, deletion, or substitution of one or more amino acids in a first JM zone 3 with the corresponding amino acids from a second JM zone 3 of a second plant LysM receptor polypeptide, and wherein the first JM zone 3, the second JM zone 3, or both correspond to amino acids 281 to 304 when aligned to SEQ ID NO: 1 or correspond to amino acids 282 to 302 when aligned to SEQ ID NO: 8; (iv) the modified kinase C-terminus region was modified by substitution of one or more amino acids in a first kinase C-terminus region with the corresponding amino acids from a second kinase C-terminus region from a NFR1 LysM receptor polypeptide, wherein the first kinase C-terminus region corresponds to amino acids T467, S471, S473, P475, G477, V496, V500, Y510, D517, 1519, V520, S523, S525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, and G621 when aligned to SEQ ID NO: 8, and/or wherein the second kinase C-terminus region corresponds to amino acids 1469, N473, T475, Q477, 1497, 1501, F511, N518, V520, L521, G524, L526, V527, A528, E529, E539, A540, N542, K543, S544, C547, D548, A549, P556, E560, E564, L568, 1570, G574, R575, R579, L583, L591, L601, E603, C605, D607, E608, S610, S613, T615, 1617, L620, and V622 when aligned to SEQ ID NO: 1; and/or (v) the modified extracellular domain was modified by substituting one or more amino acids of the first extracellular domain with one or more amino acids of a second extracellular domain, wherein the second extracellular domain has a different affinity, selectivity, and/or specificity for oligosaccharides than the first extracellular domain.

An additional aspect of the disclosure includes an expression vector, isolated DNA molecule, or recombinant nucleic acid including a modified plant LysM receptor polypeptide including a modified JM zone 4 domain, a modified JM zone 3 domain, a modified JM zone 2 domain, a modified kinase N-terminus region, and/or a modified extracellular domain operably linked to at least one expression control sequence. In a further embodiment of this aspect, (i) the modified JM zone 4 was modified by substitution of one or more amino acids in a first JM zone 4 with the corresponding amino acids from a second JM zone 4 from a LysM receptor polypeptide with ROS signaling, and wherein the first JM zone 4 includes SEQ ID NO: 6, or is selected from the group of SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and SEQ ID NO: 52, or conservative substitutions thereof, or optionally any of the preceding amino acid sequences with one, two, or three amino acid substitutions, insertions and/or deletions, and/or wherein the second JM zone 4 includes SEQ ID NO: 13, or is selected from the group of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40, or optionally any of the preceding amino acid sequences with one, two, or three amino acid substitutions, insertions and/or deletions; (ii) the modified JM zone 2 was modified by insertion, deletion, or substitution of one or more amino acids in a first JM zone 2 with the corresponding amino acids from a second JM zone 2 of a second plant LysM receptor polypeptide, and wherein the first JM zone 2, the second JM zone 2, or both correspond to amino acids 256 to 280 when aligned to SEQ ID NO: 1 or correspond to amino acids 256 to 281 when aligned to SEQ ID NO: 8; (iii) the modified JM zone 3 was modified by insertion, deletion, or substitution of one or more amino acids in a first JM zone 3 with the corresponding amino acids from a second JM zone 3 of a second plant LysM receptor polypeptide, and wherein the first JM zone 3, the second JM zone 3, or both correspond to amino acids 281 to 304 when aligned to SEQ ID NO: 1 or correspond to amino acids 282 to 302 when aligned to SEQ ID NO: 8; (iv) the modified kinase N-terminus region was modified by substitution of one or more amino acids in a first kinase N-terminus region with the corresponding amino acids from a second kinase N-terminus region from a CERK6 LysM receptor polypeptide, wherein the first kinase N-terminus region corresponds to D328, A338, K347, K348, T349, V356, T360, C364, H371, C384, V385, H395, D397, G402, Y404, H406, K410, E411, S416, S417, A425, V445, and L456 when aligned to SEQ ID NO: 1, and/or wherein the second kinase N-terminus region corresponds to A326, S336, E345, R346, A347, M354, K358, A362, R369, S382, 1383, F393, E395, S400, H402, R404, R408, D409, A414, T415, S423, 1443, and Y454 when aligned to SEQ ID NO: 8; and/or (v) the modified extracellular domain was modified by substituting one or more amino acids of the first extracellular domain with one or more amino acids of a second extracellular domain, wherein the second extracellular domain has a different affinity, selectivity, and/or specificity for oligosaccharides than the first extracellular domain.

In a further embodiment of the preceding aspects, which may be combined with any of the preceding embodiments that has an expression vector, isolated DNA molecular, or recombinant nucleic acid, the at least one expression control sequence includes a promoter selected from the group of a root specific promoter, a constitutive promoter, or a combination thereof. In an additional embodiment of this aspect, the promoter is a root specific promoter, and wherein the promoter is selected from the group of a NFR1 promoter, a NFR5 promoter, a LYK3 promoter, a CERK6 promoter, a NFP promoter, a Lotus japonicus NFR5 promoter (SEQ ID NO: 109), a Lotus japonicus NFR1 promoter (SEQ ID NO: 151), a Lotus japonicus CERK6 promoter (SEQ ID NO: 111), a Medicago truncatula NFP promoter (SEQ ID NO: 110), a Medicago truncatula LYK3 promoter (SEQ ID NO: 112), a maize metallothioneine promoter, a chitinase promoter, a maize ZRP2 promoter, a tomato LeExt1 promoter, a glutamine synthetase soybean root promoter, a RCC3 promoter, a rice antiquitin promoter, a LRR receptor kinase promoter, or an Arabidopsis pCO2 promoter. In a further embodiment of this aspect, the promoter is constitutive promoter, and wherein the promoter is selected from the group of a CaMV35S promoter, a derivative of the CaMV35S promoter, a maize ubiquitin promoter, a polyubiquitin promoter, a vein mosaic cassava virus promoter, or an Arabidopsis UBQ10 promoter.

Some aspects of the disclosure include a bacterial cell or an Agrobacterium cell including the expression vector, isolated DNA molecule, or recombinant nucleic acid of any one of the preceding embodiments.

Further aspects of the disclosure include a genetically modified plant, plant part, plant cell, or seed including the expression vector, isolated DNA molecule, or recombinant nucleic acid of any one of the preceding embodiments.

Additional aspects of the disclosure include a composition or kit including the expression vector, isolated DNA molecule, or recombinant nucleic acid of any one of the preceding embodiments, the bacterial cell or Agrobacterium cell of any one of the preceding embodiments, or the genetically modified plant, plant part, plant cell, or seed of any one of the preceding embodiments.

Yet further aspects of the disclosure include methods of initiating NFR1-mediated root nodule symbiosis signaling including: introducing a genetic alteration via the expression vector, isolated DNA molecule, or recombinant nucleic acid of any one of the preceding embodiments that has a modified kinase C-terminus region.

Still further aspects of the disclosure include methods of initiating ROS signaling including: introducing a genetic alteration via the expression vector, isolated DNA molecule, or recombinant nucleic acid of any one of the preceding embodiments that has a modified kinase N-terminus region.

In a further embodiment of the preceding aspects, which may be combined with any of the preceding embodiments that has a method of initiating NFR1-mediated root nodule symbiosis signaling or ROS signaling, the plant is a plant cell.

An additional aspect of the disclosure includes methods of generating a modified plant LysM receptor polypeptide, including: (a) aligning an amino acid sequence of a candidate receptor to SEQ ID NO: 8 to identify a first JM zone 4 corresponding to amino acids 303 to 325 of SEQ ID NO: 8, or aligning an amino acid sequence of a candidate receptor to SEQ ID NO: 1 to identify a first JM zone 4 corresponding to amino acids 305-327 of SEQ ID NO: 1, and optionally further aligning the JM zone 4 of the candidate receptor to SEQ ID NO: 13, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40, or optionally any of the preceding amino acid sequences with one, two, or three amino acid substitutions, insertions and/or deletions; (b) modifying the first JM zone 4 by substituting at least two or at least three amino acid residues in the first JM zone 4 with corresponding amino acid residues that are different in a second JM zone 4; and (c) generating the modified plant LysM receptor polypeptide wherein the first JM zone 4 has been substituted with corresponding amino acid residues from the second JM zone 4.

A further aspect of the disclosure includes methods of generating a modified plant LysM receptor polypeptide, including: (a) aligning an amino acid sequence of a candidate receptor to SEQ ID NO: 8 to identify a first JM zone 2 corresponding to amino acids 256 to 281 of SEQ ID NO: 8, or aligning an amino acid sequence of a candidate receptor to SEQ ID NO: 1 to identify a first JM zone 2 corresponding to amino acids 256-280 of SEQ ID NO: 1; (b) modifying the first JM zone 2 by substituting inserting, deleting, or substituting one or more amino acid residues in the first JM zone 2 with corresponding amino acid residues that are different in a second JM zone 2; and (c) generating the modified plant LysM receptor polypeptide wherein the first JM zone 2 has been substituted with corresponding amino acid residues from the second JM zone 2.

Yet another aspect of the disclosure includes methods of generating a modified plant LysM receptor polypeptide, including: (a) aligning an amino acid sequence of a candidate receptor to SEQ ID NO: 8 to identify a first JM zone 3 corresponding to amino acids 282 to 302 of SEQ ID NO: 8, or aligning an amino acid sequence of a candidate receptor to SEQ ID NO: 1 to identify a first JM zone 3 corresponding to amino acids 281-304 of SEQ ID NO: 1; (b) modifying the first JM zone 3 by substituting inserting, deleting, or substituting one or more amino acid residues in the first JM zone 3 with corresponding amino acid residues that are different in a second JM zone 3; and (c) generating the modified plant LysM receptor polypeptide wherein the first JM zone 3 has been substituted with corresponding amino acid residues from the second JM zone 3.

Still another aspect of the disclosure includes methods of generating a modified plant LysM receptor polypeptide, including: (a) aligning an amino acid sequence of a candidate receptor to SEQ ID NO: 8 to identify a first kinase C-terminus region corresponding to amino acids T467, S471, S473, P475, G477, V496, V500, Y510, D517, 1519, V520, S523, S525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, and G621 of SEQ ID NO: 8, or aligning an amino acid sequence of a candidate receptor to SEQ ID NO: 1 to identify a first kinase C-terminus region corresponding to amino acids 1469, N473, T475, Q477, 1497, 1501, F511, N518, V520, L521, G524, L526, V527, A528, E529, E539, A540, N542, K543, S544, C547, D548, A549, P556, E560, E564, L568, 1570, G574, R575, R579, L583, L591, L601, E603, C605, D607, E608, S610, S613, T615, 1617, L620, and V622 of SEQ ID NO: 1; (b) modifying the first kinase C-terminus region by substituting one or more amino acid residues in the first kinase C-terminus region with corresponding amino acid residues that are different in a second kinase C-terminus region; and (c) generating the modified plant LysM receptor polypeptide wherein the first kinase C-terminus region has been substituted with corresponding amino acid residues from the second kinase C-terminus region.

An additional aspect of the disclosure includes methods of generating a modified plant LysM receptor polypeptide, including: (a) aligning an amino acid sequence of a candidate receptor to SEQ ID NO: 1 to identify a first kinase N-terminus region corresponding to amino acids D328, A338, K347, K348, T349, V356, T360, C364, H371, C384, V385, H395, D397, G402, Y404, H406, K410, E411, S416, S417, A425, V445, and L456 of SEQ ID NO: 1, or aligning an amino acid sequence of a candidate receptor to SEQ ID NO: 8 to identify a first kinase N-terminus region corresponding to amino acids A326, S336, E345, R346, A347, M354, K358, A362, R369, S382, 1383, F393, E395, S400, H402, R404, R408, D409, A414, T415, S423, 1443, and Y454 of SEQ ID NO: 8; (b) modifying the first kinase N-terminus region by substituting one or more amino acid residues in the first kinase N-terminus region with corresponding amino acid residues that are different in a second kinase N-terminus region; and (c) generating the modified plant LysM receptor polypeptide wherein the first kinase N-terminus region has been substituted with corresponding amino acid residues from the second kinase N-terminus region.

Further embodiments of the preceding aspects, which may be combined with any of the preceding embodiments that has methods of generating a modified plant LysM receptor polypeptide, include the modified plant LysM receptor polypeptide produced by any of the methods of any one of the preceding embodiments, or a combination thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B compare sequences and shared cellular use of NFR1 and CERK6. FIG. 1A shows an alignment of the NFR1 (top; SEQ ID NO: 1) and CERK6 (bottom; SEQ ID NO: 8) protein sequences. Numbers on top of the alignment indicate the position of the corresponding amino acid in the NFR1 protein sequence. Numbers at the right end of each row indicate the number of amino acids contained in NFR1 or CERK6 in each row of the alignment. The ectodomain (EC) is shaded in blue (NFR1 EC=SEQ ID NO: 2; CERK6 EC=SEQ ID NO: 9), the transmembrane/juxtamembrane domains (TM/JM) are shaded in green, and the kinase domains (KD) are shaded in red (NFR1 KD=SEQ ID NO: 7; CERK6 KD=SEQ ID NO: 14). TM/JM zones 1-4 are divided by red dashed lines and respectively labeled in black; zone 1 is part of the TM (NFR1 zone 1=SEQ ID NO: 3; CERK6 zone 1=SEQ ID NO: 10), while zones 2-4 are part of the JM (NFR1 zone 2=SEQ ID NO: 4; NFR1 zone 3=SEQ ID NO: 5; NFR1 zone 4=SEQ ID NO: 6; CERK6 zone 2=SEQ ID NO: 11; CERK6 zone 3=SEQ ID NO: 12; CERK6 zone 4=SEQ ID NO: 13). FIG. 1B shows dimensionality reduction of cell transcriptomics to judge cellular use of NFR1, CERK6, or both. Hotspots of NFR1 are blue, and hotspots of CERK6 are red.

FIGS. 2A-2K show that LjNFR1 JM zone 4 is essential for symbiotic signaling. FIG. 2A shows a schematic representation of the basic NFR1-CERK6 chimera used for the creation of the chimeric constructs 1-16 (depicted in FIGS. 2B-2I), with the ectodomain (EC) shown at the top; the transmembrane (TM) and juxtamembrane (JM) domains shown in the center (TM/JM), with number 1 indicating zone 1 (NFR1 zone 1=SEQ ID NO: 3; CERK6 zone 1=SEQ ID NO: 10), and numbers 2-4 indicating JM zones 2-4 (NFR1 zone 2=SEQ ID NO: 4; NFR1 zone 3=SEQ ID NO: 5; NFR1 zone 4=SEQ ID NO: 6; CERK6 zone 2=SEQ ID NO: 11; CERK6 zone 3=SEQ ID NO: 12; CERK6 zone 4=SEQ ID NO: 13); and the kinase domain (K) shown at the bottom. The coloring indicates the source of each segment, with portions from NFR1 shaded in magenta, and portions from CERK6 shaded in green. Each chimeric construct contains the ectodomain of NFR1 and the kinase of CERK6 coupled to different NFR1-CERK6 chimeric versions of the TM/JM. A black arrow indicates the region (TM/JM) in which the structures of chimeric constructs 1-15 in FIGS. 2B-2C differ from each other.

FIG. 2B shows a box-and-whiskers plot displaying nodule formation in nfr1-1 pNin:GUS Lotus japonicus roots expressing receptor chimeras (magenta- and green-shaded constructs; labeled 1-15 on the x-axis), the full-length NFR1 construct (magenta construct; third label from left on the x-axis) or an empty vector (second label from the left on the x-axis); or in a wild type L. japonicus control (labeled “Gifu”) plants expressing an empty vector (left-most label on the x-axis). The number of nodules per plant is displayed on the y-axis. Chimeras are shaded as in FIG. 2A. Lowercase letters indicate significant differences between samples as demonstrated by a Kruskal-Wallis analysis of variance test with p<0.05. Fractions on the bottom of the boxplot indicate the number of nodulating plants out of the total plants tested for each chimeric construct. FIG. 2C shows a box-and-whiskers plot displaying nodule formation in nfr1-1 pNin:GUS Lotus japonicus roots expressing receptor chimeras (red- and dark grey-shaded constructs; labeled 1-16 on the x-axis), the full-length NFR1 construct (light grey construct; third label from left on the x-axis) or an empty vector (second label from the left on the x-axis); or in a wild type L. japonicus control (labeled “Gifu”) plants expressing an empty vector (left-most label on the x-axis). The number of nodules per plant is displayed on the y-axis. Pink nodules are plotted in pink, and white nodules are plotted in white. Lowercase letters indicate significant differences between samples as demonstrated by a Kruskal-Wallis analysis of variance test with p<0.05. Fractions on the bottom of the boxplot indicate the number of nodulating plants out of the total plants tested for each chimeric construct. FIGS. 2D-2E show microscopic images of the nodule formation/infection phenotype of nfr1-1 pNIN:GUS roots expressing an empty vector in a wild type L. japonicus control (“Gifu”) (top row), an empty vector in a nfr1-1 pNIN:GUS L. japonicus control (second row), or chimeric constructs as depicted in FIG. 2C, with FIG. 2D showing constructs 1-7 and FIG. 2E showing constructs 8-16. Bright field images of unstained samples are shown on the left; fluorescent microscopy images of the triple yellow fluorescent protein (YFP) transformation marker are shown in the second column from the left; fluorescent microscopy images of the DsRed M. loti infection marker are shown in the third column from the left; and bright field images showing GUS staining (blue) indicating NIN promoter expression (indicating symbiotic signaling) in samples expressing NIN-GUS are shown on the right. Scale bars represent 5 mm. FIG. 2F shows microscopic images of the nodule formation/infection phenotype of nfr1-1 pNIN:GUS roots expressing an nfr1 empty vector (top row), a wild type NFR1 protein (second row), or chimeric constructs 12 (third row) or 5 (bottom row) as depicted in FIG. 2B. Bright field images of unstained samples are shown on the left; fluorescent microscopy images of the triple yellow fluorescent protein (YFP) transformation marker are shown in the second column from the left; fluorescent microscopy images of the DsRed M. loti infection marker are shown in the third column from the left; and bright field images showing GUS staining (blue) indicating NIN promoter expression (indicating symbiotic signaling) in samples expressing NIN-GUS are shown on the right. Scale bars represent 5 mm. FIG. 2G shows microscopic images of the nodule formation/infection phenotype in nfr1-1 pNin:GUS Lotus japonicus roots expressing receptor chimeras labeled 1, 6, and 13 (consistent with FIG. 2C). Bright field images of unstained samples are shown on the left; fluorescent microscopy images of the triple yellow fluorescent protein (YFP) transformation marker are shown in the second column from the left; fluorescent microscopy images of the DsRed M. loti infection marker are shown in the third column from the left; and bright field images showing GUS staining (blue) indicating NIN promoter expression (indicating symbiotic signaling) in samples expressing NIN-GUS are shown on the right. Scale bars represent 5 mm. FIG. 2H shows a box-and-whiskers plot displaying infection thread formation in nfr1-1 pNin:GUS Lotus japonicus roots expressing receptor chimeras (magenta- and green-shaded constructs; bottom right two labels on the x-axis), the full-length NFR1 construct (magenta construct; third label from left on the x-axis) or an empty vector (second label from the left on the x-axis); or in a wild type L. japonicus control (labeled “Gifu”) plants expressing an empty vector (left-most label on the x-axis). The number of infection threads (ITs) per plant is displayed on the y-axis. Chimeras are depicted and shaded according to the scheme displayed at the bottom right, with the EC shown at the top, the TM/JM zones shown in the middle, and the K domain shown at the bottom, with magenta shading indicating portions from NFR1 and green shading indicating portions from CERK6. FIG. 21 shows a box-and-whiskers plot displaying infection thread formation in nfr1-1 pNin:GUS Lotus japonicus roots expressing receptor chimeras (dark grey- and red-shaded constructs; bottom right two labels on the x-axis), the full-length NFR1 construct (red construct; third label from left on the x-axis) or an empty vector (second label from the left on the x-axis); or in a wild type L. japonicus control (labeled “Gifu”) plants expressing an empty vector (left-most label on the x-axis). The number of infection threads (ITs) per cm of hairy root is displayed on the y-axis. Chimeras are depicted and shaded according to the scheme displayed at the bottom right, with the EC shown at the top, the TM/JM zones shown in the middle, and the K domain shown at the bottom, with red shading indicating portions from NFR1 and black shading indicating portions from CERK6. FIG. 2J shows confocal microscopy images of transgenic nfr1 roots expressing the construct indicated above each image. Constructs are depicted according to the scheme shown at the bottom right of FIG. 2H. Images are overlays of root autofluorescence (lasers/emission cutoffs: 405/408-498 nm (autofluorescence)) and the red fluorescent channel. Red shows M. loti MAFF bacteria. Yellow arrowheads indicate branched and scrambled root hair tips where bacteria micro-colonies are attached. Scale bars indicate 100 microns. FIG. 2K shows additional confocal microscopy images of transgenic nfr1 roots expressing the construct indicated above each image. Constructs are numbered according to the scheme shown in FIG. 2C, and empty vectors are labeled “EV”, the leftmost of which is expressed in a wild type L. japonicus control (labeled “Gifu”). Images are overlays of root autofluorescence (lasers/emission cutoffs: 405/408-498 nm (autofluorescence)) and the red fluorescent channel. Arrowheads and red indicate M. loti MAFF bacteria. In FIGS. 2D-2F, scale bars indicate 5 mm. In FIG. 2J, the scale bar indicates 100 microns. In FIG. 2K, the scale bar indicates 30 microns.

FIGS. 3A-3B show that CERK6 JM zone 4 (SEQ ID NO: 13) alone does not inhibit nodule organogenesis. FIG. 3A shows a box-and-whisker plot depicting nodule formation assays indicating that the CERK6 JM zone 4 alone embedded in the NFR1 receptor does not inhibit nodule organogenesis. The right-most portions of the plot (with top shading labeled “nfr1_1_pNin_GUS”) show nfr1-1 pNin:GUS Lotus japonicus roots expressing the NFR1-CERK6 receptor chimeras indicated under the x-axis. The chimeras are shaded as in FIG. 2A, with the ectodomain (EC) shown at the top of the chimera; the transmembrane (TM) and juxtamembrane (JM) domains shown in the center (TM/JM); and the kinase domain (K) shown at the bottom. The coloring indicates the source of each segment, with portions from NFR1 shaded in magenta, and portions from CERK6 shaded in green. “Gifu” indicates a wild type L. japonicus control. Lowercase letters indicate significant differences between samples as indicated by a Kruskal-Wallis analysis of variance test, with p<0.05. FIG. 3B shows micrographs of the nodule formation/infection phenotype of nfr1-1 pNIN:GUS roots expressing the chimeras indicated along the left. The chimera schematics in the bottom two rows correspond to the middle and far-right chimeras as in FIG. 3A. The far left micrographs are bright field images; the column labeled “YFP” shows fluorescent microscopy images of the yellow fluorescent protein (YFP) transformation marker; the column labeled “M. Loti DsRed” shows fluorescent microscopy images of the DsRed M. loti infection marker; and the column labeled “pNIN:GUS” shows bright field images showing GUS staining (blue) indicating NIN promoter expression (indicating symbiotic signaling) in samples expressing. Scale bars represent 5 mm.

FIG. 4 shows LjCERK6 zone 4- and kinase-containing chimeric receptors localized at the plasma membrane in tobacco (Nicotiana benthamiana) leaves. LjCERK6 zone 4 is shown in SEQ ID NO: 13. LjNFR1-LjCERK6(K35IN) chimeras tagged with YFP at the C-terminus and driven by a 35S promoter were transiently co-expressed with a plasma membrane marker, AtPIP2A tagged with mCherry at the C-terminus and driven by a ubiquitin promoter, in N. benthamiana leaves. The top row shows a schematic of the chimeric receptor expressed in each column. The rectangles on the far left in each schematic represent the ectodomain (EC); the series of four black-outlined hexagons in the middle of each schematic represent the transmembrane (TM) and juxtamembrane (JM) domains; and the oval on the far left of each schematic represents the kinase domain (K). The color key on the far left of the top row indicates the source of each of the EC, TM/JM, and K domains, with portions from LjNFR1 shaded in magenta, and portions from LjCERK6 shaded in green. The red stars labeled “K35iN” indicate K domains that include the K351N mutation that creates a “kinase-dead” version of the CERK6 kinase. The left column shows the full native LjNFR1 tagged with YFP; the second column from the left shows the full inactive mutant LjCERK6(K35IN) tagged with YFP; and the right-most three rows show three different chimeric receptor proteins, each with the LjNFR1 ectodomain and the LjCERK6(K35IN) kinase domain and various combinations of NFR1 and CERK6 TM/JM domain zones (middle column: zones 1-4 from LjCERK6; fourth column from the left: zones 1-3 from LjNFR1 and zone 4 from LjCERK6; right-most column: zones 1 and 4 from LjCERK6 and zones 2-3 from LjNFR1; TM/JM zone 1 is part of the TM (NFR1 zone 1=SEQ ID NO: 3; CERK6 zone 1=SEQ ID NO: 10), while zones 2-4 are part of the JM (NFR1 zone 2=SEQ ID NO: 4; NFR1 zone 3=SEQ ID NO: 5; NFR1 zone 4=SEQ ID NO: 6; CERK6 zone 2=SEQ ID NO: 11; CERK6 zone 3=SEQ ID NO: 12; CERK6 zone 4=SEQ ID NO: 13).; CERK6 zone 4=SEQ ID NO: 13). The row labeled “PM mCherry” shows fluorescent micrographs of the mCherry channel in N. benthamiana leaves co-expressing the respective YFP-tagged chimeric receptor shown along the top with AtPIP2A-mCherry, in which red indicates AtPIP2A-mCherry localized to the plasma membrane. The row labeled “chimera-YFP”shows fluorescent micrographs of the YFP channel in the same N. benthamiana leaves co-expressing the respective YFP-tagged chimeric receptor shown above with AtPIP2A-mCherry, in which green indicates expression of the YFP-tagged chimeric receptor. The row labeled “Merged” shows an overlay of the “PM mCherry” and “chimera-YFP” fluorescent micrographs in each row, in which yellow indicates co-localization of the fluorescence from each channel. Scale bars represent 50 μm.

FIGS. 5A-5C show alignment and phylogenetic analyses of protein sequences of JM zone 4 of receptor-like kinases (RLKs) in nodulating and non-nodulating species. FIG. 5A shows an alignment of CERK (top) and NFR (bottom) RLK types among accessions of nodulating and non-nodulating species in and out of the FaFaCuRo (Fabales, Fagales, Curcubitales and Rosales) clade. The CERKs are from Lotus japonicus (LjCERK6=SEQ ID NO: 15), Medicago truncatula (Medicago_CERK1=SEQ ID NO: 16), Cajanus cajan (Cajanus_020220445 aa=SEQ ID NO: 17), Phaseolus vulgaris (Phaseolus_006G006700.1 aa=SEQ ID NO: 18), Glycine max (Glycine_CP_003555584 aa=SEQ ID NO: 19), Abrus precatorius (Abrus_027343427.1 aa=SEQ ID NO: 20), Nissolia schottii (Nissolia_11875513201 aa=SEQ ID NO: 21), Arachis hypogaea (Arachis_025645378.1 aa=SEQ ID NO: 22; Arachis_025693415 aa=SEQ ID NO: 23), Castanospermum australe (Casaus_05188=SEQ ID NO: 24), Cicer arietinum (Cicer_004502028 aa=SEQ ID NO: 25), Pisum sativum (Pisum_5gl12080 aa=SEQ ID NO: 26), Lupinus angustifolius (Lupinus_XP_019425563 aa=SEQ ID NO: 27; Lupinus_XP_019455825 aa=SEQ ID NO: 30), Cercis chinensis (Cercis_CDH30701(LYK2)=SEQ ID NO: 28), Chamaecrista fasciculata (Chamaecrista_QANZ01053660=SEQ ID NO: 29; Chamaecrista_3879S02200 aa=SEQ ID NO: 33), Mimosa pudica (Mimosa_scaffold38169_cov204_SR_edit_AA=SEQ ID NO: 31; Mimosa_scaffold8583_SR_edit_AA=SEQ ID NO: 32), Trifolium pratense (Trifilum_PNY08765.1_SR_edited_=SEQ ID NO: 34), Manihot esculenta (MesCERKa=SEQ ID NO: 35; MesCERKb=SEQ ID NO: 36; MesLYKI=SEQ ID NO: 39), Parasponia andersonii (PanLYK3.1_PON42545=SEQ ID NO: 37; PanLYK3.2_PON42546=SEQ ID NO: 38), and Hordeum vulgare (HvRLK4=SEQ ID NO: 40). The NFRs are from Lotus japonicus (Lj_NFR1 aa=SEQ ID NO: 41), Medicago truncatula (Medicago_LYK3.1 aa=SEQ ID NO: 42), Glycine max (Glycin_NFR1B aa=SEQ ID NO: 43; Glycin_NFR1A.1 aa=SEQ ID NO: 44), Cajanus cajan (Cajanus_020213700 aa=SEQ ID NO:45), Phaseolus vulgaris (Phaseolus_008G211100.1 aa=SEQ ID NO: 46), Abrus precatorius (Abrus_027332267.1 aa=SEQ ID NO: 47), Cicer arietinum (Cicer_004491136 aa=SEQ ID NO: 48), Lupinus angustifolius (Lupinus_019461629 aa=SEQ ID NO: 49; Lupinus_019434083 aa=SEQ ID NO: 50), and Arachis sp. (Arachis_029150476 aa=SEQ ID NO: 51; arachis_029144024.1 aa=SEQ ID NO: 52). Red arrows indicate the Lotus japonicus receptors. Residues in blue boxes are conserved across more than 80% of the analyzed accessions. Residues shown in black are conserved in more than 50% of the analyzed accessions. Residues shown in red are conserved in less than 50% of the analyzed accessions. A sequence logo (SEQ ID NO: 113) for the analyzed accessions is shown at the bottom. The vertical black line to the left of the sequence logo displays a scale in bits, in which increased size of the residue indicates higher conservation, with the top representing 4.3 bits and the bottom representing 0.0 bits at the bottom. FIG. 5B shows an additional alignment of CERK (top) and NFR (bottom) RLK type receptor zone JM B portions of nodulating and non-nodulating species in and out of the FaFaCuRo (Fabales, Fagales, Curcubitales and Rosales) clade. The CERKs are from Lotus japonicus (LjCERK6=SEQ ID NO: 15), Medicago truncatula (Medicago truncatula CERK1=SEQ ID NO: 16), Cajanus cajan (Cajanus cajan LYK3=SEQ ID NO: 17), Phaseolus vulgaris (Phaseolus vulgaris LYK=SEQ ID NO: 18), Glycine max (Glycine max LYK3=SEQ ID NO: 19), Abrus precatorius (Abrus precatorius LYK3=SEQ ID NO: 20), Arachis hypogaea (Ah XP_025645378.1=SEQ ID NO: 22; Ah XP_02569415=SEQ ID NO: 23), Castanospermum australe (Casaus XP_05188=SEQ ID NO: 24), Cicer arietinum (Ca XP_004502028=SEQ ID NO: 25), Pisum sativum (Ps XP_5gl12080=SEQ ID NO: 26), Lupinus angustifolius (La_XP_019425563=SEQ ID NO: 27), Cercis chinensis (Cercis XP_LYK2)=SEQ ID NO: 28), Chamaecrista fasciculata (Cf XP_QANZ01053660=SEQ ID NO: 29; Cf XP_2879S02200=SEQ ID NO: 33), Mimosa pudica (Mimosa_scaffold38169_cov204=SEQ ID NO: 31; Mp XP_scaffold8584=SEQ ID NO: 32), Trifolium pratense (Tp XP_PNY08765.1=SEQ ID NO: 34), Parasponia andersonii (PanLYK3.1=SEQ ID NO: 37; PanLYK3.2=SEQ ID NO: 38), Oryza sativa (Os CERK1=SEQ ID NO: 159), Hordeum vulgare (HvRLK4=SEQ ID NO: 40), and Arabidopsis thaliana (At CERK1=SEQ ID NO: 158). The NFRs (in the grey box) are from Lotus japonicus (Lj NFR1=SEQ ID NO: 41), Medicago truncatula (Mt LYK3=SEQ ID NO: 42), Glycine max (Gm NFR1B=SEQ ID NO: 43; Glycin_NFR1A=SEQ ID NO: 44), Cajanus cajan (Cc XP_020213700=SEQ ID NO:45), Phaseolus vulgaris (Pv XP_008G211100.1=SEQ ID NO: 46), Abrus precatorius (Ap XP_027332267.1=SEQ ID NO: 47), Cicer arietinum (Ca_004491136=SEQ ID NO: 48), Lupinus angustifolius (La XP_019461629=SEQ ID NO: 49; La XP_019434083=SEQ ID NO: 50), Trifolium pratense (Tp XP_14944=SEQ ID NO: 156; Tp XP_14929=SEQ ID NO: 157) and Arachis hypogaea (Ah XP_029150476=SEQ ID NO: 51; Ah XP_029144024=SEQ ID NO: 52). Residues in solid red boxes are conserved across more than 80% of the analyzed accessions. Residues shown in black are conserved in more than 50% of the analyzed accessions. Residues shown in blue text are conserved in less than 50% of the analyzed accessions. FIG. 5C shows a phylogenetic tree created with a bootstrap value of 100 based on the alignment in FIG. 5A. Clade lines shown in green represent CERK receptor types. Clade lines shown in magenta represent the NFR receptor types. The gene name corresponding to each displayed clade is shown in purple at the end of the respective clade line.

FIGS. 6A-6E show non-polar and charged signatures in the JM B or zone 4 of NFR1 and CERK6. FIG. 6A shows an alignment of the JM zones of LjNFR1 (top; SEQ ID NO: 152) and LjCERK6 (bottom; SEQ ID NO: 153). Zone JM B is boxed on the right and labeled. Shaded red amino acids indicate the positions of the conserved amino acids between the two sequences. FIG. 6B shows an alignment between JM zone 4 of LjNFR1 (top; SEQ ID NO: 6) and LjCERK6 (bottom; SEQ ID NO: 13). The full sequence of LjNFR1 JM zone 4 is displayed in the top row (SEQ ID NO: 53). Variable residues in LjCERK6 compared to LjNFR1 are displayed in the bottom row (SEQ ID NO: 54). Dots represent conserved residues. Colors indicates the polarity of the residue, with non-polar residues shown in black, polar residues shown in green, positively-charged residues shown in blue, and negatively-charged residues shown in red. FIG. 6C shows an alignment of zone 4 compared between LjNFR1 (top; SEQ ID NO: 154) and LjCERK6 (bottom; SEQ ID NO: 155), with shaded blue amino acids indicating the positions of the conserved amino acids between the two sequences, These conserved amino acids are numbered by amino acid position above each conserved site. A schematic of an exemplary construct is shown on the left, as an example of where within the construct zone 4 is located. These conserved sites are locations of single substitutions as shown in later figures and described in the Examples. FIG. 6D shows an overlay of the NFR1 model (magenta) and the CERK6 crystal structure (green) superimposed. The dashed rectangle highlights the zone 4 region that is magnified in FIGS. 6A-C and FIG. 6E, with the shaded amino acids indicating the positions of the conserved amino acids that separate NFR-type receptors from CERK-type receptors. FIG. 6E shows magnified views of the dashed rectangle region indicated in FIG. 6D in each of the NFR1 model (magenta, left) and CERK6 crystal structure (green, right), positioned side-by-side for ease of comparison. The six conserved amino acids that separate NFR-type from CERK-type receptors are labeled in each structure, with the exception of T304 of CERK6 JM zone 4, since the crystalized CERK6 molecule starts from D306.

FIGS. 7A-7D show construct schematics and results demonstrating the role of LjNFR1 M306, A308D, and K320T in symbiotic signaling. FIG. 7A shows a schematic representation of the basic NFR1-CERK6 chimera (“Chimera A”) used for the creation of the chimeric constructs tested in FIG. 7B and FIG. 7C, with the ectodomain (EC) shown at the top; the transmembrane (TM) and juxtamembrane (JM) domains shown in the center (TM/JM), with numbers 1 to 4 indicating the different TM/JM zones; and the kinase domain (K) shown at the bottom. Zone 1 is part of the TM (NFR1 zone 1=SEQ ID NO: 3; CERK6 zone 1=SEQ ID NO: 10), while zones 2-4 are part of the JM (NFR1 zone 2=SEQ ID NO: 4; NFR1 zone 3=SEQ ID NO: 5; NFR1 zone 4=SEQ ID NO: 6; CERK6 zone 2=SEQ ID NO: 11; CERK6 zone 3=SEQ ID NO: 12; CERK6 zone 4=SEQ ID NO: 13). The coloring indicates the source of each segment, with portions from NFR1 shaded in magenta, and portions from CERK6 shaded in green. A black arrow indicates the region in the JM in which the amino acid swap mutations in FIGS. 7B-7C are located. FIG. 7B shows a box-and-whiskers plot of nodule formation in the tested constructs. The vertical axis displays nodules per plant. Data from pink (indicating functional) nodules are displayed in pink; data from white (indicating uninfected) nodules are displayed in white. The constructs from which the data sets are derived are shown across the horizontal axis. The far left data shows a control sample of an empty vector in a wild type L. japonicus background as a control (labeled “Gifu”). The second from the left shows a control sample of an empty vector expressed in the L. japonicus nfr1-1 pNIN:GUS background. The right-most nine data sets are from chimeric constructs expressed in the nfr1-1 pNIN:GUS background. The schematic constructs on the horizontal axis are shaded with the NFR1 full-length control shown in all red, NFR1 contributions to constructs shown in red, and CERK1 contributions to constructs shown in black. The labels M306T, A308D, M311V, Q316D, K320T, and N323D refer to constructs matching the schematic shown in FIG. 7A and further containing the annotated mutation (M306T, A308D, M311V, Q316D, K320T, or N323D, which were designed to each incorporate one of the variable residues shown in FIG. 6B) at roughly the position indicated by the black arrow in FIG. 7A. Different lowercase letters indicate significant differences in the formation of total nodules (pink and white) among genotypes between samples as indicated by a Kruskal-Wallis analysis of variance, with p<0.05. Fractions under the boxplots indicate the frequency of nodulating plants observed for each chimera. FIG. 7C shows the nodule formation/infection phenotypes of nfr1-1 pNIN:GUS roots expressing the chimeras containing M306T (bottom left), A308D (top right), or K320T (bottom right) amino acid swaps compared to the full-length LjNFR1 construct (top left). Bright field images are shown on the left for each construct; the middle columns show fluorescent micrographs displaying the YFP channel, indicating expression of a triple yellow fluorescent protein transformation marker (green); and the right columns show the DsRed channel, indicating expression of a M. loti infection marker (red). Scale bars indicate 5 mm. FIG. 7D shows nodule sections in either mature nodules filled with M. loti R7A in roots of nfr1 expressing full length NFR1 (left), or uninfected nodule primordium in roots of nfr1 expressing the chimeric variant shown in FIG. 7D containing the A308D substitution (right).

FIGS. 8A-8D show that the A308D and K320T amino acid swaps in LjNFR1 JM zone 4 induce massive nodule primordia formation. Constructs are labeled and/or shaded according to the scheme described above for FIG. 7B. NFR1 zone 1=SEQ ID NO: 3; CERK6 zone 1=SEQ ID NO: 10; NFR1 zone 2=SEQ ID NO: 4; NFR1 zone 3=SEQ ID NO: 5; NFR1 zone 4=SEQ ID NO: 6; CERK6 zone 2=SEQ ID NO: 11; CERK6 zone 3=SEQ ID NO: 12; CERK6 zone 4=SEQ ID NO: 13. FIG. 8A shows nodule primordia formation in nfr1-1 pNIN:GUS roots expressing an empty vector (far left), full-length LjNFR1 (second from left), or various NFR1-CERK6 receptor chimeras with various CERK6 zone 4 alternations as indicated along the horizontal axis. Lowercase letters indicate significant differences between samples as indicated by a Kruskal-Wallis analysis of variance, with p<0.05. Constructs are numbered 17-22, each corresponding to one of the annotated mutations (M306T, A308D, M311V, Q316D, K320T, or N323D) shown in FIG. 6; i.e., construct 17 contains mutation M306T, construct 18 contains mutation A308D, construct 19 contains mutation M311V, construct 20 contains mutation Q316D, construct 21 contains mutation K320T, and construct 22 contains mutation N323D. FIG. 8B shows microscopic images of the nodule formation/infection phenotype in nfr1-1 pNin:GUS Lotus japonicus roots expressing receptor chimeras labeled 17-22 as displayed and described in FIG. 8C. Bright field images of unstained samples are shown on the left; fluorescent microscopy images of the triple yellow fluorescent protein (YFP) transformation marker are shown in the second column from the left; fluorescent microscopy images of the DsRed M. loti infection marker are shown in the third column from the left; and bright field images showing GUS staining (blue) indicating NIN promoter expression (indicating symbiotic signaling) in samples expressing NIN-GUS are shown in the two columns on the right. Scale bars represent 5 mm. FIG. 8C shows microscopic images of the nodule formation/infection phenotype in nfr1-1 pNin:GUS Lotus japonicus roots expressing receptor chimeras labeled 2 (corresponding to a symbiotically functional receptor), 17 (corresponding to M306T), 18 (corresponding to A308D), and 21 (corresponding to K320T). Bright field images of unstained samples are shown on the left; fluorescent microscopy images of the triple yellow fluorescent protein (YFP) transformation marker are shown in the second column from the left; fluorescent microscopy images of the DsRed M. loti infection marker are shown in the third column from the left; and bright field images showing GUS staining (blue) indicating NIN promoter expression (indicating symbiotic signaling) in samples expressing NIN-GUS are shown on the right. Scale bars represent 5 mm. FIG. 8D shows bright field images of nodules and the nodule primordia phenotypes of nfr1-1 pNIN:GUS roots expressing the receptor variants indicated to the left of each image. Plant roots were NIN-GUS stained. Red arrowheads indicate the expression of pNin-GUS in nodule primordia illustrating the massive nodule primordia production. Scale-bars for images labeled with Empty vector, NFR1 full-length, M306T, A308D, Q316D, and K320T represent 5 mm; for the image labeled K320T, the scale bar represents 1 mm.

FIGS. 9A-9B show that the M306, A308, and K320 motif in NFR1 JM is crucial for symbiosis. FIG. 9A shows a box-and-whiskers plot displaying total nodules (pink and white) formed per plant (y-axis) in nfr1-1 pNIN:GUS roots expressing NFR1-CERK6 chimeric receptors compared to controls. The constructs from which the data sets are derived are shown across the x-axis. The far left data shows a control sample of an empty vector in a wild type L. japonicus background as a control (labeled “Gifu”). The right-most 7 data sets are from constructs expressed in the nfr1-1 pNIN:GUS background. The schematic constructs on the x-axis are shaded according to the color code in FIG. 7A, with the NFR1 full-length control shown in all magenta. The mutation notation in the three far-right data sets refer to constructs matching the schematic shown in FIG. 7A and further containing the annotated mutations at roughly the position indicated by the black arrow in FIG. 7A. Lowercase letters indicate significant differences between samples as indicated by a Kruskal-Wallis analysis of variance, with p<0.05. FIG. 9B shows micrographs of the nodule formation/infection phenotype of nfr1-1 pNIN:GUS roots expressing the tested construct indicated to the left of each image. The top row shows plants expressing the full-length NFR1. The bottom three rows show NFR1-CERK6 chimeric receptors with the structure described in FIG. 7A and with the mutations listed on the left. The left column shows bright field images. The column labeled “YFP” shows fluorescent micrographs displaying a triple yellow fluorescent protein (tYFP) transformation marker. The column labeled “M. loti DsRed” shows fluorescent micrographs displaying a DsRed M. loti infection marker. The column labeled “pNin:GUS” shows plant roots that were GUS stained, indicating expression of pNIN, a symbiotic signaling marker. Scale bars represent 5 mm.

FIGS. 10A-10D show that residues M306 or A308 from NFR1 are sufficient to induce nodule primordia and IT formation. FIG. 10A shows a schematic representation of the basic NFR1-CERK6 chimera used for the creation of the tested chimeric constructs tested in FIGS. 10B-10D, with the ectodomain (EC) shown on the top; the transmembrane (TM) and juxtamembrane (JM) domains shown in the center (TM/JM), with numbers 1 to 4 indicating the different TM/JM zones; and the kinase domain (K) shown at the bottom. The coloring indicates the source of each segment, with portions from NFR1 shaded in magenta, and portions from CERK6 shaded in green. TM/JM zone 1 is part of the TM (NFR1 zone 1=SEQ ID NO: 3; CERK6 zone 1=SEQ ID NO: 10), while zones 2-4 are part of the JM (NFR1 zone 2=SEQ ID NO: 4; NFR1 zone 3=SEQ ID NO: 5; NFR1 zone 4=SEQ ID NO: 6; CERK6 zone 2=SEQ ID NO: 11; CERK6 zone 3=SEQ ID NO: 12; CERK6 zone 4=SEQ ID NO: 13). A black arrow indicates the region in the JM in which the amino acid substitutions in FIGS. 10B-10D are located. FIG. 10B shows a box-and-whiskers plot displaying pink and white nodules formed per plant (y-axis) in nfr1-1 pNIN:GUS roots expressing NFR1-CERK6 receptor variants compared to controls. Data from pink (indicating functional) nodules are displayed in pink; data from white (indicating uninfected) nodules are displayed in white. The constructs from which the data sets are derived are shown across the x-axis. The far left data are from a control sample of an empty vector in a wild type L. japonicus background as a control (labeled “Gifu”). The right-most 10 data sets are from constructs expressed in the nfr1-1 pNIN:GUS background. The schematic constructs on the x-axis are shaded according to the color code in FIG. 10A, with the NFR1 full-length control shown in all magenta. The mutation notation in the 6 far-right data sets refer to constructs matching the schematic shown in FIG. 10A and further containing the amino acid substitution annotated under the x-axis (T340M, D360A, V309M, T318K, or D321N) at roughly the position indicated by the black arrow in FIG. 10A. Different lowercase letters indicate significant differences in the formation of total nodules (pink and white) among genotypes as indicated by a Kruskal-Wallis analysis of variance, with p<0.05. Constructs 1, 2, and 6 are labeled as such, and zone 4's location of the single substitution sites is labeled by a black arrow. Total number of nodulating plants out of total plants observed for each genotype are labeled as (nodulating/total plants). FIG. 10C shows representative bright field images of nodule primordia formation phenotype of nfr1-1 pNIN:GUS roots expressing the chimeras containing the amino acid substitution T304M (top) or D306A (bottom) as in FIGS. 10A-10B. Teal indicates NIN-GUS staining, and serves as a marker of symbiotic signaling. The top image is representative of 2 out of 6 tested plants. The bottom image is representative of 6 out of 18 tested plants. Scale bars represent 1 mm. FIG. 10D shows transmitted light micrographs overlaid with fluorescent micrographs of a DsRed M. loti infection marker in nfr1-1 pNIN:GUS roots expressing the same chimeras as in FIG. 10C and undergoing infection thread formation. The roots are expressing either the T304M (left) or D306A (middle and right) amino acid substitution. White arrows and red indicate M. loti infection. Yellow arrows indicate aborted infection threads. Scale bars indicate 100 μm (left and middle) or 50 μm (right).

FIGS. 11A-11C show the role of residues M306, A308, and K320 of NFR1; and residues T304, D306, and T318, respectively, of the CERK protein sequence in nodule organogenesis. FIG. 11A shows a schematic representation of the basic NFR1-CERK6 chimera used for the creation of the tested chimeric constructs tested in FIGS. 11B-11C, with the ectodomain (EC) shown on the left; the transmembrane (TM) and juxtamembrane (JM) domains shown in the center (TM/JM), with numbers 1 to 4 indicating the different TM/JM zones; and the kinase domain (K) shown at the right. The coloring indicates the source of each segment, with portions from NFR1 shaded in magenta, and portions from CERK6 shaded in green. TM/JM zone 1 is part of the TM (NFR1 zone 1=SEQ ID NO: 3; CERK6 zone 1=SEQ ID NO: 10), while zones 2-4 are part of the JM (NFR1 zone 2=SEQ ID NO: 4; NFR1 zone 3=SEQ ID NO: 5; NFR1 zone 4=SEQ ID NO: 6; CERK6 zone 2=SEQ ID NO: 11; CERK6 zone 3=SEQ ID NO: 12; CERK6 zone 4=SEQ ID NO: 13). A black arrow indicates the region in the JM in which the amino acid substitutions in FIGS. 11B-11C are located. FIG. 11B shows a box-and-whiskers plot displaying pink and white nodules formed per plant (y-axis) in nfr1-1 pNIN:GUS roots expressing NFR1-CERK6 receptor variants compared to controls. Data from pink (indicating functional) nodules are displayed in pink; data from white (indicating uninfected) nodules are displayed in white. The constructs from which the data sets are derived are shown across the x-axis. The far left data are from a control sample of an empty vector in a wild type L. japonicus background as a control (labeled “Gifu”). The right-most 6 data sets are from constructs expressed in the nfr1-1 pNIN:GUS background, with the second-to-left dataset representing an empty vector. CERK1 contributions to chimeric constructs are represented by dark grey, and NFR1 contributions to chimeric constructs are represented by red. The mutation notation in the two far-right data sets refer to constructs matching the schematic shown in FIG. 11A and further containing the amino acid substitution annotated under the x-axis (either the T304M and D306A motif or the T304M, D306A, and T318K motif) at roughly the position indicated by the black arrow in FIG. 11A. Different lowercase letters indicate significant differences in the formation of total nodules (pink and white) among genotypes as indicated by a Kruskal-Wallis analysis of variance, with p<0.05. Constructs are labeled along the horizontal axis, from left to right: 1, 2, 6, 33, and 34). FIG. 11C shows micrographs of the nodule formation/infection phenotype of nfr1-1 pNIN:GUS roots expressing the tested construct indicated to the left of each image. The top row shows plants expressing the full-length NFR1. The bottom two rows show NFR1-CERK6 chimera with the structure described regarding FIG. 11A with either the T304M and D306A motif (middle row) or the T304M, D306A, and T318K motif (bottom row). The left column shows bright field images. The column labeled “YFP” shows fluorescent micrographs displaying a triple yellow fluorescent protein (tYFP) transformation marker. The column labeled “M. loti DsRed” shows fluorescent micrographs displaying a DsRed M. loti infection marker. Scale bars indicate 5 mm.

FIGS. 12A-12B show that the entire HvRLK4 intracellular region is not able to complement LjNFR1 in nodule organogenesis. FIG. 12A shows a protein alignment between LjNFR1 (top sequence; SEQ ID NO: 1) and HvRLK4 (bottom sequence; SEQ ID NO: 56). Residues that are conserved between the two proteins are shown with dots. Gaps are shown with horizontal dashes. The colored shading represents the different receptor parts used for the construction of chimeric receptors, with the ectodomain (EC) shown in blue (LjNFR1 EC=SEQ ID NO: 2; HvRLK4 EC=SEQ ID NO: 150), the transmembrane (TM) and juxtamembrane (JM) domains shown in green and labelled TM/JM (LjNFR1 TM zone 1=SEQ ID NO: 3; LjNFR1 JM zone 2=SEQ ID NO: 4; L:jNFR1 JM zone 3=SEQ ID NO: 5; LjNFR1 JM zone 4=SEQ ID NO: 6; HvRLK4 TM zone 1=SEQ ID NO: 57; HvRLK4 JM zone 2=SEQ ID NO: 58; HvRLK4 JM zone 3=SEQ ID NO: 59; HvRLK4 JM zone 4=SEQ ID NO: 60), and the kinase domain (KD) shown in pink (LjNFR1 KD=SEQ ID NO: 7; HvRLK4 KD=SEQ ID NO: 61). Zones 1 of the TM and zones 2-4 of the JM are shown separated by dashed lines. FIG. 12B shows a box-and-whiskers plot displaying total pink and white nodules formed per plant (y-axis) in nfr1-1 pNIN:GUS roots expressing NFR1-RLK4 chimeric receptors compared to controls. Data from pink (indicating functional) nodules are displayed in pink; data from white (indicating uninfected) nodules are displayed in white. The constructs from which the data sets are derived are shown across the x-axis. The far left data are from a control sample of an empty vector in a wild type L. japonicus background as a control (labeled “Gifu”). The right-most 6 data sets are from constructs expressed in the nfr1-1 pNIN:GUS background, with the second-to-left dataset representing an empty vector. The schematic constructs on the x-axis are shaded according to the color code at bottom right, in which regions from NFR1 are shown in magenta, regions from RLK4 are shown in dark grey, the ectodomain (EC) is shown at the top of each construct, the transmembrane and juxtamembrane domains (TM/JM) are shown in the center, and the kinase domain (KD) is shown at the bottom. Lowercase letters indicate significant differences in the formation of total nodules (pink and white) among genotypes as indicated by a Kruskal-Wallis analysis of variance, with p<0.05. Percentages under the boxplots indicate the nodulation frequency.

FIGS. 13A-13E show that zone 4 of NFR1 maintains its symbiotic determinacy when embedded in the intracellular region of RLK4, but that its full properties are dependent on and enhanced by the remaining regions of the NFR1 TM/JM. FIG. 13A shows a schematic representation of the basic NFR1-RLK4 chimeras used for the creation of the chimeric constructs tested in FIGS. 13B-13C carrying JM zone swaps, with the ectodomain (EC) shown on the left; the transmembrane (TM) and juxtamembrane (JM) domains shown in the center (TM/JM), with number 1 indicating TM zone 1, and numbers 2-4 indicating JM zones 2-4; and the kinase domain (KD) shown at the right. The coloring indicates the source of each segment, with portions from NFR1 shaded in magenta, and portions from RLK4 shaded in dark gray. Magenta/gray color gradients indicate that the source of the TM/JM zones varied between constructs. FIG. 13B shows a box-and-whiskers plot displaying total pink and white nodules formed per plant (y-axis) in nfr1-1 pNIN:GUS roots expressing NFR1-RLK4 receptor variants compared to controls. Data from pink (indicating functional) nodules are displayed in pink; data from white (indicating uninfected) nodules are displayed in white. The constructs from which the data sets are derived are shown across the x-axis. The far left data are from a control sample of an empty vector in a wild type L. japonicus background as a control (labeled “Gifu”). The right-most 9 data sets are from constructs expressed in the nfr1-1 pNIN:GUS background, with the second-to-left dataset representing an empty vector. The schematic constructs on the x-axis are shaded according to the color code in FIG. 13A, with an NFR1 full-length positive control shown in all magenta, and an RLF4 full-length negative control shown in all dark gray. Lowercase letters indicate significant differences in the formation of total nodules (pink and white) between genotypes as indicated by a Kruskal-Wallis analysis of variance, with p<0.05. Percentages above the x-axis indicate the nodulation frequency observed in the plants tested in each respective data set. The vertical dashed black line separates the controls (left) from the four receptor variants tested in the assay described in Example 7. FIG. 13C shows micrographs of the nodule formation phenotype of nfr1-1 pNIN:GUS roots expressing the tested construct indicated to the left of each image. The construct schematics on the left are colored according to the scheme of FIG. 13A. The top row shows roots expressing full-length NFR1. The bottom two rows show roots expressing receptor chimera with the EC from NFR1 and the KD from RLK4 and either zones 1-3 from RLK4 paired with zone 4 from NFR1 (middle row), or zones 1-3 from NFR1 paired with zone 4 from RLK4 (bottom row). The left column shows bright field images. The second column of images from the left shows fluorescent micrographs displaying a DsRed M. loti infection marker. The third column of images from the left shows fluorescent micrographs displaying a triple yellow fluorescent protein (tYFP) transformation marker. The rightmost column shows GUS staining (teal), indicating pNIN expression, which serves as a marker of symbiotic signaling. Scale bars indicate 5 mm. FIG. 13D shows a box-and-whiskers plot displaying pink and white nodules formed per plant (y-axis) in nfr1-1 pNIN:GUS roots expressing NFR1-RLK4 receptor variants compared to controls. Data from pink (indicating functional) nodules are displayed in pink; data from white (indicating uninfected) nodules are displayed in white. The constructs from which the data sets are derived are shown across the x-axis. The far-left data are from a control sample of an empty vector in a wild type L. japonicus background as a control (labeled “Gifu”). The right-most three data sets are from constructs expressed in the nfr1-1 pNIN:GUS background, with the second-to-left dataset representing an empty vector. The schematic constructs on the horizontal axis are shaded with the NFR1 full-length control shown in all red. The chimeric notation in the two far-right data sets is shaded such that RLK4 contribution are in dark grey and NFR1 contributions are in red. Different lowercase letters indicate significant differences in the formation of total nodules (pink and white) among genotypes as indicated by a Kruskal-Wallis analysis of variance, with p<0.05. The empty NFR1 construct is numbered 1, a non-chimeric RLK4 construct is numbered 35, and chimeric constructs are numbered 36 and 37. FIG. 13E shows a box-and-whiskers plot displaying results of a luminescence-based assay on transgenic roots expressing receptor variants. The y-axis indicates the maximum values of Relative Luminescence Units (“RLU max values”) reported within 30 minutes upon 0.1 mg/ml chitin application. From left to right along the horizontal axis, CERK6 represents the unaltered CERK6 receptor, “tYFP_EV” represents an empty vector negative control of triple YFP, “HvRLK4” represents an RLK4 receptor from H. vulgare, and “CERK6EC™_RLK4KD” represents a chimeric construct with an ectodomain, transmembrane, and juxtamembrane from CERK6 and a kinase domain from RLK4.

FIGS. 14A-14C show crystal structures and alignments of LjCERK6 and related proteins. FIG. 14A shows preliminary crystal structures of LjCERK6 (top; green) and MtLYK3 (bottom; magenta). Zone 4 is depicted in light green on LjCERK6 and in light pink on MtLYK3. The MtLYK3 with AMP-PNP shows the protein crystallized with the nucleotide AMP-PNP (shown in orange, red, green, and blue and circled in the dashed line) as a proxy for the nucleotide-bound state. The LjCERK6 apo shows LjCERK6 crystallized without the nucleotide. FIG. 14B shows the crystal structure of MtLYK3 (magenta) with AMP-PNP (orange, red, green, and blue). Zone 4 is depicted in light pink and is labeled αB (top). FIG. 14C shows conservation of CERK6-type kinases (CERK6-types) and NFR1-type kinases (NFR1-types) based on alignments of sequences from 23 species in the Fabales order. The level of conservation is evaluated in relation to the structures using ConSurf and is indicated by color, with blue representing residues that are the most conserved across the analyzed sequences and red representing residues that are most variable across the analyzed sequences. Zone 4 is marked on both structures with red lines.

FIG. 15A-15I show results of in vitro experiments on recombinant receptor chimera proteins. FIG. 15A shows a schematic representation of Chimera A, Chimera B, Chimera C, and Chimera D. The coloring indicates the source of each segment, with portions from NFR1 shaded in magenta, and portions from CERK6 shaded in green. For each of the Chimeras, the ectodomain (EC) is shown at the top; the transmembrane (TM) and juxtamembrane (JM) domains are shown in the center (TM/JM), with number 1 indicating TM zone 1 (NFR1 zone 1=SEQ ID NO: 3; CERK6 zone 1=SEQ ID NO: 10), and numbers 2-4 indicating JM zones 2-4 (NFR1 zone 2=SEQ ID NO: 4; NFR1 zone 3=SEQ ID NO: 5; NFR1 zone 4=SEQ ID NO: 6; CERK6 zone 2=SEQ ID NO: 11; CERK6 zone 3=SEQ ID NO: 12; CERK6 zone 4=SEQ ID NO: 13); and the kinase domain (K) is shown at the bottom. Chimera A contains the ectodomain of NFR1, the TM/JM of NFR1, and the kinase domain of CERK6. Chimera B contains the ectodomain of NFR1, the TM/JM of CERK6, and the kinase domain of NFR1. Chimera C contains the ectodomain of NFR1, TM zone 1 of NFR1, JM zones 2 and 3 of NFR1, JM zone 4 of CERK6, and the kinase domain of CERK6. Chimera D contains the ectodomain of NFR1, TM zone 1 of CERK6, JM zones 2 and 3 of CERK6, JM zone 4 of NFR1, and the kinase domain of CERK6. The red square on each schematic marks the part of the receptor that is the recombinant protein purified from E. coli. FIG. 15B shows a plot displaying inflection temperatures from in vitro nano-DSF experiments on purified recombinant receptor chimera protein. The y-axes display temperature in ° C. The x-axes display the sample names. Each point marks the measured inflection temperature. “+Mg” indicates that the sample contained added magnesium. “+ATP” indicates that the sample contained added adenosine triphosphate (ATP). The sample labeled “Chimera A” shows results from Chimera A in FIG. 15A. The sample labeled “Chimera B” shows results from Chimera B in FIG. 15A. The sample labeled “Chimera C” shows results from the Chimera C in FIG. 15A). The sample labeled Chimera D shows results from Chimera D in FIG. 15A. FIGS. 15C-15E show raw nano-DSF data collected from the proteins described above, with color keys to the curves shown at right. The labels correspond to those described for FIG. 15A. FIG. 15C shows data from Chimera A (top) and Chimera B (bottom). FIG. 15D shows data from Chimera C (top) and Chimera D (bottom). FIG. 15E shows data from NFR1 (top) and CERK6 (bottom). FIG. 15F shows a native SDS-PAGE gel of purified protein. Lanes are as follows, from left to right: size ladder, BSA control, NFR1, CERK6, Chimera A, Chimera B, Chimera C, Chimera D, and NFR1 D462N (negative control). FIG. 15G shows SDS-PAGE gels showing results of radioactive kinase assay blots. The top image shows the gel stained with InstantBlue dye; the bottom image shows the overnight radioactive exposure from the same gel. Lanes are as follows, from left to right: size ladder, myelin basic protein (MBP); NFR1, NFR1+MBP; CERK6; CERK6+MBP; receptors 13/24; receptors 13/14+MBP; size ladder; MBP; NFR1; NFR1+MBP; NFR1 D462N; NFR1 D462N+MBP. FIG. 15H shows SDS-PAGE gels showing results of radioactive kinase assay blots. The top image shows the gel stained with InstantBlue dye; the bottom image shows the overnight radioactive exposure from the same gel. Lanes are as follows, from left to right: size ladder, CERK6, CERK6+myelin basic protein (MBP); NFR1; NFR1+MBP; Chimera A; Chimera A+MBP; Chimera B; Chimera B+MBP; Chimera C; Chimera C+MBP; Chimera D; Chimera D+MBP; NFR1 D462N; NFR1 D462N+MBP. FIG. 15I shows a SDS-PAGE gel and a corresponding radiograph showing results of a radioactive kinase assay. The top image shows the gel stained with InstantBlue dye; the bottom image shows the overnight radioactive exposure from the same gel. Lanes are as follows from left to right: size ladder, NFR1.

FIGS. 16A-16B show phylogenetic trees of LysM receptors in Lotus and Fabales species, created using the Neighbor-joining construction method and with a bootstrap value of 100. FIG. 16A shows a phylogenetic tree made with full length protein sequences of LysM receptor kinases and pseudokinases from Lotus japonicus. The protein names are displayed at the ends of the branches. The NFR1 clade is shown highlighted in pink. FIG. 16B shows a phylogenetic tree representing the grouping of the NFR1 clade paralogs in nodulating Fabales species based on full length protein sequences. NFR1 paralogs are highlighted in pink. CERK6 paralogs are highlighted in green. Lys7 paralogs are highlighted in purple. NFRe paralogs are highlighted in cyan. Lys2 paralogs are highlighted in yellow. The gene names are displayed at the ends of the branches.

FIGS. 17A-17C show conservation and variability among kinase domains of NFR- and CERK-type RLKs. FIG. 17A shows a phylogenetic tree of the kinase domains of NFR- and CERK-type RLKs in species in and out of the Fabales order. The clade with NFR1 homologs in Fabales nodulating species is shaded in pink. The gene names are displayed at the ends of the branches. FIG. 17B shows a protein sequence alignment among the 70% consensus kinase sequences of Fabales NFRs (top row; SEQ ID NO: 62), Fabales CERKs (middle row; SEQ ID NO: 63), and non-Fabales RLKs (bottom row; SEQ ID NO: 64). Residues conserved across species are shown as dots. Conserved residues that differ among RLK types are shown in pink boxes. Red X's in pink boxes represent residues that are variable in more than 70% of the aligned accessions. Yellow boxes labeled “E” indicate residues that correspond to the residues composing the kinase zone E in NFR1 and CERK6.

FIG. 17C shows the MtLYK3 crystal structure with annotated subdomains and features. β-sheets are numbered (ρ1, ρ2, β3, β4, β5, and β6) and α-helixes are lettered (αA, αB, αC, αD, αE, αF, αG, αG′, αH). Different colors indicate different α-helixes and β-sheets. The N-terminus is shown at the top of the image, the C-terminus is shown at the bottom of the image, and the activation loop (AL) is shown at the right of the image. The labelled DFG and HRD motifs are essential for kinase activation and phosphorylation, while the labelled YAQ motif is important for symbiosis (Gough et al. (2018). Evolutionary History of Plant LysM Receptor Proteins Related to Root Endosymbiosis. Front. Plant Sci., Sec. Plant Development and EvoDevo, Volume 9, doi[dot]org/10.3389/fpls.2018.00923).

FIGS. 18A-18D show kinase surface zones (kinase zones) in NFR1 and CERK6 that differ between NFR1-types and CERK6-types, as well as kinase zones in LYK3. FIG. 18A shows a structural homology model (made in Swiss Model) of the JM zone 4 (shown in red at the top and labeled “4”) and kinase zones of NFR1 based on the MtLYK3 crystal structure. Different colors and corresponding lettering highlight the kinase zones that vary between NFR-type RLKs and CERK-type RLKs. Zone A consists of residues that belong to the activation loop (AL; magenta); Zone B contains residues of the αG- and αG′- helixes as well as the loop that connects them (light blue); Zone C contains residues of the C-terminal tail (orange); surface D consists of three residues located in the αH-helix (purple); Zone E primarily contains residues that belong to the αC-helix (green); Zone F contains residues that belong to different loops, sheets and helixes that are in close proximity in the structure (dark blue); and finally Zone G contains residues that belong to the αG-H loop (yellow). FIG. 18B shows an alignment between the kinase domains of NFR1 (top; SEQ ID NO: 65) and CERK6 (bottom; SEQ ID NO: 66). Variable residues in CERK6 compared to NFR1 are displayed in the bottom row. Dots represent conserved residues. Colored boxes labeled A-G highlight the kinase zones, with colors corresponding to those shown in FIG. 18A. The amino acids composing each kinase zone and their positions in the protein sequence are indicated in Table 1. The vertical red dashed line indicates the border between the N- and C- terminus of the kinase domain. The N-terminus starts from residue D328 (in NFR1) or A326 (in CERK6). The C-terminus starts from residue 1467 (in NFR1) or T465 (in CERK6), which are the first residues of the respective Activation Loop in each of NFR1 or CERK6. FIG. 18C shows additional structural homology models, for CERK6 (left) and LYK3 (right). Zone 4, the glycine-rich loop, the partly structured activation loop, and the N- and C-lobes are labeled on both models. FIG. 18D shows a close-up version of zone 4 shown in FIG. 18C for both CERK6 (left) and LYK3 (right), with specific residues shaded grey in the top panels and labeled in the bottom panels.

FIGS. 19A-19E show that NFR1 kinase zones, individually or in combination with zone D, are not sufficient to induce symbiotic signaling. FIG. 19A shows a schematic representation of the basic NFR1-CERK6 chimeras used for the creation of the chimeric constructs tested in FIG. 19B carrying kinase zone alternations from CERK6 to NFR1, with the ectodomain (EC) shown on the left; the transmembrane (TM) and juxtamembrane (JM) domains shown in the center (TM/JM), with numbers 1 to 4 indicating the different TM/JM zones; and the kinase domain (K) shown at the right. The coloring indicates the source of each segment, with portions from NFR1 shaded in magenta, and portions from CERK6 shaded in green. Magenta/green colored striping indicates that the source of the zones in the K domain varied between constructs. A black arrow indicates the region in the K domain in which the “A”- “G” zone substitutions in FIG. 19B are located. FIG. 19B shows a box-and-whiskers plot displaying total pink and white nodules formed per plant (y-axis) in nfr1-1 pNIN:GUS roots expressing NFR1-CERK6 receptor variants compared to controls. The constructs from which the data sets are derived are shown across the x-axis. The far-left data are from a control sample of an empty vector in a wild type L. japonicus background as a control (labeled “Gifu”). The remaining data sets on the right are from constructs expressed in the nfr1-1 pNIN:GUS background, with the second-to-left data set representing an empty vector. The data sets labeled “Zone A“−“Zone G” refer to constructs matching the schematic shown in FIG. 19A and further containing the single zone substitution annotated under the x-axis (in which the sequence of the indicated zone of the CERK6 K domain was substituted with the sequence of the corresponding zone from NFR1, and wherein the identities and relative positions of each zone are as indicated in FIGS. 18A-18B) at roughly the position indicated by the black arrow in FIG. 19A. CERK6 contributions to constructs are shaded in black, the NFR1 empty vector is labeled as construct 1, and the construct without zone substitutions is labeled as construct 6. Different lowercase letters indicate significant differences in the formation of total nodules (pink and white) among genotypes as indicated by a Kruskal-Wallis analysis of variance, with p<0.05. Total number of nodulating plants out of total plants observed for each genotype are labeled as (nodulating/total plants). FIG. 19C shows a box-and-whiskers plot as in FIG. 19B, except that the zone substitutions in FIG. 19C each had two substitutions, in which substitutions of zone A, (third from right), zone B (second from right), or zone C (far right) of the CERK6 K domain to corresponding zone from NFR1 were respectively paired with a substitution of zone D of the CERK6 K domain to zone D from NFR1, such that both zones D and A were substituted in the data labeled “Zone D+A”, both zones D and B were substituted in the data labeled “Zone D+B”, and both zones D and C were substituted in the data labeled “Zone D+C”. Coloring of constructs and contributions to the chimeric constructs are shaded according to FIG. 19A. FIG. 19D shows micrographs of the nodule formation phenotype of nfr1-1 pNIN:GUS roots expressing the tested construct with the zone substitution(s) indicated to the left of each image. The left column shows bright field images. The column labeled “YFP” shows fluorescent micrographs displaying a yellow fluorescent protein (YFP) transformation marker. The column labeled “M. loti DsRed” shows fluorescent micrographs displaying a DsRed M. loti infection marker. The column labeled “pNin:GUS” shows plant roots that were GUS stained, indicating expression of pNIN, a symbiotic signaling marker. Scale bars indicate 5 mm. FIG. 19E shows structural modeling of receptors 27 and 29, where large portions are altered. In receptor 27, both the aC helix and the activation loop of CERK6 have been exchanged with the corresponding NFR1 regions. For receptor 29, the N-terminal region of CERK6 is combined with the C-terminal region of NFR1. Stars indicate altered areas of the NFR1 receptors (portions that are from CERK6 in these chimeras).

FIGS. 20A-20C show results of nodulation experiments focusing on the C-terminus of the NFR1 kinase domain. FIG. 20A shows a box-and-whiskers plot demonstrating that the C-terminus of the NFR1 kinase domain is essential for nodulation. The plot displays total pink and white nodules formed per plant (y-axis) in nfr1-1 pNIN:GUS roots expressing receptor variants carrying NFR1 kinase domain zones. compared to controls. Data from pink (indicating functional) nodules are displayed in coral; data from white (indicating uninfected) nodules are displayed in white. The constructs from which the data sets are derived are shown across the horizontal axis. The far left data are from a control sample of an empty vector in a wild type L. japonicus background as a control (labeled “Gifu”). The right-most 7 data sets are from constructs expressed in the nfr1-1 pNIN:GUS background, with the second-to-left dataset representing an empty vector. The schematic constructs on the horizontal axis are shaded with CERK contributions shown in dark grey, NFR1 contributions shown in dark grey, and a negative control of the EC and TM/JM zones 1-3 from NFR1 combined with the full kinase domain and JM zone 4 from CERK6. TM/JM zone 1 is part of the TM (NFR1 zone 1=SEQ ID NO: 3; CERK6 zone 1=SEQ ID NO: 10), while zones 2-4 are part of the JM (NFR1 zone 2=SEQ ID NO: 4; NFR1 zone 3=SEQ ID NO: 5; NFR1 zone 4=SEQ ID NO: 6; CERK6 zone 2=SEQ ID NO: 11; CERK6 zone 3=SEQ ID NO: 12; CERK6 zone 4=SEQ ID NO: 13). The remaining 4 data sets on the right each represent constructs with the EC and TM/JM zones 1-3 from NFR1 combined with the kinase domain and JM zone 4 from CERK6, in which regions of the kinase domain have been substituted as indicated. In the data set labeled “A+E”, a construct includes kinase zones A and E from NFR1. In the data set labeled “A”, a construct includes kinase zone A from NFR1. The data set labeled “N328-469C467-622” represents a construct with residues 328-469 from NFR1 and residues 467-622 from CERK6, corresponding to the N-terminus of the kinase domain from NFR1 and the C-terminus of the kinase domain from CERK6 (see dashed line in FIG. 18C for the border between N- and C-termini). The data set labeled “C₃₂₆₋₄₆₇N₄₆₉₋₆₂₃” represents a construct with residues 326-467 from CERK6 and residues 469-623 from NFR1, corresponding to the N-terminus of the kinase domain from CERK6 and the C-terminus of the kinase domain from NFR1. Lowercase letters indicate significant differences in the formation of total nodules (pink and white) between genotypes as indicated by a Kruskal-Wallis analysis of variance, with p<0.05. Fractions above the x-axis indicate the nodulation frequency observed in the plants tested in each respective data set, with the number of nodulating plants compared to the number of total plants. FIG. 20B shows SDS-PAGE gels showing results of radioactive kinase assay blots. For each gel imaged, the top image shows the gel stained with InstantBlue dye; the bottom image shows the overnight radioactive exposure from the same gel. Lanes are as follows, from left to right, for the top gel: size ladder, myelin basic protein (MBP); receptor 28; receptor 28+MBP; receptor 29; receptor 29+MBP; CERK6; CERK6+MBP. For the bottom gel: ladder; MBP; NFR1; NFR1+MBP; receptor 28; receptor 28+MBP; NFR1 D462N; NFR1 D462N+MBP. FIG. 20C shows micrographs of the nodule formation phenotype of nfr1-1 pNIN:GUS roots expressing the tested construct with the zone substitution(s) indicated to the left of each image. The left column shows bright field images. The column labeled “YFP” shows fluorescent micrographs displaying a yellow fluorescent protein (YFP) transformation marker. The column labeled “M. loti DsRed” shows fluorescent micrographs displaying a DsRed M. loti infection marker. Scale bars indicate 5 mm.

FIGS. 21A-21E show that individual NFR1 kinase zones function in immune signaling with different efficiencies. FIG. 21A shows a schematic representation of the basic NFR1-CERK6 receptor variant used for the creation of the chimeric constructs tested in FIGS. 21B-21C, carrying kinase zone alternations from CERK6 to NFR1, with the ectodomain (EC) shown on the left; the transmembrane (TM) and juxtamembrane (JM) domains shown in the center (TM/JM), with numbers 1 to 4 indicating the different TM/JM zones; and the kinase domain (KD) shown at the right. The coloring indicates the source of each segment, with portions from NFR1 shaded in magenta, and portions from CERK6 shaded in green. The magenta/green gradient in the KD indicates that the source regions of the KD zones varied between constructs, wherein the KD zones (A-G) correspond to those annotated in FIGS. 18A-18B. FIG. 21B shows a box-and-whiskers plot displaying results of a luminescence-based assay on transgenic roots expressing receptor variants. The y-axis indicates the maximum values of Relative Luminescence Units (RLU) reported within 30 minutes upon 0.1 mg/ml chitin application. The constructs from which the data sets are derived are shown across the x-axis. The left-most three data sets serve as controls. The far left data set is a positive control sample of an empty vector in a wild type L. japonicus background (labeled “Gifu”). The second data set from the left is a negative control sample of cerk6 hairy roots expressing the empty vector. The next data set to the right is a positive control sample of cerk6 hairy roots expressing the full-length CERK6 receptor. The right-most three data sets are the tested samples, with the second schematic from the left representing plants expressing chimeric receptors containing the EC and TM/JM domains from CERK6 and the K domain from NFR1. The data labeled “Zone A” are from plants expressing the full length CERK6 protein with Zone A swapped to that from NFR1. The data labeled “Zone C” are from plants expressing the full length CERK6 protein with Zone C swapped to that from NFR1. The curves above box plots illustrate the RLU values that were calculated for each sample within a transgenic line in a 30-minute time frame upon chitin application. Lowercase letters indicate significant differences in the RLU maximum values between transgenic lines as indicated by a Tukey analysis of variance (ANOVA) test, with p<0.05. FIG. 21C shows a box-and-whiskers plot displaying results of a luminescence-based assay as in FIG. 21B, with the left-most four data sets as described regarding FIG. 21B, but with different tested samples on the right. The data labeled “Zone D” are from plants expressing the full length CERK6 protein with Zone D swapped to that from NFR1. The data labeled “Zone E” are from plants expressing the full length CERK6 protein with Zone E swapped to that from NFR1. The data labeled “Zone F” are from plants expressing the full length CERK6 protein with Zone F swapped to that from NFR1. The data labeled “Zone G” are from plants expressing the full length CERK6 protein with Zone G swapped to that from NFR1. FIG. 21D shows a box-and-whiskers plot displaying results of a luminescence-based assay on transgenic roots expressing receptor variants, showing that combinations of NFR1 kinase zones function in immune signaling with different efficiencies. The y-axis indicates the maximum values of Relative Luminescence Units (RLU) reported within 30 minutes upon 0.1 mg/ml chitin application. The constructs from which the data sets are derived are shown across the x-axis. The left-most three data sets serve as controls. The far left data set is a positive control sample of an empty vector in a wild type L. japonicus background (labeled “Gifu”). The second data set from the left is a negative control sample of cerk6 hairy roots expressing the empty vector. The next data set to the right is a positive control sample of cerk6 hairy roots expressing the full-length CERK6 receptor. The right-most five data sets are the tested samples, with the second schematic from the left representing plants expressing a chimeric receptor with the EC and TM/JM domains from CERK6 and the K domain from NFR1. The data labeled “Zone A+G*” are from plants expressing a chimeric receptor with the full length CERK6 protein with Zones A and G swapped to those from NFR1, respectively but with zone G containing the T523D mutation. The data labeled “Zone A+D” are from plants expressing a chimeric receptor with the full length CERK6 protein with Zones A and D swapped to those from NFR1, respectively. The data labeled “Zone D+B” are from plants expressing a chimeric receptor with the full length CERK6 protein with Zones D and B swapped to those from NFR1, respectively. The data labeled “Zone D+C” are from plants expressing a chimeric receptor with the full length CERK6 protein with Zones D and C swapped to those from NFR1, respectively. The curves above box plots illustrate the RLU values that were calculated for each sample within a transgenic line in a 30-minute time frame upon chitin application. Lowercase letters indicate significant differences in the RLU maximum values between transgenic lines as indicated by a Tukey analysis of variance (ANOVA) test, with p<0.05. FIG. 21E shows the results of spontaneous nodule formation (vertical axis) for various Nanobody®-mediated complexes (showed by constructs joined in the three rightmost data sets) of combinations of NFR1, NFR5, CERK6, and receptor 6 (or “construct 6”). NFR1 constructs (numbered “1”) are represented in red, NFR5 constructs are represented in light grey, and CERK6 constructs (numbered “23”) and CERK6 contributions to receptor 6 (or “construct 6”) are shown in dark grey. Lowercase letters indicate significant differences in the RLU maximum values between transgenic lines as indicated by a Kruskal-Wallis analysis of variance (ANOVA) test, followed by Dunn's test with p<0.05.

FIGS. 22A-22J show that individual CERK6 kinase zones, or CERK6 kinase zones in combination with CERK6 kinase zones D or G, are not sufficient to induce immune signaling when coupled with the remaining zones from the NFR1 kinase domain. FIG. 22A shows a schematic representation of the basic CERK6- NFR1 receptor variant used for the creation of the chimeric constructs tested in FIGS. 22B-22D, carrying kinase zone alternations from NFR1 to CERK6, with the ectodomain (EC) shown on the left; the transmembrane (TM) and juxtamembrane (JM) domains shown in the center (TM/JM), with numbers 1 to 4 indicating the different TM/JM zones; and the kinase domain (KD) shown at the right. The coloring indicates the source of each segment, with portions from NFR1 shaded in magenta, and portions from CERK6 shaded in green. The magenta/green gradient in the KD indicates that the source regions of the KD zones varied between constructs, wherein the KD zones (A-G) correspond to those annotated in FIGS. 18A-18B. FIG. 22B shows a box-and-whiskers plot displaying results of a luminescence-based assay on transgenic roots expressing receptor variants. The y-axis indicates the maximum values of Relative Luminescence Units (RLU) reported within 30 minutes upon 0.1 mg/ml chitin application. The constructs from which the data sets are derived are shown across the x-axis. The left-most three data sets serve as controls. The far left data set is a positive control sample of an empty vector in a wild type L. japonicus background (labeled “Gifu”). The second data set from the left is a negative control sample of cerk6 hairy roots expressing the empty vector. The next data set to the right is a positive control sample of cerk6 hairy roots expressing the full-length CERK6 receptor. The right-most eight data sets are the tested zone swap samples, with the second schematic from the left representing plants expressing a chimeric receptor with the EC and TM/JM domains from CERK6 and the K domain from NFR1. The data labeled “Zone A”, “Zone B”, “Zone C”, “Zone D”, “Zone E”, “Zone F”, and “Zone G” are from plants expressing a chimeric receptor with the EC and TM/JM domains from CERK6 and the KD from NFR1, except that the sequence of the indicated KD zone has been swapped from that of NFR1 to the corresponding sequence from CERK6, respectively. The curves above box plots illustrate the RLU values that were calculated for each sample within a transgenic line in a 30-minute time frame upon chitin application. Lowercase letters indicate significant differences in the RLU maximum values between transgenic lines as indicated by a Tukey analysis of variance (ANOVA) test, with p<0.05. Constructs of the controls are shaded according to FIG. 22A. FIG. 22C shows a box-and-whiskers plot displaying results of a luminescence-based assay on transgenic roots as in FIG. 22B, but expressing different receptor variants. The left-most four samples are as in FIG. 22B. The right-most three samples represent zone swap constructs as in FIG. 22B, but in which two zones have been substituted in combination. The data labeled “Zone D+A”, “Zone D+B”, and “Zone D+C” are from plants expressing a chimeric receptor with the EC and TM/JM domains from CERK6 and the KD from NFR1, except that the sequences of the indicated KD zones have been swapped from those of NFR1 to the corresponding sequences from CERK6, respectively. The curves above box plots illustrate the RLU values that were calculated for each sample within a transgenic line in a 30-minute time frame upon chitin application. Lowercase letters indicate significant differences in the RLU maximum values between transgenic lines as indicated by a Tukey analysis of variance (ANOVA) test, with p<0.05. FIG. 22D shows a box-and-whiskers plot displaying results of a luminescence-based assay on transgenic roots as in FIGS. 22B-22C, but expressing a different receptor variant. The left-most four samples are as in FIGS. 22B-22C. The right-most sample (labeled “Zone A+G”) represents a zone swap construct as in FIG. 22C, but in which both the A and G zones have been swapped from those of NFR1 to the corresponding sequences from CERK6. The curves above box plots illustrate the RLU values that were calculated for each sample within a transgenic line in a 30-minute time frame upon chitin application. Lowercase letters indicate significant differences in the RLU maximum values between transgenic lines as indicated by a Tukey analysis of variance (ANOVA) test, with p<0.05. FIG. 22E shows a box-and-whiskers plot displaying results of a luminescence-based assay on transgenic roots as in FIGS. 22B-22D, but expressing a different receptor variant. The left-most two samples are as in FIGS. 22B-22C. “23” indicates receptor 23, which is an unaltered/un-substituted CERK6 receptor, and “24” indicates receptor 24, in which the JM-B region of the entire CERK6 receptor was substituted with the corresponding region of NFR1. The curves above box plots illustrate the RLU values that were calculated for each sample within a transgenic line in a 30-minute time frame upon chitin application. Lowercase letters indicate significant differences in the RLU maximum values between transgenic lines as indicated by a Tukey analysis of variance (ANOVA) test, with p<0.05. FIG. 22F shows a box-and-whiskers plot displaying results of a luminescence-based assay on transgenic roots expressing receptor variants, showing that the N-terminus of the CERK6 kinase domain is essential for immune signaling. The y-axis indicates the maximum values of RLU reported within 30 minutes upon 0.1 mg/ml chitin application. The constructs from which the data sets are derived are shown across the x-axis. The left-most three data sets serve as controls. The far left data set is a positive control sample of an empty vector in a wild type L. japonicus background (labeled “Gifu”). The second data set from the left is a negative control sample of cerk6 hairy roots expressing the empty vector. The next data set to the right is a positive control sample of cerk6 hairy roots expressing the full-length CERK6 receptor. The right-most three data sets are the tested samples, with the second schematic from the left representing plants expressing a chimeric receptor with the EC and TM/JM domains from CERK6 and the K domain from NFR1. The data labeled “N₃₂₈₋₄₆₉C₄₆₇₋₆₂₂” represents cerk6 plants expressing a chimeric receptor with the EC and TM/JM domains from CERK6 and a chimeric K domain, wherein the N-terminus had residues 328-469 from NFR1 and the C-terminus had residues 467-622 from CERK6. The data labeled “C₃₂₆₋₄₆₇N₄₆₉₋₆₂₃” represents cerk6 plants expressing a chimeric receptor with the EC and TM/JM domains from CERK6 and a chimeric K domain, wherein the N-terminus of the K domain contains residues 326-467 from CERK6, and the C-terminus of the K domain contains residues 469-623 from NFR1. The curves above box plots illustrate the RLU values that were calculated for each sample within a transgenic line in a 30-minute time frame upon chitin application. Lowercase letters indicate significant differences in the ROS maximum values between transgenic lines as indicated by a Tukey analysis of variance (ANOVA) test, with p<0.05. Fractions above curves indicate the number of samples responded to chitin with max ROS>2. The unaltered CERK6 receptor is labeled “23”, the positive control sample of cerk6 hairy roots expressing the full-length CERK6 receptor is labeled “30”, receptor “N328-469C467-622” is labeled as receptor 31 (or “construct 31”), and receptor “C₃₂₆₋₄₆₇N₄₆₉₋₆₂₃” is labeled as receptor 32 (or “construct 32”). CERK6 receptor contributions are indicated in dark grey here, and NFR1 receptor contributions are indicated in white here. FIG. 22G shows representations of the “N₃₂₈₋₄₆₉C₄₆₇₋₆₂₂” (left) and “C₃₂₆₋₄₆₇N₄₆₉₋₆₂₃” (right) chimera in the CERK6 crystal structure. Color indicates the variable residues between NFR1 and CERK6, with pink indicating residues of NFR1 and green indicating residues of CERK6. Gray indicates identical residues. JM zone 4 in each is labeled “z4” and the Activation Loop in each is labeled “AL”. FIGS. 22H-22J show the purification results for various residues of receptors used, both as concentrations of A280 (in mAu, along the vertical axis of the graphs) per mL of sample (horizontal axis of the graphs), and in SDS-PAGE intracellular domain. FIG. 22H shows these results for CERK6 (residues 303-599) and NFR1 (residues 263-623). FIG. 22I shows these results for NFR1 (residues 263-623, top panels) and receptor 13/24 (bottom panels). FIG. 22J shows these results for receptor 29 (top panels, C₃₂₆₋₄₆₇N₄₆₉₋₆₂₃), receptor 28 (middle panels, N₃₂₈₋₄₆₉C₄₆₇₋₆₂₂), and NFR1 (residues 263-623) D462N.

FIGS. 23A-23L show an amino acid sequence alignment of NFR1-type LysM receptor sequences from Mimosa pudica (Scaffoldl5743=SEQ ID NO: 67), Chamaecristafasciculata (2879S20281=SEQ ID NO: 68), Prosopis alba (XP_0287539017.1=SEQ ID NO: 69), Arachis duranensis (XP_020982945.1=SEQ ID NO: 70), Arachis hypogaea (XP_029150476.1=SEQ ID NO: 71), Arachis ipaensis (XP_020962820.1=SEQ ID NO: 72), Lupinus angustifolius (XP_019434083.1=SEQ ID NO: 73; XP_019461629.1=SEQ ID NO: 74), Lupinus alba (Chr04g0249871=SEQ ID NO: 75), Cicer arietinum (XP_004491136.1=SEQ ID NO: 76), Medicago truncatula (Q6UD73_LYK3_MEDTR=SEQ ID NO: 77), Pisum sativum (ARX80051.1=SEQ ID NO: 78), Lotus japonicus (CAE02590.1=SEQ ID NO: 1), Cajanus cajan (XP_020213700.2=SEQ ID NO: 79), Abrus precatorius (XP_027332267.1=SEQ ID NO: 80), Phaseolus vulgaris (XP_007141617.1=SEQ ID NO: 81), Vigna angularis (KOM46748.1=SEQ ID NO: 82), Vigna unguiculata (CP+027939826.1=SEQ ID NO: 83), Spatholobus suberectus (TKY57029.1=SEQ ID NO: 84), and Glycine max (XP_006575588.1=SEQ ID NO: 85; XP_006595821.2=SEQ ID NO: 86). FIG. 23A shows the first portion of the alignment. FIG. 23B shows the second portion of the alignment. FIG. 23C shows the third portion of the alignment. FIG. 23D shows the fourth portion of the alignment. FIG. 23E shows the fifth portion of the alignment. FIG. 23F shows the sixth portion of the alignment. FIG. 23G shows the seventh portion of the alignment. FIG. 23H shows the eighth portion of the alignment. FIG. 23I shows the ninth portion of the alignment. FIG. 23J shows the tenth portion of the alignment. FIG. 23K shows the eleventh portion of the alignment. FIG. 23L shows the twelfth portion of the alignment.

FIG. 24 shows plots of results of luminescence-based assays on nfr1 transgenic roots expressing the NFR1-CERK6 chimeric receptor indicated at the top of each plot. The schematics correspond to the layout shown in FIG. 7A, with dark grey indicating receptor domains from NFR1 and light green indicating receptor domains CERK6. The dark grey star indicates the presence of the K35iN mutation. The vertical axis indicates the ROS values of Relative Luminescence Units (RLU) reported upon application of 10⁻⁸M M. loti R7A nod factor (NF), 0.1 mg/ml chitin, or water. The horizontal axis shows the elapsed time in seconds since the application of the respective elicitor. A color key for the color of the curves is shown at the right, with each differently-colored curve corresponding to a different elicitor: red corresponds to 10-8M M. loti R7A NF, bright green corresponds to 0.1 mg/ml chitin, and blue corresponds to water. Water ROS values were low enough across all measurements that the ROS curves for water were mostly visually overlapped by the curves for NF (red) in each plot.

FIGS. 25A-25B show expression of NFR1 and CERK6 receptors with different C-terminal fluorescent tags in N. benthamiana leaves. FIG. 25A shows schematics of three expression cassettes (one shown in each of the three rows) for the combinations of C-terminal-tagged receptors used for N. benthamiana transient transformation. The black arrows at the far left indicate the Ubiquitin promoter (pUbi). The black arrows in the middle indicate the β35s promotor. tNOS indicates the NOS terminator, and t35s indicates the 35s terminator. Gray dotted lines connect expression constructs that were expressed on the same vector. The oblong green or pink shapes outlined in black indicate expression of NFR1 (pink) or CERK6 (green) receptors tagged at the C-terminus with m-Cherry (red star) or green fluorescent protein (GFP; green star). FIG. 25B shows fluorescent micrographs indicating expression of the tagged receptors depicted in FIG. 25A in the plasma membrane of N. benthamiana leaf cells. The top row shows cells expressing the top vector from FIG. 25A; the middle row shows cells expressing the middle vector from FIG. 25A; and the bottom row shows cells expressing the bottom vector from FIG. 25A. The mCherry channel is shown in the left column; the GFP channel is shown in the middle column; and a merge of the mCherry and GFP channels is shown in the right column. Scale bars indicate 100 microns.

FIGS. 26A-26B show box-and-whiskers plots displaying pink and white nodule formation in Lotus japonicus roots expressing the receptor chimeras (magenta- and green-shaded constructs) or controls (empty vector) shown along the x-axes. The y-axes indicate nodules per plant. Chimera schematics are laid out and shaded according to the keys shown to the bottom right of each graph, in which the EC is shown at the top, the TM/JM (including zones 1-4) is shown in the middle, and the K domain is shown at the bottom, with each being shaded according to its respective source (pink if from NFR1; green if from CERK6). Different lowercase letters indicate significant differences between samples as demonstrated by a Kruskal-Wallis analysis of variance test with p<0.05. Fractions on the bottom of each boxplot indicate the number of nodulating plants out of the total plants tested for each construct. FIG. 26A shows results from nfr1 cerk6 double mutant roots expressing the indicated chimeras or empty vector (right portion) or Gifu (wild-type) roots expressing the empty vector (left portion). FIG. 26B shows results from Gifu roots expressing the indicated chimeras or empty vector.

FIGS. 27A-27B show results of experiments investigating the biochemical properties of methionine306 (M306) in the context of nodule organogenesis. FIG. 27A shows a box-and-whiskers plot displaying total (pink and white) nodule formation in Lotus japonicus Gifu (wild type; left portion) or nfr1-1 pNIN:GUS (right portion) roots expressing the receptor chimeras (magenta- and green-shaded constructs) or controls (empty vector) shown along the x-axis. The y-axis indicates total nodules per plant. Chimera schematics are laid out and shaded according to the schematic shown in FIG. 7A. The labels “M306T”, “M306V”, “M306Q”, or “M306A” indicate that the construct had the listed mutation in the methionine306 position (M to T, V, Q, or A, respectively) within JM zone 4. Different lowercase letters indicate significant differences between samples as demonstrated by a Kruskal-Wallis analysis of variance test with p<0.05. Fractions on the bottom of each boxplot indicate the number of nodulating plants out of the total plants tested for each construct. FIG. 27B shows microscopic images of the nodule formation/infection phenotype of nfr1-1 pNIN:GUS roots expressing a wild type NFR1 protein (top row) or a chimeric construct with the EC and TM/JM of NFR1 and the K domain of CERK6 and containing the indicated mutation (M306Q in the middle row; M306A in the bottom row). Bright field images are shown on the left; fluorescent microscopy images of the triple yellow fluorescent protein (YFP) transformation marker are shown in the middle; and fluorescent microscopy images of the DsRed M. loti infection marker are shown on the right. Scale bars represent 5 mm.

FIGS. 28A-28D show data demonstrating that aspartic acid in zone 4 inhibits nodulation and nodule infection by creating a salt bridge with the αC-helix. FIG. 28A shows a box-and-whiskers plot displaying total (pink and white) nodule formation in Lotus japonicus Gifu (wild type; left portion) or nfr1-1 pNIN:GUS (right portion) roots expressing the receptor chimeras (magenta- and green-shaded constructs) or controls (empty vector) shown along the x-axis. The y-axis indicates total nodules per plant. Chimera schematics are laid out and shaded according to the schematic shown in FIG. 7A. The labels “A308D”, “A308E”, or “A308L” indicate that the construct had the listed mutation in the alanine at position 308 (A to D, E, or L, respectively) within JM zone 4. Different lowercase letters indicate significant differences between samples as demonstrated by a Kruskal-Wallis analysis of variance test with p<0.05. Fractions on the bottom of each boxplot indicate the number of nodulating plants out of the total plants tested for each construct. FIG. 28B shows the salt bridge (shown as a dotted line in the section shown in translucent red, blue, and green, with adjacent blue and red portions indicating polar contacts) between D306 and K365 in a preliminary CERK6 crystal structure (shown in green). JM zone 4 is shown in light green. The αC-helix of the kinase is labeled on the right. FIG. 28C shows a model of the D306A substitution in a portion of a preliminary CERK6 crystal structure (shown in green). JM zone 4 is shown in light green. FIG. 28D shows a model of crystallography structures for CERK6 and LYK3's similar salt bridge (dotted line) between orange residues E362 and K464, between the αC-helix and an “inhibitory” helix.

FIGS. 29A-29C show illustrations and results demonstrating that mutation in the NFR1 activation loop abolishes nodulation. FIG. 29A shows illustrations demonstrating different views of the two parallel helices activation loop and αC-helix, in a NFR1 kinase homology model based on the MtLYK3 structure. The two images at the top are magnified from the area in the dotted rectangle shown on the bottom image and are rotated 90 degrees in relation to each other, and show labels indicating relevant residues. In the red circle (top right), the position of E365 and E467 is highlighted. FIG. 29B shows a model of the L468R mutation in a NFR1 kinase homology model. The black arrow indicates the location of the L468R mutation. FIG. 29C shows a box-and-whiskers plot displaying total (pink and white) nodule formation in Lotus japonicus Gifu (wild type; left portion) or nfr1-1 pNIN:GUS (right portion) roots expressing the constructs indicated along the x-axis: empty vectors (left), the full length NFR1 receptor (third from left), or the full length NFR1 receptor with the L468R mutation (right). The bottom portion of the plot shows results from uninoculated samples. The top portion of the plot shows results from samples inoculated with M. loti R7A. The y-axis indicates total nodules per plant. Different lowercase letters indicate significant differences between samples as demonstrated by a Kruskal-Wallis analysis of variance test with p<0.05. Fractions on the bottom of each boxplot indicate the number of nodulating plants out of the total plants tested for each construct.

FIG. 30 shows plots of results of luminescence-based assays in cerk6 transgenic roots expressing the CERK6-NFR1 chimeric receptor indicated above each plot, demonstrating that substitution of CERK6 zone 4 alone with the corresponding NFR1 enhances ROS responses. The y-axis indicates the ROS values of Relative Luminescence Units (RLU) reported upon application of different concentrations of chitin octamer (CO8) elicitors. The colors of the plotted data correspond to the color key of elicitor concentrations shown at the right. The x-axis shows the time in seconds since application of the elicitor. Different lowercase letters indicate significant differences in the mean. The top left plot shows results from Gifu (wild type) roots expressing the empty vector. The top middle plot shows results from cerk6 roots expressing the empty vector. The remaining plots show results from cerk6 roots expressing the construct shown in the schematic above each plot, in which green indicates domains of CERK6 and magenta indicates domains of NFR1 according to the schematic layout described in FIG. 7A.

FIG. 31 shows illustrations demonstrating that the CERK6 activation loop forms an inhibitory helical conformation in the absence of ligand. The far left structure shows the CERK6 crystal structure (green). The dotted rectangle shows the area containing the two parallel helixes, the activation loop, and the αC-helix. The two images in the center are magnified from the area in the dotted rectangle shown on the left image and are rotated 90 degrees in relation to each other, and show labels indicating relevant residues. In the red circle (top right), the position of E365 and E467 is highlighted. The red circle shows the positions of E363 and E465 and the salt bridge. The far right structure shows a model of the L466R (black arrow) mutation in the CERK6 crystal structure, from roughly the same angle as the second image from the left.

### DETAILED DESCRIPTION

The following description sets forth exemplary methods, parameters, and the like. It should be recognized, however, that such description is not intended as a limitation on the scope of the present disclosure but is instead provided as a description of exemplary embodiments.

### Modified Plant LysM Receptor Polypeptides and Related Methods

An aspect of the disclosure includes a modified plant LysM receptor polypeptide including a first JM zone 4, wherein the first JM zone 4 has been modified by insertion, deletion, or substitution of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 4 with the corresponding amino acids from a second JM zone 4 of a second plant LysM receptor polypeptide or wherein the plant LysM receptor polypeptide lacks a first JM zone 4 and the plant LysM receptor polypeptide has been modified by insertion of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 4 with the corresponding amino acids from the second JM zone 4 into the corresponding site when aligning the plant LysM receptor and the second plant LysM receptor. As used herein, the terms zone 4 and JM-B are interchangeable. In a further embodiment of this aspect, the first JM zone 4, the second JM zone 4, or both correspond to amino acids 305 to 327 when aligned to SEQ ID NO: 1 or correspond to amino acids 303 to 325 when aligned to SEQ ID NO: 8. In an additional embodiment of this aspect, which may be combined with any of the preceding embodiments, the first JM zone 4 is modified by substituting at least two or at least three amino acid residues in the first JM zone 4 with corresponding amino acid residues that are different in the second JM zone 4, or the first JM zone 4 is modified by substituting three amino acid residues in the first JM zone 4 with corresponding amino acid residues that are different in the second JM zone 4. In yet another embodiment of this aspect, substitution includes deletion of an amino acid not found in the second JM zone 4 and insertion of an amino acid found in the second JM zone 4 but not in the first JM zone 4. In still another embodiment of this aspect, which may be combined with any of the preceding embodiments, the second JM zone 4 is able to initiate NFR1-mediated root nodule symbiosis signaling, ROS signaling, or different signaling than the first JM zone 4. In another embodiment of this aspect, the second JM zone 4 is able to initiate NFR1-mediated root nodule symbiosis signaling. In an additional embodiment of this aspect, the first JM zone 4 includes SEQ ID NO: 13, is selected from the group of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40, or conservative substitutions thereof, or optionally any of the preceding amino acid sequences with one, two, or three amino acid substitutions, insertions and/or deletions, or includes SEQ ID NO: 60; and second JM zone 4 includes SEQ ID NO: 6, or is selected from the group of SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and SEQ ID NO: 52, or optionally any of the preceding amino acid sequences with one, two, or three amino acid substitutions, insertions and/or deletions. In a further embodiment of this aspect, the second JM zone 4 is able to initiate ROS signaling. In still another embodiment of this aspect, the first JM zone 4 includes SEQ ID NO: 6, or is selected from the group of SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and SEQ ID NO: 52, or optionally any of the preceding amino acid sequences with one, two, or three amino acid substitutions, insertions and/or deletions, and/or wherein the second JM zone 4 includes SEQ ID NO: 13, or is selected from the group of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40, or optionally any of the preceding amino acid sequences with one, two, or three amino acid substitutions, insertions and/or deletions. In yet another embodiment of this aspect, which may be combined with any of the preceding embodiments, the substituted amino acid residues are selected from amino acid residues corresponding to M306, A308, and K320 of SEQ ID NO: 1 or from amino acid residues corresponding to T304, D306, and T318 of SEQ ID NO: 8.

A further aspect of the disclosure includes a modified plant non-NFR1 LysM receptor polypeptide engineered for NFR1-mediated root nodule symbiosis signaling including a first JM zone 4, wherein the first JM zone 4 was modified by substitution of one or more amino acids in the first JM zone 4 with the corresponding amino acids from a second JM zone 4 from an NFR1 LysM receptor polypeptide with NFR1-mediated root nodule symbiosis signaling. In an additional embodiment of this aspect, substitution includes deletion of an amino acid not found in the second JM zone 4 and insertion of an amino acid found in the second JM zone 4 but not in the first JM zone 4. In a further embodiment of this aspect, which may be combined with any of the preceding embodiments, the first JM zone 4 is modified by substituting at least two or at least three amino acid residues in the first JM zone 4 with corresponding amino acid residues that are different in the second JM zone 4. In yet another embodiment of this aspect, the first JM zone 4 is modified by substituting three amino acid residues in the first JM zone 4 with corresponding amino acid residues that are different in the second JM zone 4. In still another embodiment of this aspect, which may be combined with any of the preceding embodiments, the first JM zone 4 includes SEQ ID NO: 13, is selected from the group of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40, or conservative substitutions thereof, or optionally any of the preceding amino acid sequences with one, two, or three amino acid substitutions, insertions and/or deletions, or includes SEQ ID NO: 60, and/or wherein the second JM zone 4 includes SEQ ID NO: 6, or is selected from the group of SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and SEQ ID NO: 52, or optionally any of the preceding amino acid sequences with one, two, or three amino acid substitutions, insertions and/or deletions. In a further embodiment of this aspect, which may be combined with any of the preceding embodiments, the substituted amino acid residues are selected from amino acid residues corresponding to M306, A308, and K320 of SEQ ID NO: 1 or from amino acid residues corresponding to T304, D306, and T318 of SEQ ID NO: 8. In an additional embodiment of this aspect, which may be combined with any of the preceding embodiments, the modified plant non-NFR1 LysM receptor polypeptide further includes a first kinase C-terminus region, wherein the first kinase C-terminus region has been modified as compared to the amino acid sequence of the corresponding unmodified plant LysM receptor polypeptide by substitution of one or more amino acids in the first kinase C-terminus region with the corresponding amino acids from an NFR1 LysM receptor polypeptide.

An additional aspect of the disclosure includes a modified plant LysM receptor polypeptide with enhanced ROS signaling including a first JM zone 4, wherein the first JM zone 4 was modified by substitution of one or more amino acids in the first JM zone 4 with the corresponding amino acids from a second JM zone 4 from a LysM receptor polypeptide with ROS signaling. In a further embodiment of this aspect, the first JM zone 4 is modified by substituting at least two or at least three amino acid residues in the first JM zone 4 with corresponding amino acid residues that are different in the second JM zone 4, or the first JM zone 4 is modified by substituting three amino acid residues in the first JM zone 4 with corresponding amino acid residues that are different in the second JM zone 4. In an additional embodiment of this aspect, substitution includes deletion of an amino acid not found in the second JM zone 4 and insertion of an amino acid found in the second JM zone 4 but not in the first JM zone 4. In yet another embodiment of this aspect, which may be combined with any of the preceding embodiments, the first JM zone 4 includes SEQ ID NO: 6, or is selected from the group of SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and SEQ ID NO: 52, or optionally any of the preceding amino acid sequences with one, two, or three amino acid substitutions, insertions and/or deletions, and/or wherein the second JM zone 4 includes SEQ ID NO: 13, or is selected from the group of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40, or optionally any of the preceding amino acid sequences with one, two, or three amino acid substitutions, insertions and/or deletions. In still another embodiment of this aspect, which may be combined with any of the preceding embodiments, the substituted amino acid residues are selected from amino acid residues corresponding to M306, A308, and K320 of SEQ ID NO: 1 or from amino acid residues corresponding to T304, D306, and T318 of SEQ ID NO: 8.

In a further embodiment of the preceding aspects, which may be combined with any of the preceding embodiments, the modified plant LysM receptor polypeptide further includes a first JM zone 2, wherein the first JM zone 2 has been modified by insertion, deletion, or substitution of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 2 with the corresponding amino acids from a second JM zone 2 of a second plant LysM receptor polypeptide or wherein the plant LysM receptor polypeptide lacks a first JM zone 2 and the plant LysM receptor polypeptide has been modified by insertion of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 2 with the corresponding amino acids from the second JM zone 2 into the corresponding site when aligning the plant LysM receptor and the second plant LysM receptor, wherein substitution optionally includes deletion of an amino acid not found in the second JM zone 2 and insertion of an amino acid found in the second JM zone 2 but not in the first JM zone 2. In an additional embodiment of this aspect, the first JM zone 2, the second JM zone 2, or both correspond to amino acids 256 to 280 when aligned to SEQ ID NO: 1 or correspond to amino acids 256 to 281 when aligned to SEQ ID NO: 8. In yet another embodiment of this aspect, the first JM zone 2 and/or the second JM zone 2 includes SEQ ID NO: 11, SEQ ID NO: 58, or SEQ ID NO: 4.

In an additional embodiment of the preceding aspects, which may be combined with any of the preceding embodiments, the modified plant LysM receptor polypeptide further includes a first JM zone 3, wherein the first JM zone 3 has been modified by insertion, deletion, or substitution of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 3 with the corresponding amino acids from a second JM zone 3 of a second plant LysM receptor polypeptide or wherein the plant LysM receptor polypeptide lacks a first JM zone 3 and the plant LysM receptor polypeptide has been modified by insertion of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids from the second JM zone 3 into the corresponding site when aligning the plant LysM receptor and the second plant LysM receptor, wherein substitution optionally includes deletion of an amino acid not found in the second JM zone 3 and insertion of an amino acid found in the second JM zone 3 but not in the first JM zone 3. In an additional embodiment of this aspect, the first JM zone 3, the second JM zone 3, or both correspond to amino acids 281 to 304 when aligned to SEQ ID NO: 1 or correspond to amino acids 282 to 302 when aligned to SEQ ID NO: 8. In yet another embodiment of this aspect, the first JM zone 3 and/or the second JM zone 3 includes SEQ ID NO: 12, SEQ ID NO: 59, or SEQ ID NO: 5.

Yet another aspect of the disclosure includes a modified plant non-NFR1 LysM receptor polypeptide engineered for NFR1-mediated root nodule symbiosis signaling including the first kinase C-terminus region has been modified by insertion, deletion, or substitution of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the first kinase C-terminus region with the corresponding amino acids from a C-terminus region of a NFR1 LysM receptor polypeptide or wherein plant non-NFR1 LysM receptor polypeptide lacks a first C-terminus region and the plant non-NFR1 LysM receptor polypeptide has been modified by insertion of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the second C-terminus region into the corresponding site when aligning the plant non-NFR1 LysM receptor and the plant NFR1 LysM receptor. In a further embodiment of this aspect, which may be combined in any of the preceding embodiments, substitution includes deletion of an amino acid not found in the second kinase C-terminus region and insertion of an amino acid found in the second kinase C-terminus region but not in the first kinase C-terminus region. In a further embodiment of this aspect, the first kinase C-terminus region corresponds to amino acids T467, S471, S473, P475, G477, V496, V500, Y510, D517, 1519, V520, S523, S525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, and G621 when aligned to SEQ ID NO: 8, and/or wherein the second kinase C-terminus region corresponds to amino acids 1469, N473, T475, Q477, 1497, 1501, F511, N518, V520, L521, G524, L526, V527, A528, E529, E539, A540, N542, K543, S544, C547, D548, A549, P556, E560, E564, L568, 1570, G574, R575, R579, L583, L591, L601, E603, C605, D607, E608, S610, S613, T615, 1617, L620, and V622 when aligned to SEQ ID NO: 1. In an additional embodiment of this aspect, which may be combined with any of the preceding embodiments, the first kinase C-terminus region includes amino acids T467, S471, S473, P475, G477, V496, V500, Y510, D517, 1519, V520, S523, S525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, and G621 of SEQ ID NO: 8. In a further embodiment of this aspect, the first kinase C-terminus region is modified by substituting one or more amino acids of T467, S471, S473, P475, G477, V496, V500, Y510, D517, 1519, V520, S523, S525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, or G621 of SEQ ID NO: 8 with one or more amino acids of 1469, N473, T475, Q477, 1497, 1501, F511, N518, V520, L521, G524, L526, V527, A528, E529, E539, A540, N542, K543, S544, C547, D548, A549, P556, E560, E564, L568, 1570, G574, R575, R579, L583, L591, L601, E603, C605, D607, E608, S610, S613, T615, 1617, L620, or V622 of SEQ ID NO: 1. In yet another embodiment of this aspect, which may be combined with any of the preceding embodiments, the modified non-NFR1 LysM receptor polypeptide is able to initiate NFR1-mediated root nodule symbiosis signaling.

Still another aspect of the disclosure includes a modified plant non-CERK6 LysM receptor polypeptide engineered for immune signaling including a first kinase N-terminus region, wherein the first kinase N-terminus region has been modified by insertion, deletion, or substitution of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the first kinase N-terminus region with the corresponding amino acids from a second N-terminus region of a CERK6 LysM receptor polypeptide or wherein the non-CERK6 plant LysM receptor polypeptide lacks the first N-terminus region and the non-CERK6 plant LysM receptor polypeptide has been modified by insertion of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the second N-terminus region into the corresponding site when aligning the non-CERK6 plant LysM receptor and the CERK6 LysM receptor. In a further embodiment of this aspect, which may be combined in any of the preceding embodiments, substitution includes deletion of an amino acid not found in the second kinase N-terminus region and insertion of an amino acid found in the second kinase N-terminus region but not in the first kinase N-terminus region. In an additional embodiment of this aspect, the first kinase N-terminus region corresponds to D328, A338, K347, K348, T349, V356, T360, C364, H371, C384, V385, H395, D397, G402, Y404, H406, K410, E411, S416, S417, A425, V445, and L456 when aligned to SEQ ID NO: 1, and the second kinase N-terminus region corresponds to A326, S336, E345, R346, A347, M354, K358, A362, R369, S382, 1383, F393, E395, S400, H402, R404, R408, D409, A414, T415, S423, 1443, and Y454 when aligned to SEQ ID NO: 8. In a further embodiment of this aspect, which may be combined with any of the preceding embodiments, the first kinase N-terminus region includes D328, A338, K347, K348, T349, V356, T360, C364, H371, C384, V385, H395, D397, G402, Y404, H406, K410, E411, S416, S417, A425, V445, and L456 of SEQ ID NO: 1. In yet another embodiment of this aspect, the first kinase N-terminus region is modified by substituting one or more amino acids of D328, A338, K347, K348, T349, V356, T360, C364, H371, C384, V385, H395, D397, G402, Y404, H406, K410, E411, S416, S417, A425, V445, or L456 of SEQ ID NO: 1 with one or more amino acids of A326, S336, E345, R346, A347, M354, K358, A362, R369, S382, 1383, F393, E395, S400, H402, R404, R408, D409, A414, T415, S423, 1443, or Y454 of SEQ ID NO: 8. In still another embodiment of this aspect, which may be combined with any of the preceding embodiments, the modified plant non-CERK6 LysM receptor polypeptide is able to initiate ROS signaling.

In a further embodiment of this aspect, which may be combined with any of the preceding embodiments that has a modified plant LysM receptor polypeptide including a first JM zone 4 modified by insertion, deletion, or substitution of one or more amino acids in the first JM zone 4 with the corresponding amino acids from a second JM zone 4 able to initiate NFR1-mediated root nodule symbiosis signaling, the modified plant LysM receptor polypeptide further includes the first kinase C-terminus region has been modified by insertion, deletion, or substitution of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the first kinase C-terminus region with the corresponding amino acids from a C-terminus region of a NFR1 LysM receptor polypeptide or wherein plant non-NFR1 LysM receptor polypeptide lacks a first C-terminus region and the plant non-NFR1 LysM receptor polypeptide has been modified by insertion of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the second C-terminus region into the corresponding site when aligning the plant non-NFR1 LysM receptor and the plant NFR1 LysM receptor. In a further embodiment of this aspect, which may be combined in any of the preceding embodiments, substitution includes deletion of an amino acid not found in the second kinase C-terminus region and insertion of an amino acid found in the second kinase C-terminus region but not in the first kinase C-terminus region. In an additional embodiment of this aspect, the first kinase C-terminus region corresponds to amino acids T467, S471, S473, P475, G477, V496, V500, Y510, D517, 1519, V520, S523, S525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, and G621 when aligned to SEQ ID NO: 8, and the second kinase C-terminus region corresponds to amino acids 1469, N473, T475, Q477, 1497, 1501, F511, N518, V520, L521, G524, L526, V527, A528, E529, E539, A540, N542, K543, 5544, C547, D548, A549, P556, E560, E564, L568, 1570, G574, R575, R579, L583, L591, L601, E603, C605, D607, E608, S610, S613, T615, 1617, L620, and V622 when aligned to SEQ ID NO: 1. In yet another embodiment of this aspect, which may be combined with any of the preceding embodiments, the first kinase C-terminus region includes amino acids T467, S471, S473, P475, G477, V496, V500, Y510, D517, 1519, V520, S523, S525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, and G621 of SEQ ID NO: 8. In still another embodiment of this aspect, the first kinase C-terminus region is modified by substituting one or more amino acids of T467, S471, S473, P475, G477, V496, V500, Y510, D517, 1519, V520, S523, S525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, or G621 of SEQ ID NO: 8 with one or more amino acids of 1469, N473, T475, Q477, 1497, 1501, F511, N518, V520, L521, G524, L526, V527, A528, E529, E539, A540, N542, K543, S544, C547, D548, A549, P556, E560, E564, L568, 1570, G574, R575, R579, L583, L591, L601, E603, C605, D607, E608, S610, S613, T615, 1617, L620, or V622 of SEQ ID NO: 1. In a further embodiment of this aspect, which may be combined with any of the preceding embodiments, the modified plant LysM receptor polypeptide is able to initiate NFR1-mediated root nodule symbiosis signaling.

In an additional embodiment of this aspect, which may be combined with any of the preceding embodiments that has a modified plant LysM receptor polypeptide including a first JM zone 4 modified by insertion, deletion, or substitution of one or more amino acids in the first JM zone 4 with the corresponding amino acids from a second JM zone 4 able to ROS signaling, the modified plant LysM receptor polypeptide further includes a first kinase N-terminus region, wherein the first kinase N-terminus region has been modified by insertion, deletion, or substitution of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the first kinase N-terminus region with the corresponding amino acids from a second N-terminus region of a CERK6 LysM receptor polypeptide or wherein the non-CERK6 plant LysM receptor polypeptide lacks the first N-terminus region and the non-CERK6 plant LysM receptor polypeptide has been modified by insertion of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the second N-terminus region into the corresponding site when aligning the non-CERK6 plant LysM receptor and the CERK6 LysM receptor. In a further embodiment of this aspect, which may be combined in any of the preceding embodiments, substitution includes deletion of an amino acid not found in the second kinase N-terminus region and insertion of an amino acid found in the second kinase N-terminus region but not in the first kinase N-terminus region. In a further embodiment of this aspect, the first kinase N-terminus region corresponds to D328, A338, K347, K348, T349, V356, T360, C364, H371, C384, V385, H395, D397, G402, Y404, H406, K410, E411, S416, S417, A425, V445, and L456 when aligned to SEQ ID NO: 1, and the second kinase N-terminus region corresponds to A326, S336, E345, R346, A347, M354, K358, A362, R369, S382, 1383, F393, E395, S400, H402, R404, R408, D409, A414, T415, S423, 1443, and Y454 when aligned to SEQ ID NO: 8. In yet another embodiment of this aspect, which may be combined with any one of the preceding embodiments, the first kinase N-terminus region includes D328, A338, K347, K348, T349, V356, T360, C364, H371, C384, V385, H395, D397, G402, Y404, H406, K410, E411, S416, S417, A425, V445, and L456 of SEQ ID NO: 1. In still another embodiment of this aspect, the first kinase N-terminus region is modified by substituting one or more amino acids of D328, A338, K347, K348, T349, V356, T360, C364, H371, C384, V385, H395, D397, G402, Y404, H406, K410, E411, S416, S417, A425, V445, or L456 of SEQ ID NO: 1 with one or more amino acids of A326, S336, E345, R346, A347, M354, K358, A362, R369, S382, 1383, F393, E395, S400, H402, R404, R408, D409, A414, T415, S423, 1443, or Y454 of SEQ ID NO: 8. In a further embodiment of this aspect, which may be combined with any one of the preceding embodiments, the modified LysM receptor polypeptide is able to initiate ROS signaling.

In still another embodiment of this aspect, which may be combined with any of the preceding embodiments that has a modified plant non-NFR1 LysM receptor polypeptide, the modified plant non-NFR1 LysM receptor polypeptide further includes the first kinase C-terminus region has been modified by insertion, deletion, or substitution of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the first kinase C-terminus region with the corresponding amino acids from a C-terminus region of a NFR1 LysM receptor polypeptide or wherein plant non-NFR1 LysM receptor polypeptide lacks a first C-terminus region and the plant non-NFR1 LysM receptor polypeptide has been modified by insertion of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the second C-terminus region into the corresponding site when aligning the plant non-NFR1 LysM receptor and the plant NFR1 LysM receptor. In a further embodiment of this aspect, which may be combined in any of the preceding embodiments, substitution includes deletion of an amino acid not found in the second kinase C-terminus region and insertion of an amino acid found in the second kinase C-terminus region but not in the first kinase C-terminus region. In a further embodiment of this aspect, the first kinase C-terminus region corresponds to amino acids T467, S471, S473, P475, G477, V496, V500, Y510, D517, 1519, V520, S523, S525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, and G621 when aligned to SEQ ID NO: 8, and the second kinase C-terminus region corresponds to amino acids 1469, N473, T475, Q477, I497, I501, F511, N518, V520, L521, G524, L526, V527, A528, E529, E539, A540, N542, K543, S544, C547, D548, A549, P556, E560, E564, L568, 1570, G574, R575, R579, L583, L591, L601, E603, C605, D607, E608, S610, S613, T615, 1617, L620, and V622 when aligned to SEQ ID NO: 1. In an additional embodiment of this aspect, which may be combined with any one of the preceding embodiments, the first kinase C-terminus region includes amino acids T467, S471, S473, P475, G477, V496, V500, Y510, D517, 1519, V520, S523, S525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, and G621 of SEQ ID NO: 8. In yet another embodiment of this aspect, the first kinase C-terminus region is modified by substituting one or more amino acids of T467, S471, S473, P475, G477, V496, V500, Y510, D517, 1519, V520, S523, S525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, or G621 of SEQ ID NO: 8 with one or more amino acids of 1469, N473, T475, Q477, 1497, I501, F511, N518, V520, L521, G524, L526, V527, A528, E529, E539, A540, N542, K543, S544, C547, D548, A549, P556, E560, E564, L568, 1570, G574, R575, R579, L583, L591, L601, E603, C605, D607, E608, S610, S613, T615, 1617, L620, or V622 of SEQ ID NO: 1. In still another embodiment of this aspect, which may be combined with any one of the preceding embodiments, the modified plant non-NFR1 LysM receptor polypeptide is able to initiate NFR1-mediated root nodule symbiosis signaling.

In yet another embodiment of this aspect, which may be combined with any of the preceding embodiments that has a modified plant LysM receptor polypeptide with enhanced ROS signaling, the modified plant LysM receptor polypeptide further includes a first kinase N-terminus region, wherein the first kinase N-terminus region has been modified by insertion, deletion, or substitution of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the first kinase N-terminus region with the corresponding amino acids from a second N-terminus region of a CERK6 LysM receptor polypeptide or wherein the non-CERK6 plant LysM receptor polypeptide lacks the first N-terminus region and the non-CERK6 plant LysM receptor polypeptide has been modified by insertion of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the second N-terminus region into the corresponding site when aligning the non-CERK6 plant LysM receptor and the CERK6 LysM receptor. In a further embodiment of this aspect, which may be combined in any of the preceding embodiments, substitution includes deletion of an amino acid not found in the second kinase N-terminus region and insertion of an amino acid found in the second kinase N-terminus region but not in the first kinase N-terminus region. In an additional embodiment of this aspect, the first kinase N-terminus region corresponds to D328, A338, K347, K348, T349, V356, T360, C364, H371, C384, V385, H395, D397, G402, Y404, H406, K410, E411, S416, S417, A425, V445, and L456 when aligned to SEQ ID NO: 1, and the second kinase N-terminus region corresponds to A326, S336, E345, R346, A347, M354, K358, A362, R369, S382, 1383, F393, E395, S400, H402, R404, R408, D409, A414, T415, S423, 1443, and Y454 when aligned to SEQ ID NO: 8. In a further embodiment of this aspect, which may be combined with any one of the preceding embodiments, the first kinase N-terminus region includes D328, A338, K347, K348, T349, V356, T360, C364, H371, C384, V385, H395, D397, G402, Y404, H406, K410, E411, S416, S417, A425, V445, and L456 of SEQ ID NO: 1. In yet another embodiment of this aspect, the first kinase N-terminus region is modified by substituting one or more amino acids of D328, A338, K347, K348, T349, V356, T360, C364, H371, C384, V385, H395, D397, G402, Y404, H406, K410, E411, S416, S417, A425, V445, or L456 of SEQ ID NO: 1 with one or more amino acids of A326, S336, E345, R346, A347, M354, K358, A362, R369, S382, 1383, F393, E395, S400, H402, R404, R408, D409, A414, T415, S423, 1443, or Y454 of SEQ ID NO: 8. In still another embodiment of this aspect, which may be combined with any one of the preceding embodiments, the modified LysM receptor polypeptide is able to initiate ROS signaling.

In an additional embodiment of the preceding aspects, which may be combined with any of the preceding embodiments, the modified plant LysM receptor polypeptide further includes a first extracellular domain, wherein the first extracellular domain is modified as compared to the amino acid sequence of the corresponding unmodified plant LysM receptor polypeptide. In a further embodiment of this aspect, the first extracellular domain is modified by substituting one or more amino acids of the first extracellular domain with one or more amino acids of a second extracellular domain, and wherein the second extracellular domain has a different affinity, selectivity, and/or specificity for oligosaccharides than the first extracellular domain. The extracellular domain is critical for receptor specificity. For example, the extracellular domain of NFR1 is critical for recognition of rhizobial LCOs, while the extracellular domain of CERK6 is critical for recognition of pathogen COs. The modified LysM receptors of any of the preceding embodiments can be modified in their ectodomain as disclosed in U.S. patent application Ser. No. 17/324,354 (U.S. Pat. App. Pub. No. US-2021-03663200-A1), which is incorporated by reference for its disclosure of modifications to LysM receptor domains for engineering of recognition of LCOs. The modified LysM receptors of any of the preceding embodiments can also be modified in their ectodomain as disclosed in and U.S. patent application Ser. No. 17/267,240 (U.S. Pat. App. Pub. NO. US-2021-0233608-A1), which is incorporated by reference for its disclosure of modifications to LysM receptor domains for engineering of recognition of LCOs.

An additional aspect of the disclosure includes methods of generating a modified plant LysM receptor polypeptide, including: (a) aligning an amino acid sequence of a candidate receptor to SEQ ID NO: 8 to identify a first JM zone 4 corresponding to amino acids 303 to 325 of SEQ ID NO: 8, or aligning an amino acid sequence of a candidate receptor to SEQ ID NO: 1 to identify a first JM zone 4 corresponding to amino acids 305-327 of SEQ ID NO: 1, and optionally further aligning the JM zone 4 of the candidate receptor to SEQ ID NO: 13, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40, or optionally any of the preceding amino acid sequences with one, two, or three amino acid substitutions, insertions and/or deletions; (b) modifying the first JM zone 4 by substituting at least two or at least three amino acid residues in the first JM zone 4 with corresponding amino acid residues that are different in a second JM zone 4; and (c) generating the modified plant LysM receptor polypeptide wherein the first JM zone 4 has been substituted with corresponding amino acid residues from the second JM zone 4.

A further aspect of the disclosure includes methods of generating a modified plant LysM receptor polypeptide, including: (a) aligning an amino acid sequence of a candidate receptor to SEQ ID NO: 8 to identify a first JM zone 2 corresponding to amino acids 256 to 281 of SEQ ID NO: 8, or aligning an amino acid sequence of a candidate receptor to SEQ ID NO: 1 to identify a first JM zone 2 corresponding to amino acids 256-280 of SEQ ID NO: 1; (b) modifying the first JM zone 2 by substituting inserting, deleting, or substituting one or more amino acid residues in the first JM zone 2 with corresponding amino acid residues that are different in a second JM zone 2; and (c) generating the modified plant LysM receptor polypeptide wherein the first JM zone 2 has been substituted with corresponding amino acid residues from the second JM zone 2.

Yet another aspect of the disclosure includes methods of generating a modified plant LysM receptor polypeptide, including: (a) aligning an amino acid sequence of a candidate receptor to SEQ ID NO: 8 to identify a first JM zone 3 corresponding to amino acids 282 to 302 of SEQ ID NO: 8, or aligning an amino acid sequence of a candidate receptor to SEQ ID NO: 1 to identify a first JM zone 3 corresponding to amino acids 281-304 of SEQ ID NO: 1; (b) modifying the first JM zone 3 by substituting inserting, deleting, or substituting one or more amino acid residues in the first JM zone 3 with corresponding amino acid residues that are different in a second JM zone 3; and (c) generating the modified plant LysM receptor polypeptide wherein the first JM zone 3 has been substituted with corresponding amino acid residues from the second JM zone 3.

Still another aspect of the disclosure includes methods of generating a modified plant LysM receptor polypeptide, including: (a) aligning an amino acid sequence of a candidate receptor to SEQ ID NO: 8 to identify a first kinase C-terminus region corresponding to amino acids T467, S471, S473, P475, G477, V496, V500, Y510, D517, 1519, V520, S523, S525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, and G621 of SEQ ID NO: 8, or aligning an amino acid sequence of a candidate receptor to SEQ ID NO: 1 to identify a first kinase C-terminus region corresponding to amino acids 1469, N473, T475, Q477, 1497, 1501, F511, N518, V520, L521, G524, L526, V527, A528, E529, E539, A540, N542, K543, S544, C547, D548, A549, P556, E560, E564, L568, 1570, G574, R575, R579, L583, L591, L601, E603, C605, D607, E608, S610, S613, T615, 1617, L620, and V622 of SEQ ID NO: 1; (b) modifying the first kinase C-terminus region by substituting one or more amino acid residues in the first kinase C-terminus region with corresponding amino acid residues that are different in a second kinase C-terminus region; and (c) generating the modified plant LysM receptor polypeptide wherein the first kinase C-terminus region has been substituted with corresponding amino acid residues from the second kinase C-terminus region.

An additional aspect of the disclosure includes methods of generating a modified plant LysM receptor polypeptide, including: (a) aligning an amino acid sequence of a candidate receptor to SEQ ID NO: 1 to identify a first kinase N-terminus region corresponding to amino acids D328, A338, K347, K348, T349, V356, T360, C364, H371, C384, V385, H395, D397, G402, Y404, H406, K410, E411, S416, S417, A425, V445, and L456 of SEQ ID NO: 1, or aligning an amino acid sequence of a candidate receptor to SEQ ID NO: 8 to identify a first kinase N-terminus region corresponding to amino acids A326, S336, E345, R346, A347, M354, K358, A362, R369, S382, 1383, F393, E395, S400, H402, R404, R408, D409, A414, T415, S423, 1443, and Y454 of SEQ ID NO: 8; (b) modifying the first kinase N-terminus region by substituting one or more amino acid residues in the first kinase N-terminus region with corresponding amino acid residues that are different in a second kinase N-terminus region; and (c) generating the modified plant LysM receptor polypeptide wherein the first kinase N-terminus region has been substituted with corresponding amino acid residues from the second kinase N-terminus region.

Further embodiments of the preceding aspects, which may be combined with any of the preceding embodiments that has methods of generating a modified plant LysM receptor polypeptide, include the modified plant LysM receptor polypeptide produced by any of the methods of any one of the preceding embodiments, or a combination thereof.

### Genetically Modified Plants and Related Methods

Some aspects of the disclosure include a genetically modified plant or part thereof including the modified plant LysM receptor polypeptide of any one of the preceding embodiments that has a modified plant LysM receptor polypeptide including a first JM zone 4 modified by insertion, deletion, or substitution of one or more amino acids in the first JM zone 4 with the corresponding amino acids from a second JM zone 4 able to initiate NFR1-mediated root nodule symbiosis signaling. In a further embodiment of this aspect, the modified plant LysM receptor polypeptide includes a first JM zone 4 modified by insertion, deletion, or substitution of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 4 with the corresponding amino acids from a second JM zone 4 of a second plant LysM receptor polypeptide or wherein the plant LysM receptor polypeptide lacks a first JM zone 4 and the plant LysM receptor polypeptide has been modified by insertion of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 4 with the corresponding amino acids from the second JM zone 4 into the corresponding site when aligning the plant LysM receptor and the second plant LysM receptor, wherein the first JM zone 4 includes SEQ ID NO: 13, is selected from the group of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40, or conservative substitutions thereof, or optionally any of the preceding amino acid sequences with one, two, or three amino acid substitutions, insertions and/or deletions, or includes SEQ ID NO: 60, and/or wherein the second JM zone 4 includes SEQ ID NO: 6, or is selected from the group of SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and SEQ ID NO: 52, or optionally any of the preceding amino acid sequences with one, two, or three amino acid substitutions, insertions and/or deletions; and optionally further includes: (i) a first JM zone 2, wherein the first JM zone 2 has been modified by insertion, deletion, or substitution of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 2 with the corresponding amino acids from a second JM zone 2 of a second plant LysM receptor polypeptide or wherein the plant LysM receptor polypeptide lacks a first JM zone 2 and the plant LysM receptor polypeptide has been modified by insertion of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 2 with the corresponding amino acids from the second JM zone 2 into the corresponding site when aligning the plant LysM receptor and the second plant LysM receptor, and wherein the first JM zone 2, the second JM zone 2, or both correspond to amino acids 256 to 280 when aligned to SEQ ID NO: 1 or correspond to amino acids 256 to 281 when aligned to SEQ ID NO: 8; (ii) a first JM zone 3, wherein the first JM zone 3 has been modified by insertion, deletion, or substitution of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 3 with the corresponding amino acids from a second JM zone 3 of a second plant LysM receptor polypeptide or wherein the plant LysM receptor polypeptide lacks a first JM zone 3 and the plant LysM receptor polypeptide has been modified by insertion of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids from the second JM zone 3 into the corresponding site when aligning the plant LysM receptor and the second plant LysM receptor, and wherein the first JM zone 3, the second JM zone 3, or both correspond to amino acids 281 to 304 when aligned to SEQ ID NO: 1 or correspond to amino acids 282 to 302 when aligned to SEQ ID NO: 8; (iii) the first kinase C-terminus region has been modified by insertion, deletion, or substitution of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the first kinase C-terminus region with the corresponding amino acids from a C-terminus region of a NFR1 LysM receptor polypeptide or wherein plant non-NFR1 LysM receptor polypeptide lacks a first C-terminus region and the plant non-NFR1 LysM receptor polypeptide has been modified by insertion of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the second C-terminus region into the corresponding site when aligning the plant non-NFR1 LysM receptor and the plant NFR1 LysM receptor, wherein the first kinase C-terminus region corresponds to amino acids T467, S471, S473, P475, G477, V496, V500, Y510, D517, 1519, V520, S523, S525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, and G621 when aligned to SEQ ID NO: 8, and/or wherein the second kinase C-terminus region corresponds to amino acids 1469, N473, T475, Q477, 1497, I501, F511, N518, V520, L521, G524, L526, V527, A528, E529, E539, A540, N542, K543, S544, C547, D548, A549, P556, E560, E564, L568, 1570, G574, R575, R579, L583, L591, L601, E603, C605, D607, E608, S610, S613, T615, 1617, L620, and V622 when aligned to SEQ ID NO: 1; and/or (iv) a first extracellular domain modified by substituting one or more amino acids of the first extracellular domain with one or more amino acids of a second extracellular domain, wherein the second extracellular domain has a different affinity, selectivity, and/or specificity for oligosaccharides than the first extracellular domain. In an additional embodiment of this aspect, which may be combined with any of the preceding embodiments, the modified plant LysM receptor polypeptide is able to initiate NFR1-mediated root nodule symbiosis signaling.

Some aspects of the disclosure include a genetically modified plant or part thereof including the modified plant LysM receptor polypeptide of any one of the preceding embodiments that has a modified plant LysM receptor polypeptide including a first JM zone 4 modified by insertion, deletion, or substitution of one or more amino acids in the first JM zone 4 with the corresponding amino acids from a second JM zone 4 able to ROS signaling. In a further embodiment of this aspect, the modified plant LysM receptor polypeptide includes a first JM zone 4 modified by insertion, deletion, or substitution of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 4 with the corresponding amino acids from a second JM zone 4 of a second plant LysM receptor polypeptide or wherein the plant LysM receptor polypeptide lacks a first JM zone 4 and the plant LysM receptor polypeptide has been modified by insertion of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 4 with the corresponding amino acids from the second JM zone 4 into the corresponding site when aligning the plant LysM receptor and the second plant LysM receptor, wherein the first JM zone 4 includes SEQ ID NO: 6, or is selected from the group of SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and SEQ ID NO: 52, or optionally any of the preceding amino acid sequences with one, two, or three amino acid substitutions, insertions and/or deletions, and/or wherein the second JM zone 4 includes SEQ ID NO: 13, or is selected from the group of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40, or optionally any of the preceding amino acid sequences with one, two, or three amino acid substitutions, insertions and/or deletions; and optionally further includes: (i) a first JM zone 2, wherein the first JM zone 2 has been modified by insertion, deletion, or substitution of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 2 with the corresponding amino acids from a second JM zone 2 of a second plant LysM receptor polypeptide or wherein the plant LysM receptor polypeptide lacks a first JM zone 2 and the plant LysM receptor polypeptide has been modified by insertion of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 2 with the corresponding amino acids from the second JM zone 2 into the corresponding site when aligning the plant LysM receptor and the second plant LysM receptor, and wherein the first JM zone 2, the second JM zone 2, or both correspond to amino acids 256 to 280 when aligned to SEQ ID NO: 1 or correspond to amino acids 256 to 281 when aligned to SEQ ID NO: 8; (ii) a first JM zone 3, wherein the first JM zone 3 has been modified by insertion, deletion, or substitution of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 3 with the corresponding amino acids from a second JM zone 3 of a second plant LysM receptor polypeptide or wherein the plant LysM receptor polypeptide lacks a first JM zone 3 and the plant LysM receptor polypeptide has been modified by insertion of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids from the second JM zone 3 into the corresponding site when aligning the plant LysM receptor and the second plant LysM receptor, and wherein the first JM zone 3, the second JM zone 3, or both correspond to amino acids 281 to 304 when aligned to SEQ ID NO: 1 or correspond to amino acids 282 to 302 when aligned to SEQ ID NO: 8; (iii) a first kinase N-terminus region, wherein the first kinase N-terminus region has been modified by insertion, deletion, or substitution of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the first kinase N-terminus region with the corresponding amino acids from a second N-terminus region of a CERK6 LysM receptor polypeptide or wherein the non-CERK6 plant LysM receptor polypeptide lacks the first N-terminus region and the non-CERK6 plant LysM receptor polypeptide has been modified by insertion of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the second N-terminus region into the corresponding site when aligning the non-CERK6 plant LysM receptor and the CERK6 LysM receptor, wherein the first kinase N-terminus region corresponds to D328, A338, K347, K348, T349, V356, T360, C364, H371, C384, V385, H395, D397, G402, Y404, H406, K410, E411, S416, S417, A425, V445, and L456 when aligned to SEQ ID NO: 1, and/or wherein the second kinase N-terminus region corresponds to A326, S336, E345, R346, A347, M354, K358, A362, R369, S382, 1383, F393, E395, S400, H402, R404, R408, D409, A414, T415, S423, 1443, and Y454 when aligned to SEQ ID NO: 8; and/or (iv) a first extracellular domain modified by substituting one or more amino acids of the first extracellular domain with one or more amino acids of a second extracellular domain, wherein the second extracellular domain has a different affinity, selectivity, and/or specificity for oligosaccharides than the first extracellular domain. In an additional embodiment of this aspect, which may be combined with any one of the preceding embodiments, the modified plant LysM receptor polypeptide is able to initiate ROS signaling.

Some aspects of the disclosure include a genetically modified plant or part thereof including the modified plant LysM receptor polypeptide of any one of the preceding embodiments that has a modified plant non-NFR1 LysM receptor polypeptide. In a further embodiment of this aspect, the modified plant non-NFR1 LysM receptor polypeptide includes a first JM zone 4, wherein the first JM zone 4 was modified by substitution of one or more amino acids in the first JM zone 4 with the corresponding amino acids from a second JM zone 4 from an NFR1 LysM receptor polypeptide with NFR1-mediated root nodule symbiosis signaling, wherein the first JM zone 4 includes SEQ ID NO: 13, is selected from the group of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40, or conservative substitutions thereof, or optionally any of the preceding amino acid sequences with one, two, or three amino acid substitutions, insertions and/or deletions, or includes SEQ ID NO: 60, and/or wherein the second JM zone 4 includes SEQ ID NO: 6, or is selected from the group of SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and SEQ ID NO: 52, or optionally any of the preceding amino acid sequences with one, two, or three amino acid substitutions, insertions and/or deletions; and optionally further includes: (i) a first JM zone 2, wherein the first JM zone 2 has been modified by insertion, deletion, or substitution of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 2 with the corresponding amino acids from a second JM zone 2 of a second plant LysM receptor polypeptide or wherein the plant LysM receptor polypeptide lacks a first JM zone 2 and the plant LysM receptor polypeptide has been modified by insertion of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 2 with the corresponding amino acids from the second JM zone 2 into the corresponding site when aligning the plant LysM receptor and the second plant LysM receptor, and wherein the first JM zone 2, the second JM zone 2, or both correspond to amino acids 256 to 280 when aligned to SEQ ID NO: 1 or correspond to amino acids 256 to 281 when aligned to SEQ ID NO: 8; (ii) a first JM zone 3, wherein the first JM zone 3 has been modified by insertion, deletion, or substitution of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 3 with the corresponding amino acids from a second JM zone 3 of a second plant LysM receptor polypeptide or wherein the plant LysM receptor polypeptide lacks a first JM zone 3 and the plant LysM receptor polypeptide has been modified by insertion of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids from the second JM zone 3 into the corresponding site when aligning the plant LysM receptor and the second plant LysM receptor, and wherein the first JM zone 3, the second JM zone 3, or both correspond to amino acids 281 to 304 when aligned to SEQ ID NO: 1 or correspond to amino acids 282 to 302 when aligned to SEQ ID NO: 8; (iii) the first kinase C-terminus region has been modified by insertion, deletion, or substitution of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the first kinase C-terminus region with the corresponding amino acids from a C-terminus region of a NFR1 LysM receptor polypeptide or wherein plant non-NFR1 LysM receptor polypeptide lacks a first C-terminus region and the plant non-NFR1 LysM receptor polypeptide has been modified by insertion of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the second C-terminus region into the corresponding site when aligning the plant non-NFR1 LysM receptor and the plant NFR1 LysM receptor, wherein the first kinase C-terminus region corresponds to amino acids T467, S471, S473, P475, G477, V496, V500, Y510, D517, 1519, V520, S523, S525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, and G621 when aligned to SEQ ID NO: 8, and/or wherein the second kinase C-terminus region corresponds to amino acids 1469, N473, T475, Q477, 1497, I501, F511, N518, V520, L521, G524, L526, V527, A528, E529, E539, A540, N542, K543, S544, C547, D548, A549, P556, E560, E564, L568, 1570, G574, R575, R579, L583, L591, L601, E603, C605, D607, E608, S610, S613, T615, 1617, L620, and V622 when aligned to SEQ ID NO: 1; and/or (iv) a first extracellular domain modified by substituting one or more amino acids of the first extracellular domain with one or more amino acids of a second extracellular domain, wherein the second extracellular domain has a different affinity, selectivity, and/or specificity for oligosaccharides than the first extracellular domain. In an additional embodiment of this aspect, which may be combined with any one of the preceding embodiments, the modified plant non-NFR1 LysM receptor polypeptide is able to initiate NFR1-mediated root nodule symbiosis signaling.

Some aspects of the disclosure include a genetically modified plant or part thereof including the modified plant LysM receptor polypeptide of any one of the preceding embodiments that has a modified plant LysM receptor polypeptide with enhanced ROS signaling. In a further embodiment of this aspect, the modified plant LysM receptor polypeptide includes a first JM zone 4, wherein the first JM zone 4 was modified by substitution of one or more amino acids in the first JM zone 4 with the corresponding amino acids from a second JM zone 4 from a LysM receptor polypeptide with ROS signaling, and wherein the first JM zone 4 includes SEQ ID NO: 6, or is selected from the group of SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and SEQ ID NO: 52, or optionally any of the preceding amino acid sequences with one, two, or three amino acid substitutions, insertions and/or deletions, and/or wherein the second JM zone 4 includes SEQ ID NO: 13, or is selected from the group of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40, or optionally any of the preceding amino acid sequences with one, two, or three amino acid substitutions, insertions and/or deletions; and optionally further includes: (i) a first JM zone 2, wherein the first JM zone 2 has been modified by insertion, deletion, or substitution of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 2 with the corresponding amino acids from a second JM zone 2 of a second plant LysM receptor polypeptide or wherein the plant LysM receptor polypeptide lacks a first JM zone 2 and the plant LysM receptor polypeptide has been modified by insertion of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 2 with the corresponding amino acids from the second JM zone 2 into the corresponding site when aligning the plant LysM receptor and the second plant LysM receptor, and wherein the first JM zone 2, the second JM zone 2, or both correspond to amino acids 256 to 280 when aligned to SEQ ID NO: 1 or correspond to amino acids 256 to 281 when aligned to SEQ ID NO: 8; (ii) a first JM zone 3, wherein the first JM zone 3 has been modified by insertion, deletion, or substitution of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 3 with the corresponding amino acids from a second JM zone 3 of a second plant LysM receptor polypeptide or wherein the plant LysM receptor polypeptide lacks a first JM zone 3 and the plant LysM receptor polypeptide has been modified by insertion of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids from the second JM zone 3 into the corresponding site when aligning the plant LysM receptor and the second plant LysM receptor, and wherein the first JM zone 3, the second JM zone 3, or both correspond to amino acids 281 to 304 when aligned to SEQ ID NO: 1 or correspond to amino acids 282 to 302 when aligned to SEQ ID NO: 8; (iii) a first kinase N-terminus region, wherein the first kinase N-terminus region has been modified by insertion, deletion, or substitution of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the first kinase N-terminus region with the corresponding amino acids from a second N-terminus region of a CERK6 LysM receptor polypeptide or wherein the non-CERK6 plant LysM receptor polypeptide lacks the first N-terminus region and the non-CERK6 plant LysM receptor polypeptide has been modified by insertion of one or more amino acids, two or more amino acids, three or more amino acids, four or more amino acids, five or more amino acids, ten percent or more of the amino acids, twenty percent or more of the amino acids, thirty percent or more of the or more amino acids, forty percent or more of the or more amino acids, fifty percent or more of the or more amino acids, fifty percent or more of the or more amino acids, sixty percent or more of the or more amino acids, seventy percent or more of the or more amino acids, eighty percent or more of the amino acids, ninety percent or more of the amino acids, or all amino acids of the second N-terminus region into the corresponding site when aligning the non-CERK6 plant LysM receptor and the CERK6 LysM receptor, wherein the first kinase N-terminus region corresponds to D328, A338, K347, K348, T349, V356, T360, C364, H371, C384, V385, H395, D397, G402, Y404, H406, K410, E411, 5416, 5417, A425, V445, and L456 when aligned to SEQ ID NO: 1, and/or wherein the second kinase N-terminus region corresponds to A326, 5336, E345, R346, A347, M354, K358, A362, R369, 5382, 1383, F393, E395, 5400, H402, R404, R408, D409, A414, T415, S423, 1443, and Y454 when aligned to SEQ ID NO: 8; and/or (iv) a first extracellular domain modified by substituting one or more amino acids of the first extracellular domain with one or more amino acids of a second extracellular domain, wherein the second extracellular domain has a different affinity, selectivity, and/or specificity for oligosaccharides than the first extracellular domain. In an additional embodiment of this aspect, which may be combined with any one of the preceding embodiments, the modified plant LysM receptor polypeptide is able to initiate ROS signaling.

In a further embodiment of the preceding aspects, which may be combined with any of the preceding embodiments that has a genetically modified plant or part thereof, the plant part is a leaf, a stem, a root, a root primordia, a flower, a seed, a fruit, a kernel, a grain, a cell, or a portion thereof. In yet another embodiment of the preceding aspects, which may be combined with any of the preceding embodiments, the plant is selected from the group of cassava (e.g., manioc, yucca, Manihot esculenta), yam (e.g., Dioscorea rotundata, Dioscorea alata, Dioscorea trifida, Dioscorea sp.), sweet potato (e.g., Ipomoea batatas), taro (e.g., Colocasia esculenta), oca (e.g., Oxalis tuberosa), corn (e.g., maize, Zea mays), rice (e.g., indica rice, japonica rice, aromatic rice, glutinous rice, Oryza sativa, Oryza glaberrima), wild rice (e.g., Zizania spp., Porteresia spp.), barley (e.g., Hordeum vulgare), sorghum (e.g., Sorghum bicolor), millet (e.g., finger millet, fonio millet, foxtail millet, pearl millet, barnyard millets, Eleusine coracana, Panicum sumatrense, Panicum milaceum, Setaria italica, Pennisetum glaucum, Digitaria spp., Echinocloa spp.), teff (e.g., Eragrostis tej), oat (e.g., Avena sativa), triticale (e.g., X Triticosecale Wittmack, Triticosecale schlanstedtense Wittm., Triticosecale neoblaringhemii A. Camus, Triticosecale neoblaringhemii A. Camus), rye (e.g., Secale cereale, Secale cereanum), wheat (e.g., common wheat, spelt, durum, einkorn, emmer, kamut, Triticum aestivum, Triticum spelta, Triticum durum, Triticum urartu, Triticum monococcum, Triticum turanicum, Triticum spp.), Trema spp. (e.g., Trema cannabina, Trema cubense, Trema discolor, Trema domingensis, Trema integerrima, Trema lamarckiana, Trema micrantha, Trema orientalis, Trema philippinensis, Trema strigilosa, Trema tomentosa, Trema levigata), apple (e.g., Malus domestica, Malus pumila, Pyrus malus), pear (e.g., Pyrus communis, Pyrus×bretschneideri, Pyrus pyrifolia, Pyrus sinkiangensis, Pyrus pashia, Pyrus spp.), plum (e.g., Mirabelle, greengage, damson, Prunus domestica, Prunus salicina, Prunus mume), apricot (e.g., Prunus armeniaca, Prunus brigantine, Prunus mandshurica), peach (e.g., Prunus persica), almond (e.g., Prunus dulcis, Prunus amygdalus), walnut (e.g., Persian walnut, English walnut, black walnut, Juglans regia, Juglans nigra, Juglans cinerea, Juglans californica), strawberry (e.g., Fragaria×ananassa, Fragaria chiloensis, Fragaria virginiana, Fragaria vesca), raspberry (e.g., European red raspberry, black raspberry, Rubus idaeus L., Rubus occidentalis, Rubus strigosus), blackberry (e.g., evergreen blackberry, Himalayan blackberry, Rubus fruticosus, Rubus ursinus, Rubus laciniatus, Rubus argutus, Rubus armeniacus, Rubus plicatus, Rubus ulmifolius, Rubus allegheniensis, Rubus subgenus Eubatus sect. Moriferi & Ursini), red currant (e.g., white currant, Ribes rubrum), black currant (e.g., cassis, Ribes nigrum), gooseberry (e.g., Ribes uva-crispa, Ribes grossulari, Ribes hirtellum), melon (e.g., watermelon, winter melon, casabas, cantaloupe, honeydew, muskmelon, Citrullus lanatus, Benincasa hispida, Cucumis melo, Cucumis melo cantalupensis, Cucumis melo inodorus, Cucumis melo reticulatus), cucumber (e.g., slicing cucumbers, pickling cucumbers, English cucumber, Cucumis sativus), pumpkin (e.g., Cucurbita pepo, Cucurbita maxima), squash (e.g., gourd, Cucurbita argyrosperma, Cucurbita ficifolia, Cucurbita maxima, Cucurbita moschata), grape (e.g., Vitis vinifera, Vitis amurensis, Vitis labrusca, Vitis mustangensis, Vitis riparia, Vitis rotundifolia), bean (e.g., Phaseolus vulgaris, Phaseolus lunatus, Vigna angularis, Vigna radiate, Vigna mungo, Phaseolus coccineus, Vigna umbellate, Vigna acontifolia, Phaseolus acutifolius, Vicia faba, Vicia faba equine, Phaseolus spp., Vigna spp.), soybean (e.g., soy, soya bean, Glycine max, Glycine soja), pea (e.g., Pisum spp., Pisum sativum var. sativum, Pisum sativum var. arvense), pea (e.g., Pisum spp., Pisum sativum var. sativum, Pisum sativum var. arvense), chickpea (e.g., garbanzo, Bengal gram, Cicer arietinum), cowpea (e.g., Vigna unguiculata), pigeon pea (e.g., Arhar/Toor, cajan pea, Congo bean, gandules, Caganus cajan), lentil (e.g., Lens culinaris), Bambara groundnut (e.g., earth pea, Vigna subterranea), lupin (e.g., Lupinus spp.), pulses (e.g., minor pulses, Lablab purpureaus, Canavalia ensiformis, Canavalia gladiate, Psophocarpus tetragonolobus, Mucuna pruriens var. utilis, Pachyrhizus erosus), Medicago spp. (e.g., Medicago sativa, Medicago truncatula, Medicago arborea), Lotus spp. (e.g., Lotus japonicus), forage legumes (e.g., Leucaena spp., Albizia spp., Cyamopsis spp., Sesbania spp., Stylosanthes spp., Trifolium spp., Vicia spp.), indigo (e.g., Indigofera spp., Indigofera tinctoria, Indigofera suffruticosa, Indigofera articulata, Indigofera oblongifolia, Indigofera aspalthoides, Indigofera suffruticosa, Indigofera arrecta), legume trees (e.g., locust trees, Gleditsia spp., Robinia spp., Kentucky coffeetree, Gymnocladus dioicus, Acacia spp., Laburnum spp., Wisteria spp.), or hemp (e.g., cannabis, Cannabis sativa).

Further aspects of the present disclosure relate to methods of producing the genetically modified plant or part thereof of any one of the preceding embodiments that has a modified plant LysM receptor polypeptide including a first JM zone 4 modified by insertion, deletion, or substitution of one or more amino acids in the first JM zone 4 with the corresponding amino acids from a second JM zone 4 able to initiate NFR1-mediated root nodule symbiosis signaling or that has a modified plant non-NFR1 LysM receptor polypeptide, including introducing a genetic alteration to the plant including a first nucleic acid sequence encoding the modified plant LysM receptor polypeptide or the modified plant non-NFR1 LysM receptor polypeptide. In an additional embodiment of this aspect, the nucleic acid sequence is operably linked to a promoter, wherein the promoter is a root specific promoter, an inducible promoter, a constitutive promoter, or a combination thereof. In a further embodiment of this aspect, the promoter is selected from the group of a NFR1 promoter, a NFR5 promoter, a LYK3 promoter, a CERK6 promoter, a NFP promoter, a Lotus japonicus NFR5 promoter (SEQ ID NO: 109), a Lotus japonicus NFR1 promoter (SEQ ID NO: 151), a Lotus japonicus CERK6 promoter (SEQ ID NO: 111), a Medicago truncatula NFP promoter (SEQ ID NO: 110), a Medicago truncatula LYK3 promoter (SEQ ID NO: 112), a maize metallothioneine promoter, a chitinase promoter, a maize ZRP2 promoter, a tomato LeExt1 promoter, a glutamine synthetase soybean root promoter, a RCC3 promoter, a rice antiquitin promoter, a LRR receptor kinase promoter, or an Arabidopsis pCO2 promoter. In yet another embodiment of this aspect, the promoter is selected from the group of a CaMV35S promoter, a derivative of the CaMV35S promoter, a maize ubiquitin promoter, a polyubiquitin promoter, a vein mosaic cassava virus promoter, or an Arabidopsis UBQ10 promoter. In yet another embodiment of this aspect, the nucleic acid sequence is inserted into the genome of the plant so that the nucleic acid sequence is operably linked to an endogenous promoter, and wherein the endogenous promoter is a root specific promoter.

Additional aspects of the present disclosure relate to methods of producing the genetically modified plant or part thereof of any one of the preceding embodiments that has a modified plant LysM receptor polypeptide including a first JM zone 4 modified by insertion, deletion, or substitution of one or more amino acids in the first JM zone 4 with the corresponding amino acids from a second JM zone 4 able to initiate NFR1-mediated root nodule symbiosis signaling or that has a modified plant non-NFR1 LysM receptor polypeptide, including genetically modifying the plant or part thereof by transforming the plant or part thereof with one or more gene editing components that target an endogenous nuclear genome sequence encoding an endogenous plant LysM receptor polypeptide or plant non-NFR1 LysM receptor polypeptide to genetically modify a first JM zone 4 by insertion, deletion, or substitution of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 4 with the corresponding amino acids from a second JM zone 4 of a second plant LysM receptor polypeptide or wherein the plant LysM receptor polypeptide lacks a first JM zone 4 and the plant LysM receptor polypeptide has been modified by insertion of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 4 with the corresponding amino acids from the second JM zone 4 into the corresponding site when aligning the plant LysM receptor and the second plant LysM receptor, and optionally further including: (i) genetically modifying a first JM zone 2 by insertion, deletion, or substitution of one or more amino acids in the first JM zone 2 with the corresponding amino acids from a second JM zone 2 of a second plant LysM receptor polypeptide; (ii) genetically modifying a first JM zone 3 by insertion, deletion, or substitution of one or more amino acids in the first JM zone 3 with the corresponding amino acids from a second JM zone 3 of a second plant LysM receptor polypeptide; (iii) genetically modifying a first kinase C-terminus region by substitution of one or more amino acids in the first kinase C-terminus region with the corresponding amino acids from a second kinase C-terminus region from a NFR1 LysM receptor polypeptide; and/or (iv) genetically modifying a first extracellular domain by substituting one or more amino acids of the first extracellular domain with one or more amino acids of a second extracellular domain, wherein the second extracellular domain has a different affinity, selectivity, and/or specificity for oligosaccharides than the first extracellular domain. In a further embodiment of this aspect, the one or more gene editing components include a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence.

Further aspects of the present disclosure relate to methods of producing the genetically modified plant or part thereof of any one of the preceding embodiments that has a modified plant LysM receptor polypeptide including a first JM zone 4 modified by insertion, deletion, or substitution of one or more amino acids in the first JM zone 4 with the corresponding amino acids from a second JM zone 4 able to ROS signaling or a modified plant LysM receptor polypeptide with enhanced ROS signaling, including introducing a genetic alteration to the plant including a first nucleic acid sequence encoding the modified plant LysM receptor polypeptide or the modified plant LysM receptor polypeptide with enhanced ROS signaling. In an additional embodiment of this aspect, the nucleic acid sequence is operably linked to a promoter, wherein the promoter is a root specific promoter, an inducible promoter, a constitutive promoter, or a combination thereof. In a further embodiment of this aspect, the promoter is selected from the group of a NFR1 promoter, a NFR5 promoter, a LYK3 promoter, a CERK6 promoter, a NFP promoter, a Lotus japonicus NFR5 promoter (SEQ ID NO: 109), a Lotus japonicus NFR1 promoter (SEQ ID NO: 151), a Lotus japonicus CERK6 promoter (SEQ ID NO: 111), a Medicago truncatula NFP promoter (SEQ ID NO: 110), a Medicago truncatula LYK3 promoter (SEQ ID NO: 112), a maize metallothioneine promoter, a chitinase promoter, a maize ZRP2 promoter, a tomato LeExt1 promoter, a glutamine synthetase soybean root promoter, a RCC3 promoter, a rice antiquitin promoter, a LRR receptor kinase promoter, or an Arabidopsis pCO2 promoter. In yet another embodiment of this aspect, the promoter is selected from the group of a CaMV35S promoter, a derivative of the CaMV35S promoter, a maize ubiquitin promoter, a polyubiquitin promoter, a vein mosaic cassava virus promoter, or an Arabidopsis UBQ10 promoter. In yet another embodiment of this aspect, the nucleic acid sequence is inserted into the genome of the plant so that the nucleic acid sequence is operably linked to an endogenous promoter, and wherein the endogenous promoter is a root specific promoter.

Additional aspects of the present disclosure relate to methods of producing the genetically modified plant or part thereof of any one of the preceding embodiments that has a modified plant LysM receptor polypeptide including a first JM zone 4 modified by insertion, deletion, or substitution of one or more amino acids in the first JM zone 4 with the corresponding amino acids from a second JM zone 4 able to ROS signaling or a modified plant LysM receptor polypeptide with enhanced ROS signaling, including genetically modifying the plant or part thereof by transforming the plant or part thereof with one or more gene editing components that target an endogenous nuclear genome sequence encoding an endogenous plant LysM receptor polypeptide to genetically modify a first JM zone 4 by insertion, deletion, or substitution of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 4 with the corresponding amino acids from a second JM zone 4 of a second plant LysM receptor polypeptide or wherein the plant LysM receptor polypeptide lacks a first JM zone 4 and the plant LysM receptor polypeptide has been modified by insertion of one or more amino acids, two or more amino acids, three or more amino acids, four or more amino acids, five or more amino acids, seven or more amino acids, nine or more amino acids, eleven or more amino acids, fifteen or more amino acids, twenty or more amino acids, or all amino acids in the first JM zone 4 with the corresponding amino acids from the second JM zone 4 into the corresponding site when aligning the plant LysM receptor and the second plant LysM receptor, and optionally to further including: (i) genetically modifying a first JM zone 2 by insertion, deletion, or substitution of one or more amino acids in the first JM zone 2 with the corresponding amino acids from a second JM zone 2 of a second plant LysM receptor polypeptide; (ii) genetically modifying a first JM zone 3 by insertion, deletion, or substitution of one or more amino acids in the first JM zone 3 with the corresponding amino acids from a second JM zone 3 of a second plant LysM receptor polypeptide; (iii) genetically modifying a first kinase N-terminus region by substitution of one or more amino acids in the first kinase N-terminus region with the corresponding amino acids from a second kinase N-terminus region from a NFR1 LysM receptor polypeptide; and/or (iv) genetically modifying a first extracellular domain by substituting one or more amino acids of the first extracellular domain with one or more amino acids of a second extracellular domain, wherein the second extracellular domain has a different affinity, selectivity, and/or specificity for oligosaccharides than the first extracellular domain. In a further embodiment of this aspect, the one or more gene editing components include a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence.A control as described herein can be a control sample or a reference sample from a wild-type, an azygous, or a null-segregant plant, species, or sample or from populations thereof. A reference value can be used in place of a control or reference sample, which was previously obtained from a wild-type, azygous, or null-segregant plant, species, or sample or from populations thereof or a group of a wild-type, azygous, or null-segregant plant, species, or sample. A control sample or a reference sample can also be a sample with a known amount of a detectable composition or a spiked sample.

### Expression Vectors, Isolated DNA Molecules, or Recombinant Nucleic Acids; Cells, Compositions, or Kits Including the Same; and Related Methods

A further aspect of the disclosure includes an expression vector, isolated DNA molecule, or recombinant nucleic acid including a modified plant LysM receptor polypeptide including a modified JM zone 4 domain, a modified JM zone 3 domain, a modified JM zone 2 domain, a modified kinase C-terminus region, and/or a modified extracellular domain operably linked to at least one expression control sequence. In an additional embodiment of this aspect, (i) the modified JM zone 4 was modified by substitution of one or more amino acids in a first JM zone 4 with the corresponding amino acids from a second JM zone 4 from an NFR1 LysM receptor polypeptide with NFR1-mediated root nodule symbiosis signaling, wherein the first JM zone 4 includes SEQ ID NO: 13, is selected from the group of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40, or conservative substitutions thereof, or optionally any of the preceding amino acid sequences with one, two, or three amino acid substitutions, insertions and/or deletions, or includes SEQ ID NO: 60, and/or wherein the second JM zone 4 includes SEQ ID NO: 6, or is selected from the group of SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and SEQ ID NO: 52, or optionally any of the preceding amino acid sequences with one, two, or three amino acid substitutions, insertions and/or deletions; (ii) the modified JM zone 2 was modified by insertion, deletion, or substitution of one or more amino acids in a first JM zone 2 with the corresponding amino acids from a second JM zone 2 of a second plant LysM receptor polypeptide, and wherein the first JM zone 2, the second JM zone 2, or both correspond to amino acids 256 to 280 when aligned to SEQ ID NO: 1 or correspond to amino acids 256 to 281 when aligned to SEQ ID NO: 8; (iii) the modified JM zone 3 was modified by insertion, deletion, or substitution of one or more amino acids in a first JM zone 3 with the corresponding amino acids from a second JM zone 3 of a second plant LysM receptor polypeptide, and wherein the first JM zone 3, the second JM zone 3, or both correspond to amino acids 281 to 304 when aligned to SEQ ID NO: 1 or correspond to amino acids 282 to 302 when aligned to SEQ ID NO: 8; (iv) the modified kinase C-terminus region was modified by substitution of one or more amino acids in a first kinase C-terminus region with the corresponding amino acids from a second kinase C-terminus region from a NFR1 LysM receptor polypeptide, wherein the first kinase C-terminus region corresponds to amino acids T467, S471, S473, P475, G477, V496, V500, Y510, D517, 1519, V520, S523, S525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, and G621 when aligned to SEQ ID NO: 8, and/or wherein the second kinase C-terminus region corresponds to amino acids 1469, N473, T475, Q477, 1497, 1501, F511, N518, V520, L521, G524, L526, V527, A528, E529, E539, A540, N542, K543, S544, C547, D548, A549, P556, E560, E564, L568, 1570, G574, R575, R579, L583, L591, L601, E603, C605, D607, E608, S610, S613, T615, 1617, L620, and V622 when aligned to SEQ ID NO: 1; and/or (v) the modified extracellular domain was modified by substituting one or more amino acids of the first extracellular domain with one or more amino acids of a second extracellular domain, wherein the second extracellular domain has a different affinity, selectivity, and/or specificity for oligosaccharides than the first extracellular domain.

An additional aspect of the disclosure includes an expression vector, isolated DNA molecule, or recombinant nucleic acid including a modified plant LysM receptor polypeptide including a modified JM zone 4 domain, a modified JM zone 3 domain, a modified JM zone 2 domain, a modified kinase N-terminus region, and/or a modified extracellular domain operably linked to at least one expression control sequence. In a further embodiment of this aspect, (i) the modified JM zone 4 was modified by substitution of one or more amino acids in a first JM zone 4 with the corresponding amino acids from a second JM zone 4 from a LysM receptor polypeptide with ROS signaling, and wherein the first JM zone 4 includes SEQ ID NO: 6, or is selected from the group of SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and SEQ ID NO: 52, or optionally any of the preceding amino acid sequences with one, two, or three amino acid substitutions, insertions and/or deletions, and/or wherein the second JM zone 4 includes SEQ ID NO: 13, or is selected from the group of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40, or optionally any of the preceding amino acid sequences with one, two, or three amino acid substitutions, insertions and/or deletions; (ii) the modified JM zone 2 was modified by insertion, deletion, or substitution of one or more amino acids in a first JM zone 2 with the corresponding amino acids from a second JM zone 2 of a second plant LysM receptor polypeptide, and wherein the first JM zone 2, the second JM zone 2, or both correspond to amino acids 256 to 280 when aligned to SEQ ID NO: 1 or correspond to amino acids 256 to 281 when aligned to SEQ ID NO: 8; (iii) the modified JM zone 3 was modified by insertion, deletion, or substitution of one or more amino acids in a first JM zone 3 with the corresponding amino acids from a second JM zone 3 of a second plant LysM receptor polypeptide, and wherein the first JM zone 3, the second JM zone 3, or both correspond to amino acids 281 to 304 when aligned to SEQ ID NO: 1 or correspond to amino acids 282 to 302 when aligned to SEQ ID NO: 8; (iv) the modified kinase N-terminus region was modified by substitution of one or more amino acids in a first kinase N-terminus region with the corresponding amino acids from a second kinase N-terminus region from a CERK6 LysM receptor polypeptide, wherein the first kinase N-terminus region corresponds to D328, A338, K347, K348, T349, V356, T360, C364, H371, C384, V385, H395, D397, G402, Y404, H406, K410, E411, S416, S417, A425, V445, and L456 when aligned to SEQ ID NO: 1, and/or wherein the second kinase N-terminus region corresponds to A326, S336, E345, R346, A347, M354, K358, A362, R369, S382, 1383, F393, E395, S400, H402, R404, R408, D409, A414, T415, S423, 1443, and Y454 when aligned to SEQ ID NO: 8; and/or (v) the modified extracellular domain was modified by substituting one or more amino acids of the first extracellular domain with one or more amino acids of a second extracellular domain, wherein the second extracellular domain has a different affinity, selectivity, and/or specificity for oligosaccharides than the first extracellular domain.

In a further embodiment of the preceding aspects, which may be combined with any of the preceding embodiments that has an expression vector, isolated DNA molecular, or recombinant nucleic acid, the at least one expression control sequence includes a promoter selected from the group of a root specific promoter, a constitutive promoter, or a combination thereof. In an additional embodiment of this aspect, the promoter is a root specific promoter, and wherein the promoter is selected from the group of a NFR1 promoter, a NFR5 promoter, a LYK3 promoter, a CERK6 promoter, a NFP promoter, a Lotus japonicus NFR5 promoter (SEQ ID NO: 109), a Lotus japonicus NFR1 promoter (SEQ ID NO: 151), a Lotus japonicus CERK6 promoter (SEQ ID NO: 111), a Medicago truncatula NFP promoter (SEQ ID NO: 110), a Medicago truncatula LYK3 promoter (SEQ ID NO: 112), a maize metallothioneine promoter, a chitinase promoter, a maize ZRP2 promoter, a tomato LeExt1 promoter, a glutamine synthetase soybean root promoter, a RCC3 promoter, a rice antiquitin promoter, a LRR receptor kinase promoter, or an Arabidopsis pCO2 promoter. In a further embodiment of this aspect, the promoter is constitutive promoter, and wherein the promoter is selected from the group of a CaMV35S promoter, a derivative of the CaMV35S promoter, a maize ubiquitin promoter, a polyubiquitin promoter, a vein mosaic cassava virus promoter, or an Arabidopsis UBQ10 promoter.

Some aspects of the disclosure include a bacterial cell or an Agrobacterium cell including the expression vector, isolated DNA molecule, or recombinant nucleic acid of any one of the preceding embodiments.

Further aspects of the disclosure include a genetically modified plant, plant part, plant cell, or seed including the expression vector, isolated DNA molecule, or recombinant nucleic acid of any one of the preceding embodiments.

Additional aspects of the disclosure include a composition or kit including the expression vector, isolated DNA molecule, or recombinant nucleic acid of any one of the preceding embodiments, the bacterial cell or Agrobacterium cell of any one of the preceding embodiments, or the genetically modified plant, plant part, plant cell, or seed of any one of the preceding embodiments.

Yet further aspects of the disclosure include methods of initiating NFR1-mediated root nodule symbiosis signaling including: introducing a genetic alteration via the expression vector, isolated DNA molecule, or recombinant nucleic acid of any one of the preceding embodiments that has a modified kinase C-terminus region.

Still further aspects of the disclosure include methods of initiating ROS signaling including: introducing a genetic alteration via the expression vector, isolated DNA molecule, or recombinant nucleic acid of any one of the preceding embodiments that has a modified kinase N-terminus region.

In a further embodiment of the preceding aspects, which may be combined with any of the preceding embodiments that has a method of initiating NFR1-mediated root nodule symbiosis signaling or ROS signaling, the plant is a plant cell.

### LysM Receptors and NFR1, LYK3, and CERK6

LysM receptors may be defined as proteins that contain three tandem LysM domains in their extracellular region, namely LysM1, LysM2, and LysM3, which are present in this order on the protein sequence and separated by CxC motifs. The LysM1 domain is located toward the N-terminal end of the protein sequence, and is preceded by an N-terminal signal peptide. Moving into the cell, the LysM domains are followed by a single-pass transmembrane (TM) domain, a juxtamembrane (JM) domain, and an intracellular kinase or pseudokinase domain. Most plant LysM receptors, including NFR1, contain an intracellular kinase domain. Examples of LysM-RLK receptors can be NFP, NFR5, LYK10, LYR3, LYK4, LYK3, NFR1, lykX (LysM-RLK), CERK1, and other receptors known to one of skill in the art.

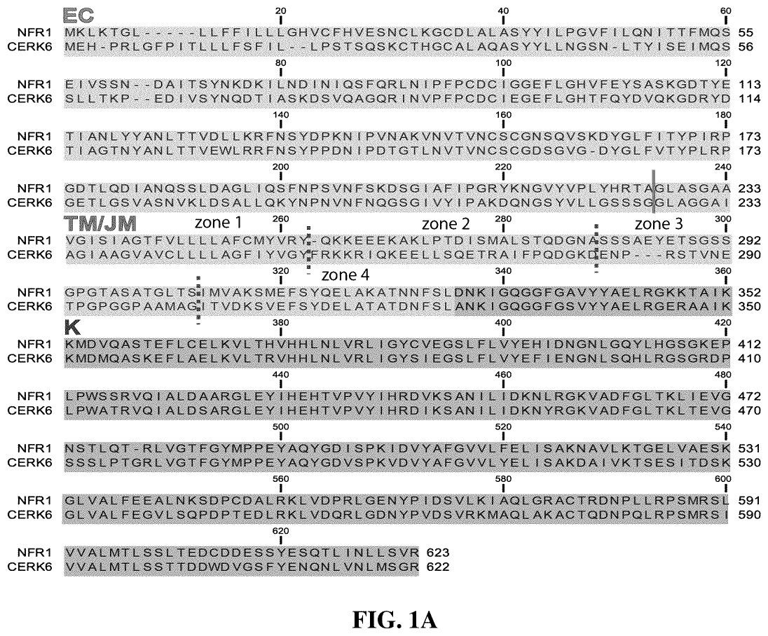

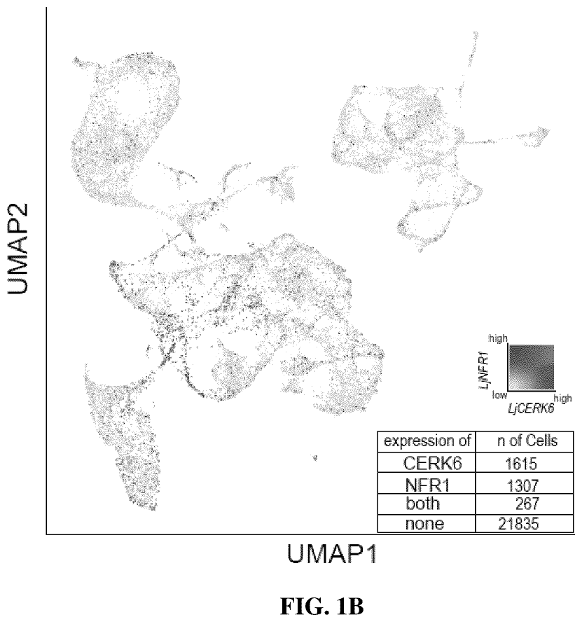

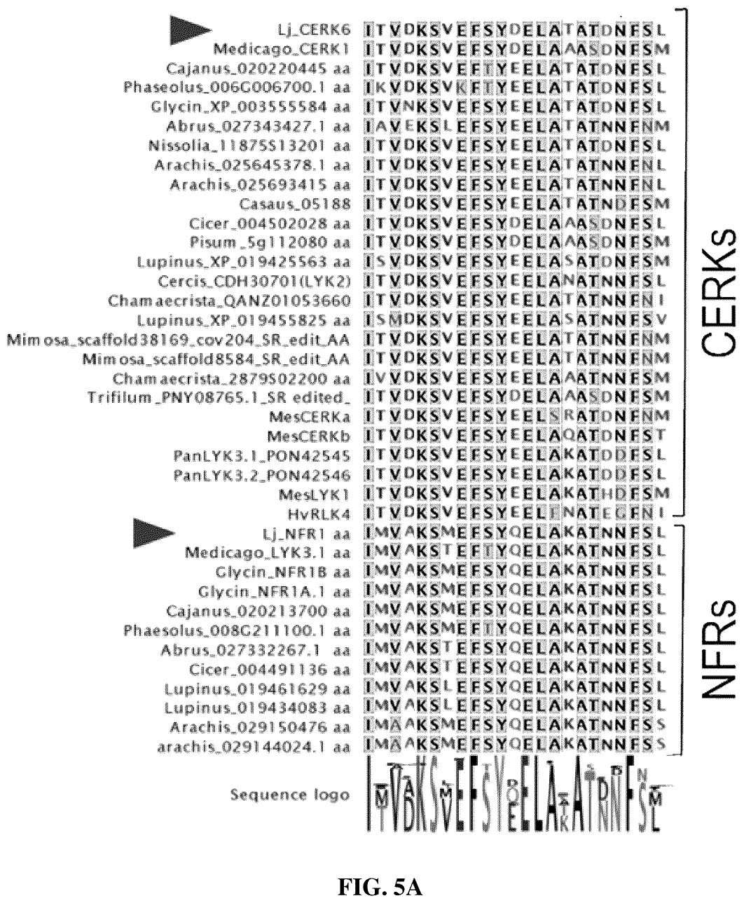

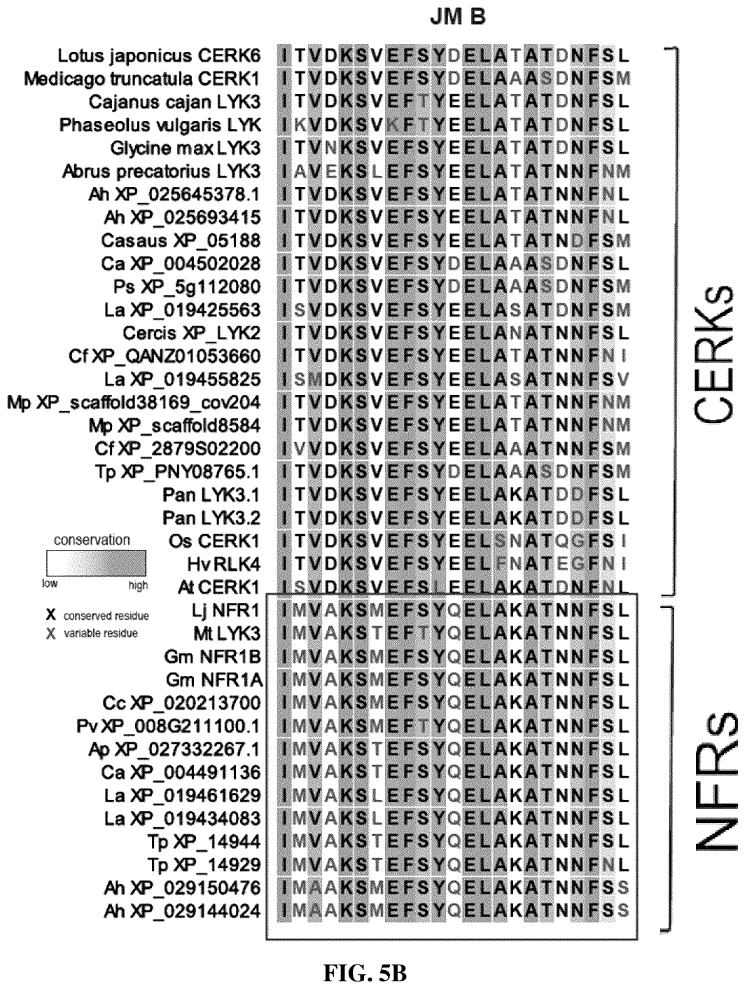

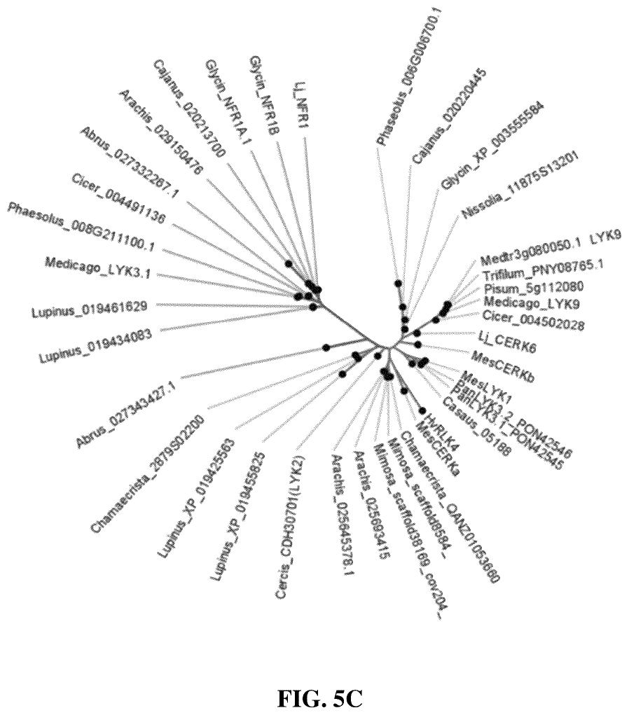

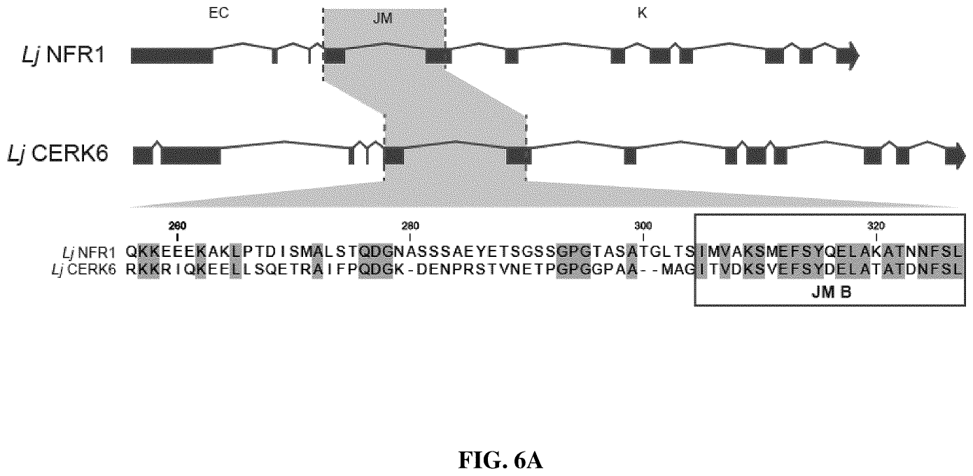

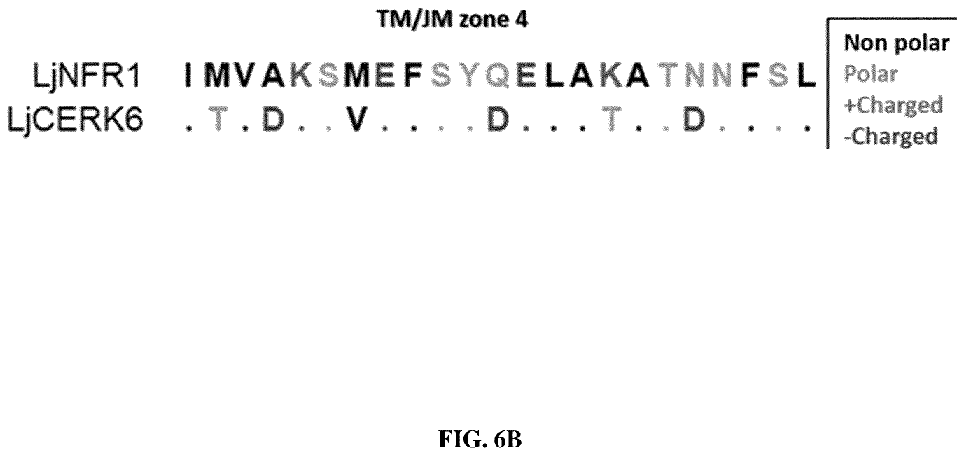

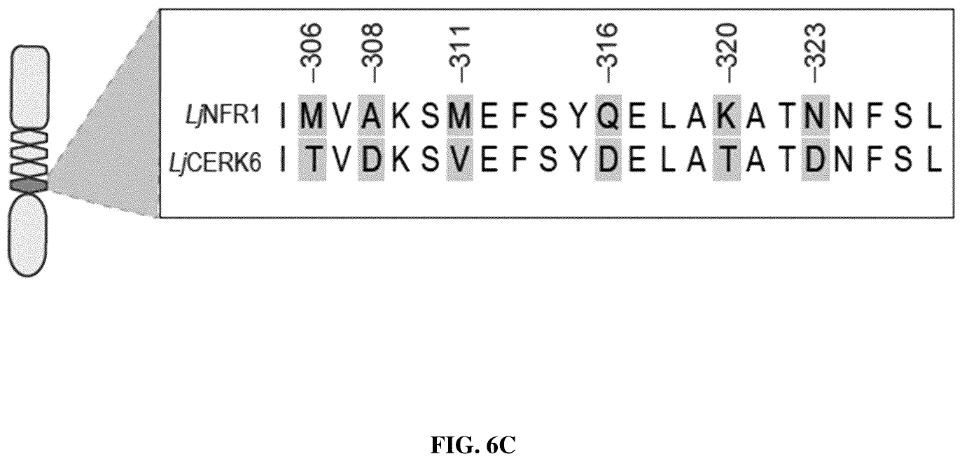

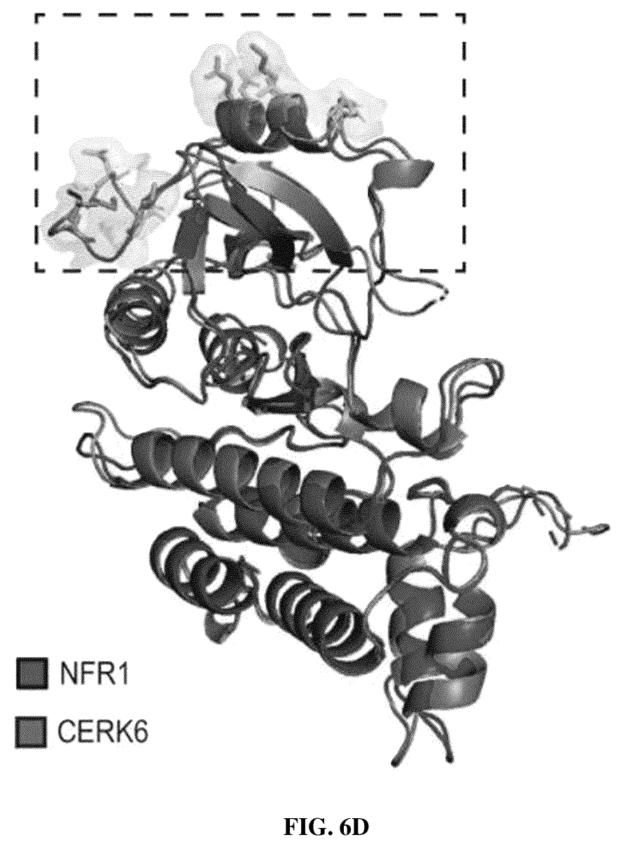

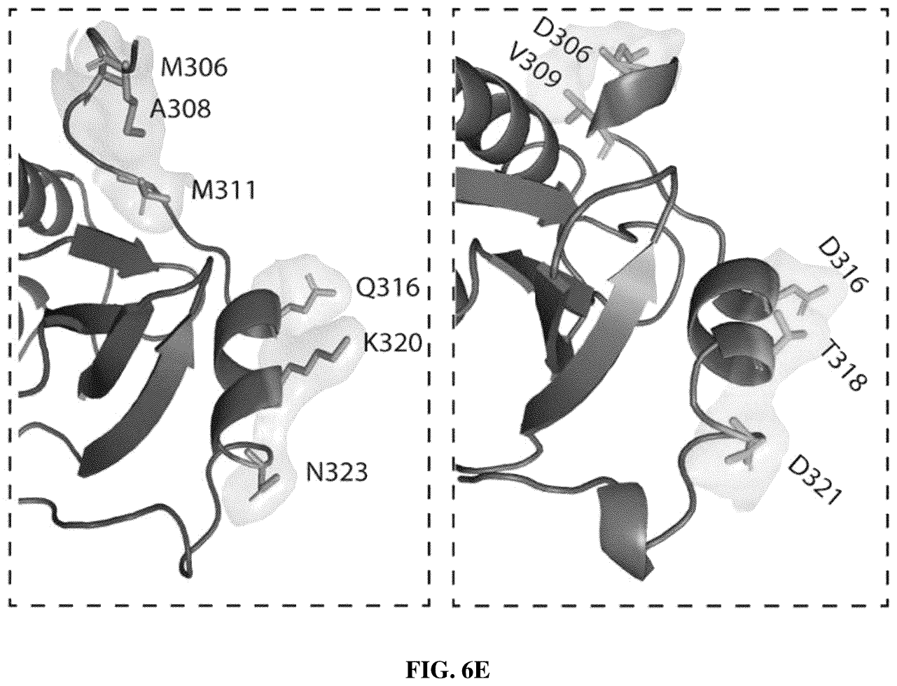

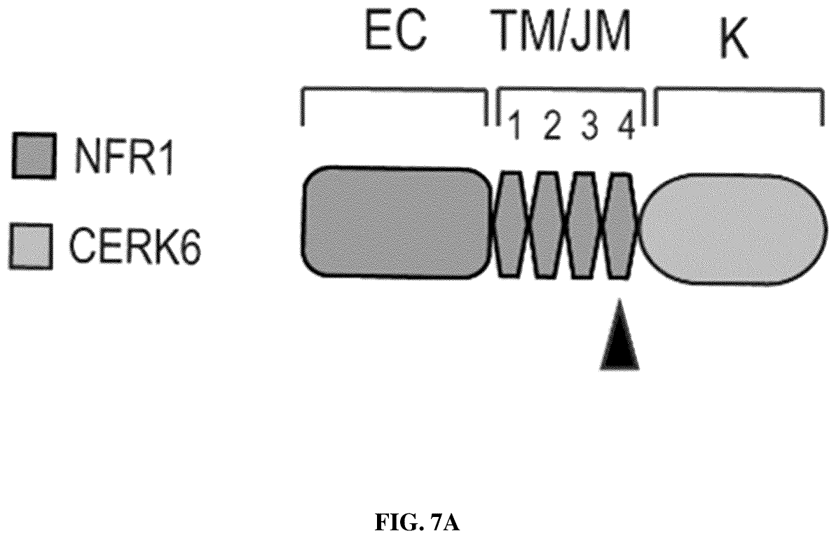

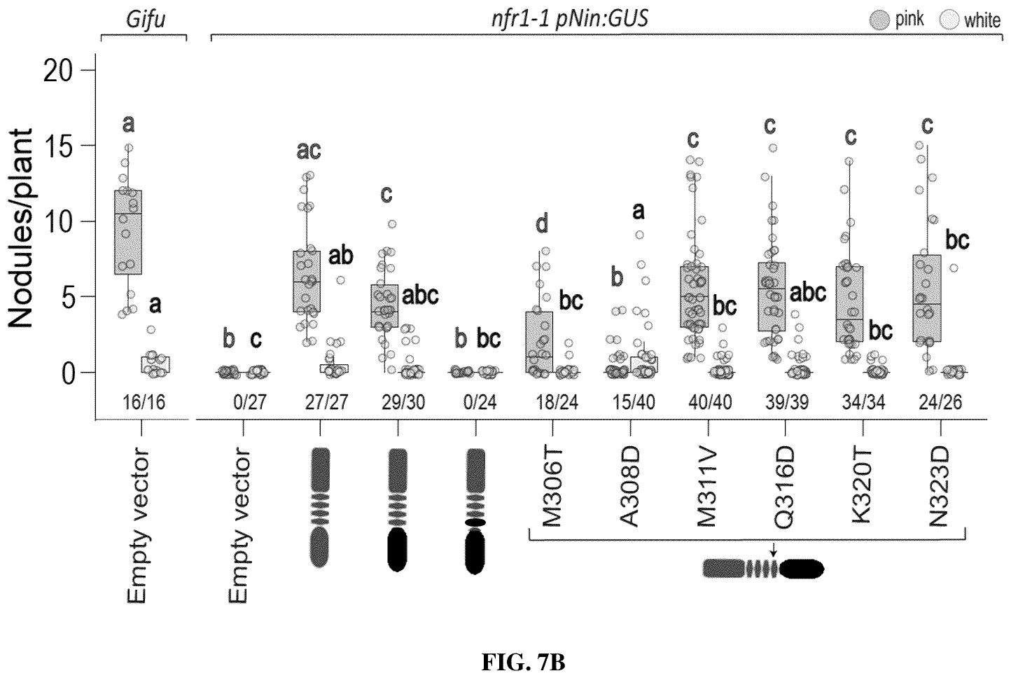

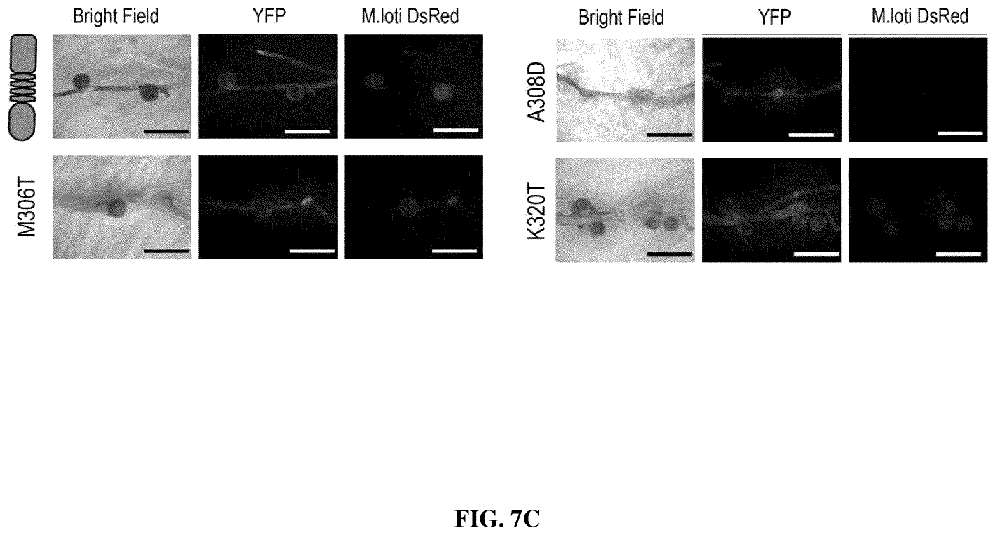

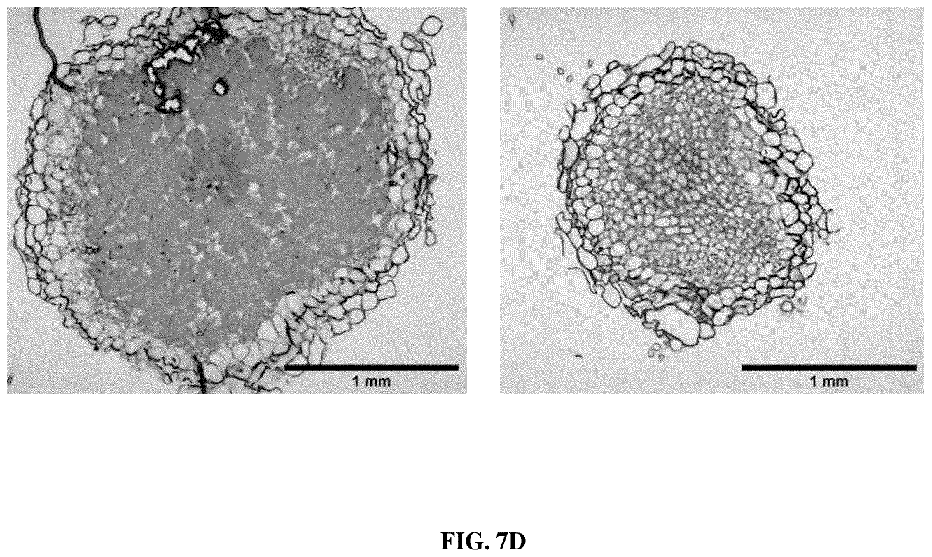

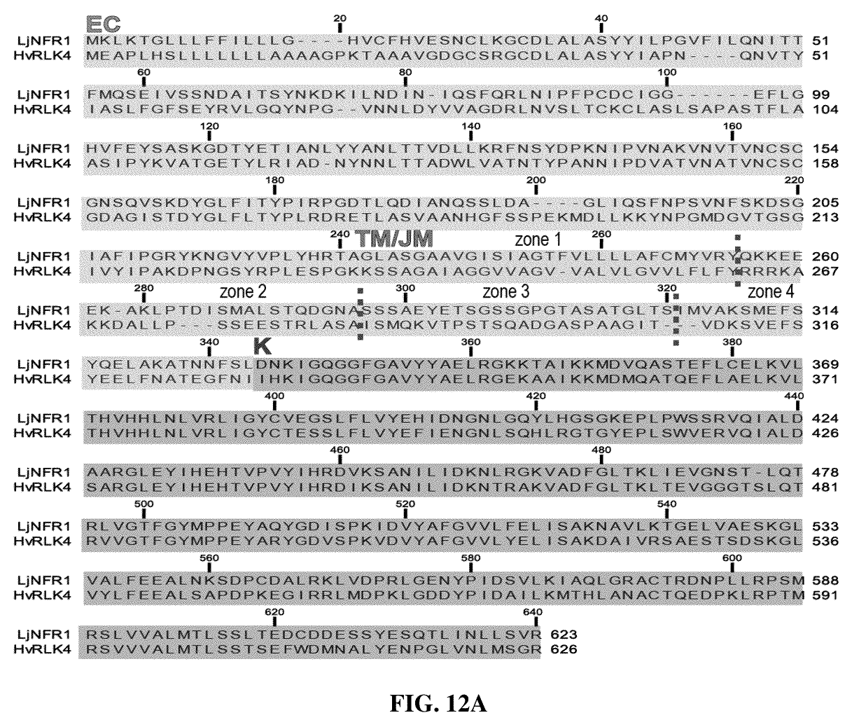

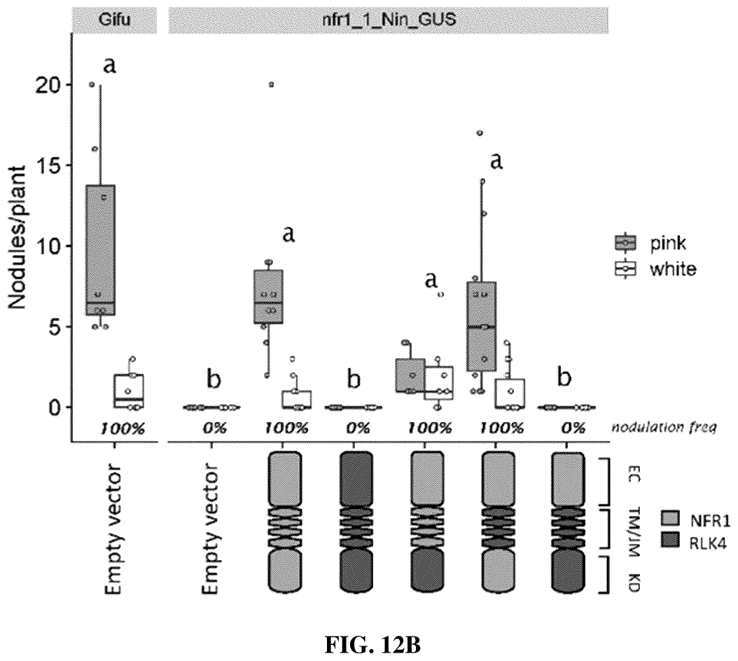

As shown in FIG. 7A, the TM domain includes zone 1, while the JM domain includes zone 2, zone 3, and zone 4. As used herein, the terms zone 4 and JM-B are interchangeable. FIG. 1A provides an alignment of Lotus japonicus NFR1 and Lotus japonicus CERK6, showing zones 1-4 with their corresponding sequences. FIG. 12A provides a similar alignment of Lotus japonicus NFR1 and Hordeum vulgare RLK4. Of these zones, zone 4 was identified as being critical for NFR1-mediated root nodule symbiosis signaling in NFR1. FIGS. 5A and 5B provide an alignment of JM zone 4 for CERKs (top) and NFRs (bottom) in multiple plant species, both in the nitrogen-fixing clade (NFC; also known as FaFaCuRo clade) nodulating and non-nodulating). Similarly, FIGS. 6B and 6C provide an alignment of zone 4 of Lotus japonicus NFR1 and Lotus japonicus CERK6.

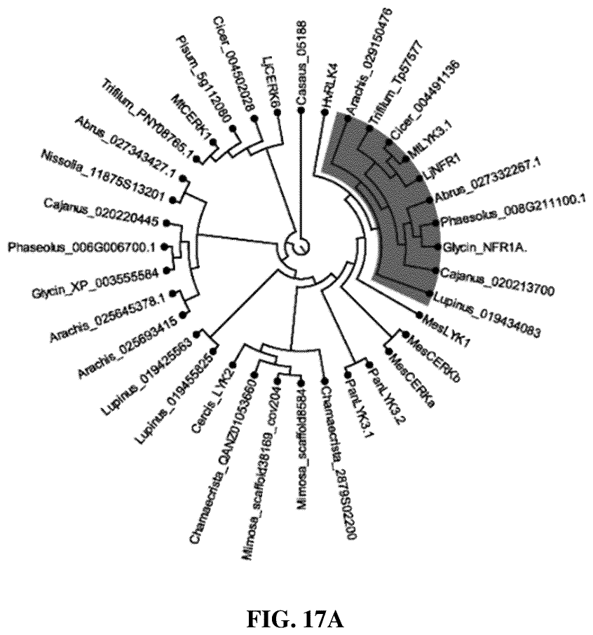

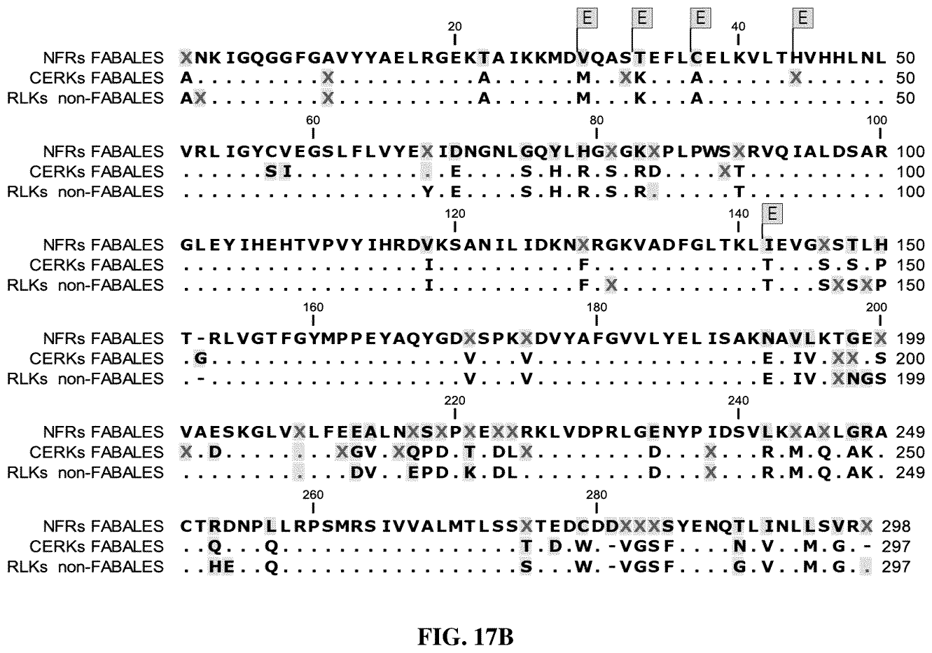

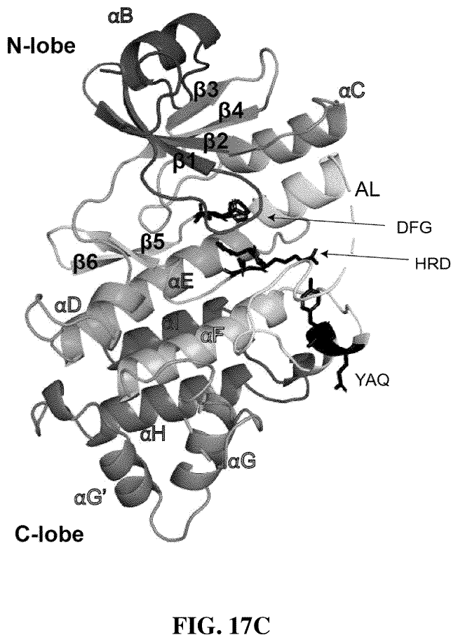

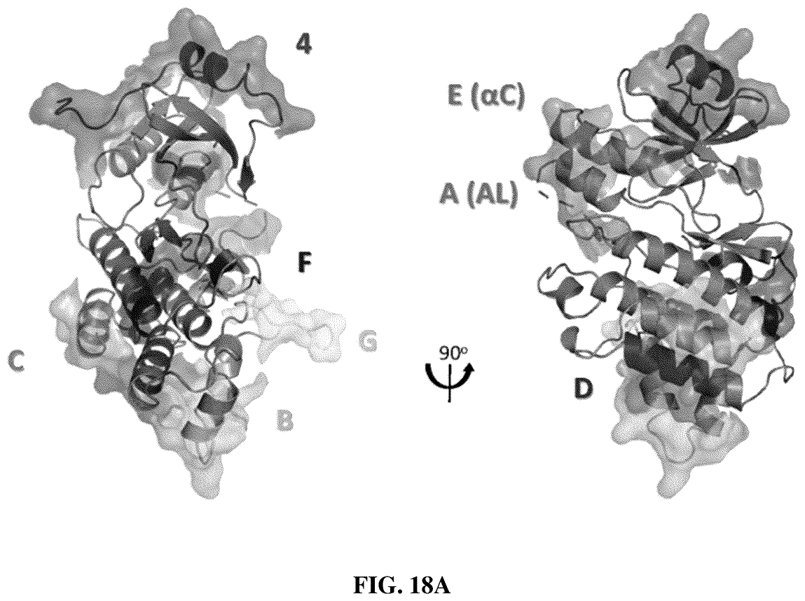

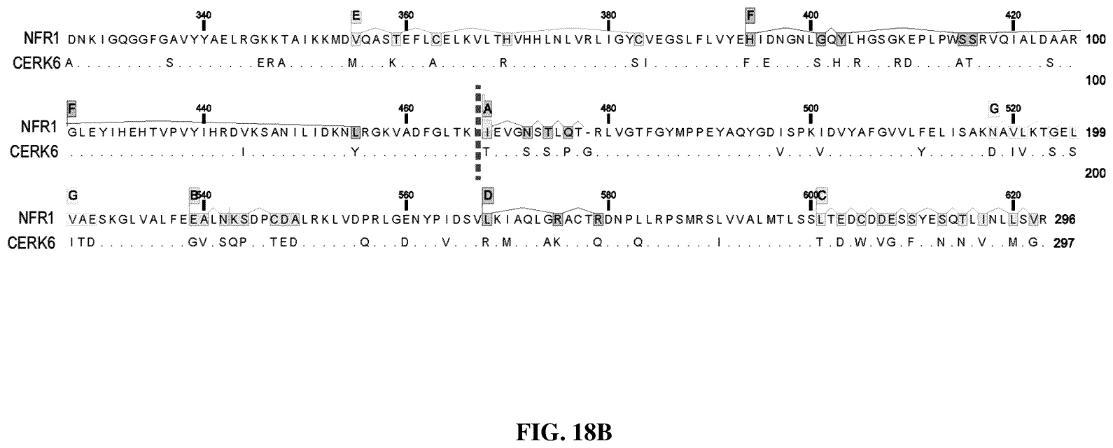

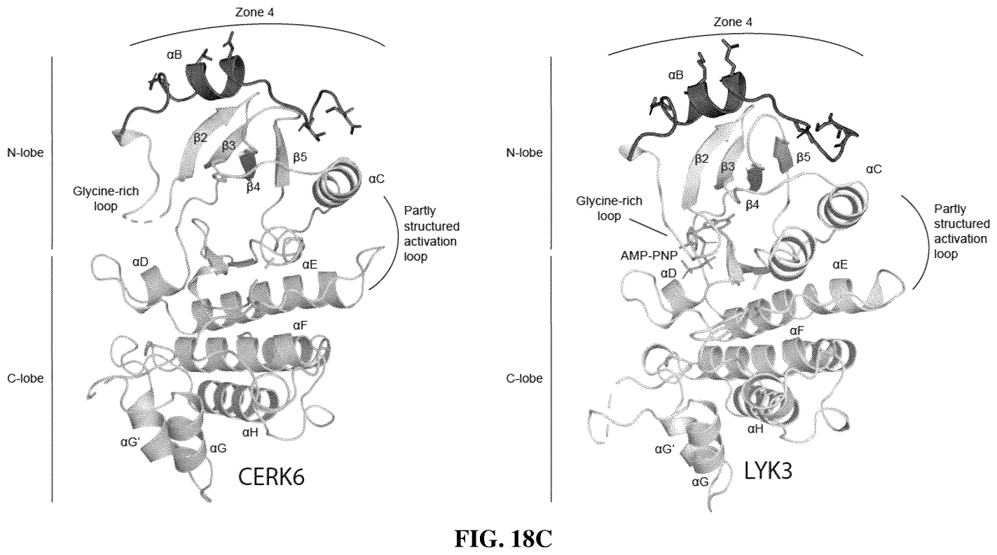

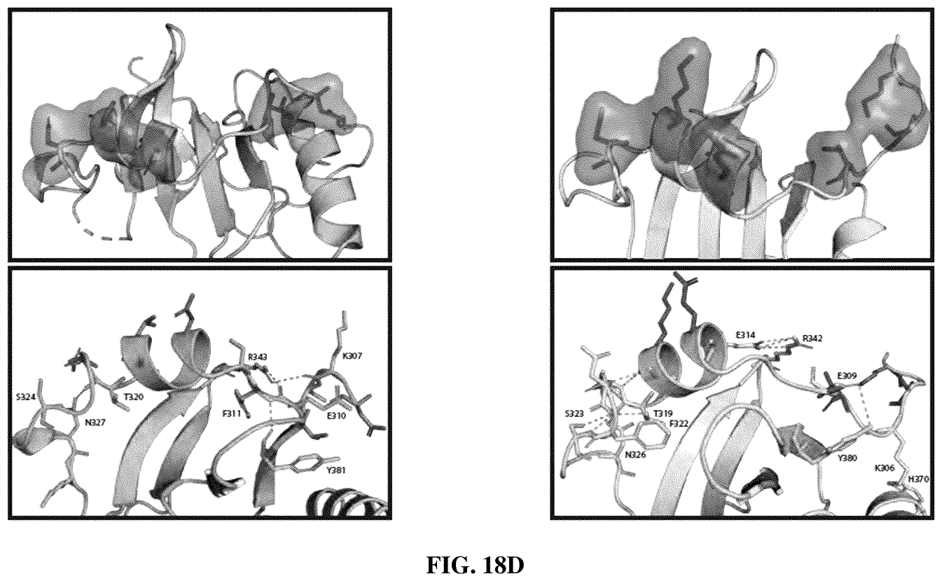

The intracellular kinase domain (KD) includes multiple zones as well. FIG. 17B provides an alignment of consensus kinase domains of Fabales NFRs (top), Fabales CERKs (middle), and non-Fabales NFRs (bottom). FIG. 18B provides an alignment of the Lotus japonicus NFR1 KD and the Lotus japonicus CERK6 KD, which illustrates the individual zones (indicated by color and label). Zone A consists of residues that belong to the activation loop (AL); Zone B contains residues of the αG- and αG′- helixes as well as the loop that connects them; Zone C contains residues of the C-terminal tail; surface D consists of three residues located in the αH-helix; Zone E primarily contains residues that belong to the αC-helix; Zone F contains residues that belong to different loops, sheets and helixes that are in close proximity in the structure; and finally Zone G contains residues that belong to the αG-H loop. There are also two larger regions in the KD, which are the N-terminus, involved in immunity signalling (e.g., ROS signalling), and the C-terminus, involved in NFR1-mediated root nodule symbiosis signaling. Table 1 provides the details of the residues in each zone and lobe for both the Lotus japonicus NFR1 KD and the Lotus japonicus CERK6 KD.

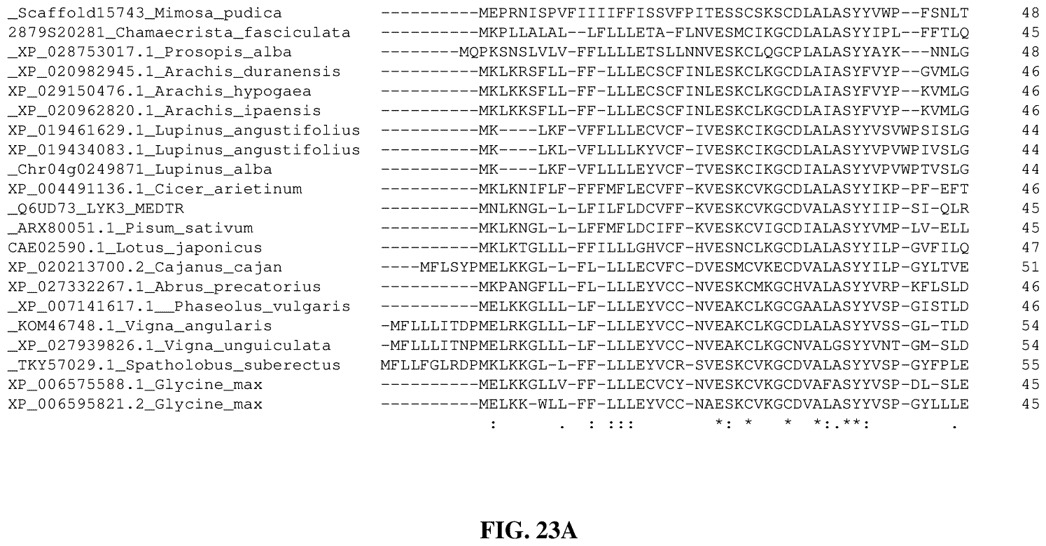

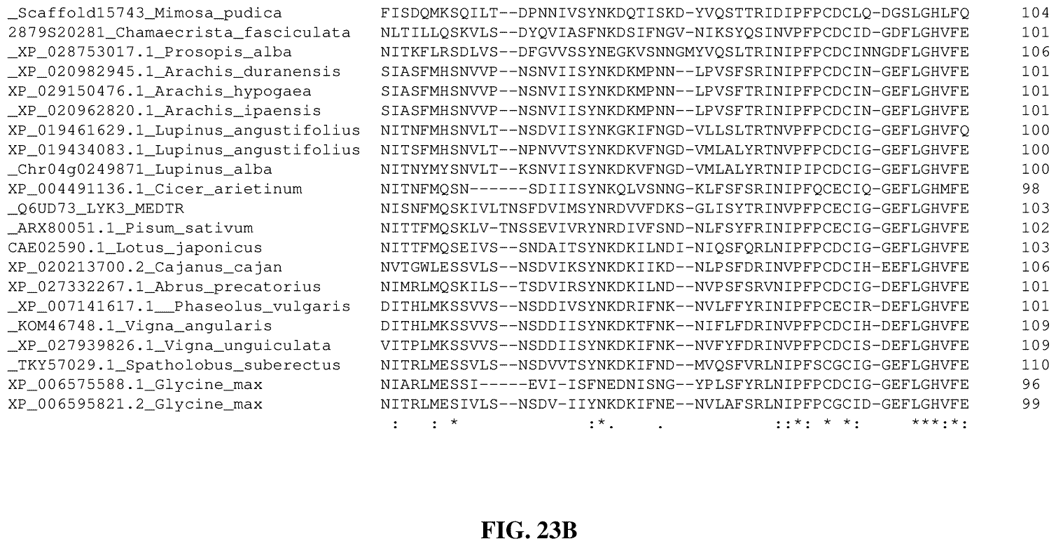

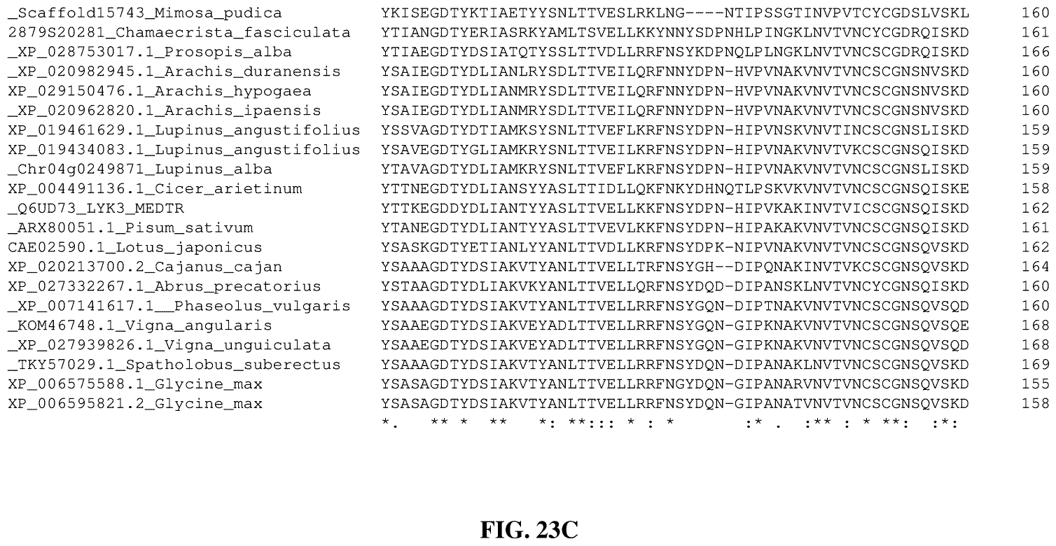

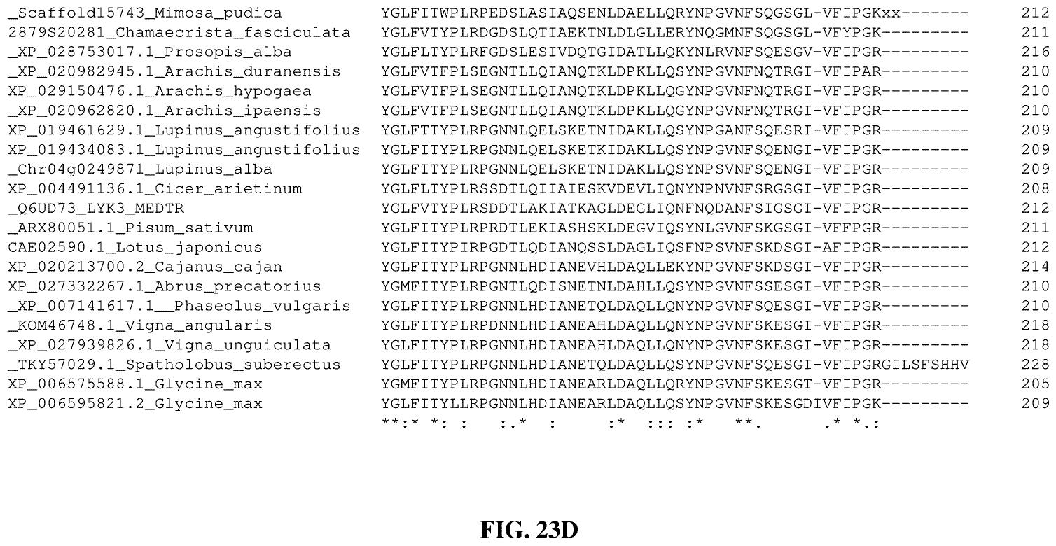

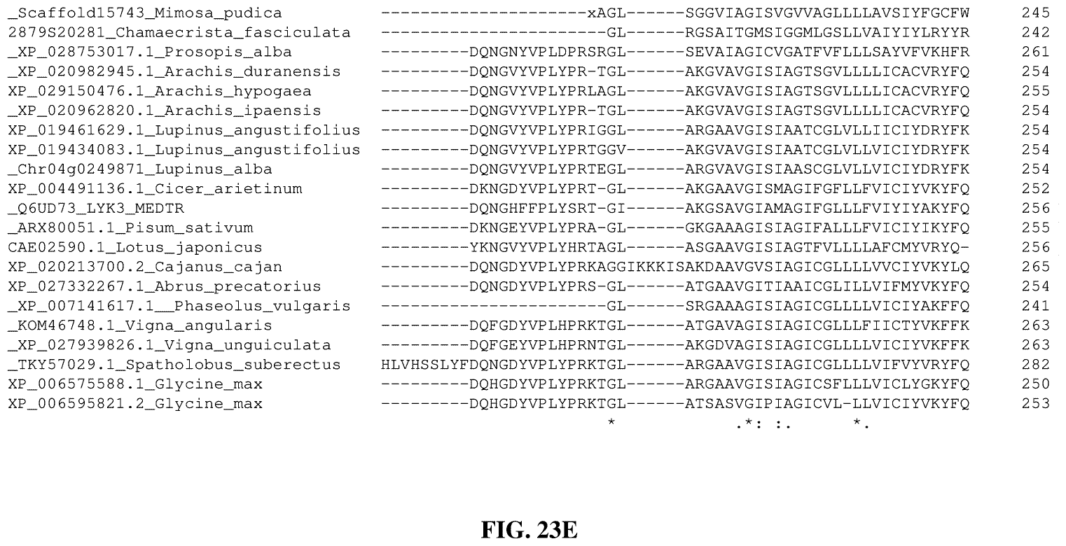

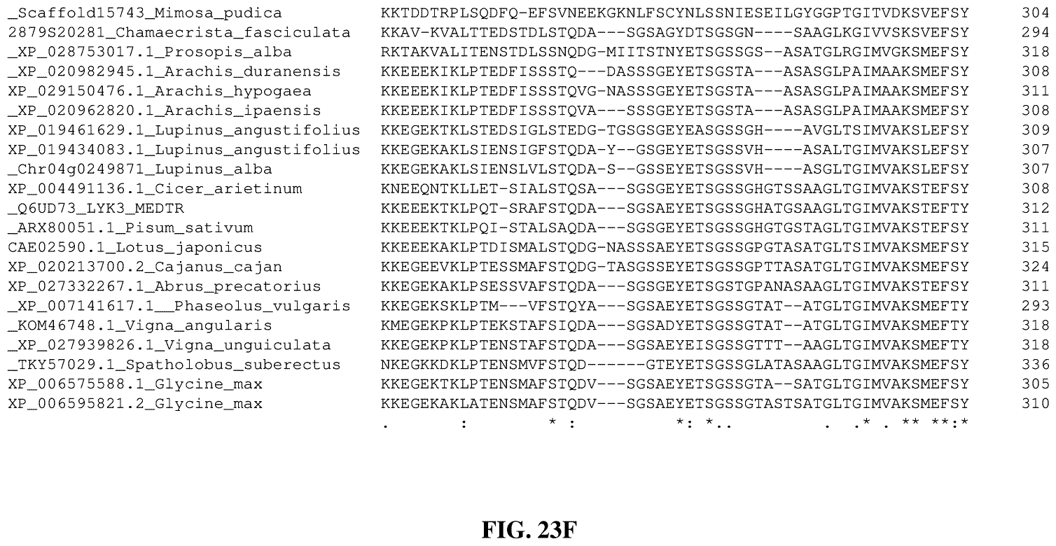

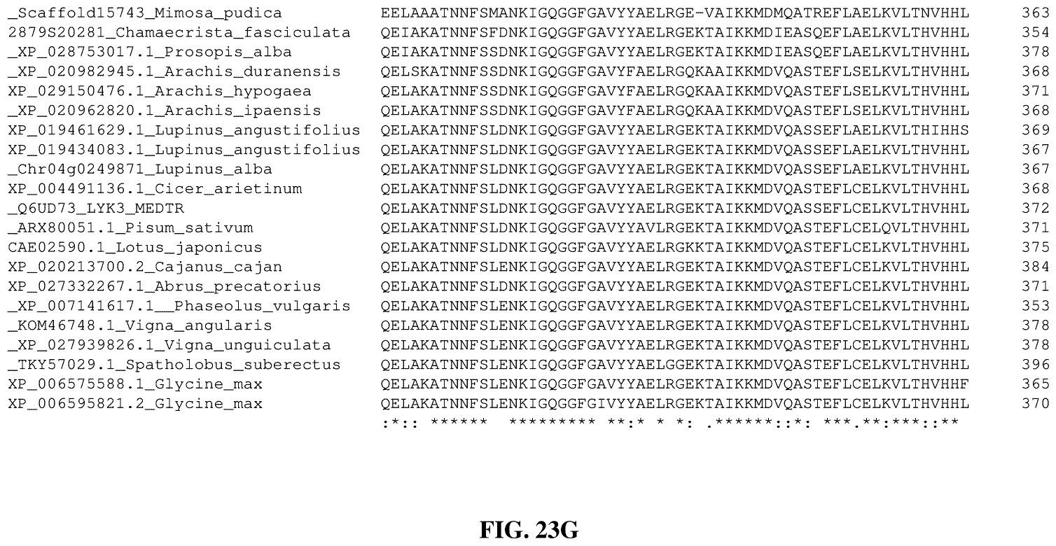

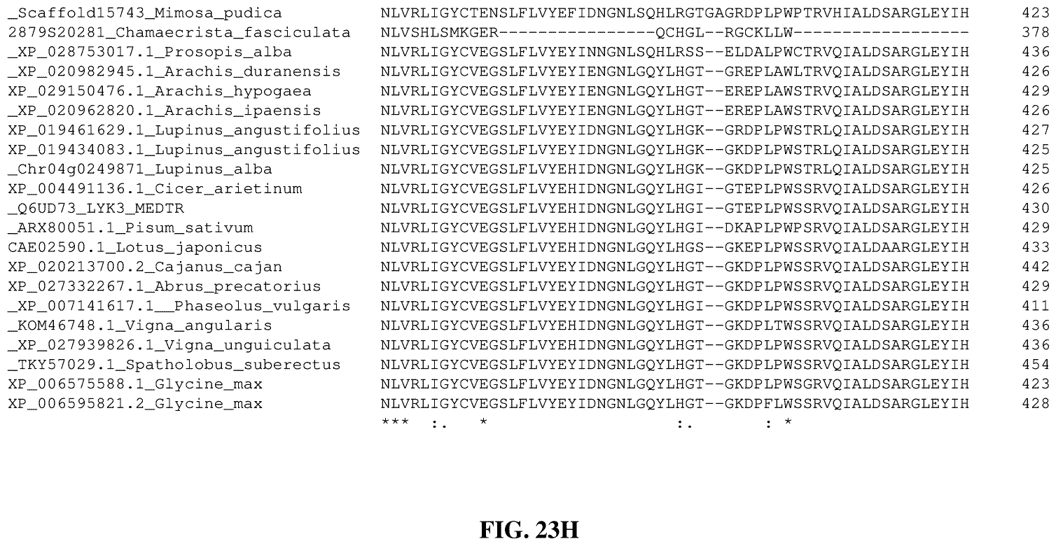

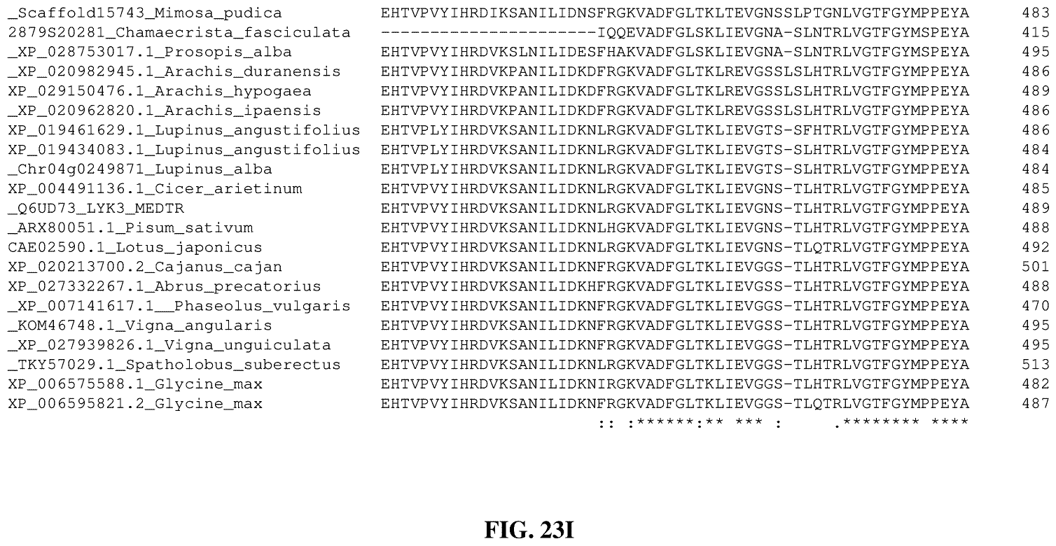

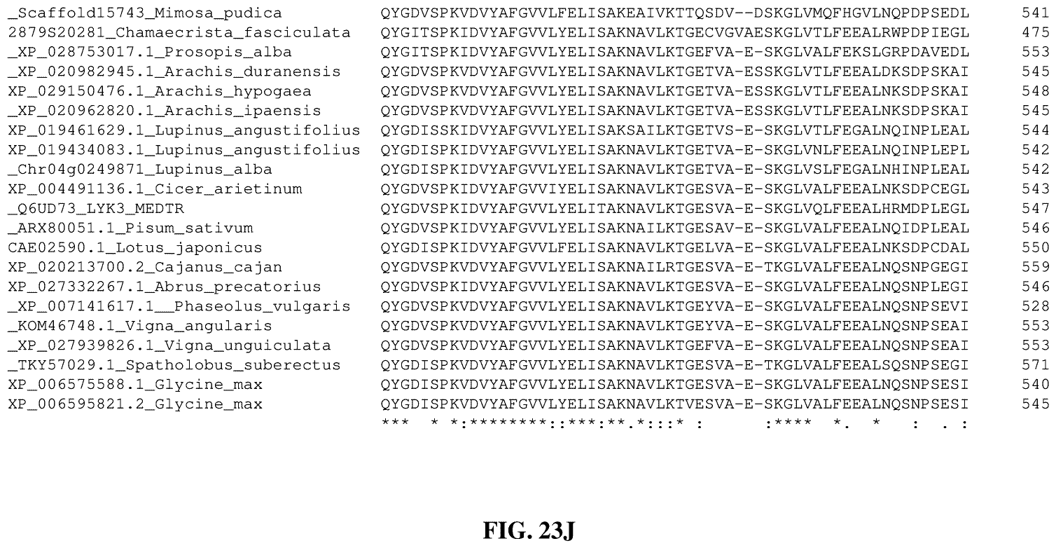

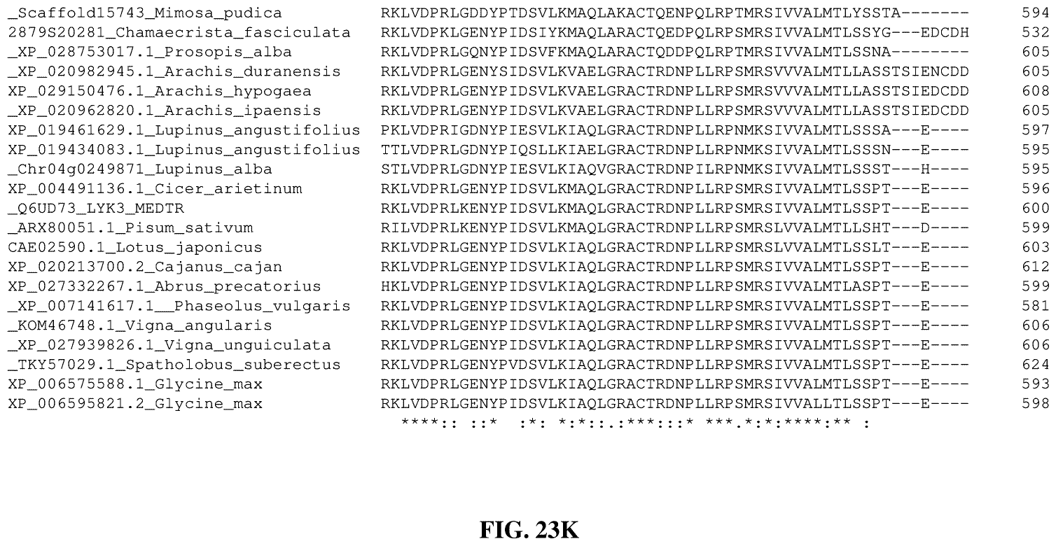

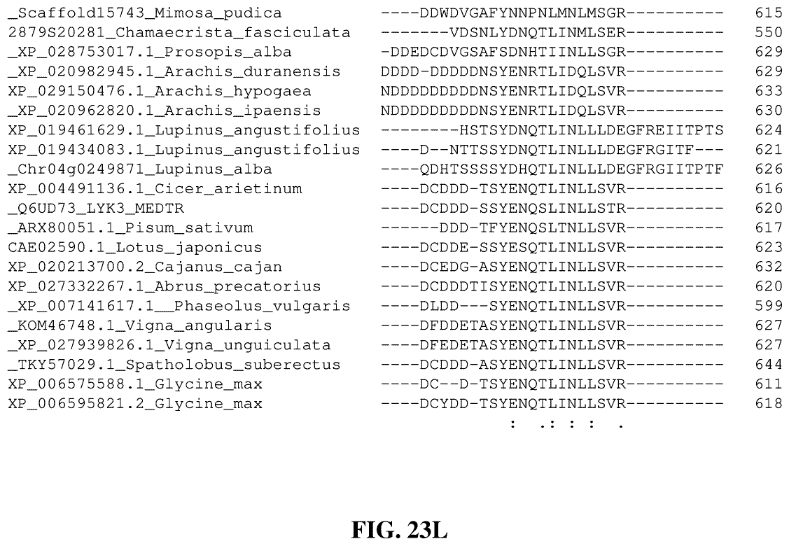

Lohmann et al., 2010 presents additional characteristics of NFR1-type receptors (Lohmann GV, Shimoda Y, Nielsen MW, Jergensen FG, Grossmann C, Sandal N, Serensen K, Thirup S, Madsen LH, Tabata S, Sato S, Stougaardi J, Radutoiu S. Evolution and regulation of the Lotus japonicus LysM receptor gene family. Mol Plant Microbe Interact. 2010; 23(4):510-21). In short, NFR1-type receptors are translated from multi-exon genes. Further, NFR1-type receptors contain all the subdomains typical for an intracellular kinase. NFR1-type receptors may also be referred to as LYK3-type receptors, as Lotus japonicus NFR1 and Medicago truncatula LYK3 both belong to this receptor type. An alignment of NFR1-type LysM receptors is shown in FIGS. 23A-23L.

The present disclosure provides the crystal structures of Lotus japonicus CERK6/303-599 D460N and Medicago truncatula LYK3/302-597 D459N with AMP-PNP (phosphoaminophosphonic acid-adenylate ester) in large table form, which further illustrate the embodiments. These structures are hereby incorporated by reference, and are described in Table I: CERK6 (for Lotus japonicus CERK6/303-599 D460N) and Table II: LYK3 (for Medicago truncatula LYK3/302-597 D459N with AMP-PNP), respectively. Further provided in the Examples of the present disclosure is exploration of the crystal structures containing these residues, along with descriptions of these residues' interactions with the kinase N-lobe.

### Plant Breeding Methods

Plant breeding begins with the analysis of the current germplasm, the definition of problems and weaknesses of the current germplasm, the establishment of program goals, and the definition of specific breeding objectives. The next step is the selection of germplasm that possess the traits to meet the program goals. The selected germplasm is crossed in order to recombine the desired traits and through selection, varieties or parent lines are developed. The goal is to combine in a single variety or hybrid an improved combination of desirable traits from the parental germplasm. These important traits may include higher yield, field performance, improved fruit and agronomic quality, resistance to biological stresses, such as diseases and pests, and tolerance to environmental stresses, such as drought and heat.

Each breeding program should include a periodic, objective evaluation of the efficiency of the breeding procedure. Evaluation criteria vary depending on the goal and objectives, but should include gain from selection per year based on comparisons to an appropriate standard, overall value of the advanced breeding lines, and number of successful cultivars produced per unit of input (e.g., per year, per dollar expended, etc.). Promising advanced breeding lines are thoroughly tested and compared to appropriate standards in environments representative of the commercial target area(s) for three years at least. The best lines are candidates for new commercial cultivars; those still deficient in a few traits are used as parents to produce new populations for further selection. These processes, which lead to the final step of marketing and distribution, usually take five to ten years from the time the first cross or selection is made.

The choice of breeding or selection methods depends on the mode of plant reproduction, the heritability of the trait(s) being improved, and the type of cultivar used commercially (e.g., F₁ hybrid cultivar, inbred cultivar, etc.). For highly heritable traits, a choice of superior individual plants evaluated at a single location will be effective, whereas for traits with low heritability, selection should be based on mean values obtained from replicated evaluations of families of related plants. The complexity of inheritance also influences the choice of the breeding method. Backcross breeding is used to transfer one or a few genes for a highly heritable trait into a desirable cultivar (e.g., for breeding disease-resistant cultivars), while recurrent selection techniques are used for quantitatively inherited traits controlled by numerous genes, various recurrent selection techniques are used. Commonly used selection methods include pedigree selection, modified pedigree selection, mass selection, and recurrent selection.

Pedigree selection is generally used for the improvement of self-pollinating crops or inbred lines of cross-pollinating crops. Two parents which possess favorable, complementary traits are crossed to produce an F₁. An F₂ population is produced by selfing one or several F₁s or by intercrossing two F₁s (sib mating). Selection of the best individuals is usually begun in the F₂ population; then, beginning in the F₃, the best individuals in the best families are selected. Replicated testing of families, or hybrid combinations involving individuals of these families, often follows in the F₄ generation to improve the effectiveness of selection for traits with low heritability. At an advanced stage of inbreeding (i.e., F₆ and F₇), the best lines or mixtures of phenotypically similar lines are tested for potential release as new cultivars.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding (i.e., recurrent selection) may be used to transfer genes for a simply inherited, highly heritable trait into a desirable homozygous cultivar or line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

In addition to phenotypic observations, the genotype of a plant can also be examined. There are many laboratory-based techniques available for the analysis, comparison and characterization of plant genotype; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), Simple Sequence Repeats (SSRs, which are also referred to as Microsatellites), Fluorescently Tagged Inter-simple Sequence Repeats (ISSRs), Single Nucleotide Polymorphisms (SNPs), Genotyping by Sequencing (GbS), and Next-generation Sequencing (NGS).

Molecular markers, or “markers”, can also be used during the breeding process for the selection of qualitative traits. For example, markers closely linked to alleles or markers containing sequences within the actual alleles of interest can be used to select plants that contain the alleles of interest. The use of markers in the selection process is often called genetic marker enhanced selection or marker-assisted selection. Methods of performing marker analysis are generally known to those of skill in the art.

Mutation breeding may also be used to introduce new traits into plant varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in Principles of Cultivar Development: Theory and Technique, Walter Fehr (1991), Agronomy Books, 1 (https://lib.dr.iastate.edu/agron_books/1).

The production of double haploids can also be used for the development of homozygous lines in a breeding program. Double haploids are produced by the doubling of a set of chromosomes from a heterozygous plant to produce a completely homozygous individual. For example, see Wan, et al., Theor. Appl. Genet., 77:889-892, 1989.

Additional non-limiting examples of breeding methods that may be used include, without limitation, those found in Principles of Plant Breeding, John Wiley and Son, pp. 115-161 (1960); Principles of Cultivar Development: Theory and Technique, Walter Fehr (1991), Agronomy Books, 1 (https://lib.dr.iastate.edu/agron_books/1), which are herewith incorporated by reference.

### Molecular Biological Methods to Produce Transgenic Plant Cells, Plant Parts, and Plants

One aspect of the present disclosure provides transgenic plant cells, plant parts, or plants including a modified juxtamembrane (JM) zone 4, and optionally further including a modified JM zone 2, a modified JM zone 3, a modified extracellular domain, and/or a modified kinase C-terminus region or a modified kinase N-terminus region. The modified LysM receptors of the present disclosure are either able to able to initiate NFR1-mediated root nodule symbiosis signaling or able to initiate ROS signaling. In addition, the present disclosure provides isolated DNA molecules of vectors and gene editing components used to produce transgenic plants of the present disclosure.

The JM zone 4 corresponds to amino acids 305 to 327 when aligned to SEQ ID NO: 1 or corresponds to amino acids 303 to 325 when aligned to SEQ ID NO: 8. In an additional embodiment of this aspect, the first JM zone 4 is selected from the group of SEQ ID NO: 13, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 60, SEQ ID NO: 6, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, or SEQ ID NO: 52. In another embodiment of this aspect, the first JM zone 4 includes substituted amino acid residues that are selected from amino acid residues corresponding to M306, A308, and K320 of SEQ ID NO: 1 or from amino acid residues corresponding to T304, D306, and T318 of SEQ ID NO: 8. In a further embodiment of this aspect, the residues of JM zone 4 may be modified (e.g., by insertion, deletion, alteration, etc.) in such a way that the modified residues do not correspond to the residues of a different JM zone 4 from a different LysM receptor.

The JM zone 2 corresponds to amino acids 256 to 280 when aligned to SEQ ID NO: 1 or corresponds to amino acids 256 to 281 when aligned to SEQ ID NO: 8. In yet another embodiment of this aspect, the JM zone 2 includes SEQ ID NO: 11, SEQ ID NO: 58, or SEQ ID NO: 4. In a further embodiment of this aspect, the residues of JM zone 2 may be modified (e.g., by insertion, deletion, alteration, etc.) in such a way that the modified residues do not correspond to the residues of a different JM zone 2 from a different LysM receptor.

The JM zone 3 corresponds to amino acids 281 to 304 when aligned to SEQ ID NO: 1 or corresponds to amino acids 282 to 302 when aligned to SEQ ID NO: 8. In yet another embodiment of this aspect, the JM zone 3 includes SEQ ID NO: 12, SEQ ID NO: 59, or SEQ ID NO: 5. In a further embodiment of this aspect, the residues of JM zone 3 may be modified (e.g., by insertion, deletion, alteration, etc.) in such a way that the modified residues do not correspond to the residues of a different JM zone 3 from a different LysM receptor.

The modified extracellular domain (ectodomain) can be modified as disclosed in U.S. patent application Ser. No. 17/324,354 (U.S. Pat. App. Pub. No. US-2021-03663200-A1) or in U.S. patent application Ser. No. 17/267,240 (U.S. Pat. App. Pub. NO. US-2021-0233608-A1).

The kinase C-terminus region corresponds to amino acids T467, S471, S473, P475, G477, V496, V500, Y510, D517, 1519, V520, S523, S525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, and G621 when aligned to SEQ ID NO: 8, or corresponds to amino acids 1469, N473, T475, Q477, 1497, 1501, F511, N518, V520, L521, G524, L526, V527, A528, E529, E539, A540, N542, K543, S544, C547, D548, A549, P556, E560, E564, L568, 1570, G574, R575, R579, L583, L591, L601, E603, C605, D607, E608, S610, S613, T615, 1617, L620, and V622 when aligned to SEQ ID NO: 1.

The kinase N-terminus region corresponds to D328, A338, K347, K348, T349, V356, T360, C364, H371, C384, V385, H395, D397, G402, Y404, H406, K410, E411, S416, S417, A425, V445, and L456 when aligned to SEQ ID NO: 1, or corresponds to A326, S336, E345, R346, A347, M354, K358, A362, R369, S382, 1383, F393, E395, S400, H402, R404, R408, D409, A414, T415, S423, 1443, and Y454 when aligned to SEQ ID NO: 8.

Transformation and generation of genetically altered monocotyledonous and dicotyledonous plant cells is well known in the art. See, e.g., Weising, et al., Ann. Rev. Genet. 22:421-477 (1988); U.S. Pat. No. 5,679,558; Agrobacterium Protocols, ed: Gartland, Humana Press Inc. (1995); Wang, et al. Acta Hort. 461:401-408 (1998), and Broothaerts, et al. Nature 433:629-633 (2005). The choice of method varies with the type of plant to be transformed, the particular application, and/or the desired result. The appropriate transformation technique is readily chosen by the skilled practitioner.

Any methodology known in the art to delete, insert or otherwise modify the cellular DNA (e.g., genomic DNA and organelle DNA) can be used in practicing the compositions, methods, and processes disclosed herein. As an example, the CRISPR/Cas-9 system and related systems (e.g., TALEN, ZFN, ODN, etc.) may be used to insert a heterologous gene to a targeted site in the genomic DNA or substantially edit an endogenous gene to express the heterologous gene or to modify the promoter to increase or otherwise alter expression of an endogenous gene through, for example, removal of repressor binding sites or introduction of enhancer binding sites. For example, a disarmed Ti plasmid, containing a genetic construct for deletion or insertion of a target gene, in Agrobacterium tumefaciens can be used to transform a plant cell, and thereafter, a transformed plant can be regenerated from the transformed plant cell using procedures described in the art, for example, in EP 0116718, EP 0270822, PCT publication WO 84/02913 and published European Patent application (“EP”) 0242246. Ti-plasmid vectors each contain the gene between the border sequences, or at least located to the left of the right border sequence, of the T-DNA of the Ti-plasmid. Of course, other types of vectors can be used to transform the plant cell, using procedures such as direct gene transfer (as described, for example in EP 0233247), pollen mediated transformation (as described, for example in EP 0270356, PCT publication WO 85/01856, and U.S. Pat. No. 4,684,611), plant RNA virus-mediated transformation (as described, for example in EP 0 067 553 and U.S. Pat. No. 4,407,956), liposome-mediated transformation (as described, for example in U.S. Pat. No. 4,536,475), and other methods such as the methods for transforming certain lines of corn (e.g., U.S. Pat. No. 6,140,553; Fromm et al., Bio/Technology (1990) 8, 833-839); Gordon-Kamm et al., The Plant Cell, (1990) 2, 603-618), rice (Shimamoto et al., Nature, (1989) 338, 274-276; Datta et al., Bio/Technology, (1990) 8, 736-740), and the method for transforming monocots generally (PCT publication WO 92/09696). For cotton transformation, the method described in PCT patent publication WO 00/71733 can be used. For soybean transformation, reference is made to methods known in the art, e.g., Hinchee et al. (Bio/Technology, (1988) 6, 915) and Christou et al. (Trends Biotech, (1990) 8, 145) or the method of WO 00/42207.

Genetically altered plants of the present disclosure can be used in a conventional plant breeding scheme to produce more genetically altered plants with the same characteristics, or to introduce the genetic alteration(s) in other varieties of the same or related plant species. Seeds, which are obtained from the altered plants, preferably contain the genetic alteration(s) as a stable insert in chromosomal DNA or as modifications to an endogenous gene or promoter. Plants including the genetic alteration(s) in accordance with this disclosure include plants including, or derived from, root stocks of plants including the genetic alteration(s) of this disclosure, e.g., fruit trees or ornamental plants. Hence, any non-transgenic grafted plant parts inserted on a transformed plant or plant part are included in this disclosure.

Genetic alterations of the disclosure, including in an expression vector or expression cassette, which result in the expression of an introduced gene or altered expression of an endogenous gene will typically utilize a plant-expressible promoter. A ‘plant-expressible promoter’ as used herein refers to a promoter that ensures expression of the genetic alteration(s) of this disclosure in a plant cell. Examples of constitutive promoters that are often used in plant cells are the cauliflower mosaic (CaMV) 35S promoter (Kay et al. Science, 236, 4805, 1987), the minimal CaMV 35S promoter (Benfey & Chua, Science, (1990) 250, 959-966), various other derivatives of the CaMV 35S promoter, the figwort mosaic virus (FMV) promoter (Richins, et al., Nucleic Acids Res. (1987) 15:8451-8466)the maize ubiquitin promoter (Christensen & Quail, Transgenic Res, 5, 213-8, 1996), the polyubiquitin promoter (Ljubql, Maekawa et al. Mol Plant Microbe Interact. 21, 375-82, 2008), the vein mosaic cassava virus promoter (International Application WO 97/48819), and the Arabidopsis UBQ10 promoter, Norris et al. Plant Mol. Biol. 21, 895-906, 1993).

Additional examples of promoters directing constitutive expression in plants are known in the art and include: the strong constitutive 35S promoters (the “35S promoters”) of the cauliflower mosaic virus (CaMV), e.g., of isolates CM 1841 (Gardner et al., Nucleic Acids Res, (1981) 9, 2871-2887), CabbB S (Franck et al., Cell (1980) 21, 285-294) and CabbB JI (Hull and Howell, Virology, (1987) 86, 482-493); promoters from the ubiquitin family (e.g., the maize ubiquitin promoter of Christensen et al., Plant Mol Biol, (1992) 18, 675-689), the gos2 promoter (de Pater et al., The Plant J (1992) 2, 834-844), the emu promoter (Last et al., Theor Appl Genet, (1990) 81, 581-588), actin promoters such as the promoter described by An et al. (The Plant J, (1996) 10, 107), the rice actin promoter described by Zhang et al. (The Plant Cell, (1991) 3, 1155-1165); promoters of the figwort mosaic virus (FMV) (Richins, et al., Nucleic Acids Res. (1987) 15:8451-8466), promoters of the Cassava vein mosaic virus (WO 97/48819; Verdaguer et al., Plant Mol Biol, (1998) 37, 1055-1067), the pPLEX series of promoters from Subterranean Clover Stunt Virus (WO 96/06932, particularly the S4 or S7 promoter), an alcohol dehydrogenase promoter, e.g., pAdhlS (GenBank accession numbers X04049, X00581), and the TR1′ promoter and the TR2′ promoter (the “TR1′ promoter” and “TR2′ promoter”, respectively) which drive the expression of the 1′ and 2′ genes, respectively, of the T DNA (Velten et al., EMBO J, (1984) 3, 2723-2730).

Alternatively, a plant-expressible promoter can be a tissue-specific promoter, i.e., a promoter directing a higher level of expression in some cells or tissues of the plant, e.g., in root epidermal cells or root cortex cells. In preferred embodiments, LysM receptor promoters will be used. Non-limiting examples include NFR1 promoters, NFR5 promoters, LYK3 promoters, NFP promoters, the Lotus japonicus NFR5 promoter (SEQ ID NO: 109), the Lotus japonicus NFR1 promoter (SEQ ID NO: 151), the Medicago truncatula NFP promoter (SEQ ID NO: 110), the Lotus japonicus CERK6 promoter (SEQ ID NO: 111), and the Medicago truncatula LYK3 promoter (SEQ ID NO: 112). In additional preferred embodiments, root specific promoters will be used. Non-limiting examples include the promoter of the maize metallothioneine (De Framond et al, FEBS 290, 103.-106, 1991 Application EP 452269), the chitinase promoter (Samac et al. Plant Physiol 93, 907-914, 1990), the glutamine synthetase soybean root promoter (Hirel et al. Plant Mol. Biol. 20, 207-218, 1992), the RCC3 promoter (PCT Application WO 2009/016104), the rice antiquitin promoter (PCT Application WO 2007/076115), the LRR receptor kinase promoter (PCT application WO 02/46439), the maize ZRP2 promoter (U.S. Pat. No. 5,633,363), the tomato LeExt1 promoter (Bucher et al. Plant Physiol. 128, 911-923, 2002), and the Arabidopsis pCO2 promoter (Heidstra et al, Genes Dev. 18, 1964-1969, 2004). These plant promoters can be combined with enhancer elements, they can be combined with minimal promoter elements, or can comprise repeated elements to ensure the expression profile desired.

Examples of constitutive promoters that are often used in plant cells are the cauliflower mosaic (CaMV) 35S promoter (Kay et al. Science, 236, 4805, 1987), and various derivatives of the promoter, virus promoter vein mosaic cassava (International Application WO 97/48819), the maize ubiquitin promoter (Christensen & Quail, Transgenic Res, 5, 213-8, 1996), polyubiquitin (Ljubql, Maekawa et al. Mol Plant Microbe Interact. 21, 375-82, 2008) and Arabidopsis UBQ10 (Norris et al. Plant Mol. Biol. 21, 895-906, 1993).

In some embodiments, further genetic alterations to increase expression in plant cells can be utilized. For example, an intron at the 5′ end or 3′ end of an introduced gene, or in the coding sequence of the introduced gene, e.g., the hsp70 intron. Other such genetic elements can include, but are not limited to, promoter enhancer elements, duplicated or triplicated promoter regions, 5′ leader sequences different from another transgene or different from an endogenous (plant host) gene leader sequence, 3′ trailer sequences different from another transgene used in the same plant or different from an endogenous (plant host) trailer sequence.

An introduced gene of the present disclosure can be inserted in host cell DNA so that the inserted gene part is upstream (i.e., 5′) of suitable 3′ end transcription regulation signals (i.e., transcript formation and polyadenylation signals). This is preferably accomplished by inserting the gene in the plant cell genome (nuclear or chloroplast). Preferred polyadenylation and transcript formation signals include those of the nopaline synthase gene (Depicker et al., J. Molec Appl Gen, (1982) 1, 561-573), the octopine synthase gene (Gielen et al., EMBO J, (1984) 3:835-845), the SCSV or the Malic enzyme terminators (Schunmann et al., Plant Funct Biol, (2003) 30:453-460), and the T DNA gene 7 (Velten and Schell, Nucleic Acids Res, (1985) 13, 6981-6998), which act as 3′ untranslated DNA sequences in transformed plant cells. In some embodiments, one or more of the introduced genes are stably integrated into the nuclear genome. Stable integration is present when the nucleic acid sequence remains integrated into the nuclear genome and continues to be expressed (i.e., detectable mRNA transcript or protein is produced) throughout subsequent plant generations. Stable integration into the nuclear genome can be accomplished by any known method in the art (e.g., microparticle bombardment, Agrobacterium-mediated transformation, CRISPR/Cas9, electroporation of protoplasts, microinjection, etc.).

The term recombinant or modified nucleic acids refers to polynucleotides which are made by the combination of two otherwise separated segments of sequence accomplished by the artificial manipulation of isolated segments of polynucleotides by genetic engineering techniques or by chemical synthesis. In so doing one may join together polynucleotide segments of desired functions to generate a desired combination of functions.

As used herein, the term “overexpression” refers to increased expression (e.g., of mRNA, polypeptides, etc.) relative to expression in a wild type organism (e.g., plant) as a result of genetic modification and can refer to expression of heterologous genes at a sufficient level to achieve the desired result such as increased yield. In some embodiments, the increase in expression is a slight increase of about 10% more than expression in wild type. In some embodiments, the increase in expression is an increase of 50% or more (e.g., 60%, 70%, 80%, 100%, etc.) relative to expression in wild type. In some embodiments, an endogenous gene is upregulated. In some embodiments, an exogenous gene is upregulated by virtue of being expressed. Upregulation of a gene in plants can be achieved through any known method in the art, including but not limited to, the use of constitutive promoters with inducible response elements added, inducible promoters, high expression promoters (e.g., PsaD promoter) with inducible response elements added, enhancers, transcriptional and/or translational regulatory sequences, codon optimization, modified transcription factors, and/or mutant or modified genes that control expression of the gene to be upregulated in response to a stimulus such as cytokinin signaling.

Where a recombinant nucleic acid is intended for expression, cloning, or replication of a particular sequence, DNA constructs prepared for introduction into a host cell will typically include a replication system (e.g., vector) recognized by the host, including the intended DNA fragment encoding a desired polypeptide, and can also include transcription and translational initiation regulatory sequences operably linked to the polypeptide-encoding segment. Additionally, such constructs can include cellular localization signals (e.g., plasma membrane localization signals). In preferred embodiments, such DNA constructs are introduced into a host cell's genomic DNA, chloroplast DNA or mitochondrial DNA.

In some embodiments, a non-integrated expression system can be used to induce expression of one or more introduced genes. Expression systems (expression vectors) can include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Signal peptides can also be included where appropriate from secreted polypeptides of the same or related species, which allow the protein to cross and/or lodge in cell membranes, cell wall, or be secreted from the cell.

Selectable markers useful in practicing the methodologies disclosed herein can be positive selectable markers. Typically, positive selection refers to the case in which a genetically altered cell can survive in the presence of a toxic substance only if the recombinant polynucleotide of interest is present within the cell. Negative selectable markers and screenable markers are also well known in the art and are contemplated by the present disclosure. One of skill in the art will recognize that any relevant markers available can be utilized in practicing the compositions, methods, and processes disclosed herein.

Screening and molecular analysis of recombinant strains of the present disclosure can be performed utilizing nucleic acid hybridization techniques. Hybridization procedures are useful for identifying polynucleotides, such as those modified using the techniques described herein, with sufficient homology to the subject regulatory sequences to be useful as taught herein. The particular hybridization techniques are not essential to this disclosure. As improvements are made in hybridization techniques, they can be readily applied by one of skill in the art. Hybridization probes can be labeled with any appropriate label known to those of skill in the art. Hybridization conditions and washing conditions, for example temperature and salt concentration, can be altered to change the stringency of the detection threshold. See, e.g., Sambrook et al. (1989) vide infra or Ausubel et al. (1995) Current Protocols in Molecular Biology, John Wiley & Sons, NY, N.Y., for further guidance on hybridization conditions.

Additionally, screening and molecular analysis of genetically altered strains, as well as creation of desired isolated nucleic acids can be performed using Polymerase Chain Reaction (PCR). PCR is a repetitive, enzymatic, primed synthesis of a nucleic acid sequence. This procedure is well known and commonly used by those skilled in this art (see Mullis, U.S. Pat. Nos. 4,683,195, 4,683,202, and 4,800,159; Saiki et al. (1985) Science 230:1350-1354). PCR is based on the enzymatic amplification of a DNA fragment of interest that is flanked by two oligonucleotide primers that hybridize to opposite strands of the target sequence. The primers are oriented with the 3′ ends pointing towards each other. Repeated cycles of heat denaturation of the template, annealing of the primers to their complementary sequences, and extension of the annealed primers with a DNA polymerase result in the amplification of the segment defined by the 5′ ends of the PCR primers. Because the extension product of each primer can serve as a template for the other primer, each cycle essentially doubles the amount of DNA template produced in the previous cycle. This results in the exponential accumulation of the specific target fragment, up to several million-fold in a few hours. By using a thermostable DNA polymerase such as the Taq polymerase, which is isolated from the thermophilic bacterium Thermus aquaticus, the amplification process can be completely automated. Other enzymes which can be used are known to those skilled in the art.

Nucleic acids and proteins of the present disclosure can also encompass homologues of the specifically disclosed sequences. Homology (e.g., sequence identity) can be 50%-100%. In some instances, such homology is greater than 80%, greater than 85%, greater than 90%, or greater than 95%. The degree of homology or identity needed for any intended use of the sequence(s) is readily identified by one of skill in the art. As used herein percent sequence identity of two nucleic acids is determined using an algorithm known in the art, such as that disclosed by Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the BLASTN, BLASTP, and BLASTX, programs of Altschul et al. (1990) J. Mol. Biol. 215:402-410. BLAST nucleotide searches are performed with the BLASTN program, score=100, wordlength=12, to obtain nucleotide sequences with the desired percent sequence identity. To obtain gapped alignments for comparison purposes, Gapped BLAST is used as described in Altschul et al. (1997) Nucl. Acids. Res. 25:3389-3402. When utilizing BLAST and Gapped BLAST programs, the default parameters of the respective programs (BLASTN and BLASTX) are used. See www.ncbi.nih.gov. One of skill in the art can readily determine in a sequence of interest where a position corresponding to amino acid or nucleic acid in a reference sequence occurs by aligning the sequence of interest with the reference sequence using the suitable BLAST program with the default settings (e.g., for BLASTP: Gap opening penalty: 11, Gap extension penalty: 1, Expectation value: 10, Word size: 3, Max scores: 25, Max alignments: 15, and Matrix: blosum62; and for BLASTN: Gap opening penalty: 5, Gap extension penalty:2, Nucleic match: 1, Nucleic mismatch−3, Expectation value: 10, Word size: 11, Max scores: 25, and Max alignments: 15).

Preferred host cells are plant cells. Recombinant host cells, in the present context, are those which have been genetically modified to contain an isolated nucleic molecule, contain one or more deleted or otherwise non-functional genes normally present and functional in the host cell, or contain one or more genes to produce at least one recombinant protein. The nucleic acid(s) encoding the protein(s) of the present disclosure can be introduced by any means known to the art which is appropriate for the particular type of cell, including without limitation, transformation, lipofection, electroporation or any other methodology known by those skilled in the art.

“Isolated”, “isolated DNA molecule” or an equivalent term or phrase is intended to mean that the DNA molecule or other moiety is one that is present alone or in combination with other compositions, but altered from or not within its natural environment. For example, nucleic acid elements such as a coding sequence, intron sequence, untranslated leader sequence, promoter sequence, transcriptional termination sequence, and the like, that are naturally found within the DNA of the genome of an organism are not considered to be “isolated” so long as the element is within the genome of the organism and at the location within the genome in which it is naturally found. However, each of these elements, and subparts of these elements, would be “isolated” from its natural setting within the scope of this disclosure so long as the element is not within the genome of the organism in which it is naturally found, the element is altered from its natural form, or the element is not at the location within the genome in which it is naturally found. Similarly, a nucleotide sequence encoding a protein or any naturally occurring variant of that protein would be an isolated nucleotide sequence so long as the nucleotide sequence was not within the DNA of the organism from which the sequence encoding the protein is naturally found in its natural location or if that nucleotide sequence was altered from its natural form. A synthetic nucleotide sequence encoding the amino acid sequence of the naturally occurring protein would be considered to be isolated for the purposes of this disclosure. For the purposes of this disclosure, any transgenic nucleotide sequence, i.e., the nucleotide sequence of the DNA inserted into the genome of the cells of a plant, alga, fungus, or bacterium, or present in an extrachromosomal vector, would be considered to be an isolated nucleotide sequence whether it is present within the plasmid or similar structure used to transform the cells, within the genome of the plant or bacterium, or present in detectable amounts in tissues, progeny, biological samples or commodity products derived from the plant or bacterium.

Having generally described the compositions, methods, and processes of this disclosure, the same will be better understood by reference to certain specific examples, which are included herein to further illustrate the disclosure and are not intended to limit the scope of the invention as defined by the claims.

### EXAMPLES

The present disclosure is described in further detail in the following examples which are not in any way intended to limit the scope of the disclosure as claimed. The attached figures are meant to be considered as integral parts of the specification and description of the disclosure. The following examples are offered to illustrate, but not to limit the claimed disclosure. Examples 1-7 describe the identification of zone 4 of the NFR1 juxtamembrane domain (NFR1 zone 4=SEQ ID NO: 6) as a determinant of root nodule symbiosis and target for engineering in non-legume receptors. Example 8 describes the crystal structures of CERK6 and LYK3 proteins. Example 9 describes biochemical analyses of native, mutant, and chimeric NFR1-CERK6 proteins. Examples 10-12 describe the identification of the N terminal region of CERK6 as being critical for immunity. Example 13 describes investigating the role of zone 4 in activating symbiotic signaling. Example 14 describes experiments investigating the role of the CERK6 kinase domains in root hair infection thread formation. Example 15 describes experiments investigating ROS production after treatment with Nod factors in lines expressing NFR1 variants containing CERK6 kinase. Example 16 describes experiments investigating whether NFR1 variants containing CERK6 kinase form inhibitory dimers with the native CERK6. Example 17 describes experiments investigating the NFR1 zone 4 biochemical properties that are required for symbiotic signaling. Example 18 describes experiments investigating the effects on nodulation of mutations in the NFR1 activation loop (AL). Example 19 describes experiments investigating the role of CERK6 juxtamembrane in immune signaling. Example 20 describes experiments investigating the structure of the CERK6 activation loop in the absence of the ligand.

### Example 1: LjNFR1 JM Zone 4 is Essential for Nodule Organogenesis

Example 1 describes that zone 4 of the NFR1 protein from Lotus japonicus (LjNFR1) juxtamembrane domain (JM; NFR1 zone 4=SEQ ID NO: 6) is essential for nodule organogenesis.

### Materials and Methods

### Cloning of TM/JM Chimeras

A level 2 pIV10 expression vector (Márquez, A. J., and J. Stougaard, eds. 2005. Lotus Japonicus Handbook. Dordrecht: Springer) containing an expression cassette of nuclear localized tripleYFP driven by the constitutive promoter pUbi (pUbi::tYFP-NLS) serving as transformation marker was used for Hairy Root transformation. The chimeric constructs or chimeric variants were positioned downstream of pNFR1. Golden gate cloning (Weber, Ernst, Carola Engler, Ramona Gruetzner, Stefan Werner, and Sylvestre Marillonnet. 2011. “A Modular Cloning System for Standardized Assembly of Multigene Constructs” edited by J. Peccoud. PLoS ONE 6(2):e16765. doi: 0.1371/journal.pone.0016765.) was used to create the different receptor versions. The modules utilized to assemble the receptor chimeras were the following: LjCERK6 and LjNFR1 (genomic sequences containing introns): Ectodomain (EC), Transmembrane/Juxtamembrane domain (TM/JM), TM/JM zones 1-4 (TM/JM zone 1 is part of the TM (NFR1 zone 1=SEQ ID NO: 3; CERK6 zone 1=SEQ ID NO: 10), while zones 2-4 are part of the JM (NFR1 zone 2=SEQ ID NO: 4; NFR1 zone 3=SEQ ID NO: 5; NFR1 zone 4=SEQ ID NO: 6; CERK6 zone 2=SEQ ID NO: 11; CERK6 zone 3=SEQ ID NO: 12; CERK6 zone 4=SEQ ID NO: 13), and Kinase domain.

### Bacteria and Culture Conditions

E. coli TOP10 (ThermoFisher Scientific) were used for molecular cloning and grown in LB medium at 37° C. Agrobacterium rhizogenes strain AR1193 was used for hairy root transformations. Agrobacterium strains were grown in LB medium at 28° C. M. loti strain R7A dsRED, used for nodulation assay, was grown in YMB at 28 degrees C.

### Plant Materials

The Lotus japonicus ecotype Gifu wild type and mutant line nfr1-1 pNin-gus were used for nodulation assays. Plants were grown at 21° C. (16/8-hour light/dark conditions).

### Hairy Root Transformation

For scarification, Lotus seeds were either immersed in sulfuric acid for 15 minutes or treated with sandpaper. Seeds were then surface sterilized for 3 minutes with 3% sodium hypochlorite, washed 3 times in ddH₂O and dispersed on wet filter paper for germination. Three-day-old seedlings were transferred to square agar plates solidified with 0.8% Gelrite (Duchefa Biochemi) supplemented with 12 Gamborg's B5 nutrient solution (Duchefa Biochemi). A. rhizogenes AR1193 strains carrying the construct of interest were grown for three days on LB Agar containing Ampicillin, Rifampicin, and Spectinomycin (final concentration of each antibiotic was 100 μg/ml). For each construct the cells grown on one plate were resuspended in 4 mL YMB (5 g/L mannitol, 0.5 g/L yeast extract, 0.5 g/L K₂HPO₄, 0.2 g/L MgSO4·7H₂O, 0.1 g NaCl, pH=6.8). The bacterial suspension was then used to transform 6-day-old seedlings using a 1 mL syringe with a needle (Sterican® Ø0.40×20 mm), punching the hypocotyl and placing a droplet on the root emerging from the wound. Square plates containing the transformed seedlings were sealed and left in the dark for one day and then moved to 21° C. under 16/8-hour light/dark conditions. After three weeks, primary roots were removed and seedlings with transformed roots were transferred to Magenta™ vessels (Sigma-Aldrich) filled with lightweight expanded clay aggregate (Leca®, 2-4 mm; Saint-Gobain Weber A/S) supplemented with 80 mL nitrogen-free 1/4×B&D nutrient solution.

### Nodulation Assay

If the roots were inoculated, nodules were counted five weeks after the transfer to Magenta™ vessels (Sigma-Aldrich) and inoculation with Rhizobia (equivalent to nine weeks after hairy root transformations). All nodulation assays in roots transformed using hairy root transformation occurred in Magenta™ vessels (Sigma-Aldrich). Pictures were acquired with a Leica M165FC Fluorescent Stereo Microscope equipped with the Leica DFC310 FX digital color camera.

A Kruskal-Wallis test (Kruskal et al. (1952) J Am Stat Assoc 47, 583-621) followed by Dunn's post-hoc test (Dunn (1964) Technometrics 6, 241-252) was used for statistical analysis of nodule counts.

Plant hairy roots were incubated in the GUS buffer consisting of: NaPO4 (pH 7.0) 100 mM, EDTA (pH 8.0) 10 mM, K Ferricyanide 1 mM, K Ferrocyanide 1 mM, Triton X-100 0.1%, X-Gluc (5-bromo-4-chloro-3-indolyl-beta-D-glucuronic acid, cyclohexylammonium salt) 0.5 mg/ml at 37° C. overnight in the dark. The roots were washed with 70% EtOH and kept at 4° C. for several days after inspection.

### Cell Use of Receptors

To identify whether NFR1 and CERK6 are expressed in the same root cells, data on single cell root transcriptomics (provided in: Frank et al. (2023) Single-cell analysis identifies genes facilitating rhizobium infection in Lotus japonicus. Nat Commun 14, 7171) were analyzed.

### Results

Previous studies investigating which regions of the NFR1 and CERK6 receptors contributed to immune or symbiotic signaling identified that the region containing transmembrane domain (TM) and juxtamembrane domain (JM) was crucial for root nodulation (see FIG. 1A) (Bozsoki et al. (2020) Science 369(6504), 663-670). It was also determined that cells are capable of expressing both NFR1 and CERK6 (see FIG. 1B). The TM and JM were known from other single-pass receptors to have different structures and functions, i.e., in oligomeric complex formations, interaction with partners or regulation of the kinase activity (Boran (2012) Biochemical Society Transactions, 40(1):195-99; Kwon et al. (2018); JBC, 293(14):5102-16; Maeda et al. (2018) Biophys. J., 114(4):893-903; Parat et al. (2010) Biochemistry, 49(22):4601-10). In order to identify which residues of the TM/JM regions of NFR1 and CERK6 were determinants for symbiotic signaling, the TM/JM was divided into 4 zones labeled 1-4 (see FIG. 1A; TM/JM zone 1 is part of the TM (NFR1 zone 1=SEQ ID NO: 3; CERK6 zone 1=SEQ ID NO: 10), while zones 2-4 are part of the JM (NFR1 zone 2=SEQ ID NO: 4; NFR1 zone 3=SEQ ID NO: 5; NFR1 zone 4=SEQ ID NO: 6; CERK6 zone 2=SEQ ID NO: 11; CERK6 zone 3=SEQ ID NO: 12; CERK6 zone 4=SEQ ID NO: 13)), and a series of thirteen chimeras were created in order to test for capacity to restore nodulation of the Lotus japonicus nfr1 mutant.

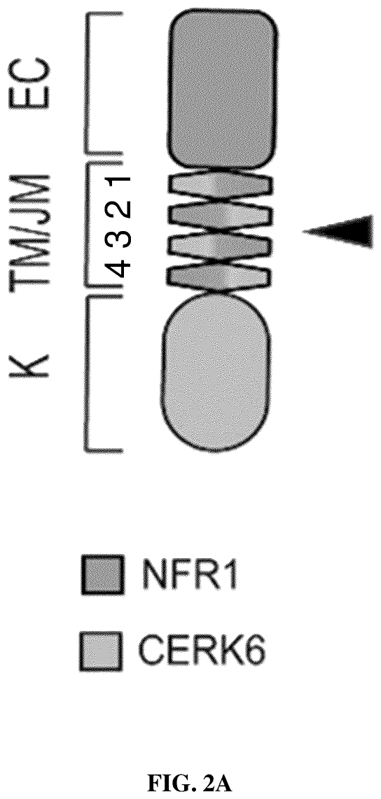

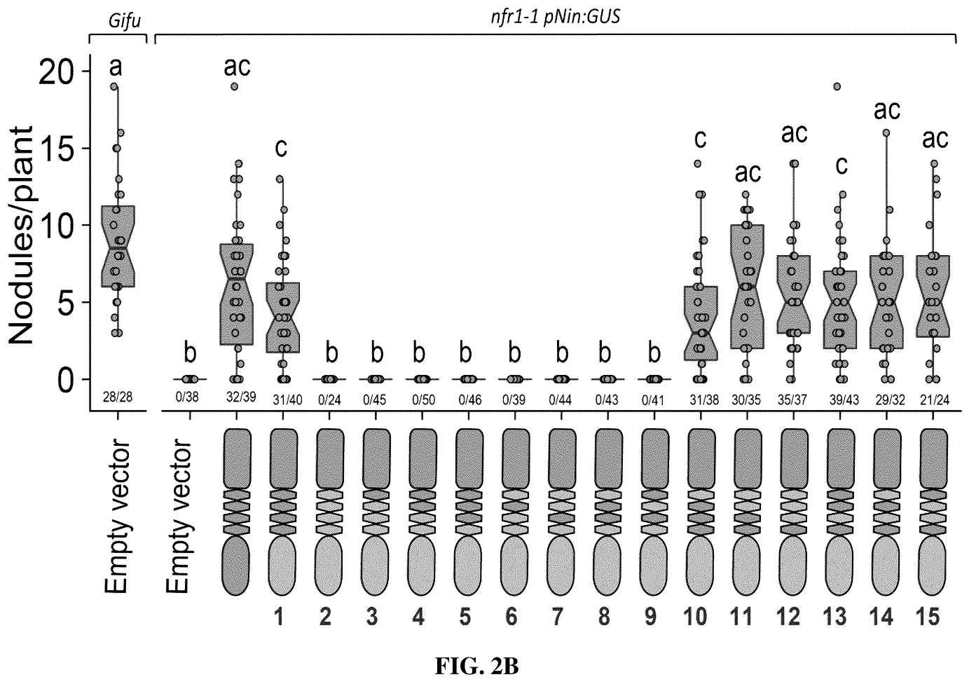

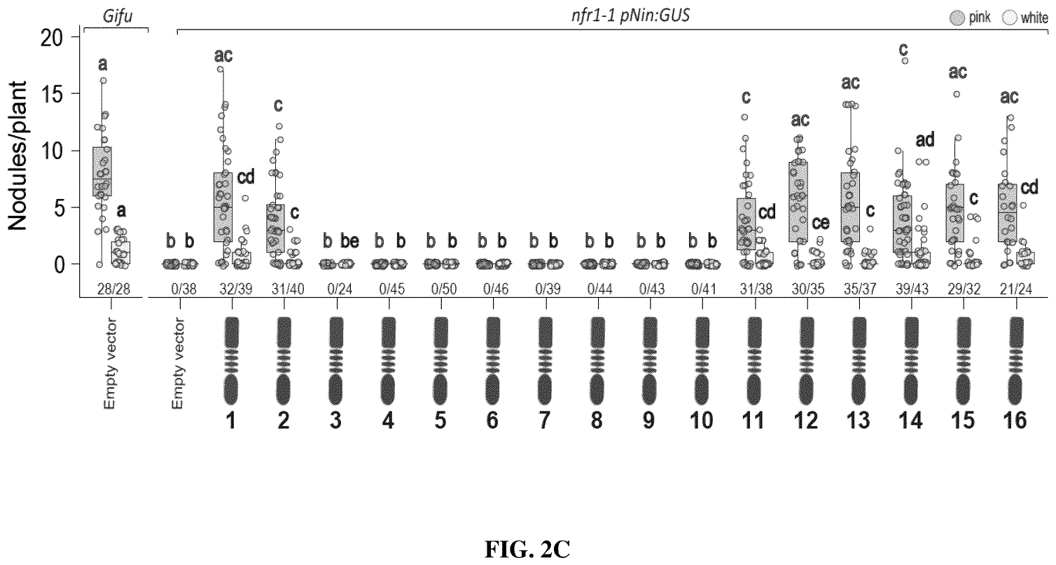

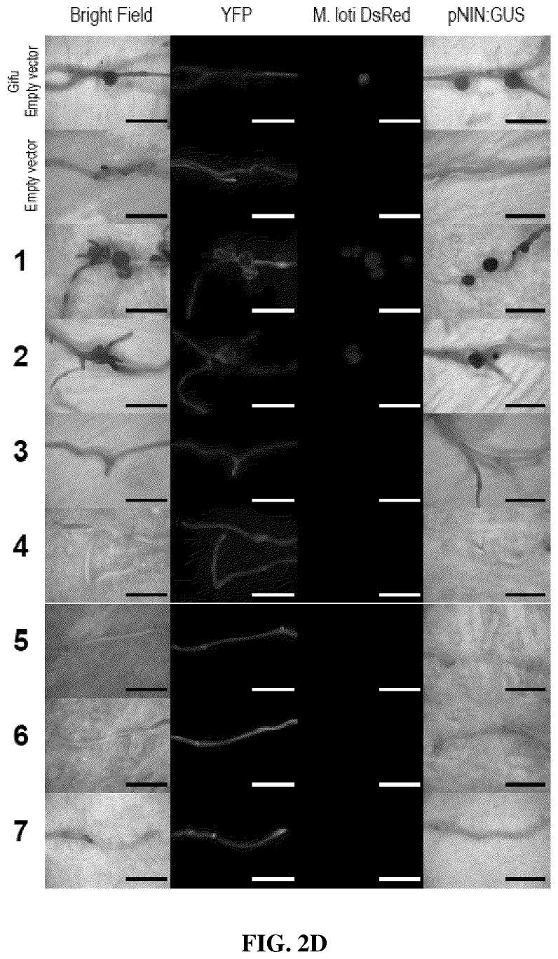

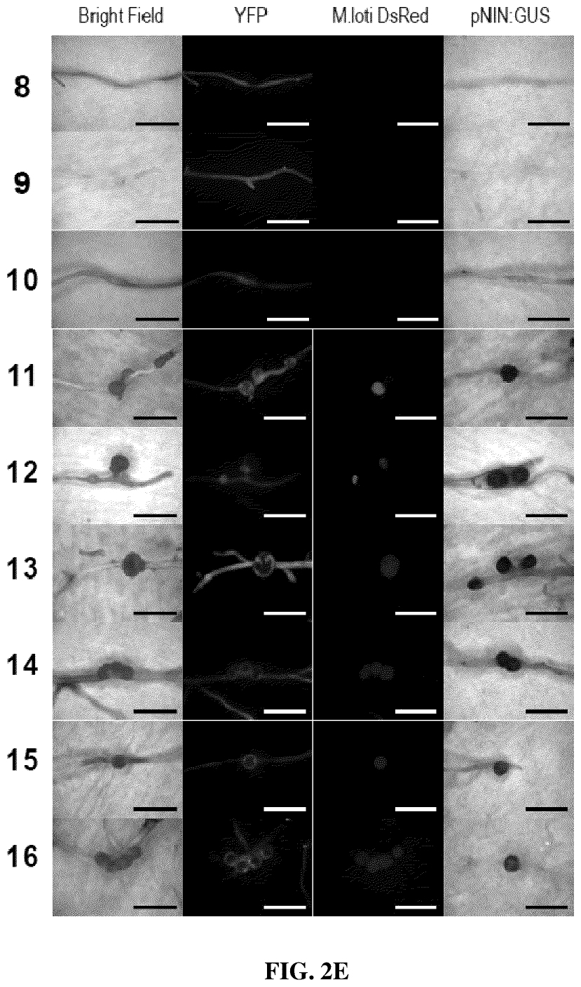

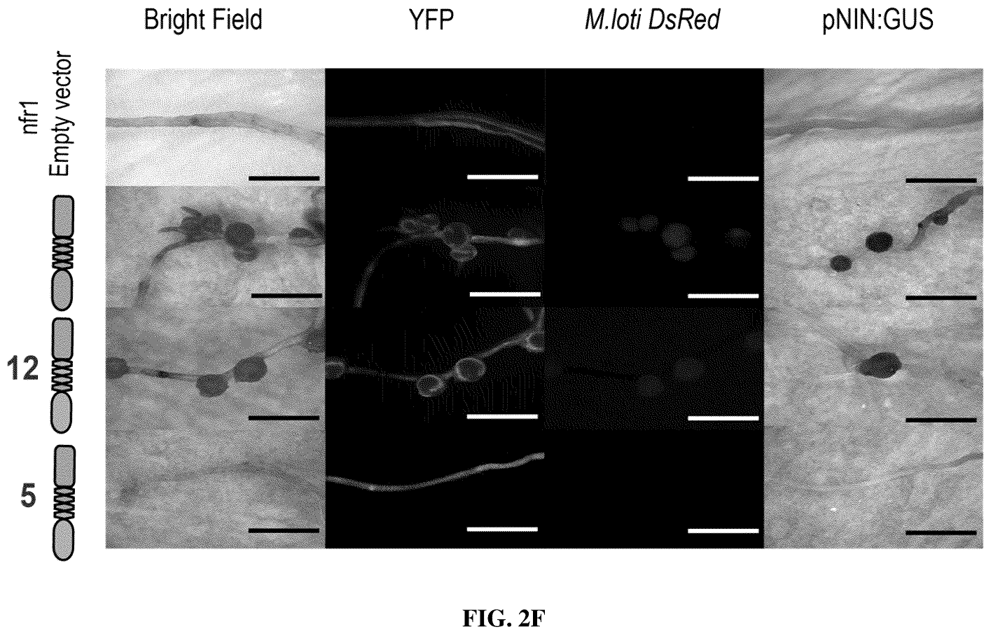

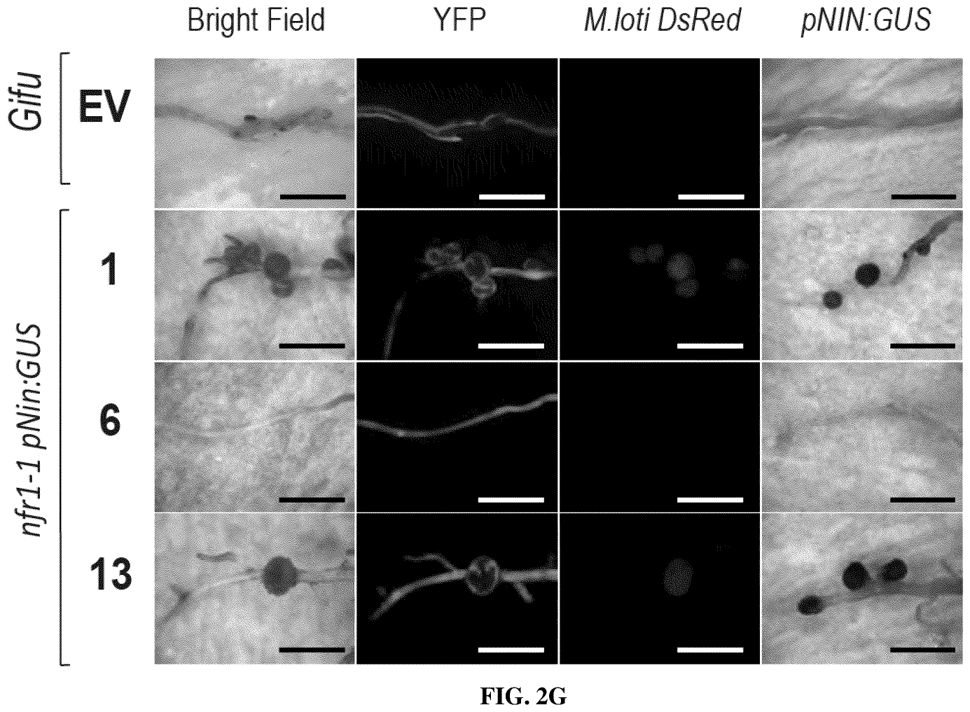

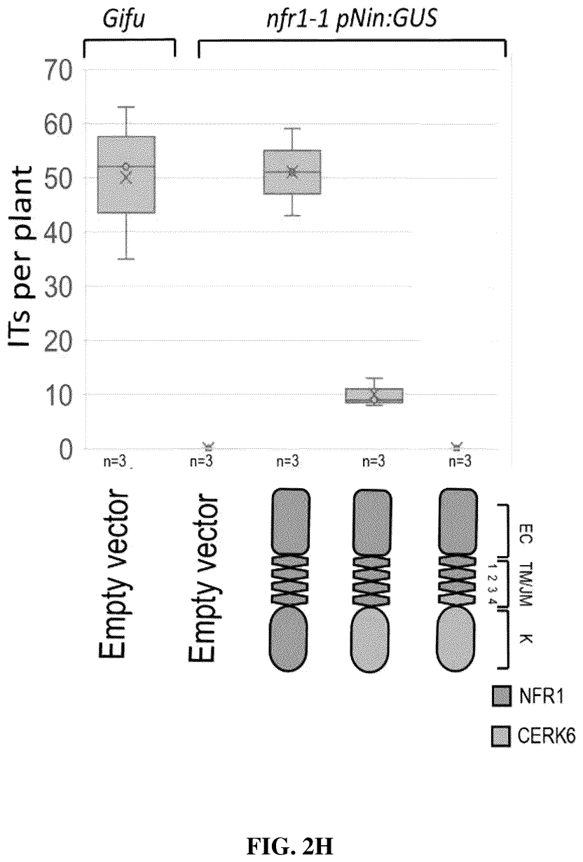

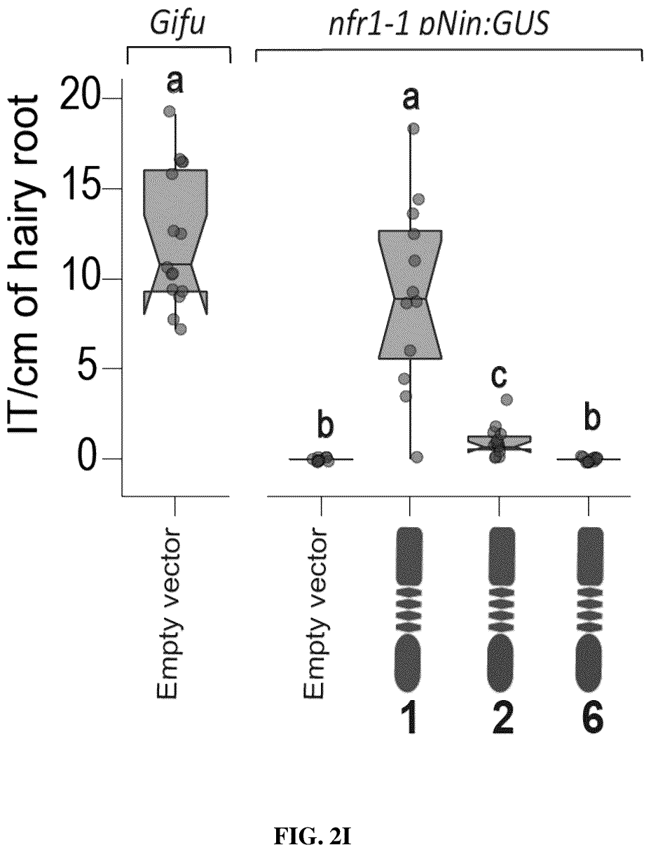

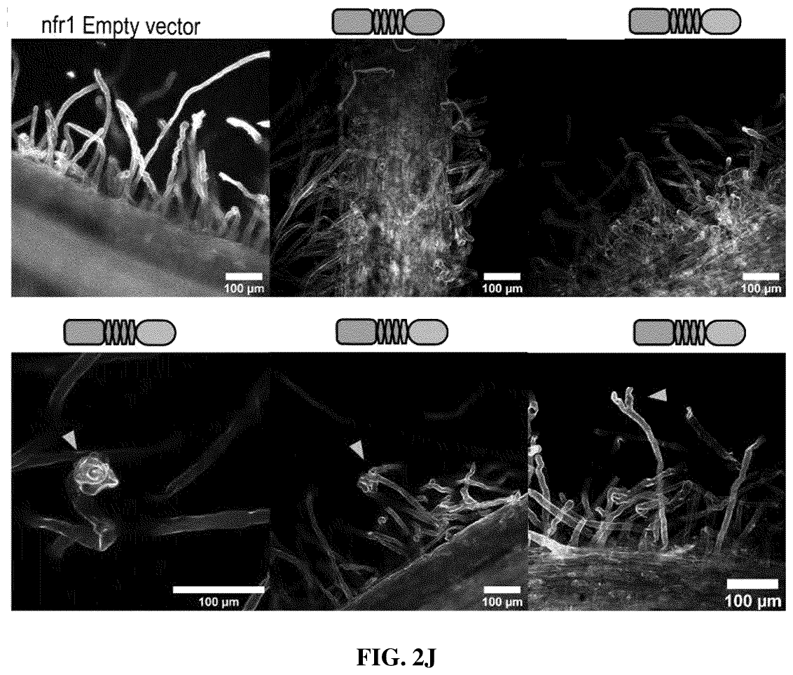

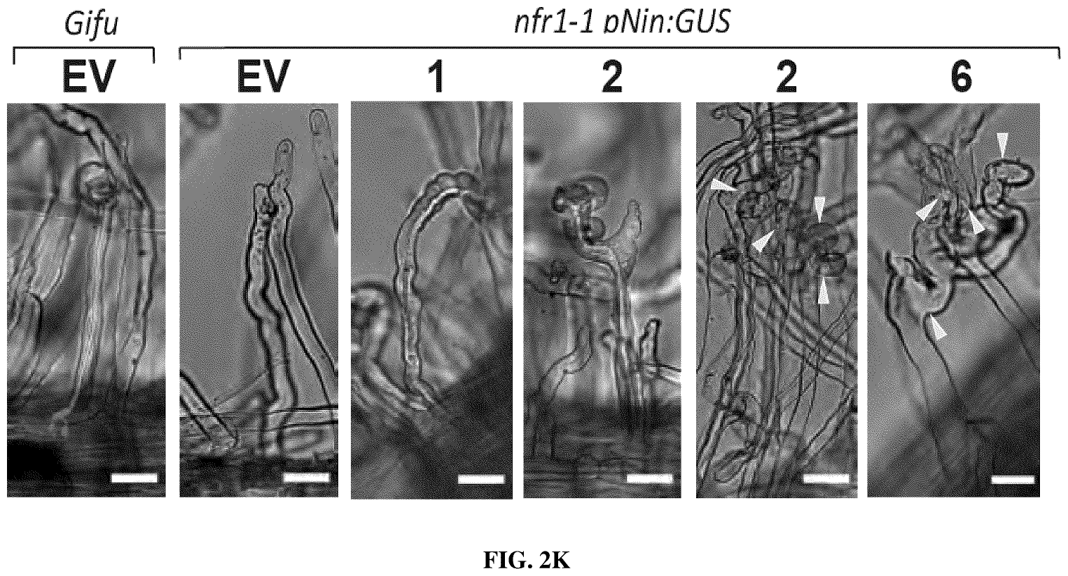

These chimeric constructs each contained the ectodomain of NFR1 and the kinase of CERK6 coupled to different NFR1-CERK6 chimeric versions of TM/JM (see FIG. 2A) All constructs were driven by the native Lotus japonicus NFR1 promoter.

In order to determine if the chimeric receptors had the capacity to activate symbiotic signaling and root nodulation, Lotus japonicus nfr1-1 mutant plants expressing a pNIN:GUS symbiotic marker were used as background for hairy root transformation, and nodule formation was assessed at 35 dpi with M. loti R7A dsRed (FIGS. 2B, 2C).

This full-dissection study, in which the TM/JM region was dissected in 4 zones and the role of each part was studied, identified that only constructs containing the LjNFR1 zone 4 were able to activate pNIN:GUS expression and induce nodule formation (see FIGS. 2B-2G). The average number of nodules was in general not significantly different from the full-length NFR1 construct (see FIGS. 2B, 2C, and visually in FIGS. 2D-2G). All complementing constructs (i.e., those with LjNFR1 zone 4) induced a full symbiotic phenotype, in which pink nodules expressing leghemoglobin and white nodule primordia formed (see FIG. 2F, middle two rows).

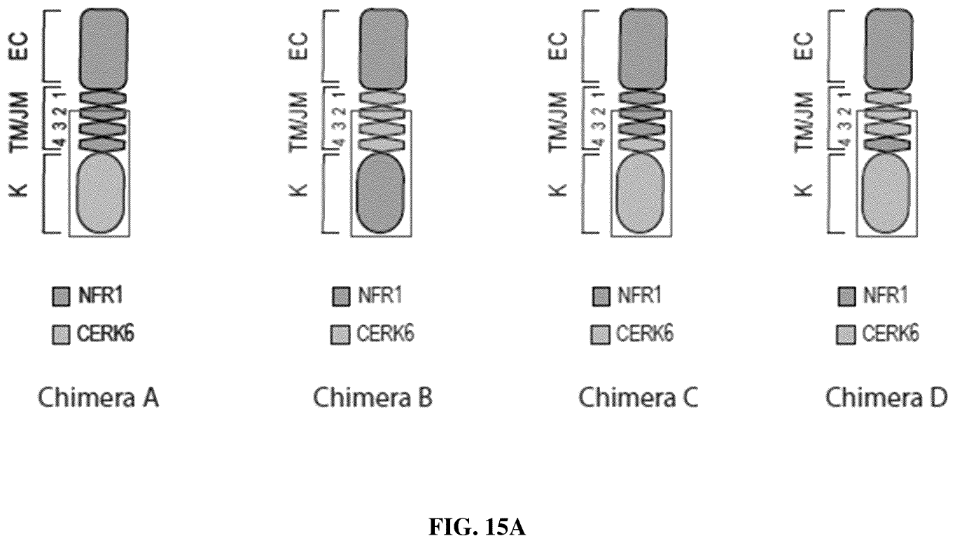

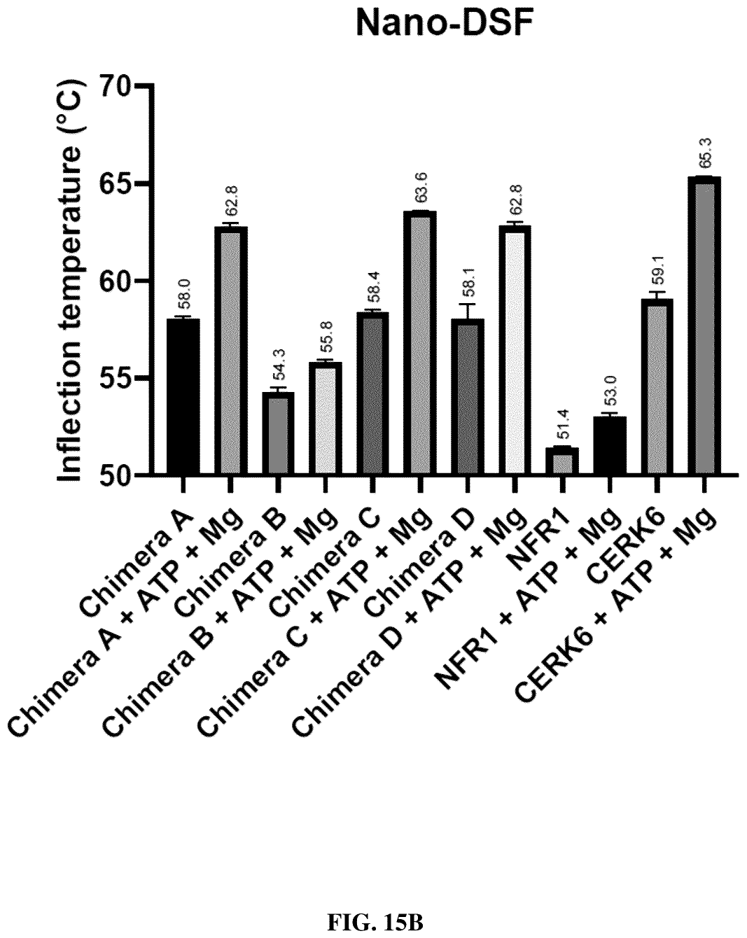

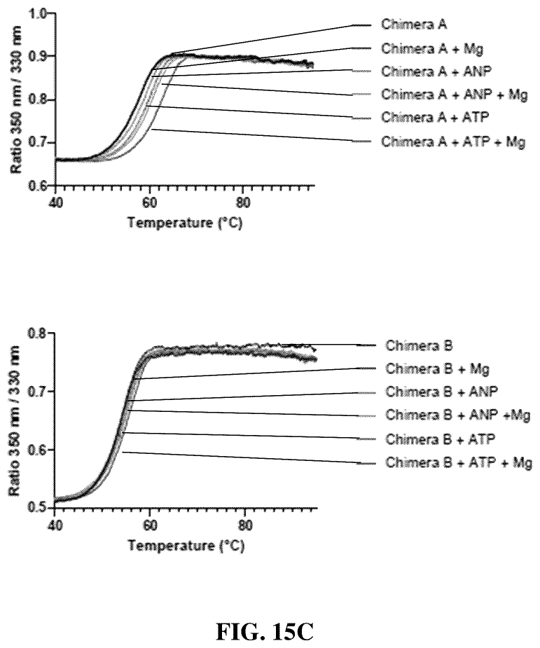

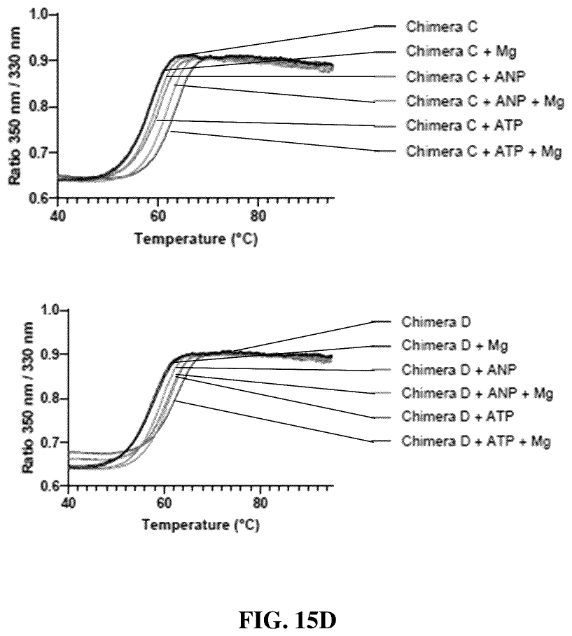

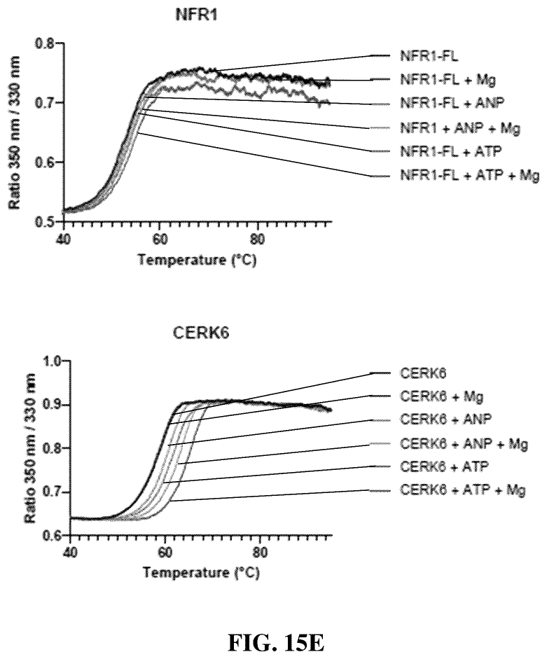

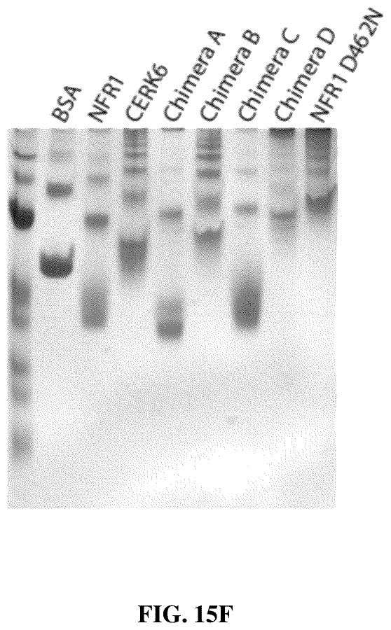

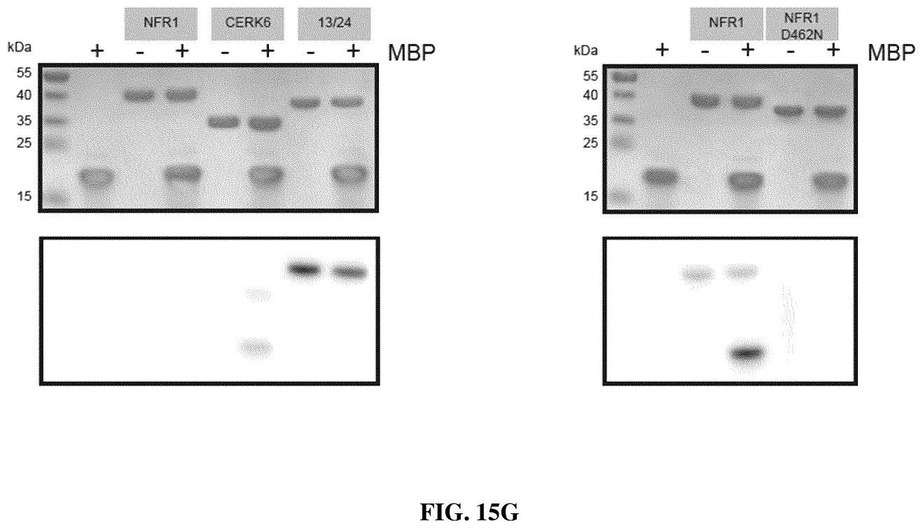

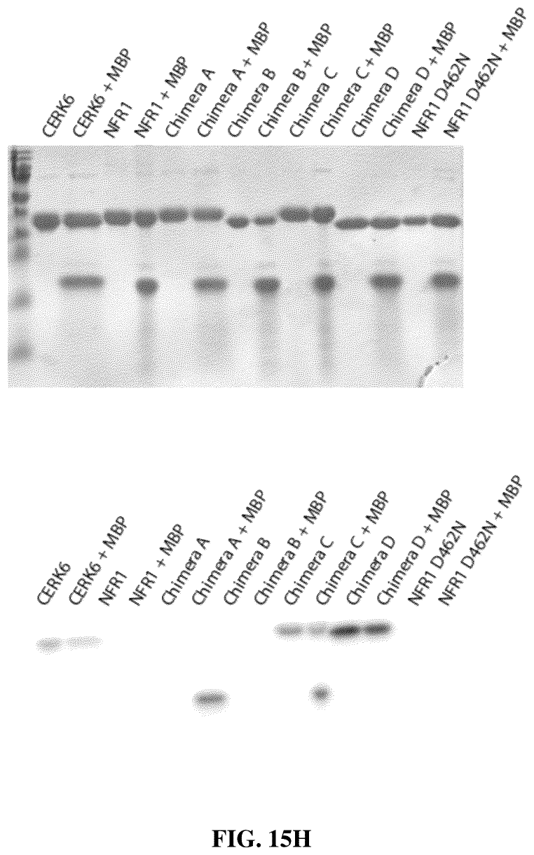

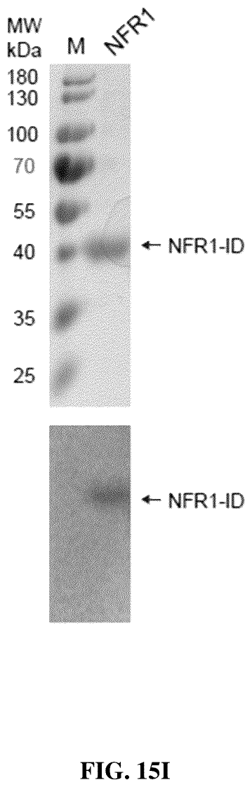

Detailed microscopic investigation revealed that M. loti infection of nodules and NIN promoter expression had been induced (pNin:GUS expression) in all roots expressing the complementing constructs (FIG. 2D-2G). Together, these results indicated that zone 4 of NFR1 was crucial for nodulation, and that the corresponding zone of CERK6 when associated with the CERK6 kinase failed to induce symbiotic signaling (see receptors 3-10 of FIGS. 2D-2E). The intracellular part of Chimeras expressed in E. coli that contained the combination of zone 4 and kinase of CERK6 were found to be stable in in vitro stability assays, which suggests that these chimeric receptors could be expressed and fold correctly. This can be seen from the nano-DSF curves for Chimera A (FIG. 15C) and Chimera D (FIG. 15D).

It was hypothesized that these results could be explained if i) zone 4 of CERK6 has an inhibitory role for root nodule symbiosis (RNS), or ii) CERK6 zone 4 and kinase, together, represent a signaling module that is not favorable for symbiosis signaling.

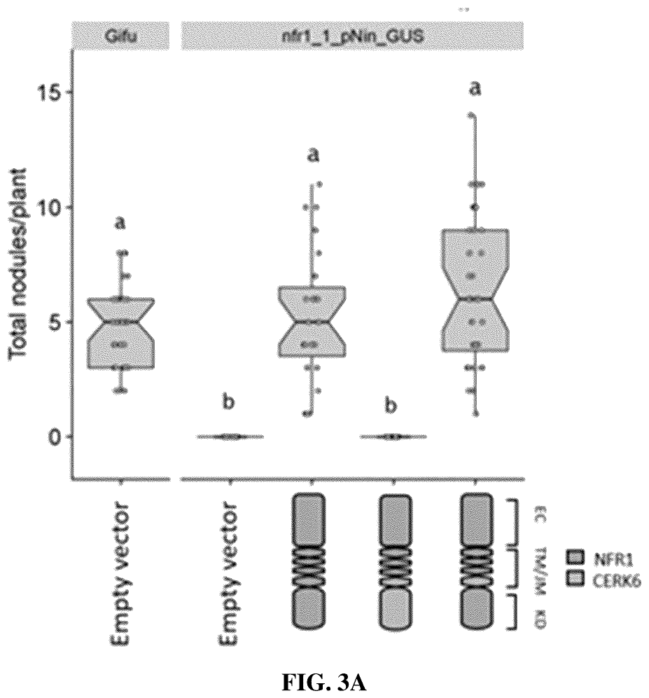

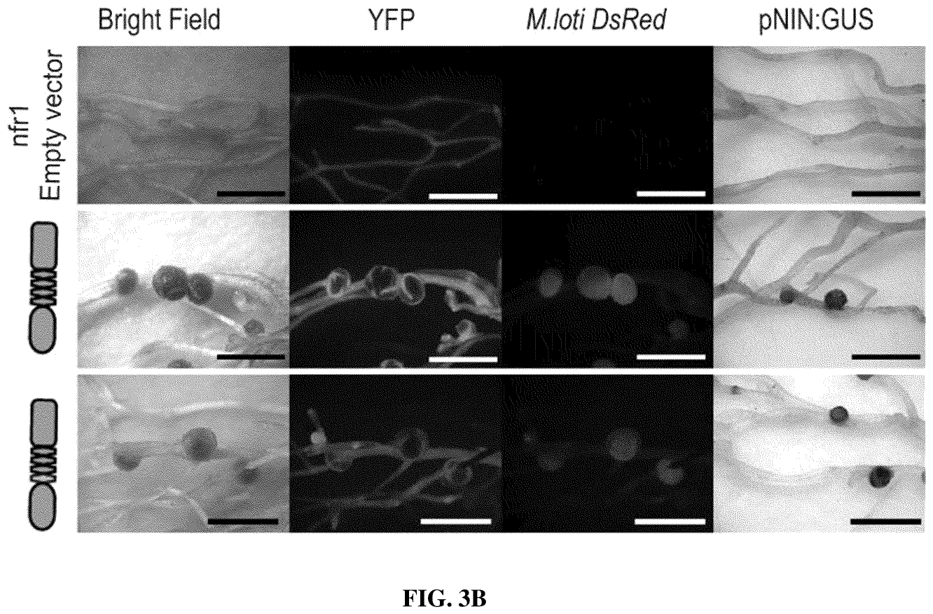

To investigate whether the CERK6 JM zone 4 alone had an inhibitory role in root nodule symbiosis, a NFR1 receptor variant chimera was constructed as above, but in which zone 4 was replaced by the corresponding region of CERK6 and tested for the ability to initiate nodule organogenesis in nfr1-1 pNIN:GUS mutant line. All tested plants formed functional and infected nodules similar to the control full-length NFR1 construct (FIG. 3A, FIG. 3B). This suggests that the CERK6 zone 4 alone was not inhibitory when embedded into the NFR1 receptor but was inhibitory for nodulation when followed by the kinase of CERK6, as it was present in all CERK-type receptors.

### Example 2: Chimeric Receptors Containing the Zone 4 and Kinase from LjCERK6 Localized at the Plasma Membrane of Tobacco Leaf Cells

The following example describes experiments demonstrating that chimeric receptors containing LjCERK6 zone 4 and kinase localized at the plasma membrane of tobacco leaf cells, indicating that the combination of CERK6 zone 4 and kinase impaired the receptor's functionality but not its expression and/or localization.

### Materials and Methods

### Cloning of LjCERK6 Zone 4 Chimeras

A pICH binary vector backbone (Weber et al. 2011) was used for N. benthamiana localization assays, containing a plasma membrane localized AtPIP2A-mCherry driven by pUbi (pUbi::PMmCherry) serving as a localization control. The chimeric constructs were cloned downstream of the β35s and were fused with a module encoding eYFP and terminated by T35s. The modules utilized to assembly the receptor chimeras were the following: LjCERK6 and LjNFR1 (genomic sequences containing introns): Ectodomain (EC), Transmembrane/Juxtamembrane domain (TM/JM), TM/JM zones 1-4 (TM/JM zone 1 is part of the TM (NFR1 zone 1=SEQ ID NO: 3; CERK6 zone 1=SEQ ID NO: 10), while zones 2-4 are part of the JM (NFR1 zone 2=SEQ ID NO: 4; NFR1 zone 3=SEQ ID NO: 5; NFR1 zone 4=SEQ ID NO: 6; CERK6 zone 2=SEQ ID NO: 11; CERK6 zone 3=SEQ ID NO: 12; CERK6 zone 4=SEQ ID NO: 13), and Kinase domain.

### Bacteria and Culture Conditions

E. coli TOP10 (ThermoFisher Scientific) cells were used for molecular cloning and grown in LB medium at 37° C. Agrobacterium tumefaciens line AGL1 was used for tobacco infiltration and was grown in histidine-enriched LB or/and LA at 28 degrees C.

Plant materials and Nicotiana benthamiana infiltration [0180] 3 week-old Nicotiana benthamiana plants were used for infiltration. A. tumefaciens AGL1 cells carrying the respective construct was cultured in LB medium overnight at 28 degrees C. at 180 rpm. The inoculi were centrifuged and the pellet resuspended in MQ sterile water to an OD600=0.6-0.8. The bacteria suspension was used to infiltrate 3-4 week-old N. benthamiana plants with a 1 mL syringe

### Co-Localization

At 2 days post infiltration, tobacco leaf discs were depicted utilizing Zeiss LSM780 confocal microscope with the following excitation/emission parameters: a) YFP: 514/520-560 nm, b) mCherry 561/570-600 nm and c) bright field. The photos were edited using Fiji (ImageJ).

### Results

The lack of complementation of the nfr1 mutant by chimeric receptors containing the CERK6 zone 4 and CERK6 kinase (see Example 1, FIGS. 2B-2C, FIGS. 2F-2G, and FIGS. 3A-3B) posed the question of whether the CERK6 JM zone 4 (CERK6 JM zone 4=SEQ ID NO: 13) and/or CERK6 kinase had a negative impact on protein folding, stability, and/or localization to the plasma membrane.

In order to investigate if nonfunctional receptor chimeras properly folded and localized at the plasma membrane, a heterologous overexpression system was used to co-express mCherry-tagged AtPIP2A, which localizes to plasma membranes, with YFP-tagged chimeric receptors in tobacco (Nicotiana benthamiana) leaves.

NFR1 and CERK6 receptor proteins are normally found in low abundance in L. japonicus roots, and tagged receptors expressed from native NFR1 and CERK6 promoters are not detectable by fluorescence-based microscopy. Because overexpression of LjCERK6 kinase has been shown to induce cell death (Bozsoki et al. (2020) Science 369(6504), 663-670), a kinase-dead mutant of CERK6 kinase (K35IN) was used in the chimeras in this assay in place of the wild-type CERK6 kinase domain described in Example 1. Co-expression and subsequent co-localization microscopy of YFP-tagged chimeras with mCherry-tagged AtPIP2A experiments were performed to determine if the YFP-tagged chimeras co-localized with mCherry-tagged AtPIP2A at the plasma membrane.

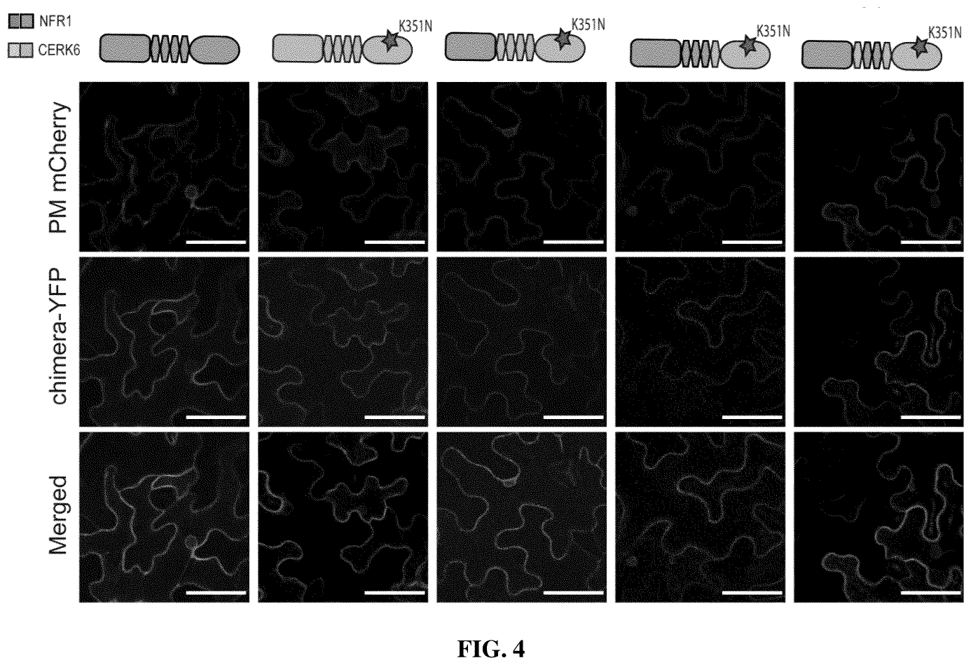

Five YFP-tagged receptors were expressed separately in the co-expression system described above: The full-length native LjNFR1 tagged with YFP (top row in FIG. 4); the full-length inactive mutant LjCERK6(K35IN) tagged with YFP (second row in FIG. 4); and three chimeric receptor proteins, each with the LjNFR1 ectodomain (EC) and the LjCERK6(K35IN) kinase domain (K) and various combinations of NFR1 and CERK6 TM/JM domain zones (bottom three rows in FIG. 4). Protein expression and localization to the plasma membrane was observed for all analyzed constructs (FIG. 4). This indicated that the analyzed chimeric receptor proteins were produced and folded and localized to the plasma membrane, meaning that the combination of the zone 4 and kinase domains from CERK6 did not impair the expression or localization of the receptor. Together with the results from Example 1, these results indicated that the combination of the zone 4 and kinase domains from CERK6 impaired the receptor's functionality, but not its expression or localization.

### Example 3: Residues in LjNFR1 JM Zone 4 are Critical for Symbiotic Signaling

The following example describes in-depth phylogenomic and functional analyses demonstrating that residues in LjNFR1 JM zone 4 were critical for symbiotic signaling. In order to dissect if the entire zone 4 of NFR1 is important, or if individual amino acids are sufficient or critical for root nodule symbiosis, alignments of NFR1 and CERK6 homologs were analyzed, and chimeric constructs containing amino acid swaps were constructed and analyzed.

### Materials and Methods

### Cloning of LjNFR1 Zone 4 Chimeras

A level 2 pIV10 expression vector 9 (Marquez, A. J., and J. Stougaard, eds. 2005. Lotus Japonicus Handbook. Dordrecht: Springer) was used for Hairy Root transformation, containing an expression cassette of nuclear localized tripleYFP driven by the constitutive promoter pUbi (pUbi::tYFP-NLS) serving as transformation marker. The chimeric constructs or chimeric variants were positioned downstream of pNFR1. Golden gate cloning took place utilizing T4 ligase and BpiI or BsaI (ThermoFisherScientific) depending on the level of the cloning modules (Weber et al. 2011). The modules utilized to assembly the receptor chimeras were the following: LjCERK6 and LjNFR1 (genomic sequences containing introns): Ectodomain (EC), Transmembrane/Juxtamembrane domain (TM/JM), TM/JM zones 1-4 (TM/JM zone 1 is part of the TM (NFR1 zone 1=SEQ ID NO: 3; CERK6 zone 1=SEQ ID NO: 10), while zones 2-4 are part of the JM (NFR1 zone 2=SEQ ID NO: 4; NFR1 zone 3=SEQ ID NO: 5; NFR1 zone 4=SEQ ID NO: 6; CERK6 zone 2=SEQ ID NO: 11; CERK6 zone 3=SEQ ID NO: 12; CERK6 zone 4=SEQ ID NO: 13)), Kinase domain and mutagenized zone 4.

### GUS Staining and Visualization

Plant hairy roots were incubated in the GUS buffer consisting of: NaPO4 (pH 7.0) 100 mM, EDTA (pH 8.0) 10 mM, K Ferricyanide 1 mM, K Ferrocyanide 1 mM, Triton X-100 0.1%, X-Gluc (5-bromo-4-chloro-3-indolyl-beta-D-glucuronic acid, cyclohexylammonium salt) 0.5 mg/ml at 37 degree C. overnight in the dark. The roots were washed with 70% EtOH and kept at 4 degrees C. for several days after inspection.

### Sequence Alignments and Phylogenetic Analyses

Alignment and phylogenetic trees were created with CLC Main Workbench, Qiagen. Phylogenetic trees: Tree construction method: Neighbor joining, Protein distance measure: Jukes-Cantor Bootstrap analysis: 100 replicates.

### Other Methods

Bacteria and culture conditions, plant materials, N. benthamiana infiltration, and other methods were conducted as described in Example 1 unless stated otherwise.

### Results

Alignments of NFR1 and CERK6 homologs in 17 nodulating and 2 non-nodulating species were analyzed to identify conserved residues (FIGS. 23A-23L). Six amino acids that were conserved in NFR-type receptors (corresponding to residues M306, A308, M311, Q316, K320, and N323 in the NFR1 protein sequence) but variable in CERK types in zone 4 were identified to separate the NFR-type from the CERK-type receptors, with the only exceptions being the Parasponia LYK3.1 and LYK3.2, which were clustered with the CERK types (FIGS. 5A-5C). This separation is illustrated in a phylogenomic tree in FIG. 5C where the zone 4 of NFR-type receptors (in magenta) is separated from the zone 4 in CERK types (in green).

These differences in amino acids have consequences on the polarity and charge of this particular region (FIG. 6B). Only six residues differ in L. japonicus between NFR1 and CERK6 (FIGS. 6A, 6C). Two residues, M306 and A308, were highly conserved (FIGS. 6A-6C) and were only present in NFR receptors. These were observed to create a hydrophobic non-polar region (IMVAK motif) that is not observed in CERK types due to the presence of polar and negatively charged T304 and D306 in the corresponding positions. In general, the entire NFR1 zone 4 appears to be highly conserved across Fabales nodulating species while the CERK is more variable but always keeps its negatively charged amino acid signature. In Lotus, this characteristic non-polar versus charged state of NFR1 and CERK6 zone 4, respectively, is present and shown in the alignment in FIG. 6B. When observing the NFR1 model structure and CERK6 crystal structure, it was seen that zone 4 was located in the N-terminus and on top of the kinase domain (FIG. 6D). The first three variable residues of NFR1/CERK6; M306/T304, A308/D306, and M311/V309, were located in a loop, while the last three, Q316/D314, K320/T318, and N323/D321 were located in the αB-helix (FIG. 6E). The position and orientation of these two triplets created two possibly distinct protein-protein interaction surface candidates.

Additional studies further dissected the role of individual amino acids of NFR1 zone 4 in symbiosis. A construct containing the EC and TM/JM of NFR1 coupled to the kinase of CERK6 (FIG. 7A) was used as a basis for individual amino acid swaps between NFR1 to CERK6. Six amino acid swap constructs were constructed based on the variable residues in LjCERK6 compared to LjNFR1 as shown in FIG. 6E.

Nodule formation measurements indicated that nfr1-1 pNin:GUS plants expressing the chimeric constructs carrying the M311V, Q316D, K320T, or N323D amino acid swaps were able to complement the nfr1-1 mutant similarly to the NFR1 full-length control (as indicated by the lack of a significant reduction in the number of total nodules in these amino acid swap constructs compared to the NFR1 full-length control), whereas constructs carrying the M306T or A308D amino acid swaps were unable to complement the nfr1-1 mutant (as indicated by a significant reduction in the number of total nodules compared to the NFR1 full-length control) (FIG. 7B). This indicated that the M311, Q316, K320, and N323D residues (which were conserved in NFR1 receptors) were not critical for nodule formation. On the other hand, a significant reduction in the number of total nodules was observed for the constructs that contained M306T, and A308D variations (FIG. 7B), implying that the M306 and A308 residues of NFR1 were important for symbiosis. A more detailed investigation of the nodule functionality revealed that only 9 out of 40 plants expressing the A308D amino acid swap construct were able to form pink nodules, while 15 out of the 40 tested plants formed only white and uninfected primordia (FIGS. 7B-7C). The pink color observed in mature nitrogen-fixing nodules was due to the presence of leghemoglobins, which result in the accumulation of iron-containing pigment (Appleby (1984) Annual Review of Plant Physiology 35(1):443-78; Ott et al. (2005) Current Biology 15(6):531-35). Consequently, pink nodules are considered to be infected and functional, nitrogen-fixing nodules. The combination of the small size of the nodules (primordia) and their lack of pink color suggests that the A308D amino acid swap construct impaired the coordination of infection thread elongation into the nodule that would induce nodule maturation and release of the rhizobia into the nodule cells. This indicated that M306 and A308 played a critical role in nodule organogenesis and that A308 contributed to the signaling pathway that leads to nodule infection.

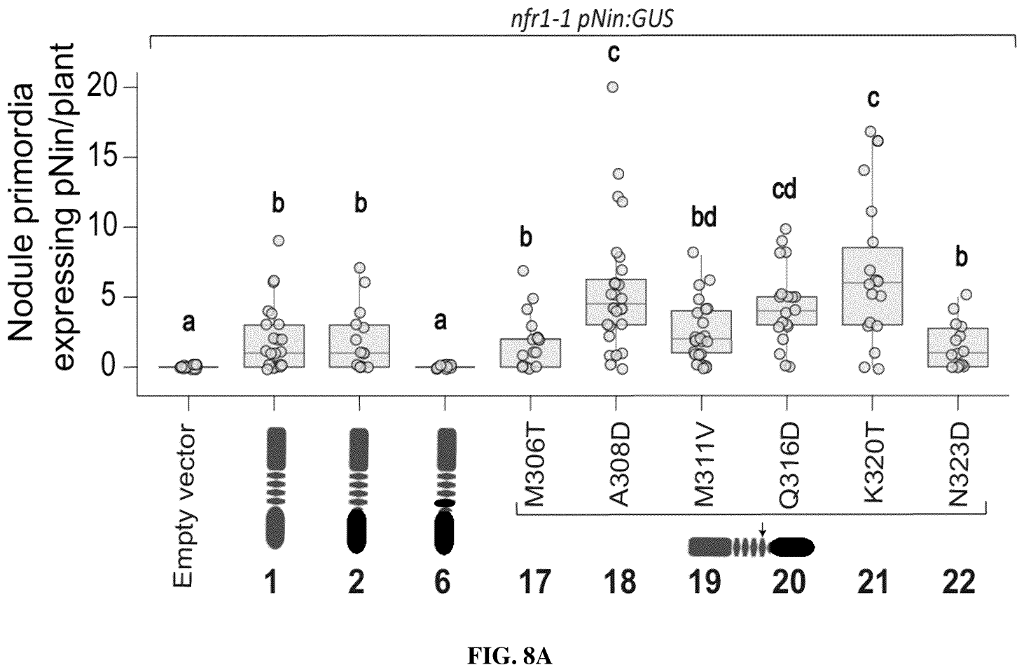

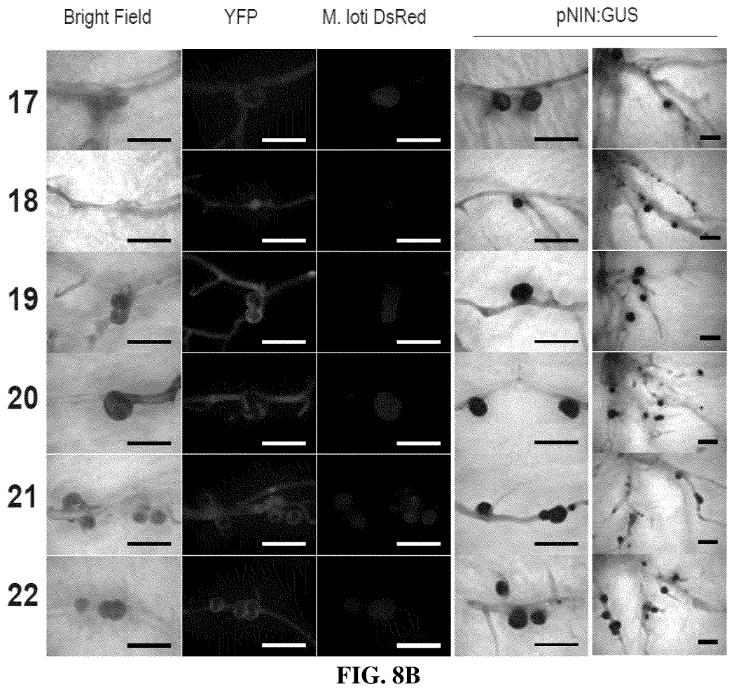

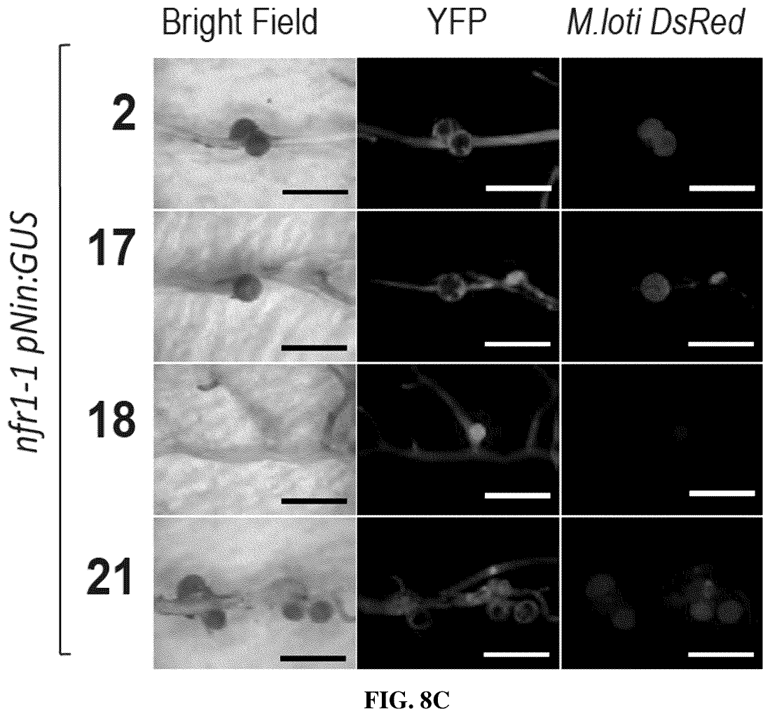

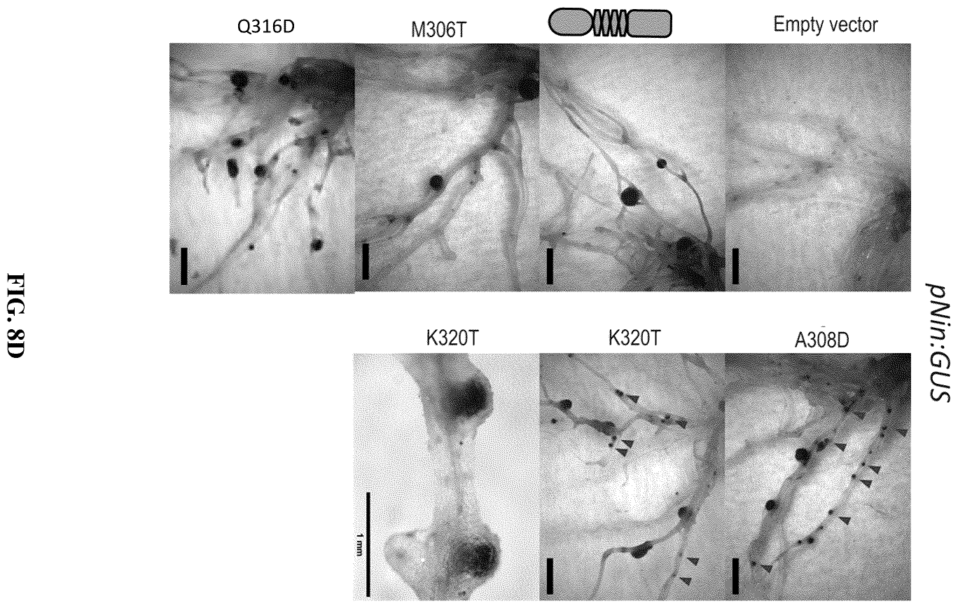

A further investigation following GUS staining of the roots for visualization of NIN promoter expression revealed that roots expressing the A308D or K320T amino acid swaps induced a larger number of nodule primordia when compared to the rest of the analyzed constructs, as statistical analysis showed significant difference in the number of nodule primordia (see lowercase letter c in FIG. 8A). Microscopic imaging revealed that these primordia were very small and did not emerge out of the main root (FIG. 7C). Counting the Nin-expressing primordia revealed a significant increase in the number of symbiotic events initiated but arrested before primordia emergence from the root in the roots expressing receptors 18- A308D, 20-Q316D and 21-K320T (FIGS. 8A, 8B, and 8D). This excess of primordia expressing Nin suggests an increase in the number of symbiotic events initiated from the receptors, while their arrest before emergence from the root could be a result of a reduced rhizobial infection as well as the initiation of the root- and shoot-control of symbiosis operating in legume roots. Because the small primordia did not emerge out of the main root, it was not feasible to identify and quantify the primordia by eye on unstained roots expressing the A308D or K320T amino acid swaps. Nonetheless, the expression of pNIN:GUS illustrated that these were genuine symbiotic organogenetic events induced by the receptors with the A308D or K320T amino acid swaps (FIG. 8D). These results indicated that the A308 and K320 residues played an important role in the infection or signaling pathway(s) required for coordinating organogenesis and infection. These results also indicate that residues present in the JM-B of NFR1 coordinate the downstream signalling events that lead to nodule organogenesis and rhizobial infection in concert.

### Example 4: Residue Motifs in LJNFR1 JM Zone 4 are Critical for Symbiotic Signaling

The following example describes that residue motifs in LJNFR1 JM zone 4 are critical for symbiotic signaling. Example 3 describes that individual residues in NFR1 JM zone 4 have an impact on organogenesis and nodule infection (see, e.g., FIGS. 7B-7C, FIGS. 8A-8D). To identify which amino acid motif(s) is or are crucial for root nodule symbiosis, three receptor variants were constructed and tested for their capacity to restore nodulation in the nfr1-1 pNIN:GUS mutant.

### Materials and Methods

Cloning of LjNFR1 zone 4 chimeras, bacteria and culture conditions, plant materials, Hairy Root transformation, nodulation assays, and other methods were conducted as described in Example 1 unless stated otherwise.

### Results

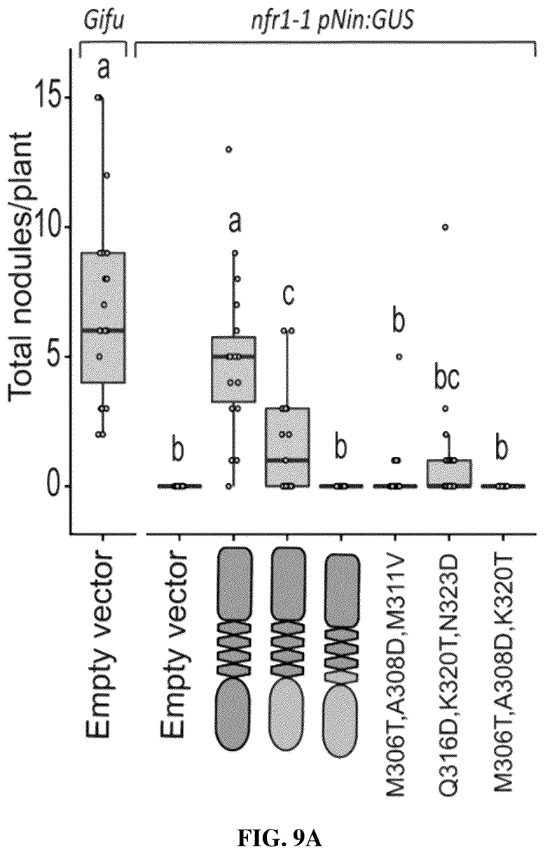

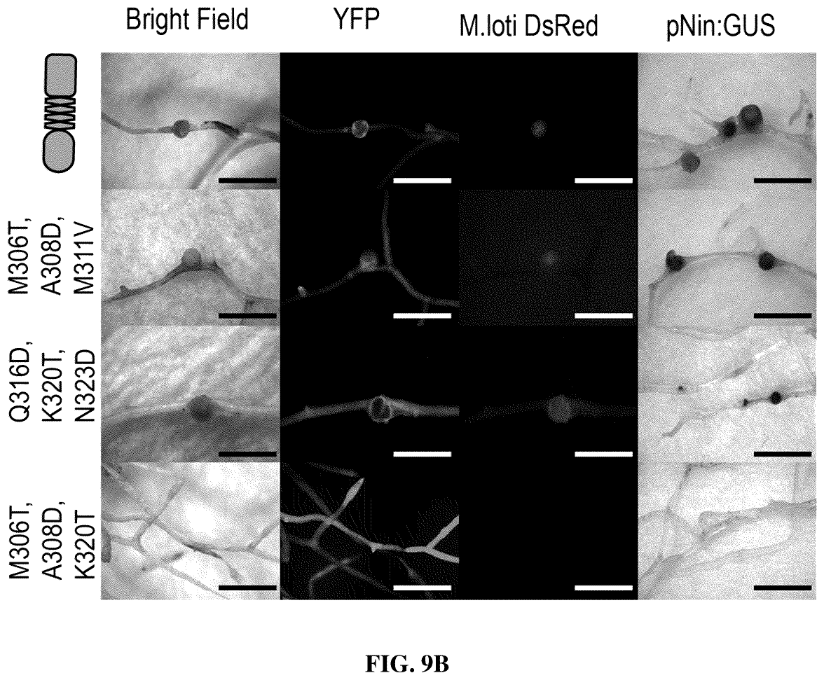

The construct with the EC and TM/JM of NFR1 coupled with the kinase of CERK6 (Chimera A) was used as a basis for the construction of the receptor variants. For each variant, three-residue combinations were changed from the residues found in NFR1 to those found in CERK6 (see FIG. 7A, FIG. 9A, and FIG. 9B): one variant contained the mutations M306T, A308D, and M31iV; a second variant contained the mutations Q316D, K320T, and N323D; and a third variant contained the mutations M306T, A308D, and K320T.

Chimera A containing the substitutions Q316D, K320T and N323D in the αB-helix of zone 4 had very low efficiency in formation of nodules compared with the full-length NFR1, with only 4 out of 38 characterized plants that contained the substitutions Q316D, K320T and N323D found able to produce nodules (at a maximum of about 5 nodules per plant) (FIG. 9A), all of which were small in size (see FIG. 9B). The chimera containing the M306T, A308D and M31iV substitutions in the zone 4 loop also had a statistically significant reduction in nodule formation, when compared with the full-length NFR1, as only 8 out of 31 characterized plants that contained the M306T, A308D and M311V substitutions were able to engage in symbiosis (FIG. 9A). The remaining chimera, with the substituted residues M306T, A308D and K320T, was not able to induce organogenesis or NIN activation on the transformed roots, indicating that the combination of these three residues was crucial for root nodule symbiosis (FIG. 9A and FIG. 9B).

Next, the role of individual amino acids of NFR1 zone 4 in symbiosis was dissected. Chimera A (containing the EC and TM/JM from NFR1 and the K domain from CERK6) was used this time to individually swap the six residues from NFR1 to CERK6 (see schematic representation in FIG. 7A). It was found that nfr1-1 pNIN:GUS plants expressing the chimeric constructs that carried the M311V, Q316D, K320T, or N323D changes were able to complement the mutant in a similar way to the NFR1 full-length control (FIG. 7B). This indicated that these residues, which were conserved in NFR receptors, were not critical for symbiosis. On the other hand, a significant reduction in the number of total nodules was observed for the constructs that contained the M306T or A308D substitutions (FIG. 7B). A more detailed investigation of the nodule functionality revealed that only 4 out of 40 analyzed plants that expressed the construct with the A308D substitution were able to form pink, functional nodules, while 15 out of 40 plants formed only white and uninfected primordia (FIG. 7B-7D). This indicated that M306 and A308 play critical roles in nodule organogenesis and that A308 contributed to the signaling pathway leading to nodule infection.

### Example 5: Residues in NFR1 JM Zone 4 are Sufficient to Induce Root Hair Infection and Nodule Primordia Formation

The following example describes gain-of-function experiments indicating that particular residues in LJNFR1 JM zone 4 are sufficient to induce symbiotic signaling. Example 3 showed that substitution of the JM M306 or A308 residues from the residues found in NFR1 to those found in CERK6 impairs organogenesis and root hair infection (see FIGS. 7A-7D). In order to evaluate whether these residues are individually sufficient to initiate the root nodule symbiotic pathway, a gain of function study was performed.

### Materials and Methods

Cloning of LjNFR1 zone 4 chimeras, bacteria and culture conditions, plant materials, Hairy Root transformation, nodulation assays and other methods were conducted as described in Example 1 unless stated otherwise.

### Results

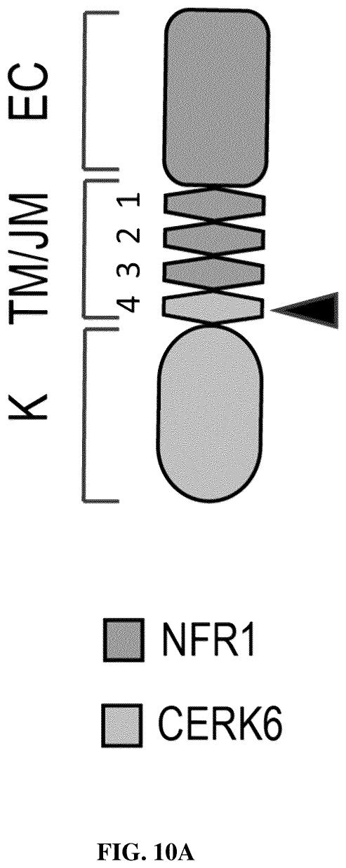

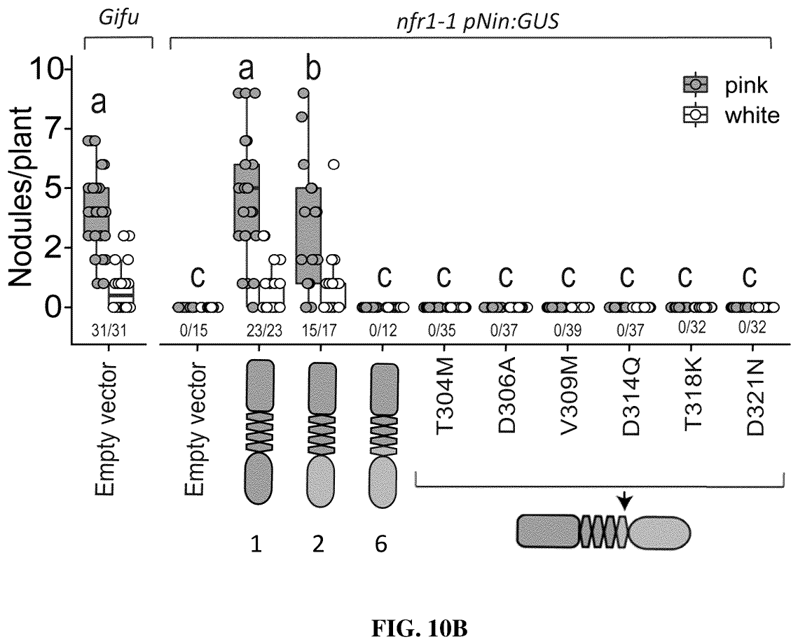

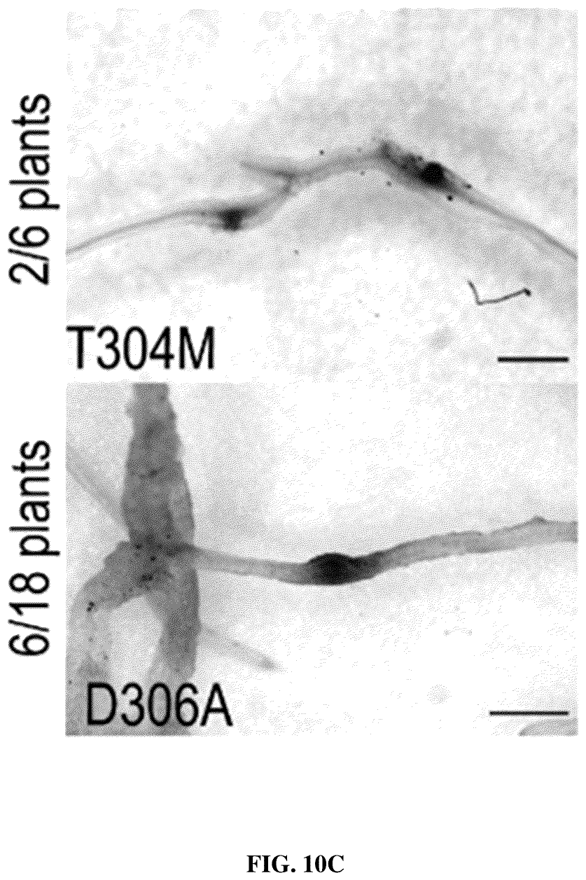

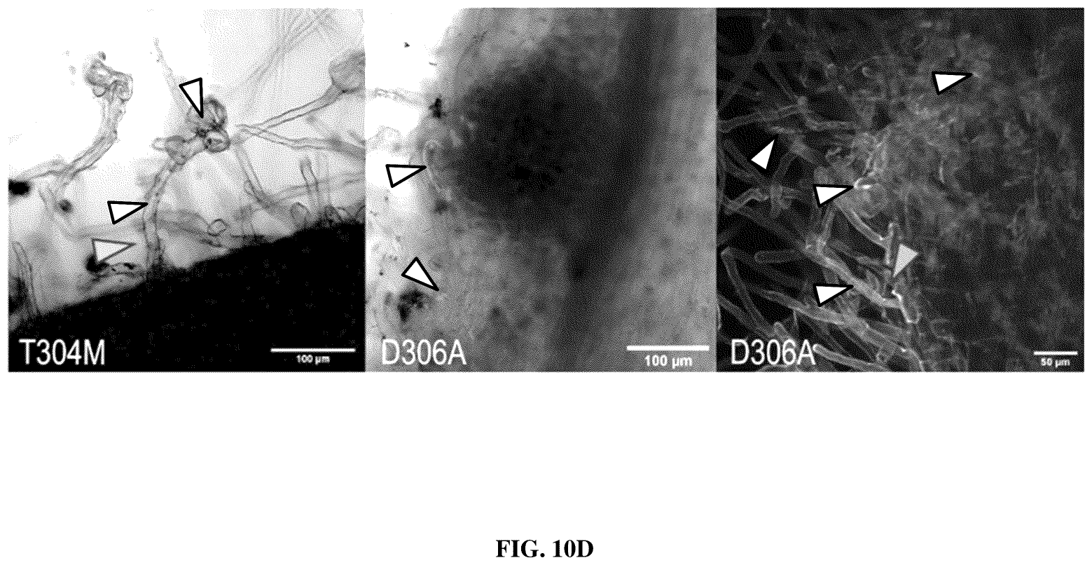

Receptor variants were designed as shown in FIG. 10A. In a background of a receptor chimera that contained the EC and TM/JM zones 1-3 from NFR1 and the K and JM zone 4 from CERK6, individual amino acids present in zone 4 were exchanged with the corresponding amino acids from the zone 4 sequence of NFR1. These receptor versions were tested for their capacity to initiate nodule organogenesis and nodule infection in nfr1-1 pNIN:GUS roots.

None of the individual substitutions tested (T340M, D360A, V309M, T318K, and D321N) was sufficient to restore root nodule symbiosis in the nfr1-1 mutant background (FIG. 10B). However, detailed analysis of the transgenic roots after GUS staining revealed that NIN promoter activation and infection thread formation was present in the transgenic roots expressing either the T304M or D306A receptor variants (FIG. 10C). In total, 2 out of 6 analyzed plants carrying the T304M, and 6 out of 18 analyzed plants carrying the D306A substitutions, had the capacity to induce NIN expression, primordia formation, and infection thread formation (FIG. 10C, FIG. 10D). Only the infection threads that were on the primordia surface were elongated into the cortical cell layers; the rest were aborted in the epidermis (FIG. 10D). This indicated that residues T304M and D306A in LjNFR1 JM zone 4 were each sufficient to induce at least symbiotic signaling (FIG. 10D).

### Example 6: A Minimal Motif of Two Residues in NFR1 Zone 4 is Sufficient to Induce Functional Nodule Organogenesis

The following example describes experiments demonstrating that particular residue motifs in LJNFR1 JM zone 4 were sufficient to induce functional nodule organogenesis. In order to determine the minimal JM zone 4 motif that would allow NFR1 to engage both organogenesis and root infection, the functionality of additional receptor variants was tested.

### Materials and Methods

Cloning of LjNFR1 zone 4 chimeras, bacteria and culture conditions, plant materials, Hairy Root transformation, nodulation assays and other methods were conducted as described in Example 3 unless stated otherwise.

### Results

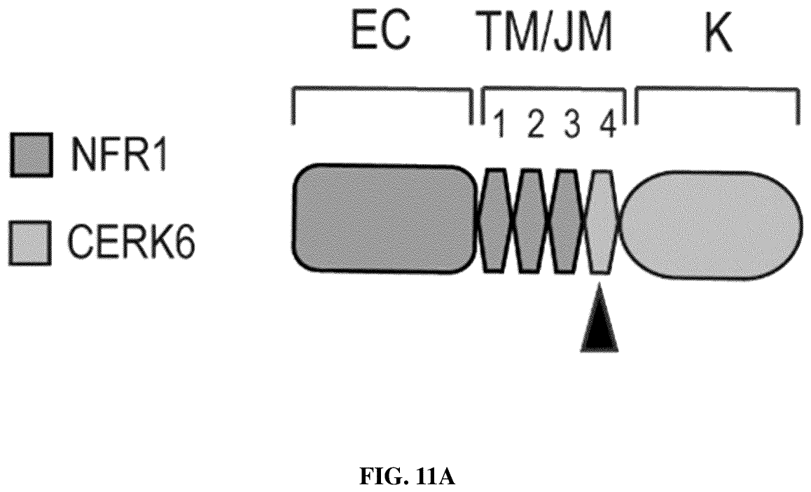

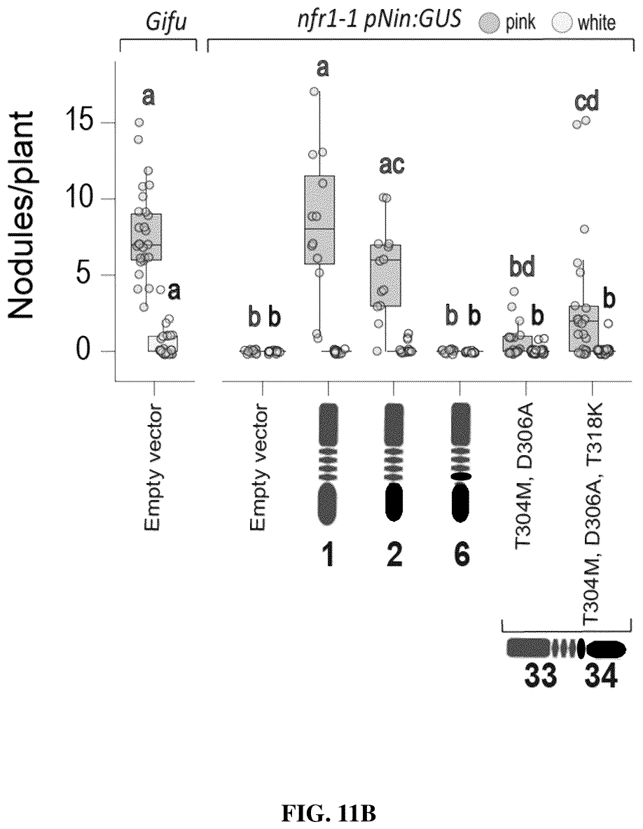

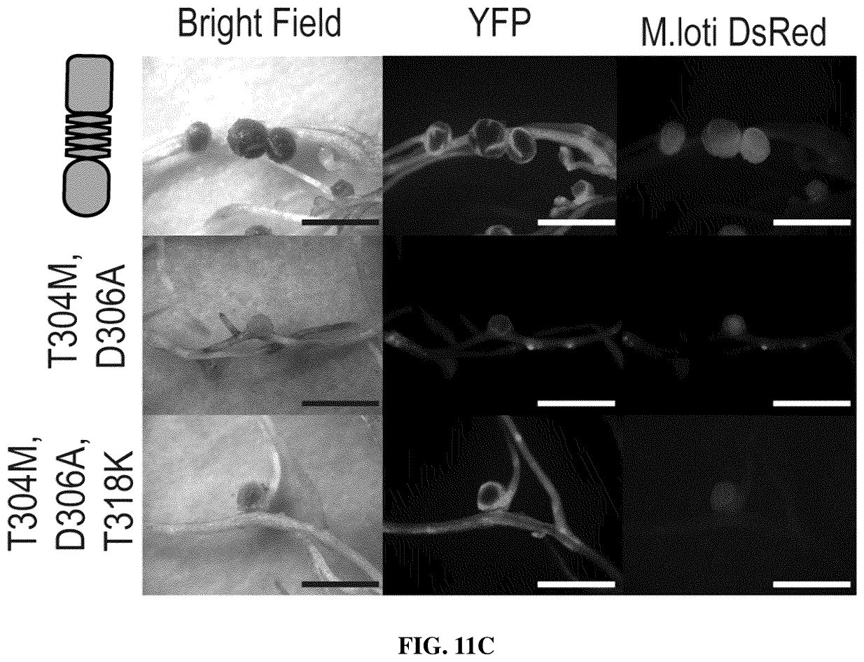

To determine the minimal zone 4 motif that allows NFR1 to engage both in root hair infection and nodule organogenesis, the functionality of receptor variants in which combinations of CERK6 zone 4 residues were substituted by the corresponding NFR1 were tested. The chimera containing the EX and TM/JM zones 1-3 from NFR1 and JM zone 4 and the K domain from CERK6 (hereinafter the “CC”) chimera was used as a basis to construct the receptor variants (see schematic representation in FIG. 11A) that were later tested for their capacity to initiate nodule organogenesis and nodule infection in nfr1-1 pNIN:GUS roots.

Chimera carrying both the T304M and D306A substitutions had the ability to induce nodule organogenesis and nodule infection in 9 out of 23 tested plants, whereas chimera carrying the T304M, D306A, and T318K substitutions showed an improved complementation capacity, with 15 out of 23 tested plants nodulating (FIG. 11B). The efficiency of complementation for these chimeras was lower compared to the full length NFR1 or the chimeras that contain the combination of NFR1 zone 4 and CERK6 kinase, but higher than the chimeras that contain the CERK6 zone 4, indicating that residues M306, A308, and K320 contribute to optimal signaling (FIG. 11B). Finally, the receptor chimera with the T304M and D306A substitutions, as well as the receptor chimera with the T304M, D306A, and T318K substitutions were both able to rescue organogenesis and the infection phenotype in nfr1-1 pNIN:GUS transgenic roots (FIG. 11C), demonstrating that the three residues of JM-B are main signatures driving symbiosis. In summary, the initiation of organogenesis and infection was dependent on the presence of M306 and A308, while K320 contributes to more efficient signaling, since the combination of M306 and A308 alone was sufficient to initiate functional nodule organogenesis, while their combination with K308 produced both greater nodulation frequency and nodulation capacity.

### Example 7: Nodulation Signature Enables Intracellular RLK4 to Induce Root Nodule Symbiosis Signaling in Lotus japonicus Roots

The following example describes experiments demonstrating that a nodulation signature enables intracellular RLK4 to induce root nodule symbiosis signaling in Lotus japonicus roots. Materials and Methods Cloning of LjNFR1 zone 4 chimeras

A level 2 pIV10 expression vector (Mirquez, A. J., and J. Stougaard, eds. 2005. Lotus Japonicus Handbook. Dordrecht: Springer) was used for Hairy Root transformation, containing an expression cassette of nuclear localized tripleYFP driven by the constitutive promoter pUbi (pUbi::tYFP-NLS) serving as transformation marker. The chimeric constructs or chimeric variants were positioned downstream of pNFR1. Golden gate cloning (Weber et al. (2011) PLoS ONE 6(2):e16765) was used for creating the different receptor versions. The modules utilized to assembly the receptor chimeras were the following: HvRLK4 and LjNFR1 (coding sequence (CDS) for RLK4, and genomic sequences containing introns for NFR1): Ectodomain (EC), Transmembrane/Juxtamembrane domain (TM/JM), TM/JM zones 1-4 (TM/JM zone 1 is part of the TM (NFR1 zone 1=SEQ ID NO: 3; HvRLK4 zone 1=SEQ ID NO: 57), while zones 2-4 are part of the JM (NFR1 zone 2=SEQ ID NO: 4; NFR1 zone 3=SEQ ID NO: 5; NFR1 zone 4=SEQ ID NO: 6; HvRLK4 zone 2=SEQ ID NO: 58; HvRLK4 zone 3=SEQ ID NO: 59; HvRLK4 zone 4=SEQ ID NO: 60)), and Kinase domain.

### Sequence Alignments

Alignments were created with CLC Main Workbench, Qiagen. P Protein distance measure: Jukes-Cantor.

### ROS Measurements

Lotus japonicus Gifu and cerk6 mutant plants were transformed with Agrobacterium rhizogenes containing an empty vector (“IYFP_EV”) or chimeric CERK6-NFR1 or RLK4-NFR1 receptor constructs. Plants with hairy roots were grown in magenta containers containing Leca substrate and B&D medium with KNO3 3 mM for 21 days. Only the transformed hairy roots were kept (detected by fluorescence under UV light since the transgenic roots have tripleYFP incorporated into their genome) and cut into 1-2 cm pieces. Equal amounts of the cut roots were collected into white 96-well flat-bottomed polystyrene plates (Greiner Bio-One) with the goal of having one hairy root in one well. They were kept for overnight incubation in sterile MQ water. The next day, for the ROS measurement, the water in every well was replaced by reaction Master Mix consisting of: 0.5 mM L-023 (Wako Chemicals), 5 μg/mL horseradish peroxidase (Sigma), and 0.1 mg/ml chitin. Luminescence in the 30-40 min assays was recorded with a Varioskan Flash Multimode Reader (Thermo Scientific) as Relative Luminescence Units (RLU). In one repetition, at least three wells were measured for each treatment for every genotype.

### Other Methods

Bacteria and culture conditions, plant materials, Hairy Root transformation, nodulation assays and other methods were conducted as described in Example 1 unless stated otherwise.

### Results

HvRLK4, an RLK LysM receptor polypeptide from barley (Hordeum vulgare) was chosen as an engineering target because its kinase domain shares 70% and 78% identity with LJNFR1 and LjCERK6, respectively (FIG. 12A). However, the full-length RLK4 was not able to initiate symbiosis signaling in the nfr1-1 mutant (FIG. 12B), raising the possibility of engineering RLK4 to function in symbiosis when equipped with symbiotically proficient regions from NFR1. In order to determine whether RLK4 can be engineered to function in symbiosis, the functionality of additional receptor variants was tested.

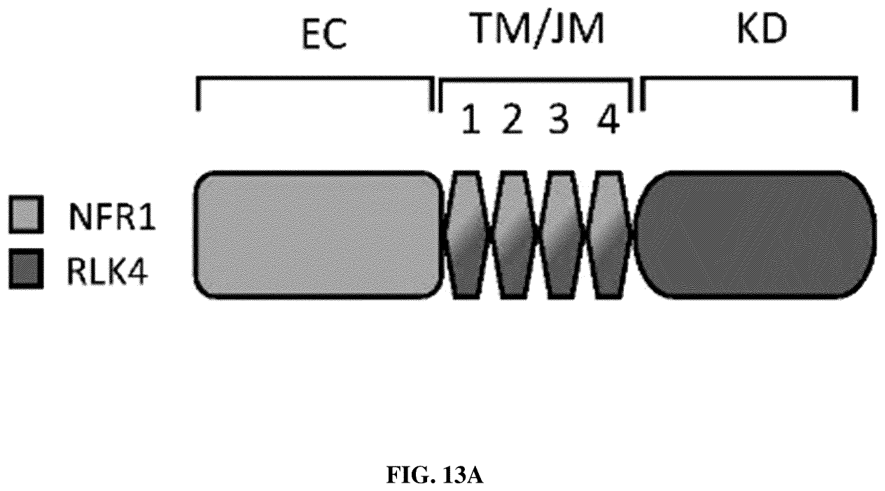

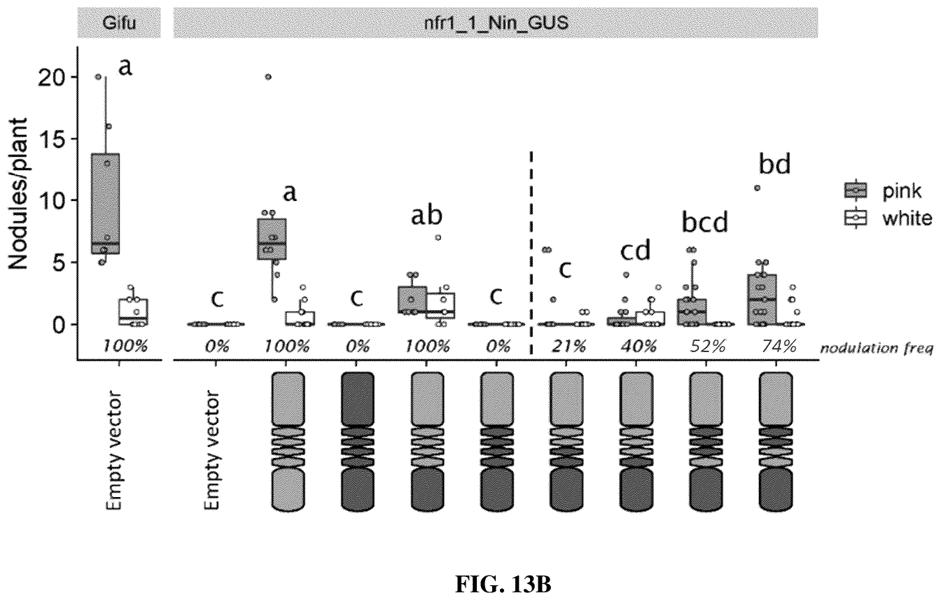

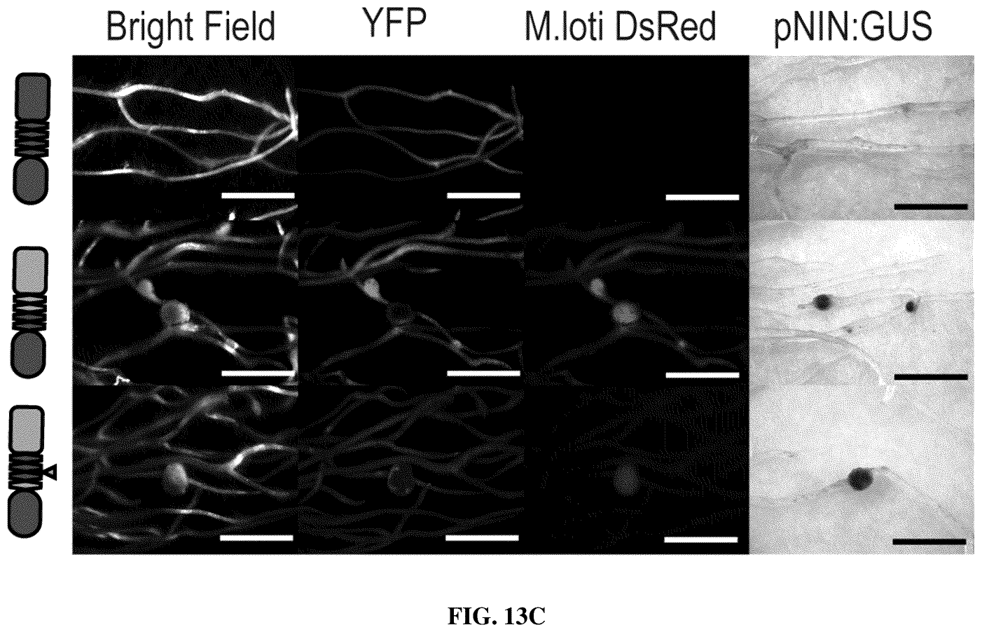

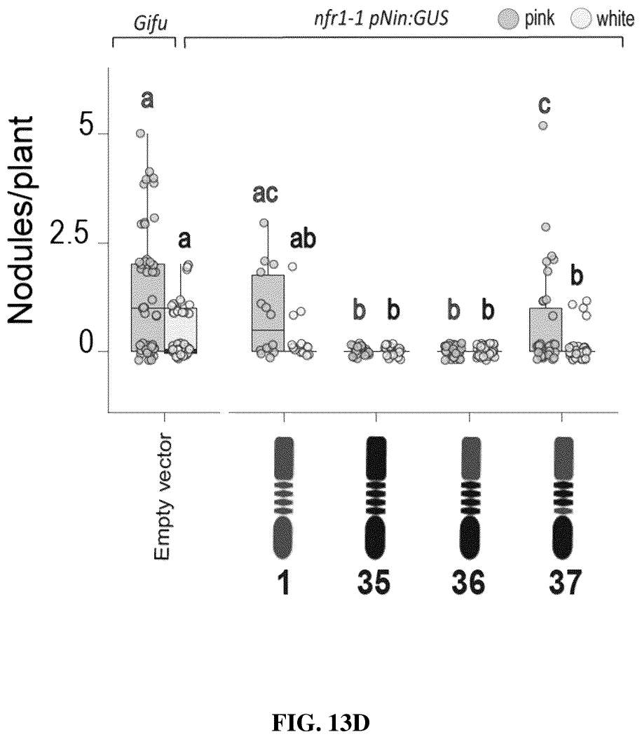

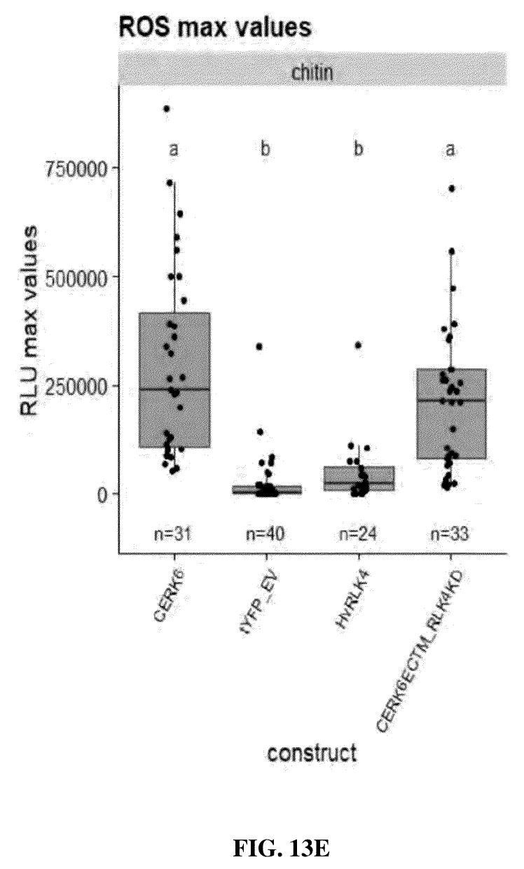

In the variants tested in this Example, domains from NFR1 and RLK4 were combined into chimeric receptors. Receptor variants were designed as shown in FIG. 12B or FIG. 13A. These receptor variants were tested for their ability to induce nodule formation in nfr1-1 pNIN:GUS transgenic roots.

As with the chimeric receptors containing the kinase domain of CERK6 discussed in the Examples above, chimeric receptors containing either the RLK4 KD or TM/JM in combination with the remaining regions of NFR1 were able to restore nodulation in the nfr1-1 background (FIG. 12B). However, this functionality was lost when both the KD and TM/JM of RLK4 were together in a given receptor (FIG. 12B). This indicated that the RLK4 receptor resembled CERK6, and implied that the findings from CERK6-NFR1 chimera discussed in the above Examples were similarly applicable for engineering RLK4 for root nodule symbiosis.

Alignment of the NFR1, CERK6 and RLK4 protein sequences (see FIGS. 5A-5B) revealed that the variant six amino acids identified in zone 4 of CERK proteins compared to zone 4 of NFR1 (see FIG. 6) from species of the Fabales order, such as Lotus japonicus, were partially conserved in RLK4. In more detail, the residues T304, D306, and V309 present in CERK6 zone 4 were conserved in RLK4 and correspond to T308, D310, and V313, respectively, of the RLK4 protein sequence; the residue D314 of CERK6 corresponds to E318 of the RLK4 protein sequence, it is variable between CERK6 and RLK4 but it maintains its negative-charge character; the residue T318 of CERK6 corresponds to N322 of the RLK4 protein sequence; and finally the residue D321 of CERK6 corresponds to E325 of the RLK4 protein sequence, it is variable between CERK6 and RLK4 but it maintains its negative-charge character. Correspondingly, chimera containing the TM/JM and kinase of RLK4 were unable to produce functional symbiosis in terms of nodulation (see FIG. 13B, left side).

The alignment in FIG. 12A further identified that the entire JM (i.e., zones 2-4) of HvRLK4 was shorter (5 residues) than that of LJNFR1, indicating a different impact on signaling might exist.

In order to test the functional impact of combining different regions from the JM of RLK4 with zone 4 of NFR1, four additional NFR1-RLK4 chimeric receptor constructs were created (FIG. 13A). The four constructs each contained the EC from NFR1, the KD from RLK4, and at least one TM/JM zone from NFR1 (see the right-most four schematics at the bottom of FIG. 13B); all four were able to restore nodulation of the nfr1-1 mutant, albeit to different extents (FIG. 13B). The receptor variant that contained both the TM (zone 1) and JM zone 2 of NFR1 and JM zones 3-4 from RLK4 induced nodule formation in 3 out of 14 plants tested (21%; shown immediately to the right of the vertical dashed line in FIG. 13B). The receptor variant that contained zones 1-3 of NFR1 and zone 4 from RLK4 induced nodulation in 6 of 15 tested plants (40%; shown second-to-right from the vertical dashed line in FIG. 13B). The receptor variant that contained zone 4 of NFR1 and zones 1-3 from RLK4 induced nodulation in 11 of 21 tested plants (52%; shown third-to-right from the vertical dashed line in FIG. 12B). In contrast, the receptor variant that contained zones 3-4 of NFR1 and zones 1-2 from RLK4 induced nodulation in 14 of 19 tested plants (74%; shown at the far right in FIG. 13B), representing an increase compared to the previous three constructs. Finally, all tested transformed roots that showed symbiotic signaling activation from this set of four constructs were further found to both express pNIN and to form pink nodules infected with M. loti R7a expressing DsRed (FIG. 13C). Another, independent substitution of JM zone 4 of RLK4 for that of NFR1 also resulted in construct 37 rescuing the nodulation phenotype (FIG. 13D). In comparing performance between RLK4 and CERK6 receptor variants with luminescence-based assays, it was found that a chimeric construct containing CERK6 contribution as the ectodomain+the membrane domain and HvRLK4 contribution as the kinase domain responded to chitin.

Together, these results indicated that zone 4 of NFR1 maintained its symbiotic determinacy when embedded in the intracellular region of RLK4, but that its full properties were dependent and enhanced by the remaining regions of the NFR1 TM/JM.

### Example 8: Crystal Structures of CERK6 and LYK3 Proteins

The following example describes the crystal structures of the kinase domain of CERK6 and LYK3 proteins. Structures for LjCERK6/303-599 D460N and MtLYK3/302-597 D459N with AMP-PNP (phosphoaminophosphonic acid-adenylate ester) have been solved. AMP-PNP is an ATP analogue.

### Materials and Methods

### Expression and Purification of LysM Receptor Kinases and Chimeras of LysM Receptor Kinases

Lotus japonicus CERK6 D460N kinase (residues 303-599) and Medicago truncatula LYK3 D459N (residues 302-597) were codon optimized for expression in E. coli (Genscript) and N-terminally fused with a histidine tag, an arginine tag, a SUMO tag and a 3C protease cleavage site.

For expression, Rosetta 2 (Sigma-Aldrich) competent cells were transformed with the constructs and grown to OD600=0.6 in LB medium supplemented with the relevant antibiotics at 370 C with shaking. Then the culture was chilled on ice for 30 minutes before expression was induced by adding 0.2 mM IPTG and incubating at 190 C with shaking for 20 hours. The cells were harvested at 4400 g for 15 minutes, resuspended in LB medium and pelleted again at 3050 g for 10 minutes. The pellet was resuspended in lysis buffer (50 mM Tris-HCL pH 8.0, 500 mM NaCl, 10% glycerol, 10 mM imidazole, 5 mM BME) and lysed by sonication. Cleared supernatant was subjected to nickel immobilized metal affinity chromatography (Ni-IMAC) on a 1 ml Protino Ni-NTA column (Macherey-Nagel) equilibrated in lysis buffer. The sample was loaded onto the column using a peristaltic pump, then the column was washed with 20 column volumes 20 mM imidazole before it was eluted in 12 ml 200 mM imidazole. 3C protease and,-phosphatase was added to the eluate in an approximately 1:50 molar ratio and it was dialyzed against 1 L of dialysis buffer (50 mM Tris-HCL pH 8.0, 250 mM NaCl, 5% glycerol, 5 mM BME, 1 mM MnC12) overnight at 4° C. The dialysis product was subjected to another Ni-IMAC to remove the protease and the digested tags before the protein was purified in a final gel filtration on a Superdex75 increase 10/300 GL column or a Superdex200 increase 10/300 GL column (GE Healthcare) equilibrated in gel filtration buffer (25 mM Tris-HCL pH 8.0, 150 mM NaCl, 5 mM BME). All purification steps were analyzed by SDS-PAGE and elution fractions pooled accordingly.

### Crystallization

Crystals from LjCERK6/303-599 D460N and MtLYK3/302-597 with AMP-PNP were obtained in a sitting drop vapor diffusion setup. The following crystallization conditions were used for each protein: LjCERK6/303-599 D460N: 0.1 M imidazole pH 8.0, 5% PEG 8000, 10 mg/ml protein; MtLYK3/302-597 D459N with AMP-PNP: 0.2 M sodium formate, 0.1 M bis-tris propane pH 8.5, 20% w/v PEG 3350, 3 mM AMP-PNP.

### Crystallography

Crystals were fished and cryo protected as follows: LjCERK6/303-599 D460N: 25% ethylene glycol in mother liquor; MtLYK3/302-597 D459N with AMP-PNP: 20% glycerol in mother liquor.

The synchrotron data for MtLYK3/302-597 D459N with AMP-PNP were collected at beamline P13 operated by EMBL Hamburg at the PETRA III storage ring (DESY, Hamburg, Germany). The data for LjCERK6/303-599 D460N were collected at beamline BioMAX at the MAX IV storage ring (Lund, Sweden).

### Data Analysis and Modeling

For both structures, the phase problem was solved by molecular replacement using Phaser (McCoy et al. (2007) J Appl Crystallogr 40, 658-674). Refinement was performed in PHENIX (Liebschner et al. (2019) Acta Cryst. D75, 861-877) and manual model building was carried out in Coot (Emsley et al. (2004) Acta Crystallogr. D60, 2126-2132). Figures of the crystal structures were prepared using PyMol Molecular Graphics system ver. 2.3.2 (Schr6dinger, LCC).

### Results

### Preliminary Structures

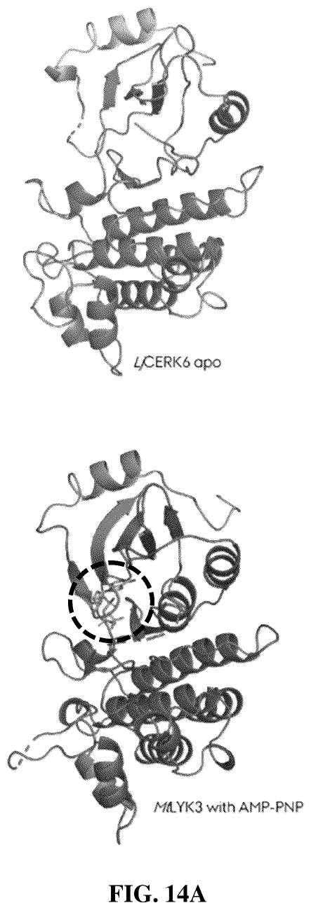

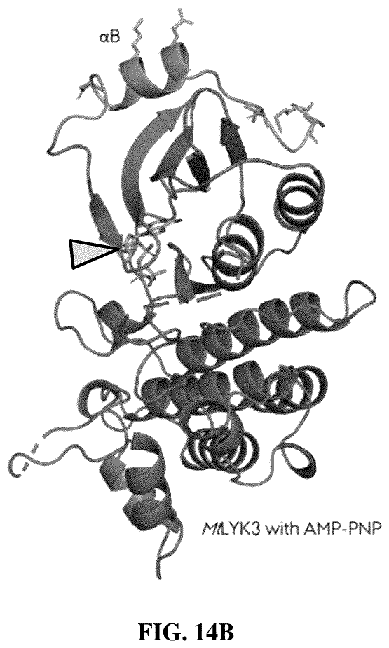

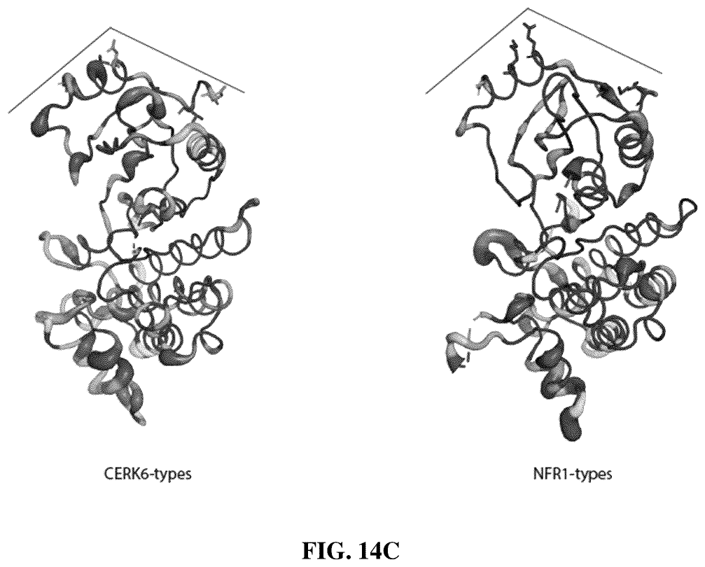

Preliminary structures of CERK6 from Lotus japonicus (LjCERK6) and LYK3 from Medicago truncatula (MtLYK3) with AMP-PNP are shown in FIG. 14A. These structures allowed a comparison of both symbiotic (MtLYK3) and immune (LjCERK6) receptor kinases, from which differences were subsequently identified.

Most of the JM zone 4 was a well-defined alpha helix that was part of the kinase core fold. This region was annotated as aB in FIG. 14B. CERK6-type kinases and NFR1-type kinases differ very little in this zone, with only 6 variant residues between LjNFR1 and LjCERK6 (see FIGS. 6A-6E). The structure shown in in FIG. 14B made it clear that these 6 residues were all pointing outwards in MtLYK3. As shown in FIG. 6D, this was also the case for LjCERK6. These structures indicated that the JM zone 4 could be a good candidate for a point of contact for downstream signaling. It further implied that it was possible to engineer this zone between NFR1-types and CERK6-types without destroying the integrity of the structure and destabilizing the protein. In order to assess conservation within CERK6-type kinases and NFR1-type kinases, alignments of CERK6-type kinase and NFR1-type kinase sequences from 23 species in the Fabales order were made. Conservation at the sequence level was evaluated in relation to the structures to see the relative spatial locations of the variable regions (FIG. 14C). The kinase core was very conserved (shown in blue in FIG. 14C), while more variable regions (shown in green, yellow, orange, and red in FIG. 14C) were generally found on the outside of the structure. Areas in the lower C-terminus (shown in the lower left regions of the structures depicted in FIG. 14C seemed to generally vary substantially in both CERK6-type kinases and NFR1-type kinases. In contrast, zone 4 (marked with red lines in FIG. 14C) was very conserved in NFR1-type kinases, but varied substantially in CERK6-type kinases. The strict conservation in NFR1-type kinases pointed towards a conserved function in nodulation.

### Example 9: Biochemistry of Chimeric NFR1-CERK6 Proteins

The following example describes biochemical analyses of recombinant chimeric NFR1-CERK6 proteins.

### Materials and Methods.

### Nano-DSF

The chimeras as well as apo NFR1 kinase and apo CERK6 kinase were tested on nano DSF to assess their stability and binding capacity. They were furthermore tested for kinase activity in a radioactive kinase assay and run on native PAGE.

Thermal stability of purified proteins was assayed by nano DSF using a Tycho instrument (NanoTemper Technologies) in Tycho NT.6 Capillaries (NanoTemper Technologies). The ratio between the intrinsic fluorescence intensities at 330 nm and 350 nm was measured over a temperature gradient (35-90° C.) yielding a fluorescence transition curve from which an inflection temperature could be derived. Protein was assayed at a 10 μM concentration, while nucleotide and magnesium was added at a 1 mM concentration. Samples were incubated 10 minutes on ice before measurements.

### Radioactive Kinase Assays

4 μg of each protein were incubated with 200 nCi [γ32-P]ATP (PerkinElmer) in 5 mM Tris-HCl pH 8.0, 30 mM NaCL, 5 mM MgCl2 and 20 mM cold ATP at room temperature for 1 hour. The samples were then separated on a 15% SDS-PAGE gel which was stained with InstantBlue (Expedion) before it was was left to expose overnight on a TranScreen HE (BioMax). The phosphor plates were scanned with a Typhoon FLA 9500 (Cytiva).

### Native Gels

Polyacrylamide gel electrophoresis (native PAGE) was performed using self-cast 12% gels and ran on a Mini-PROTEAN tetra cell system (Bio-rad) at 4° C. 4 μg protein was loaded in each lane and the gel was run at 130 V for 3-4 hours.

### Results

In vitro biochemical experiments were completed to assess general stability, binding capacity and activity of the kinase domains of the chimeras purified from E. coli.

### Nano-DSF

Nano-DSF experiments were performed on the intracellular part of the following chimeric receptors to test their general stability and their ability to bind nucleotide (FIGS. 15B-15E). Protein schematics of Chimera A, Chimera B, Chimera C, and Chimera D are shown in FIG. 15A. The intracellular domains of NFR1 and CERK6 were also tested with nano-DSF.

The stability and nucleotide binding capabilities of Chimera A, Chimera C, and Chimera D was seen both from the raw nano-DSF data (FIG. 15C-15E) and from the inflection temperatures plotted in FIG. 15B, which all increased by more than 2° C. when ATP was added together with magnesium. This was also the case for CERK6. It was, however, not the case for Chimera B, which only showed a 1.5° C. increase in inflection temperature upon addition of ATP and magnesium (FIG. 15B and FIG. 15D), indicating weak or no binding. NFR1 was known to bind nucleotide since it is an active kinase (FIG. 15H and FIG. 151) and thus this weak stabilization on nano-DSF is thought to reflect sufficient binding of nucleotide to support kinase activity. For Chimera B, there was no demonstrated kinase activity and thus there might actually have been no binding of nucleotide.

### Native Gel

From size exclusion chromatography and SDS-PAGE gels, the purified protein was concluded to be of high purity. To assess if the protein was homogenous, it was run on a native PAGE gel (FIG. 15F). From the distinct ladder patterns on the gel, it was concluded that the proteins were heterogeneous. Protein kinases are known to be able to autophosphorylate and transphosphorylate during expression in and purification from E. coli, but these potential phosphorylations were removed with phosphatase when the proteins were dialyzed and should therefore not have given rise to this heterogeneity. Instead, it would be due to different oligomeric states formed in solution. When comparing the ladder pattern for each of the proteins, it was seen that Chimera A and Chimera C showed the same pattern as NFR1, while Chimera B and to some extent Chimera D showed the same pattern as CERK6 (FIG. 15F). This indicated that zone 2 and 3 were important for the oligomerization of the kinases, while zone 4 in this case did not seem to play a role.

### Kinase Assay

The intracellular domains of the chimeras were tested for kinase activity together with the intracellular domains of CERK6, NFR1, and NFR1 D462N (negative control). Each protein was tested for autophosphorylation and for transphosphorylation of MBP (FIG. 15H). From FIG. 15H it can be seen that CERK6 produced both auto- and transphosphorylation activity, while NFR1 only seemed to have very weak transphoshorylation activity. This contrasted with earlier experiments with NFR1, in which NFR1 was found to indeed have both auto- and transphosphorylation activity (FIG. 15H). It can be concluded that the seemingly weak activity of NFR1 was due to the dynamic range of the Typhoon scanner. The activity was simply less than the other kinases on the gel and thus the signal drowned when it was included in the same assay with more efficient kinases. The NFR1 D462N negative control and Chimera B showed no kinase activity (FIG. 15F). All the other chimeras showed kinase activity (FIG. 15F). Chimera A showed mostly transphosphorylation activity, while Chimera C and Chimera D also showed a lot of autophosphorylation activity (FIG. 15F).

### Example 10: Phylogenetic Analysis Reveals Conserved and Variable Motifs in LysM RLK Homologs

The following example describes phylogenetic analyses revealing that certain motifs in LysM RLK homologs are conserved while others are variable.

### Materials and Methods

### Selecting Receptors for Analysis

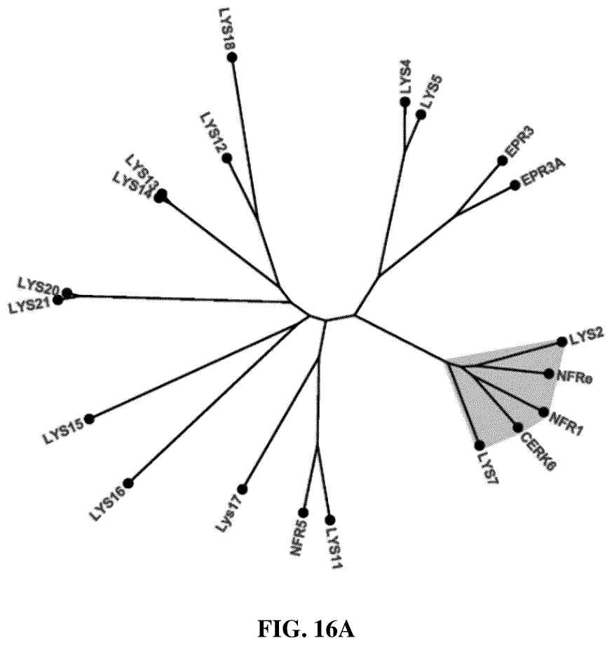

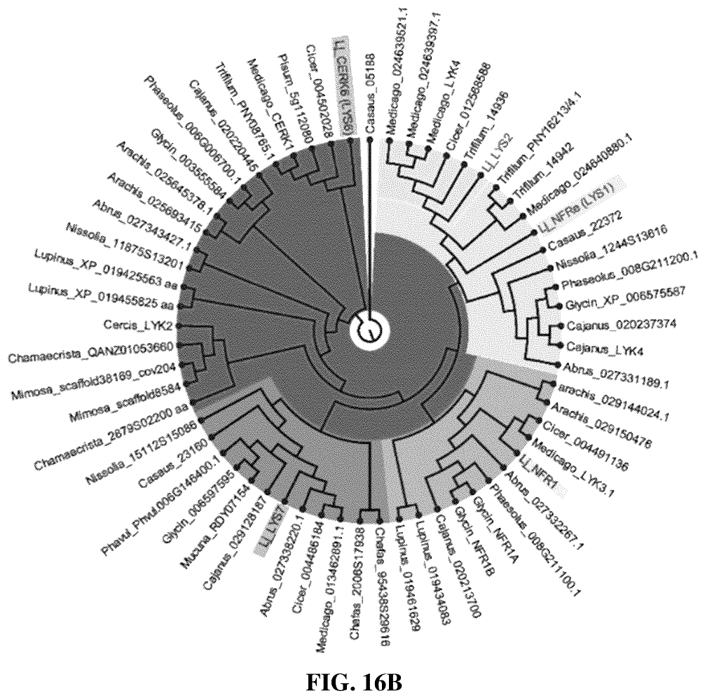

NFR1 and CERK6 of Lotus japonicus share high homology, with the full length proteins being 65% identical, and the kinase domains being 80% identical. They also belong to the same subfamily of LysM receptors with active kinase (NFR1 clade; shown in pink in FIG. 16A) together with Lys2, Lys7, and NFRe (FIG. 16A) (Lohmann et al., (2010) MPMI, 23(4):510-21). Because receptors belonging to this clade share a high level of identity, it can be challenging when analyzing their protein sequences to pinpoint residues that control the functions of NFR1 and CERK6 in symbiosis and immunity. A phylogenetic analysis of these receptors across different plant species was performed to reduce the number of candidate residues and regions. The rationale behind this analysis was that these species have conserved determinants of signaling specificity for symbiosis and immunity.

### Genome Queries

For the analysis, a collection of DNA and protein data was used. Sequences were retracted with blasting from the NCBI and an internal database. As queries, the ectodomain, transmembrane and juxtamembrane domains of LjNFR1 genomic or protein sequence were used. Homologues from Fabales order (i.e. homologues from the species Abrus precatorius, Arachis hypogaea, Cajanus cajan, Casaus castanospermum, Cercis spp., Cicer arietinum, Glycine max, Lupinus angustifolius, Medicago truncatula, Nissolia schottii, Pisum sativum, Chamaechrista spp., Phaseolus vulgaris, Mimosa pudica, and Trifolium spp.) were used for the study. In addition, the following protein sequences from non-Fabales species were added to the analysis: HvRLK4, MesCERKa, MesCERKb, MesLYKI, PanLYK3.1, and PanLYK3.2.

### Phylogenetic Analyses

Upon gene annotations (exons/introns recognition and annotation) and translation of the coding sequences into protein, protein alignments and phylogenetic trees were created utilizing the CLC Main Workbench (Qiagen). Filtering of the different accessions was performed by taking into consideration the following criteria: a) retracted genes had to contain 11-13 exons, b) retracted or in silico translated proteins had to contain three LysM domains separated by CXC motif, c) a transmembrane domain, and d) an active kinase domain (containing DFG motif and activation loop). Alignment and phylogenetic trees were created with CLC Main Workbench, Qiagen. Phylogenetic trees: Tree construction method: Neighbor joining, Protein distance measure: Jukes-Cantor Bootstrap analysis: 100 replicates.

### Homology Modeling

The model in FIG. 18A was made with homology modelling utilizing the MtLYK3 crystal structure, and the C-terminal tail was created with de novo modelling. NFR1 homology model was created automatically with the SWISS-MODEL software (Waterhouse et al. (2018) Nucleic Acids Res. 46, W296-W303).

### Results

After applying the filtering steps described in the phylogenetic analyses methods above, the sequences were categorized into five groups based on their homology with L. japonicus NFR1, CERK6, LYS7, NFRe and LYS2 or the presence or absence of protein motifs that characterize the previous receptors (such as the lack of the “YAQ” motif in NFRe and Lys2) (FIG. 16B).

Next, the candidate residues or regions containing possible determinants of specificity for NFR1 and CERK6 were identified from the protein alignments. A phylogenetic tree containing the NFR1 and CERK6 homologs in Fabales and closest relatives from non-Fabales species was constructed (FIG. 17B). Homologs of NFR1 from Fabales were grouped in the same clade that separates from the ancestral CERK clade containing CERK6 (shown shaded in pink in FIG. 17A).

Separate protein alignments were made for NFR1 (Fabales) homologs, CERK6 (Fabales) homologs, and RLKs (non-Fabales) homologs, respectively, and the corresponding 70% consensus sequences were identified. The 70% consensus represented the protein sequence for which, for each position, a given amino acid was conserved in more than 70% of the aligned accessions, whereas “X” was noted for the residues that varied in more than 70% of the accessions (FIG. 17B). The kinase zones in FIG. 18A were illustrated in an NFR1 kinase model made with homology modeling utilizing the MtLYK3 crystal structure.

Alignment of the three 70% consensus protein sequences (FIG. 17B) enabled classification of RLK kinase motifs based on their conservation pattern. Crystal structures of MtLYK3 and LjCERK6 kinases (FIG. 17C) revealed that residues within these motifs were surface exposed, which enabled their classification into groups that create kinase zones for putative inter- or intramolecular protein-protein interactions. Those motifs were categorized into groups defined as kinase zones A-G, and are illustrated in the NFR1 kinase model in FIG. 18A. FIG. 18B illustrates the residues in each kinase zone, which are listed in Table 1. In addition, FIG. 18B shows the division into N- and C-terminus (indicated by red-dashed line) for cloning purposes. Surface A (i.e., Zone A) contains residues that belong to the activation loop (AL). Surface B (i.e., Zone B) contains residues of the αG- and αG′-helixes as well as the loop that connects them. Surface C (i.e., Zone C) contains residues of the C-terminal tail. Surface D (i.e., Zone D) contains three residues located in the αH-helix. Surface E (i.e., Zone E) primarily contains residues that belong to the αC-helix. Surface F (i.e., Zone F) contains residues that belong to different loops, sheets and helixes that were in close proximity in the structure. Finally, surface G (i.e., Zone G) contains residues that belong to the αG-H loop (FIGS. 18A-18B). Each surface shown in in FIG. 18A consisted of surface-exposed residues that created regions for putative in-cis or in-trans protein-protein interactions. In FIG. 17C, the order of the beta-sheets, helixes and loops in the crystal structures is illustrated. These motif candidates were further analyzed in planta for their role in symbiosis and immunity based on functionality as described in Examples 11 and 12, as well as analyzed for their role in structure in Example 13.

TABLE 1
Amino acids composing the kinase zones of NFR1 and CERK6. Amino
acids composing each kinase zone and their positions in the protein
sequence are indicated. The corresponding amino acid and its position in
NFR1 (SEQ ID NO: 1) or CERK6 (SEQ ID NO: 8) protein sequence) is
indicated by a separation with a slash (/).

| | | No. |
|---|---|---|
| Surface | NFR1/CERK6 | residues |
| A | I469/T467, N473/S471, T475/S473, | 5 |
| | Q477/P475, —/G477 | |
| B | E539/G538, A540/V539, N542/S541, | 8 |
| | K543/Q542, S544/P543, C547/T546, | |
| | D548/E547, A549/D548 | |
| C | L601/T600, E603/D602, C605/W604, | 11 |
| | D607/V606, E608/G607, S610/F609, | |
| | S613/N612, T615/N614, I617/V616, | |
| | L620/M619, V622/G621 | |
| D | L568/R567, R575/K574, R579/Q578 | 3 |
| E | V356/M354, T360/K358, C364/A362, | 6 |
| | H371/R369, C384/S382, I469/4367 | |
| F | H395/F393, G402/S400, Y404/H402, | 6 |
| | S416/A414, S417/T415, L456/Y454 | |
| G | N518/D517, V520/I519, L521/V520, | 8 |
| | G524/S523, L526/S525, V527/I526, | |
| | A528/T527, E529/D528 | |
| N- | D328/A326, A338/S336, K347/E345, | 23 |
| terminus | K348/R346, T349/A347, V356/M354, | |
| | T360/K358, C364/A362, H371/R369, | |
| | C384/S382, V385/I383, H395/F393, | |
| | D397/E395, G402/S400, Y404/H402, | |
| | H406/R404, K410/R408, E411/D409, | |
| | S416/A414, S417/T415, A425/S423, | |
| | V445/I443, L456/Y454 | |
| C- | I469/T467, N473/S471, T475/S473, | 40 |
| terminus | Q477/P475, —/G477, I497/V496, | |
| | I501/V500, F511/Y510, N518/D517, | |
| | V520/I519, L521/V520, G524/S523, | |
| | L526/S525, V527/I526, A528/T527, | |
| | E529/D528, E539/G538, A540/V539, | |
| | N542/S541, K543/Q542, S544/P543, | |
| | C547/T546, D548/E547, A549/D548, | |
| | P556/Q555, E560/D559, E564/V563, | |
| | L568/R567, I570/M569, G574/A573, | |
| | R575/K574, R579/Q578, L583/Q582, | |
| | L591/I590, L601/T600, E603/D602, | |
| | C605/W604, D607/V606, E608/G607, | |
| | S610/F609, S613/N612, T615/N614, | |
| | I617/V616, L620/M619, V622/G621 | |

### Example 11: The Role of Kinase Surfaces in Symbiotic Signaling

The following example describes experiments investigating the role of the NFR1 and CERK6 kinase domains in symbiotic signaling.

### Materials and Methods

### Cloning of NFR1-CERK6 Chimeras

A level 2 pIV10 expression vector (Márquez, A. J., and J. Stougaard, eds. 2005. Lotus Japonicus Handbook. Dordrecht: Springer) was used for Hairy Root transformation, containing an expression cassette of nuclear localized tripleYFP driven by the constitutive promoter pUbi (pUbi::tYFP-NLS) serving as transformation marker. The chimeric constructs or chimeric variants were positioned downstream of pNFR1. Golden gate cloning (Weber et al. 2011) was used to create different receptor versions. The modules utilized to assembly the receptor chimeras were the following: LjCERK6 and LjNFR1 (genomic sequences containing introns): Ectodomain (EC), Transmembrane/Juxtamembrane domain (TM/JM), TM/JM zones 1-4 (TM/JM zone 1 is part of the TM (NFR1 zone 1=SEQ ID NO: 3; CERK6 zone 1=SEQ ID NO: 10), while zones 2-4 are part of the JM (NFR1 zone 2=SEQ ID NO: 4; NFR1 zone 3=SEQ ID NO: 5; NFR1 zone 4=SEQ ID NO: 6; CERK6 zone 2=SEQ ID NO: 11; CERK6 zone 3=SEQ ID NO: 12; CERK6 zone 4=SEQ ID NO: 13)), Kinase domain and mutagenized kinase domain.

### Crystallography

Crystallography was performed for receptors corresponding to constructs designated 27 and 29, according to methodology in Example 8.

### Homology Modeling

Modeling for homology between crystallographic models was performed according to methodology in Example 10.

### Other Methods

Bacteria and culture conditions, plant materials, Hairy Root transformation, nodulation assays and other methods were conducted as described in Example 1 unless stated otherwise.

### Results

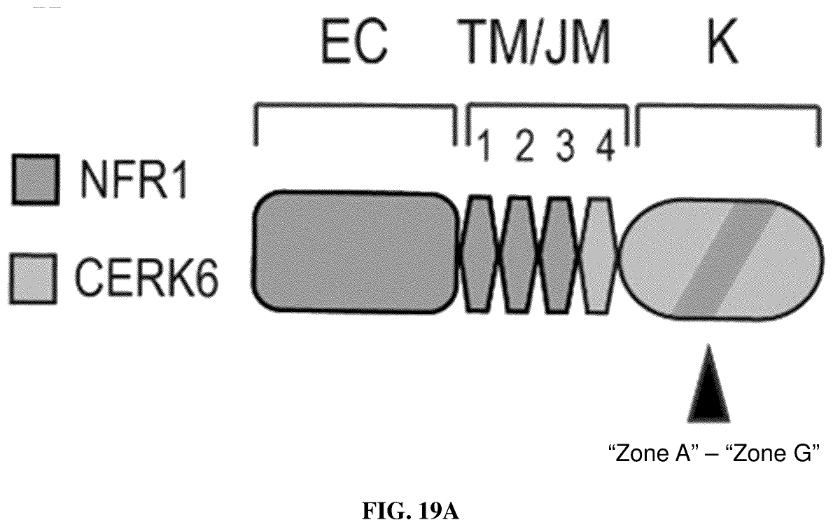

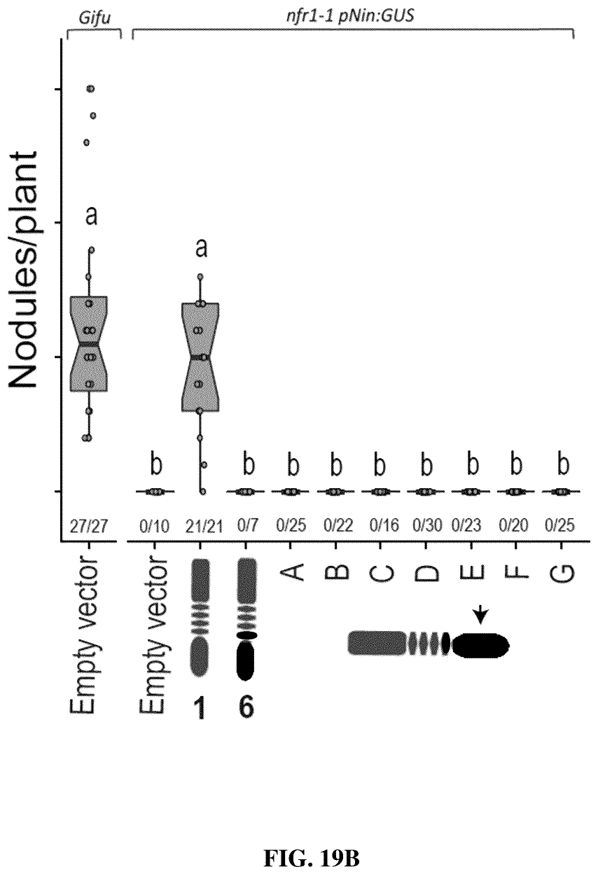

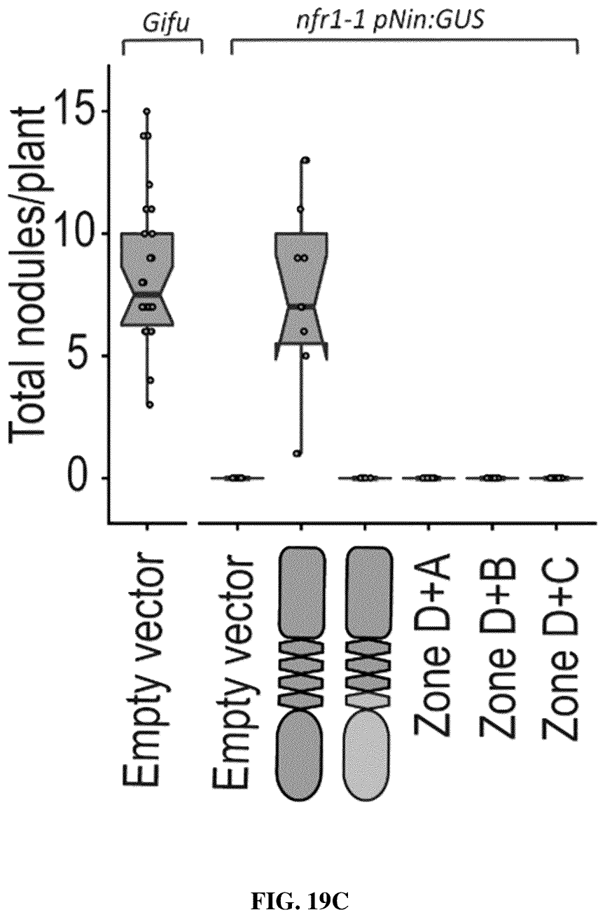

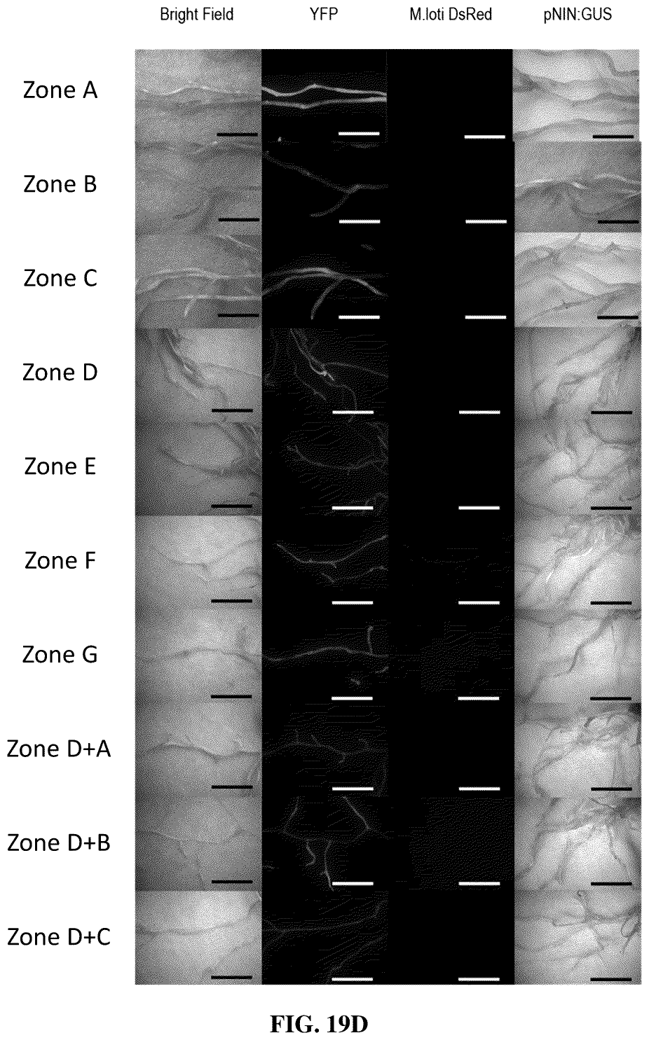

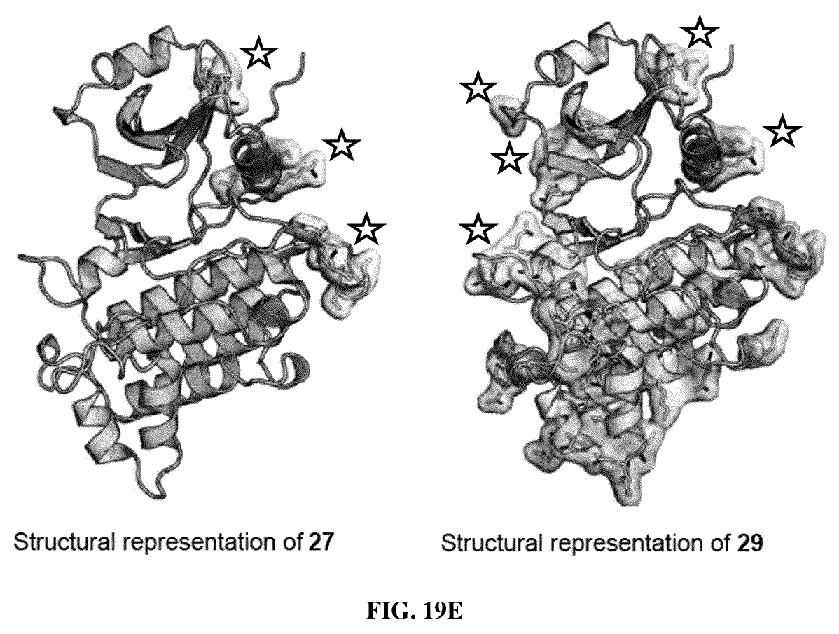

A series of additional chimeric NFR1 receptor versions were created, in which each contained both zone 4 and the kinase domain from CERK6, but in which the sequences of the kinase surfaces (zones A through G, as identified in FIGS. 18A-18B) were exchanged from that of CERK6 to that of NFR1 (as shown schematically in FIG. 19A), either individually (FIG. 19B) or in combination (FIG. 19C).

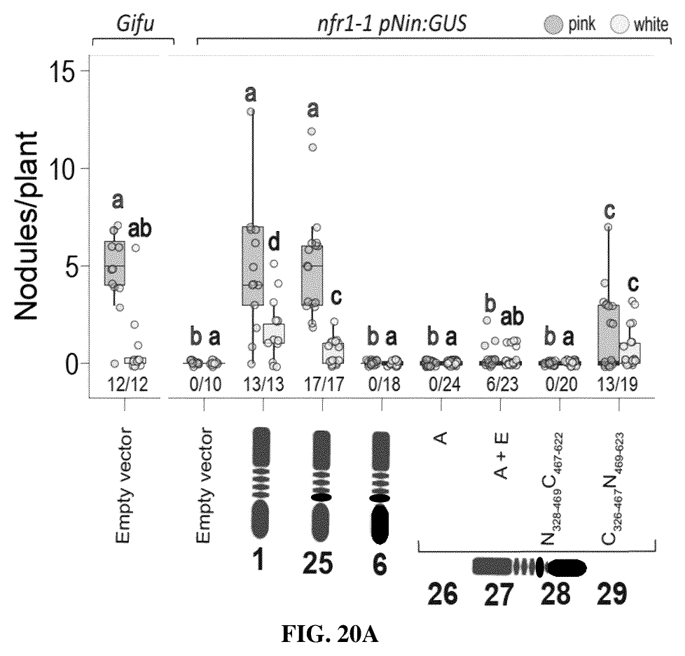

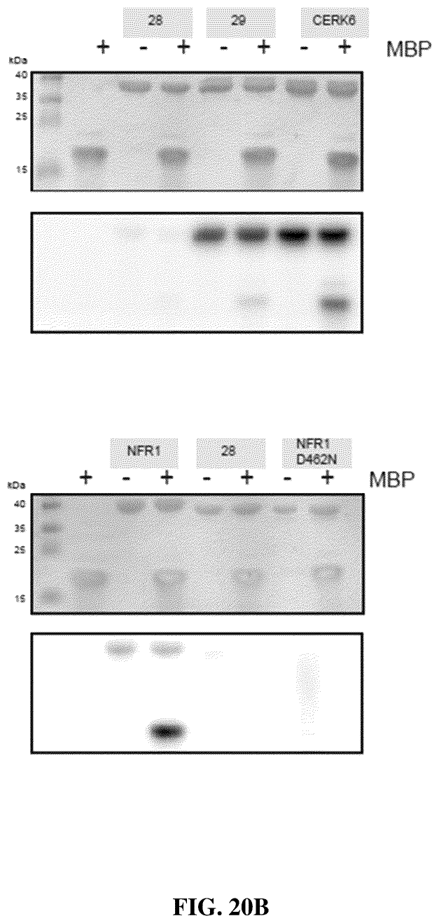

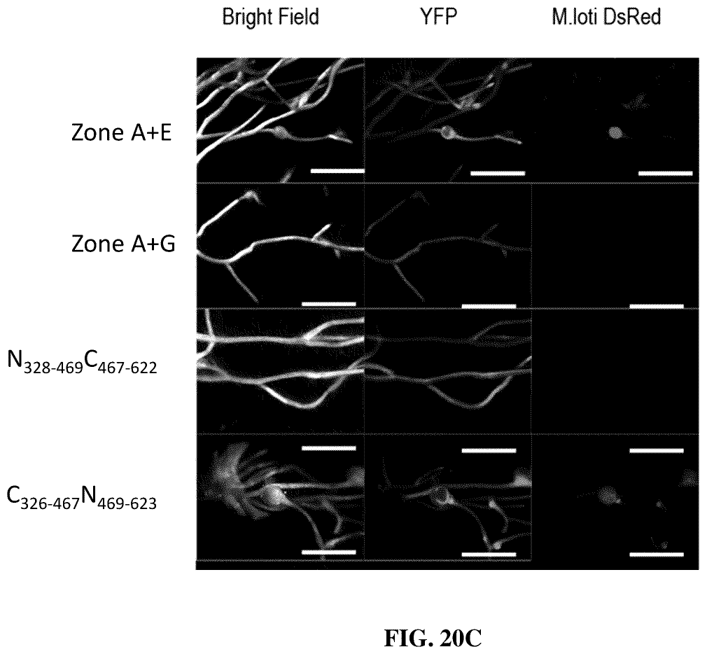

A series of further variants were created based on the schemes shown in FIG. 20A. The receptor variant named “Zone A+E” was designed to examine the role of the Activation Loop (zone A) and the αC-helix (zone E), two kinase regions known for their catalytic and regulatory properties. The receptor variant named “Zone A+G” was designed to examine the role of the Activation Loop (zone A) and the αFG-loop (zone G), both of which have been reported to be phosphorylated in NFR1, and a receptor variant named “Zone A” was additionally tested in FIG. 20A (construct 26) to account for the individual effect of Zone A. The receptor variant named “N₃₂₈₋₄₆₉C₄₆₇₋₆₂₂” had the N-terminus of the kinase domain from NFR1 and the C-terminus of the kinase domain from CERK6. The receptor variant named “C₃₂₆₋₄₆₇N₄₆₉₋₆₂₃” had the N-terminus of the kinase domain from CERK6 and the C-terminus of the kinase domain from NFR1. The border between N- and C- termini of these kinases, at N₃₂₈₋₄₆₉C₄₆₇₋₆₂₂ and C₃₂₆₋₄₆₇N₄₆₉₋₆₂₃, respectively, is highlighted with a dashed red line in FIG. 18B.

Example 1 described experiments showing that chimeric NFR1 receptors containing the both zone 4 and the kinase domain from CERK6 were not able to complement symbiosis in the nfr1-1 mutant, but that chimeric NFR1 receptors containing zone 4 of CERK6 and kinase of NFR1 were symbiotically proficient receptors (see, e.g., FIG. 3). This indicated that the NFR1 kinase domain, unlike that of CERK6, contained specific residues or regions that could overcome the limitations imposed by zone 4 of CERK6. In order to identify these, a series of chimera based on swaps in the kinase domain were constructed and tested for their capacity to restore nodulation in nfr1-1 pNIN:GUS mutant, as shown in FIG. 19. A series of further variants was constructed to test the functionality of combinations of different regions, as shown in FIG. 20A. The role of larger kinase surfaces was also tested to indicate whether they could possibly serve as docking sites for NFR1 interacting partners or regions that control the specificity in an allosteric manner, as shown in FIG. 20A.

NFR1 Kinase Zones, Individually or in Combination with Zone D, are not Sufficient to Induce Signaling

None of the tested receptor versions with individual kinase domain zones exchanged from CERK6 to NFR1 were able to induce nodule organogenesis at 35 days post infection after inoculation with M. loti R7a (see data sets labeled “Zone A”, “Zone B”, “Zone C”, “Zone D”, “Zone E”, “Zone F”, and “Zone G” in FIG. 19B). Further, none of the tested receptor versions with combinations of kinase domain zones exchanged from CERK6 to NFR1 were able to induce nodule organogenesis at 35 days post infection after inoculation with M. loti R7a, either (see data sets labeled “Zone D+A”, “Zone D+B”, and “Zone D+C” in FIG. 19C). It was also found that none of the tested receptor versions (either with individual or combinations of kinase domain zones exchanged) were able to induce pNIN activation at 35 days post infection after inoculation in roots expressing pNIN:GUS (FIG. 19D).

Surprisingly, not even the chimeras containing the Activation Loop (zone A or zone D+A) from NFR1 were able to induce nodulation (FIG. 19B or FIG. 19C, respectively), even though this region is known to be important for symbiosis (Nakagawa et al. (2011) The Plant Journal 65(2):169-80; Murakami et al. (2018) ELife 7:e33506).

### The C-Terminus of the NFR1 Kinase Domain is Essential for Nodulation

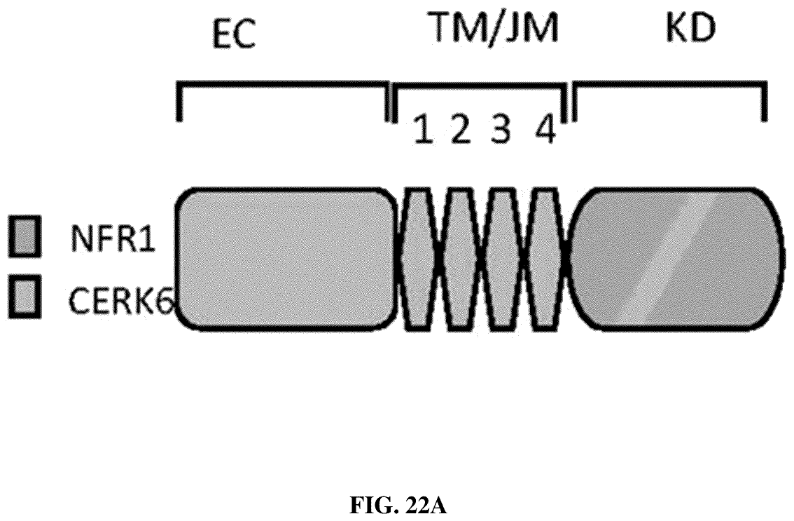

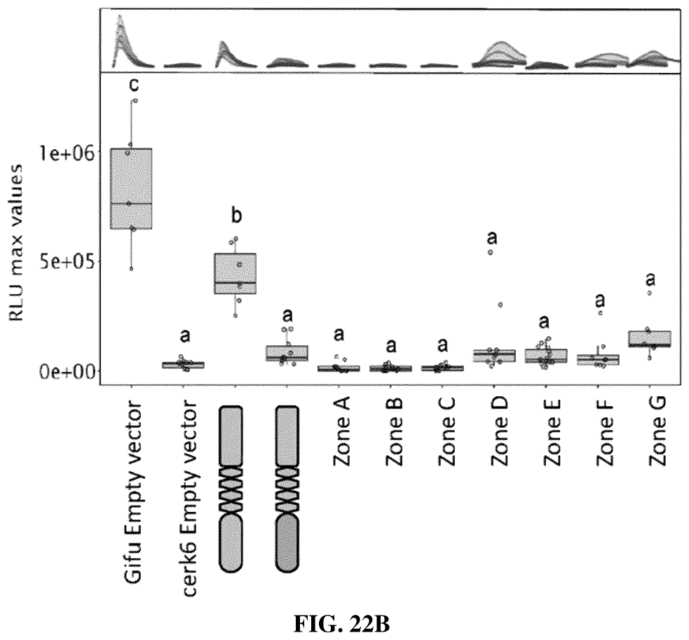

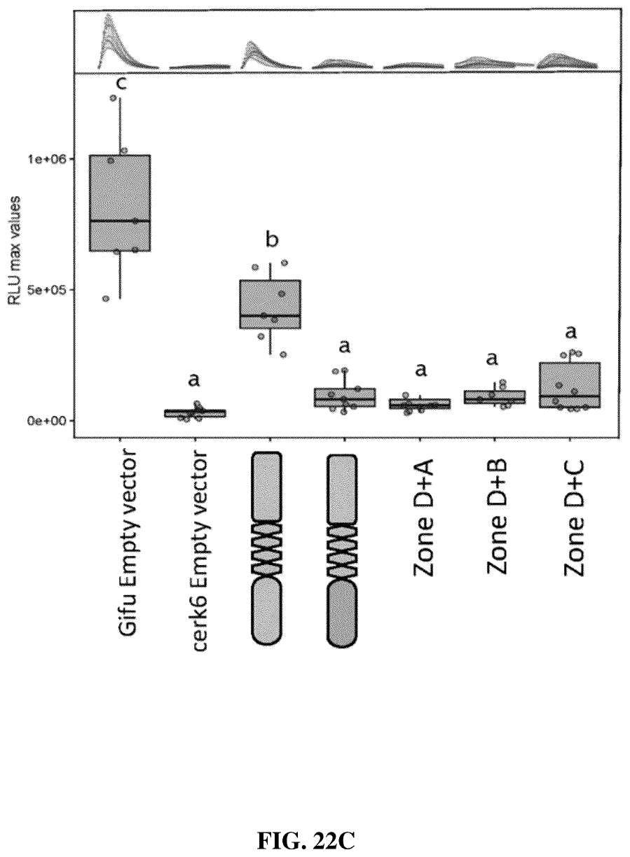

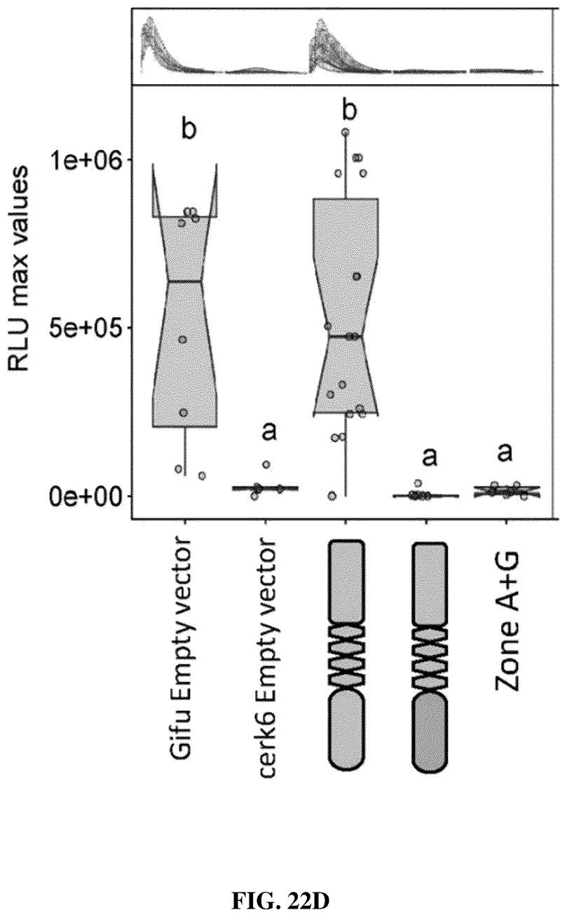

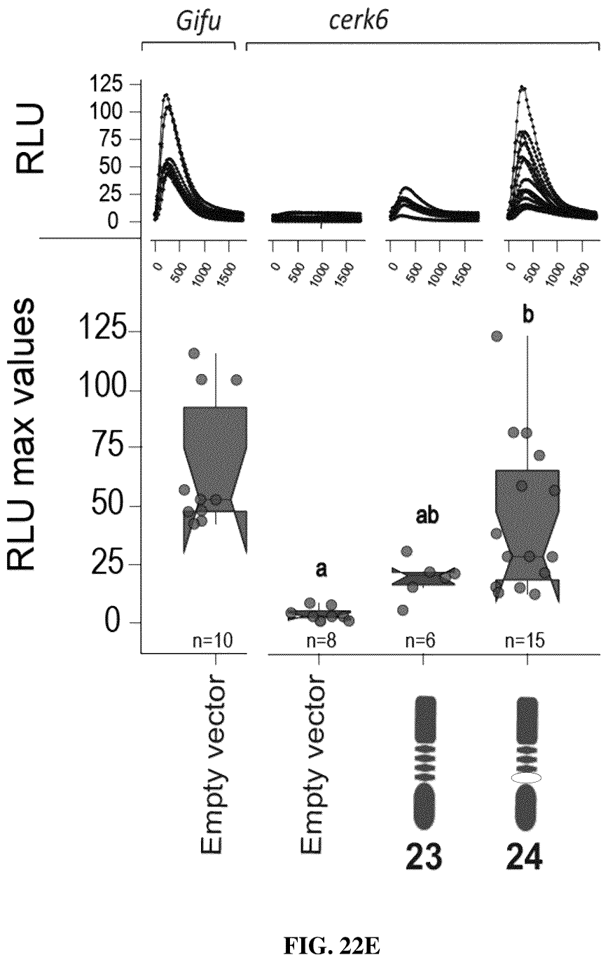

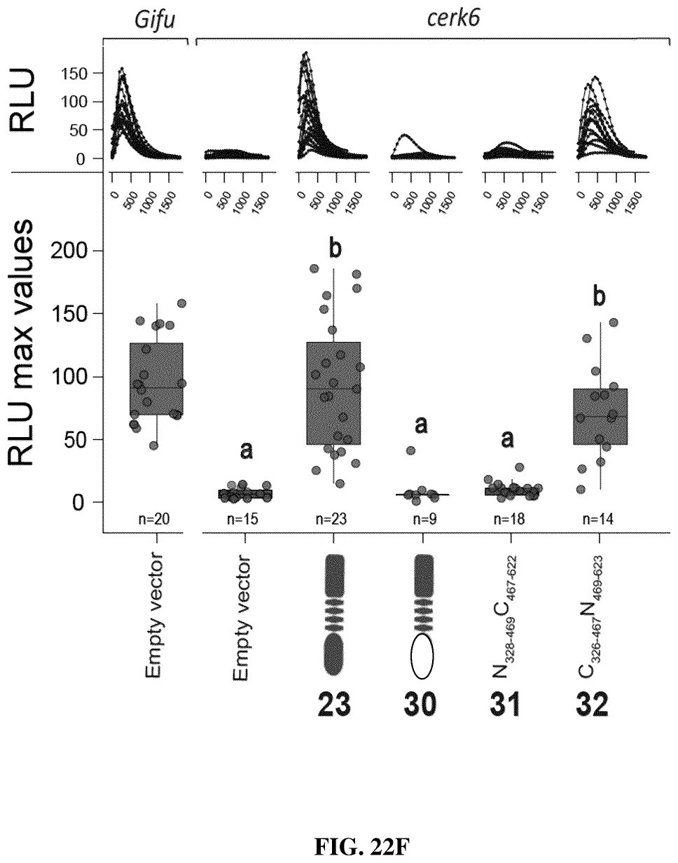

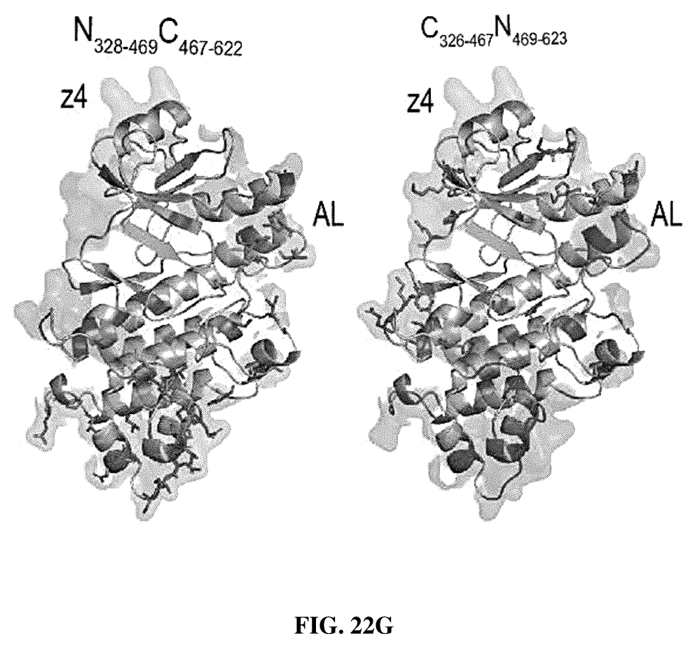

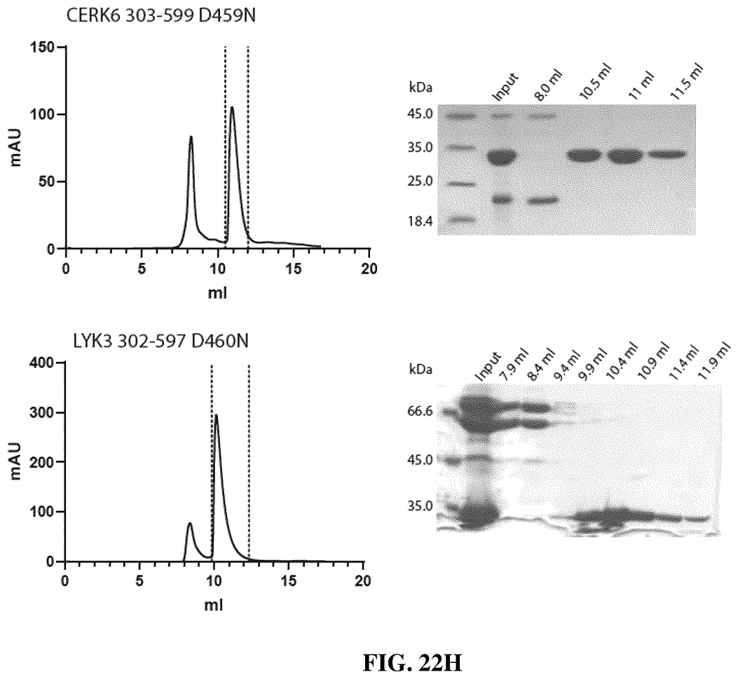

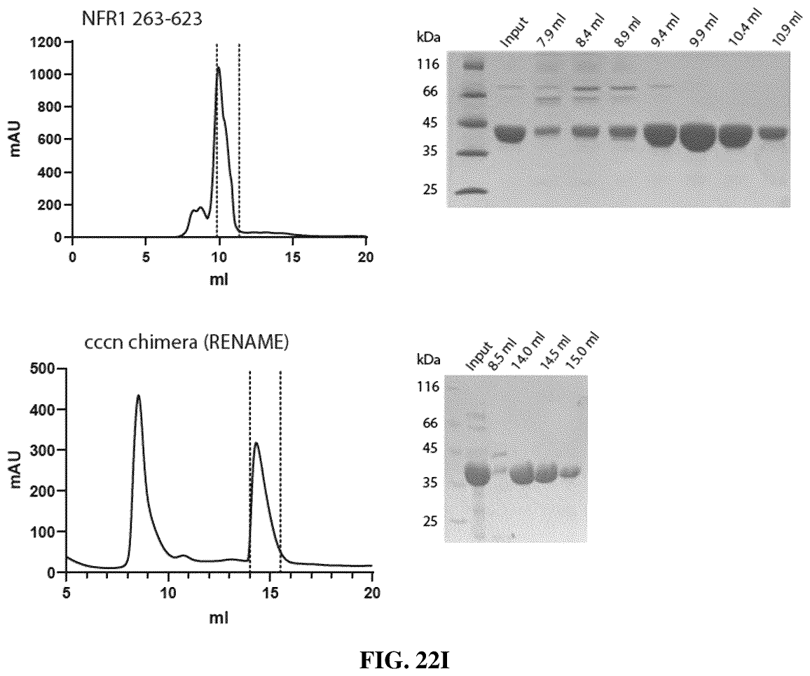

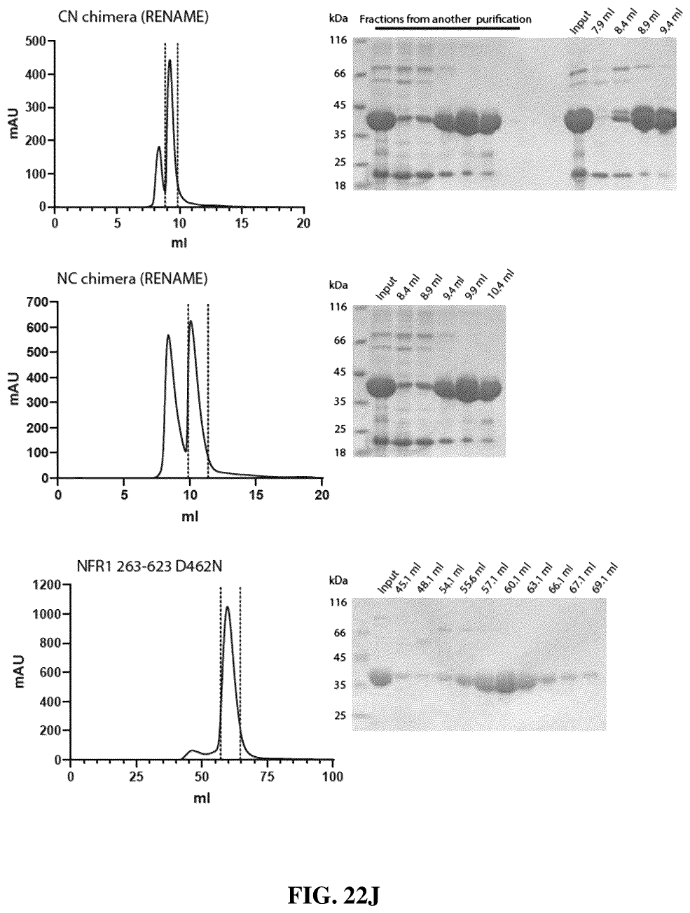

Nodulation experiments on the additional variants shown in FIG. 20A demonstrated that the combination of the Activation Loop and αC-helix of NFR1 (zone A+E) was sufficient to induce nodule organogenesis in nfr1-1 pNIN:GUS hairy roots in 6 out of 23 tested plants (26%), which formed a maximum of 2 nodules (FIG. 20A). However, neither the variant with the combination of the Activation Loop and the αFG-loop of NFR1 (zone A+G), nor the variant with the CERK6 C-terminal region (N₃₂₈₋₄₆₉C₄₆₇₋₆₂₂) (also referred to herein as receptor 28 or construct 28), were able to induce nodule formation (FIG. 20A and FIG. 20C). Surprisingly, however, the variant with the CERK6 N-terminal region and the NFR1 C-terminal region (C₃₂₆₋₄₆₇N₄₆₉₋₆₂₃) (also referred to herein as receptor 29 or construct 29) was functional and enabled 13 out of 19 tested plants (68%) to form nodules (FIG. 20A) to a level higher than receptor 27 (FIG. 19E). The CERK6 crystal structures for C₃₂₆₋₄₆₇N₄₆₉₋₆₂₃ and N₃₂₈₋₄₆₉C₄₆₇₋₆₂₂ are depicted in FIG. 22G, showing residues varying between these two receptors. These results indicated that the C-terminus of the NFR1 kinase present in receptor C₃₂₆₋₄₆₇N₄₆₉₋₆₂₃ was critical for root nodule symbiosis, and/or that C-terminus of CERK6 coupled with the CERK6 JM zone 4 inhibited signaling in nodulation. These results indicated that the larger surface of the C-terminus could possibly serve as docking sites for NFR1 interacting partners or regions that control the specificity in an allosteric manner. It was tested whether such large alterations as those of N₃₂₈₋₄₆₉C₄₆₇₋₆₂₂ and C₃₂₆₋₄₆₇N₄₆₉₋₆₂₃ might interfere with protein expression or folding, but both were found to be produced and active when expressed in E. coli (FIGS. 22H-22J). Additionally, N₃₂₈₋₄₆₉C₄₆₇₋₆₂₂ (receptor 28) showed greatly decreased catalytic capacity both for auto- and substrate phosphorylation, while C₃₂₆₋₄₆₇N₄₆₉₋₆₂₃ (receptor 29) maintained the auto-P capacity and diminished the ability to phosphorylate the MBP substrate (FIG. 20B). A similar alteration in the balance between auto- and substrate-phosphotransfer was observed for receptors 13/24 where the JM-B of NFR1 was coupled to the native CERK6 (FIG. 15G). The C-terminal regions of the kinases are known docking sites for downstream partners (Wang et al. (2014) Structural insights into the negative regulation of BRIl signaling by BRIl-interacting protein BKIl. Cell Res 24, 1328-1341; Yamada et al. (2016) The Arabidopsis CERK1-associated kinase PBL27 connects chitin perception to MAPK activation. EMBO J. 2016 Nov. 15; 35(22):2468-2483); thus, it is thought that JM-B and C-terminal regions can coordinate the interaction of the core kinase with inhibitor(s) for symbiosis and/or inhibit interaction with a downstream partner for symbiosis, possibly NFR5.

### Example 12: The Role of Kinase Surfaces in Immune Signaling

The following example describes experiments investigating the role of the NFR1, HvRLK4, and CERK6 kinase domains in immune signaling.

### Materials and Methods

### ROS Measurements

Lotus japonicus Gifu and cerk6 mutant plants were transformed with Agrobacterium rhizogenes containing the respective empty vector or chimeric CERK6-NFR1 receptor constructs. Plants with hairy roots were grown in magenta containers containing Leca substrate and B&D medium with KNO3 3 mM for 21 days. Only the transformed hairy roots were kept (detected by fluorescence under UV light since the transgenic roots have tripleYFP incorporated into their genome) and cut into 1-2 cm pieces. Equal amounts of the cut roots were collected into white 96-well flat-bottomed polystyrene plates (Greiner Bio-One) trying to put one hairy root into one well. They were kept for overnight incubation in sterile MQ water. The next day, for the ROS measurement, the water in every well was replaced by reaction Master Mix consisting of: 0.5 mM L-023 (Wako Chemicals), 5 μg/mL horseradish peroxidase (Sigma), and 0.1 mg/ml chitin. Luminescence in the 30-40 min assays was recorded with a Varioskan Flash Multimode Reader (Thermo Scientific). In one repetition, at least three wells were measured for each treatment for every genotype.

### Other Methods

Cloning of CERK6 receptor variants containing the NFR1 kinase zones or the HvRLK4 ectodomain and membrane domains, bacteria and culture conditions, plant materials, Hairy Root transformation, nodulation assays and other methods were conducted as described in Example 11 unless stated otherwise.

### Results

Previous studies (e.g., Bozsoki et al., (2017) PNAS E8118-E8127; Bozsoki et al. (2020) Science 369, 663-670) showed that the NFR1 receptor, unlike CERK6, was not able to induce an immune response. This was explained by the fact that the NFR1 ectodomain does not recognize chitin, and its kinase lost the capacity to activate an immune signaling pathway. The CERK6 kinase domain, on the other hand, had both the capacity to activate symbiotic signaling (such as when combined with remaining NFR1 regions, as in, for example, FIG. 2B, FIG. 7B, FIGS. 9B-9C, FIG. 10B, and FIG. 11B) and immunity (when combined with the ectodomain of CERK6; as shown in FIG. 1 of Bozsoki et al. (2020) Science, 369(6504), 663-670). To identify which regions of CERK6 kinase were essential for immunity, a series of chimeric receptors were created, expressed in a cerk6 mutant, and characterized for capacity to induce ROS production after chitin treatment.

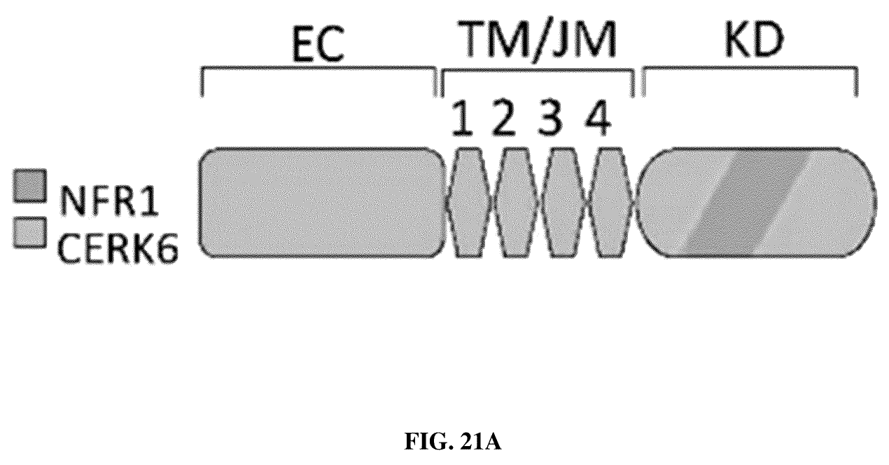

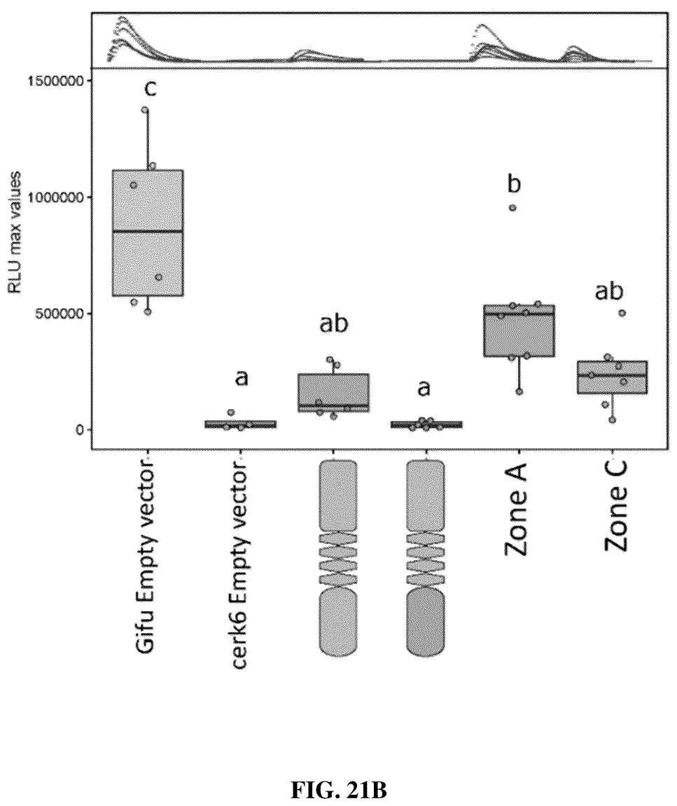

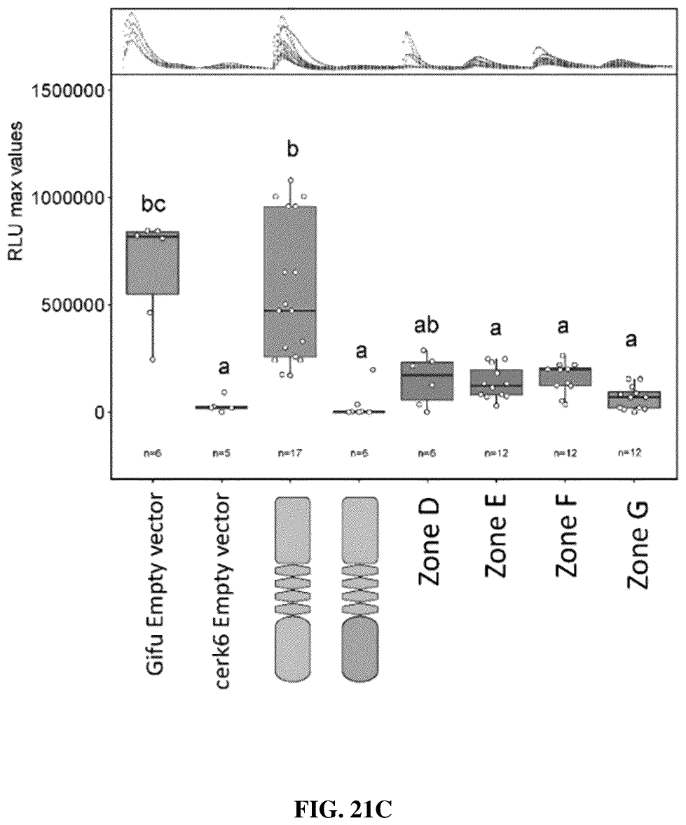

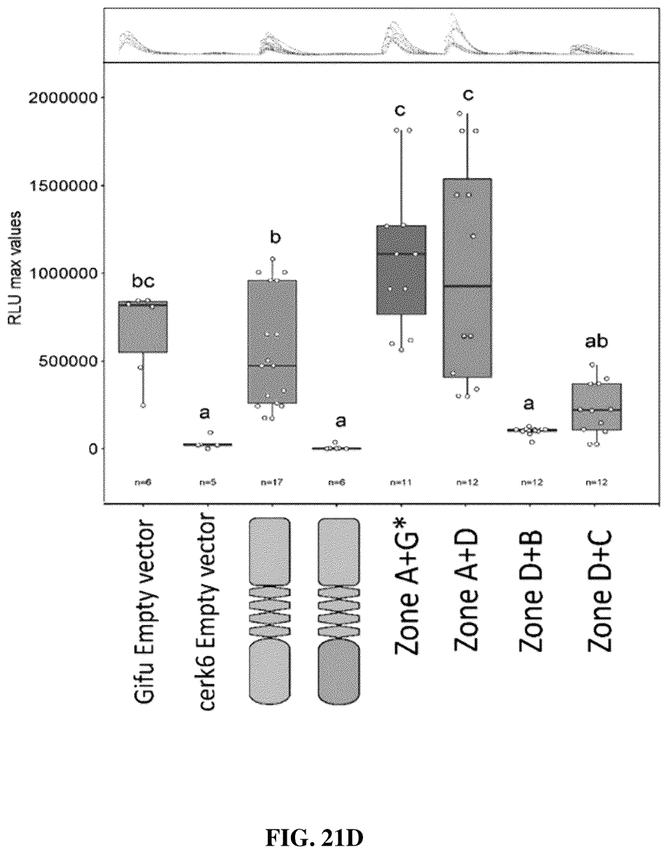

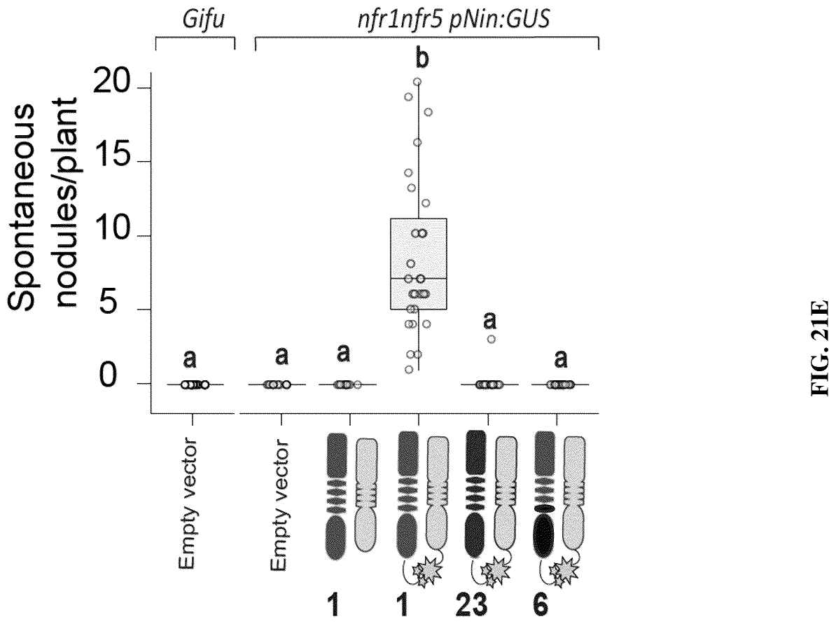

A series of CERK6 receptor variants containing the NFR1 kinase zones (see annotation in FIGS. 18A-18B) were designed according to the schematic representations in FIG. 21A and FIG. 22A.

Individual NFR1 Kinase Zones Function in Immune Signaling with Different Efficiencies

Experiments were undertaken to determine if specific regions of CERK6 that vary compared to NFR1 were critical in immune signaling. A series of ten CERK6 receptor variants containing NFR1 kinase zones (see FIGS. 21B-21C for annotations of the kinase zones) were constructed as shown in FIG. 21A. Six chimeras contained individual NFR1 zones (FIGS. 21B-21C) and four contained NFR1 zones in combinations (FIG. 21D). Assessing the immune response in hairy roots by ROS measurement was experimentally demanding; thus, these receptor variants were tested in separate experiments, each with its own positive and negative controls. These controls were: i) wild-type hairy roots expressing the empty vector, and cerk6 hairy roots expressing the full-length CERK6, both serving as positive controls; and ii) cerk6 hairy roots expressing the empty vector as the negative control.

The chimeric receptors containing zone A (Activation Loop or AL) or C (C-terminal tail) of NFR1 were still able to induce reactive oxygen species (ROS), as indicated by an increase in Relative Luminescence Units (RLU) reported within 30 minutes upon 0.1 mg/ml chitin application (FIG. 21B), indicating that these regions of NFR1, even if differing greatly from the corresponding regions of CERK6, did not have a negative impact on immune signaling. It is important to note that in this experiment, the cerk6 roots expressing the CERK6 full-length receptor showed a low level of ROS response when compared to wild-type roots (FIG. 21B); thus, it was difficult to directly compare the level of ROS production induced by the chimeric constructs.

Next, the impact of individual zones D, E, F, and G from NFR1 were each tested (FIG. 21C). This revealed a severe reduction in ROS production for all tested chimeric receptors. Chimeric receptors that incorporated zone G of NFR1 had the lowest capacity for ROS production (FIG. 21C).

Combinations of NFR1 Kinase Zones Function in Immune Signaling with Different Efficiencies

The four chimeras containing NFR1 zones in different combinations were tested for their ability to induce ROS production (FIG. 21D). NFR1 zone A was combined with either zone D or zone G(T523D) (annotated as zones A+D and A+G*, respectively, in FIG. 21D). The T523D mutation was designed to create a phospho-mimic version of zone G, in order to resemble the state of zone G observed in NFR1. Lastly Zone D was combined with either zone B or C (annotated as zone D+B and D+C, respectively, in FIG. 21D). Zone D+B and zone D+C were observed to have a negative impact in immune response, as zone D+B resulted in no observed ROS and zone D+C resulted in only a low intensity ROS response (FIG. 21D). In contrast, zone A+D and zone A+G* each performed with great efficiency, both producing a ROS response higher in intensity compared to Gifu (wild type L. japonicus) and CERK6 complementation (FIG. 21D).

Collectively, these results showed that signaling from the CERK6 kinase domain was highly sensitive to changes, given that exchanging individual or combined surfaces with those from NFR1 (C, D, E, F, G, D+B, D+C; FIGS. 21A-21C and FIG. 21D) had a negative impact or abolished ROS production. NFR1 zone A alone (FIG. 21B) and the combinations A+D or A+G* (FIG. 21D) were exceptions to this sensitivity, as these constructs were able to induce a high level of reactive oxygen species, suggesting that NFR1 zone A, which contained the Activation Loop, was more effective than the corresponding zone from CERK6.

Individual CERK6 Kinase Zones, or CERK6 Kinase Zones in Combination with CERK6 Kinase Zones D or G, are not Sufficient to Induce Immune Signaling when Coupled with the Remaining Zones from the NFR1 Kinase Domain.

Additional experiments were undertaken to explore if specific regions of CERK6 that vary from NFR1 were sufficient to induce immune signaling. Following the same logic as above, a chimeric CERK6 receptor containing the NFR1 kinase domain, which was not able to activate immune signaling due to the presence of NFR1 kinase (FIG. 22B-22D), was used as a basis for constructing additional CERK6-NFR1 chimeric constructs carrying kinase zone alternations from NFR1 to CERK6, in which kinase domain zones from NFR1 were substituted with the corresponding one(s) from CERK6 (see schematic representation in FIG. 22A), which were then tested for their ability to induce ROS production in transgenic Lotus japonicus roots upon chitin application. The sequences of kinase domain zones A to G from NFR1 were exchanged individually (FIG. 22B) or in combinations (D+A, D+B, and D+C in FIG. 22C; and A+G in FIG. 22D). The chimera with the zone A+G substitution (FIG. 22D) allowed examination of the role of the Activation Loop (zone A) in combination with the αFG-loop (zone G). This was an interesting combination because zone A is phosphorylated in both CERK6 and NFR1, but zone G is phosphorylated in NFR1 but not in CERK6.

These chimeric kinase receptors were expressed in cerk6 mutants, and transformed roots were exposed to chitin to monitor receptor functionality. Production of ROS in the tested and control roots was monitored after chitin treatment for up to 30 minutes. Roots of wild-type plants and those of cerk6 mutants expressing the full-length CERK6 (FIGS. 22B-22D, left-most and third-to-right columns, respectively) showed ROS production within 3 minutes, as expected. Mutant roots expressing each of the tested chimeric constructs (right portions of each of FIGS. 22B-22E) behaved similarly to the cerk6 roots transformed with the empty vector (second from left in each of FIGS. 22B-22D), and failed to induce ROS. It was noted that a small number of plants expressing the chimeric receptors with zones D+C (FIG. 22C), D (FIG. 22B), F (FIG. 22B), or G (FIG. 22B) of CERK6 were able to induce a delayed, low-intensity response (see shifted response curves at the top of FIGS. 22B-22C). These results indicated that the tested zones of CERK6 kinase, whether individually or in various combinations, were not sufficient to restore the lack of immune signaling in the NFR1 kinase. This indicated that either larger or multiple surfaces of CERK6 contribute to the downstream signaling.

### The N-Terminus of the CERK6 Kinase Domain is Essential for Immune Signaling

Thus, a series of experiments was designed to explore the impact of exchanging larger surfaces in the kinase domain to those of CERK6. The variant named C326-46₇N469-623 had the EC and TM/JM of CERK6 and a chimeric K domain with the N-terminus of the K domain from CERK6 and the C-terminus of the K domain from NFR1. The receptor variant named N328-469C467-622 had the EC and TM/JM of CERK6 and a chimeric K domain with the N-terminus of the K domain from NFR1 and the C-terminus of the K domain from CERK6. The border between N- and C- termini is highlighted in FIG. 18B with a dashed red line. Interestingly, the C326-467N469-623 construct was able to induce a canonical ROS response after chitin application, comparable to the response reported in the wild-type and complementation CERK6 controls (right-most data compared to the left-most data and third-from-left data with RLU displayed in FIG. 22F). These results suggested that the N-terminus of the CERK6 kinase was essential for immune signaling, and/or that the NFR1 N-terminus was inhibitory.

Interestingly, the intracellular variant of C326-467N469-623 but not N328-469C467-622 coupled to CERK6 ectodomain (receptors 31 and 32) was also permissive for chitin immune signalling (FIG. 22F, receptors 31 and 32), indicating that the intracellular form of C326-467N469-623 (and its related derived receptor 32) can perform a dual function that is dependent on the type of the ectodomain. N328-469C467-622 was nonfunctional in both symbiosis and immunity.

In comparing performance between RLK4 and CERK6 receptor variants with luminescence-based assays, it was found that a CERK6 chimeric construct containing a HvRLK4 kinase domain was able to induce a response to chitin, whereas HvRLK4 normally does not (FIG. 13E) This indicated that the RLK4 kinase functioned similarly to the CERK6 kinase.

Following up on the testing of constructs with chimeric receptor zones described above, the JM-B, or zone 4, region of the entire CERK6 receptor was replaced with the corresponding region of NFR1 and compared its signalling capacity to CERK6 (FIG. 22E- receptor 23) when expressed in cerk6 mutants from native Cerk6 promoter (FIG. 22E- receptor 24). It was found that both receptors induced production of reactive oxygen species (ROS) after chitin treatment; however, receptor 24 led to an increased level of ROS compared to full-length CERK6 (FIG. 22E). This indicated that JM-B of NFR1 can alter the signalling capacity of CERK6 kinase.

### Example 13: JM-B Residues Provide Stabilizing Interactions for the Kinase Core

The following example describes experiments investigating the role of the JM-B motif, or the zone 4 motif, on stability of the kinase.

### Materials and Methods

Cloning, plant cultivation, hairy root transformation, Agrobacterium growth conditions, crystal production, and crystal analysis were as described in the above Examples.

### Results

The intracellular domains of Lotus japonicus CERK6 (residues 303-599) and Lotus japonicus NFR1 (residues 263-623) (FIGS. 22H-22I, top panels) were expressed and purified. When introducing a stabilizing mutation in the DFG motif, well-diffracting crystals of CERK6 D460N were obtained, but no crystals of NFR1 D462N were obtained (FIG. 22J, bottom panel). Instead, a structure of a symbiotic kinase was obtained for the NFR1 ortholog from Medicago truncatula LYK3 D459N (residues 302-597). The atomic structures of both CERK6 and LYK3 were determined to 2.3 Å and 2.5 Å resolution, respectively (FIG. 18C).

The structures revealed a classic kinase fold, and were striking in their similarity (RMSD=0.724 Å) despite the fact that LYK3 were crystallized with a nucleotide and CERK6 was not. When examining a superimposition of the two structures, it was found that the largest difference between them was located at the N-lobe where LYK3 is slightly rotated compared to CERK6. It is though that this rotation might be induced by the nucleotide binding.

The JM-B has previously been considered as a part of the juxtamembrane, which is usually depicted as a flexible region connecting the catalytic kinase domain to the transmembrane domain and the ectodomain. However, these CERK6 and LYK3 structures revealed JM-B to be clearly defined in the density as a loop structure followed by a helix positioned above the anti-parallel beta sheet (b2-b5) of the kinase N-lobe. Importantly, residues in the JM-B that were conserved (across both classes of chitin and Nod factor receptors) interacted with residues in the N-lobe (FIG. 18D). In LYK3, two residues located in the N-terminal of JM-B, K306 and E309 interacted with H370 and Y380, respectively located in the C-terminal look of αC. The JM-B helix interacted with the loop connecting β2 and β3 (E314 contacts R342) and a series of sidechain-backbone interactions involving T319, F322, S323 and N326 stabilize the helix (FIG. 18D, right panels). Corresponding residues of CERK6 were involved in coordinating the JM-B to the loop connecting β2 and β3 and to β5 and also in the sidechain-backbone interactions keeping the loop C-terminal of the helix in place. However, if LYK3 structure was stabilized primarily by sidechain interactions, the same result was obtained in CERK6 by primarily backbone interactions (FIG. 18D, left panels). The fact that the conserved residues in JM-B of both LYK3 and CERK6 were so specifically coordinated to the kinase N-lobe means that the JM-B is in fact not just a flexible region, but instead an integral part of the kinase core.

Three of JM-B residues that differ between CERK6 and NFR1 are located in the N-terminal loop, while the remaining three were on the helix, sidechains being surface exposed. In contrast to the rest of the JM-B residues, none of these made contact to the N-lobe of the kinase. This enabled receptor engineering without risk of compromising the structural integrity of the chimeras.

### Example 14: CERK6 Kinase Disrupts Root Hair Infection

The following example describes experiments investigating the role of the CERK6 kinase domains in root hair formation.

### Materials and Methods

Cloning, plant cultivation, hairy root transformation, and Agrobacterium growth conditions were as described in the above Examples. Nodulation assays were conducted as described in Example 11 unless stated otherwise.

Infection thread numbers were quantified at 35 days post inoculation with M. loti MAFF using a Zeiss Axioplan microscope with 546/575-640 excitation/emission (nm) parameters for detecting M. loti bacteria.

### Results

Nod factor receptors control both the developmental processes that take place during root nodule symbiosis, root nodule organogenesis, and root hair infection via infection thread formation and elongation. These two programs are tightly coordinated and the absence or impairment of one has an impact on the other (Miri et al. (2019) New Phytologist, 222(3):1523-37; Yano et al. (2008), PNAS 105(51):20540-45). Expression of NFR1 in the epidermis is critical for initiation of the root hair infection program. The Lotus nfr1 mutant is unable to initiate root hair deformation, bacteria attachment to the root, and the subsequent formation of micro-colonies in response to rhizobial infection (Radutoiu et al. (2003) Nature 425(6958):585-92).

Moreover, a constitutively active receptor complex formed by the nanobody-linked NFR1-NFR5 complex has the capacity to activate root nodule organogenesis but has a major negative impact on root infection (Rubsam et al. (2023) Science 379(6629):272-77). Together, these indicate that signaling from NFR1 and NFR5 needs to be tightly regulated.

In this study, it was identified that a chimera that contained the kinase domain from CERK6 and the EC and TM/JM from NFR1 had a reduced efficiency for root nodule organogenesis. Further, a chimera that contained the kinase domain and JM zone 4 from CERK6 and the EC and TM/JM zones 1-3 from NFR1 was nonfunctional compared to full-length NFR1 (FIG. 2B). Next, the impact of CERK6 kinase and zone 4 on infection by rhizobia was explored. The infection threads (ITs) that were formed in transformed roots 35 dpi with M. loti MAFF were quantified. The M. loti MAFF strain has dsRed incorporated into its chromosome, giving it the ability to detect bacteria present in micro-colonies and ITs. Typical root hair curling, micro-colonies, and elongated ITs were observed in wild type transformed with the empty vector, and in nfr1 transformed with the NFR1 complementation construct (full-length NFR1), but not in the nfr1 roots transformed with the empty vector (FIGS. 2H-2K).

A significant reduction in IT formation was observed for plants expressing the construct containing the kinase domain from CERK6 and the EC and TM/JM from NFR1, while no ITs were observed when the CERK6 kinase was coupled with the CERK6 zone 4 and the EC and TM/JM zones 1-3 from NFR1 (FIG. 2H). Multiple micro-colonies entrapped by curling root hairs were observed in transgenic roots expressing either chimeric receptor (FIG. 2J). This is surprising and indicated that the first root hair responses were initiated even in the absence of root nodule organogenesis in the presence of a chimeric receptor containing both zone 4 and kinase of CERK6. Furthermore, these results provide evidence that the CERK6 kinase functions less optimally compared to the NFR1 kinase during root hair infection.

Potential interaction of the JM-B and C-terminal region with inhibitor(s) for symbiosis and/or inhibition of interaction with a downstream partner was investigated by testing whether a GFP nanobody-tagged receptor 6, which was symbiotically inactive (FIG. 2G), would be able to induce spontaneous nodules when expressed together with the NFR5-GFP in the nfr1nfr5 mutant. It was found that, as previously reported (Rubsam et al. (2023), Science 379, 272-277), a nanobody-mediated complex of NFR1 and NFR5 induced nodule organogenesis in the absence of rhizobia, but not when CERK6 or receptor 6 were brought together with NFR5 (FIG. 21E). Collectively, these results pointed towards an independent role of the JM-B and C-terminal region of the kinases studied here in coordinating the balance between auto and substrate-phosphorylation and that signalling in symbiosis was promoted when the intracellular region adopted a conformation which favours auto-phosphorylation likely necessary for the interaction with NFR5.

### Example 15: NFR1 Variants Containing CERK6 Kinase do not Induce ROS Production after Treatment with Nod Factors

The following example describes experiments investigating ROS production after treatment with Nod factors in lines expressing NFR1 variants containing CERK6 kinase.

### Materials and Methods

Materials and methods were as described in the above Examples. For ROS measurement, 0.1 mg/ml chitin or 10⁻⁸M. loti R7A Nod factor were used.

### Results

The CERK6 kinase is essential for immune signaling resulting in robust ROS production in response to chitin in Lotus roots (Bozsoki et al. (2020) Science, 369(6504):663-70). In the present study, NFR1 chimeric receptors that contain the CERK6 kinase have been used to rescue the symbiotic phenotype in nfr1 roots, and they were identified to have a reduced functionality (FIG. 2B). It was hypothesized that impairment of root hair infection and nodulation in nfr1 lines expressing these chimeras could be a result of the activation of an immune response that inhibits rhizobial colonization. To test this, the production of ROS in transformed nfr roots expressing NFR1-CERK6 chimeric constructs after NF application was quantified using a luminescence-based assay. The capacity of these roots to activate ROS production after chitin treatment was also tested.

The tested constructs included: one chimeric construct with the EC and TM/JM from NFR1 and the K domain from CERK6; one chimeric construct with the EC from NFR1 and the TM/JM and K domains from CERK6; one chimeric construct with the EC and TM/JM zones 1-3 from NFR1 and JM zone 4 and the K domain from CERK6; one chimeric construct with the EC and JM zone 4 from NFR1 and TM/JM zones 1-3 and the K domain from CERK6; and one chimeric construct with the EC and K domains from NFR1 and the TM/JM from CERK6, all expressed under the NFR1 promotor. An additional negative control containing the EC and TM/JM domains from NFR1 and the CERK6 kinase domain with the kinase-dead (K351N) mutation was also included in this assay.

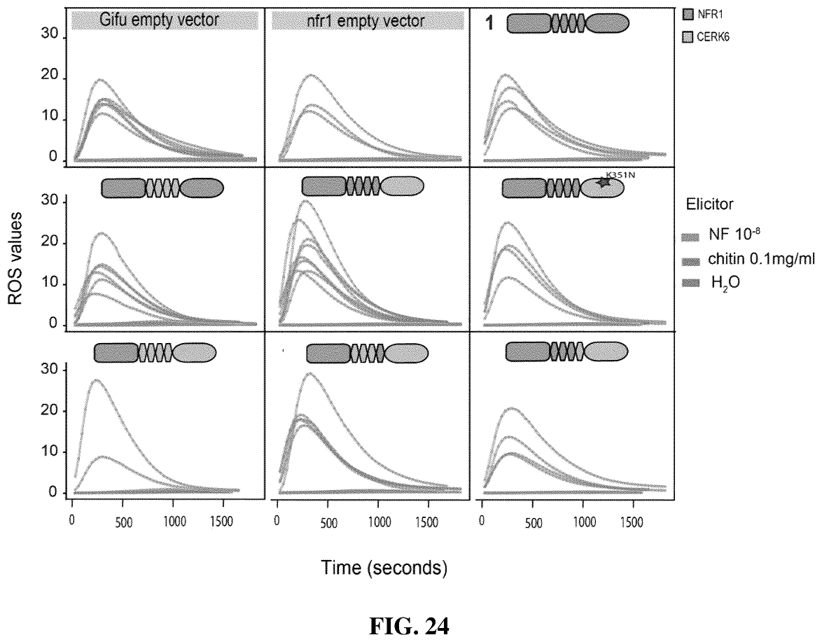

It was found that none of the tested roots, irrespective of the expressed construct, was able to induce ROS within 30 minutes after 10⁻⁸M M. loti R7A NF application. By contrast, all roots responded normally to chitin, since native CERK6 was present in the nfr1 mutant (FIG. 24), indicating that NFR1 variants containing CERK6 intracellular domains do not induce ROS production upon NF treatment. These results supported the hypothesis that impairment of the symbiotic phenotype in the presence of chimeric NFR1-CERK6 receptors containing different CERK6 intracellular domains does not occur due to activation of a ROS response.

### Example 16: NFR1 Variants Containing CERK6 Kinase do not Form Inhibitory Dimers with the Native CERK6

The following example describes experiments investigating whether NFR1 variants containing CERK6 kinase form inhibitory dimers with the native CERK6.

### Materials and Methods

Materials and methods were as described in the above Examples unless noted otherwise.

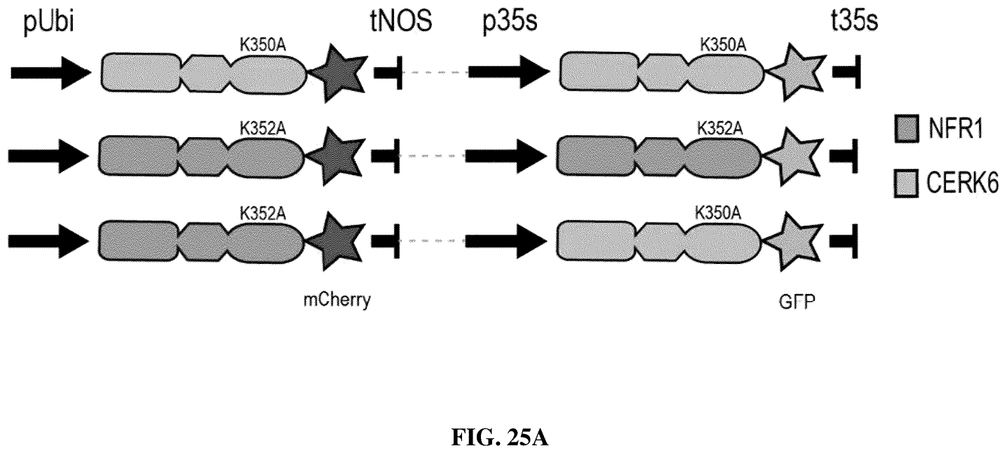

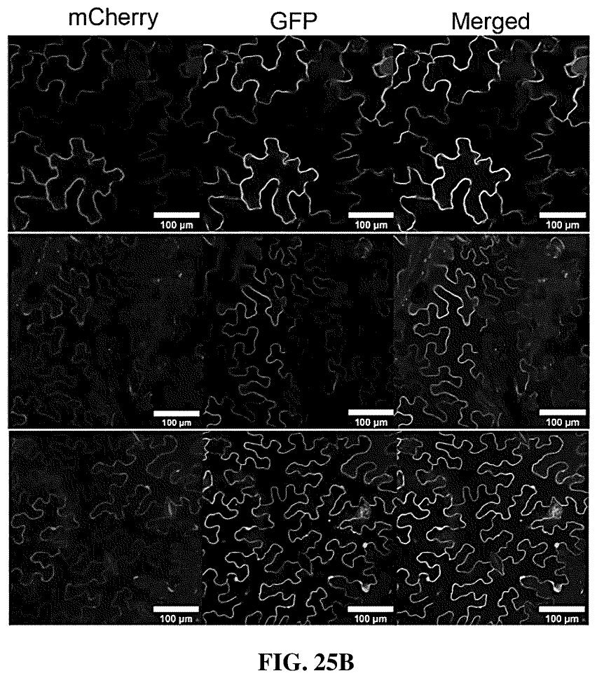

To test the hypothesis that NFR1 or CERK6 form homo- or heterodimers with each other, an in vitro co-immunoprecipitation assay is created for receptors expressed in N. benthamiana leaves, using a binary vector for transient transformation, in which chimeric receptors or receptor variants are tagged with eGFP on the C-terminal and expressed with a 35s promotor and 35s terminator, or tagged with mCherry on the C-terminal and expressed with a Ubiquitin (Ubi) promoter and NOS terminator. Fusion of the receptor variants with variable fluorescent tags, in this case GFP or mCherry, provides the opportunity to detect co-immunoprecipitated receptor molecules by performing Western blot analyses with the use of different antibodies. FIG. 25A shows an overview of the combinations with the two expression cassettes positioned in tandem in the same level 2 vector used for transient transformation of tobacco leaves. NFR1 (K352A) or CERK6 (K350A) kinase-dead variants are used in this assay to prevent cell death due to overexpression of the kinases.

### Results

RLK receptors form homo- and heteromeric complexes at the plasma membrane in order to activate their kinase. Often the formation of such complexes is forced by interactions between intracellular domains (Maeda et al. (2018) Biophys. J., 114(4):893-903; Petutschnig et al. (2010) JBC, 285(37):28902-11; Rubsam et al. (2023) Science, 379(6629):272-77). It has not been yet proven that NFR1 or CERK6 forms homodimers for signaling transduction in planta but there is evidence that they interact with their respective homomers to promote phosphorylation (Madsen et al. (2011) The Plant J., 65(3):404-17).

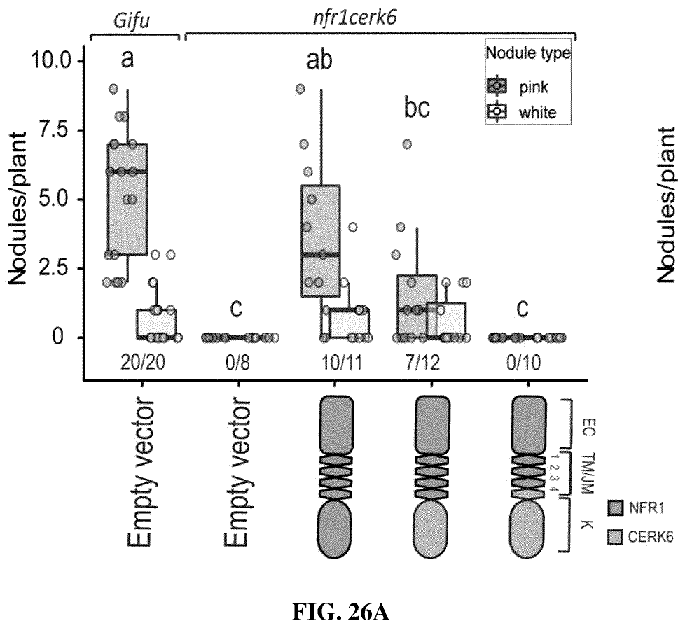

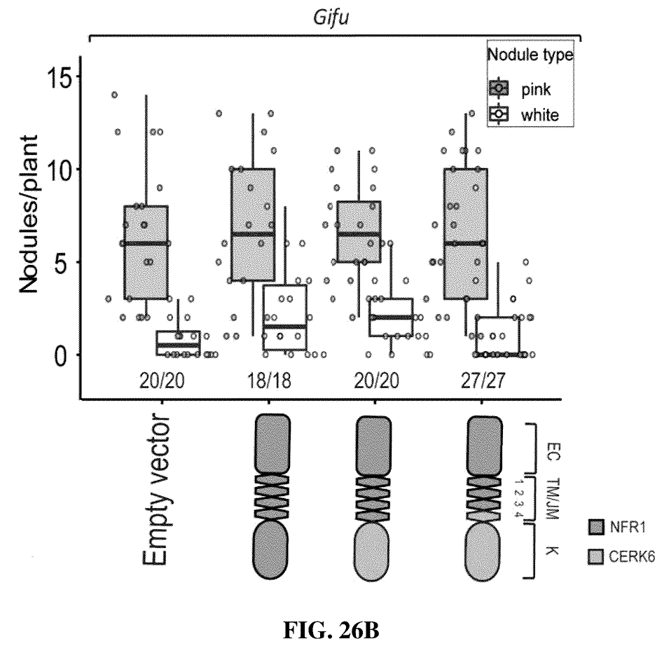

All the receptor variants were expressed and localized in the plasma membrane (FIG. 25B). First, it was hypothesized that CERK6 forms homodimers to activate immune signaling. If this hypothesis were true, it would suggest that the NFR1 receptor variants containing intracellular domains of CERK6 would be able to interact with native CERK6 molecules. To investigate whether NFR1-CERK6 chimeras form inhibitory interactions with the native CERK6 expressed in the nfr1 mutant and thus impair symbiosis, the chimeras were tested for their capacity to rescue nodulation in the nfr1cerk6 double mutant after inoculation with M. loti R7A dsRed. A 35 dpi evaluation of nodule formation revealed that both chimeric constructs containing either the CERK6 kinase with the remaining domains from NFR1, or the CERK6 kinase and zone 4 with the remaining domains from NFR1 behaved similarly in the single (FIG. 2B) and double (FIG. 26A) mutant backgrounds.

It was then hypothesized that NFR1 and CERK6 can form heterodimers through their intracellular domains, which might inhibit the function of NFR1 in symbiosis. To investigate whether NFR1-CERK6 chimeras inhibit the function of native NFR1 expressed in the Gifu wild type and thus lead to impaired nodulation, the chimeras were expressed in Gifu and nodulation events were assessed 35 dpi with M. loti R7A dsRed. All transformed Gifu roots induced nodulation with the same efficiency (FIG. 26B). In conclusion, nodulation was not impaired due to putative interactions between NFR1, CERK6, and NFR1-CERK6 chimeras.

### Example 17: NFR1 Zone 4 Biochemical Properties are Required for Symbiotic Signaling

The following example describes experiments investigating the NFR1 zone 4 biochemical properties that are required for symbiotic signaling.

### Materials and Methods

Materials and methods were as described in the above Examples unless noted otherwise.

### Results

The present study underlined the importance of NFR1 M306 and A308 in root nodule symbiosis signaling. In the presence of CERK6 kinase, substitutions of the NFR1 methionine or alanine with the corresponding threonine or aspartic acid from CERK6 impaired nodulation, probably due to a shift from a non-polar to a polar state. Methionine residues can act as “ROS sensors” by having the property of being oxidized to methionine sulfoxide (MetO), and therefore, they can regulate kinase function in the cytoplasm (Aledo (2019) Protein Science 28(10):1785-96.). In the reduced form, methionine can establish interactions with aromatic residues (phenylalanine, tyrosine, and tryptophan) that are present in close proximity, both inter- and intramolecularly, and can regulate their phosphorylation (Lewis et al. (2016) Nature Chemical Biology 12(10):860-66; Reid, Lindley, and Thornton (1985) FEBS Letters 190(2):209-13; Valley et al. (2012) JBC 287(42):34979-91; Veredas, Cant6n, and Aledo (2017) Scientific Reports 7(1):40403). Alanine is a small, non-polar residue that plays a significant role in the stabilization of protein conformations, giving a hydrophobic character to the corresponding protein region (Rohl, Fiori, and Baldwin (1999) PNAS 96(7):3682-87).

The role of M306 and A308 during symbiosis was examined by introducing mutagenized forms of Chimera A in the nfr1 mutant and testing for genetic complementation (see schematic representation in FIG. 7A). Three separate mutations were created for M306, resulting in threonine (M306T), valine (M306V), glutamine (M306Q) or alanine (M306A) substitutions. Threonine, which is the corresponding residue in CERK6, is a polar residue; its substitution was designed to investigate the significance of the non-polar state of M306. Valine is a residue similar in size to threonine; its substitution was designed to investigate if the small size of M306 is essential for nodulation. Glutamine is a MetO mimic; its substitution was designed to investigate if the oxidation state of M306 affects nodulation. Finally, substitution to alanine was designed to abolish all specific properties of methionine.

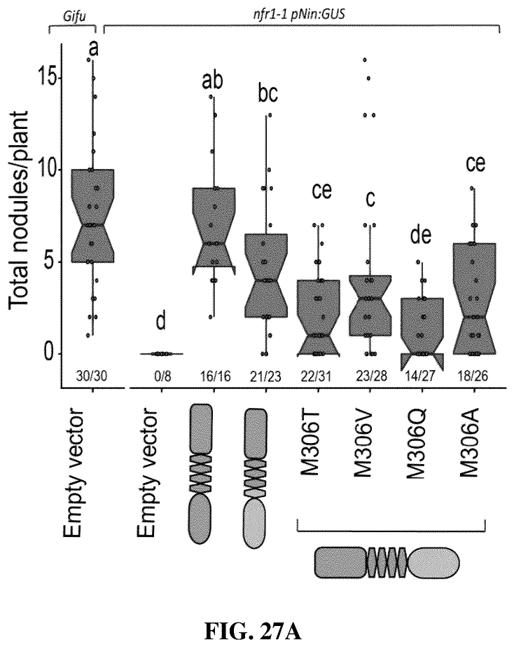

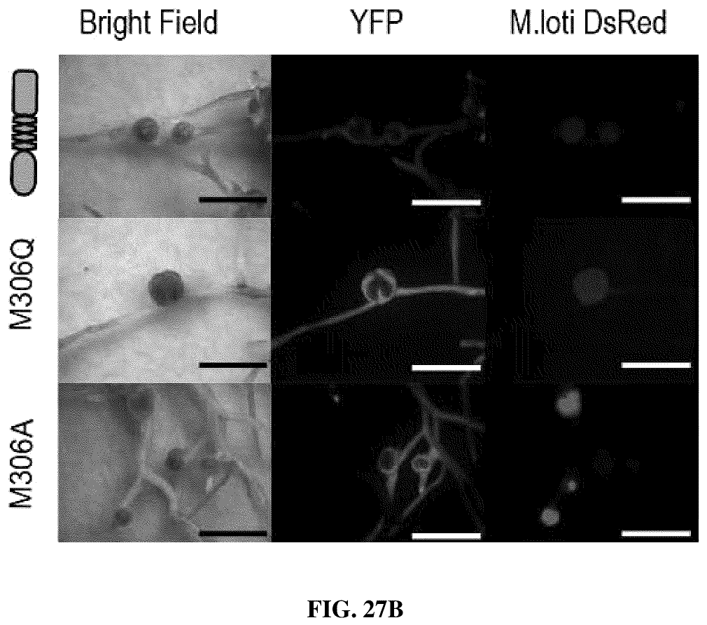

All four substitutions of the M306 resulted in a diminished symbiotic capacity when compared to Chimera A. The number of nodulating plants, as well as the number of nodules per plant was reduced (FIG. 27A). The receptor variant that carries the M306Q mutation had the lowest symbiotic efficiency, with a statistically significant reduction in the number of nodules: only 14 out of 27 tested plants were able to form nodules (FIGS. 27A-27B). This indicated that when M306 was fixed in an oxidized state (MetO) it impaired symbiotic signaling, perhaps due to the lack of its ability to interact with aromatic residues. Another hypothesis is that M306 regulates kinase activity in presence or absence of ROS due to alternations between oxidized and reduced states, something that was disrupted when it was fixed in a MetO state.

The role of A308 was investigated through mutation to aspartic acid (A308D), glutamic acid (A308E), or leucine (A308L) substitutions. Aspartic acid and glutamic acid, with the first being present in CERK6, are negatively charged residues; these substitutions were designed to investigate if the hydrophobic character of A308 is essential for signaling. The substitution to leucine, a larger and hydrophobic residue, was designed to investigate if the small size of A308 is crucial for its function.

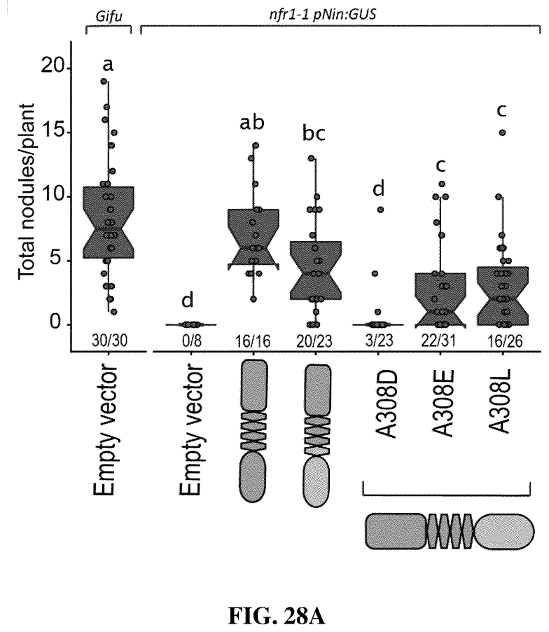

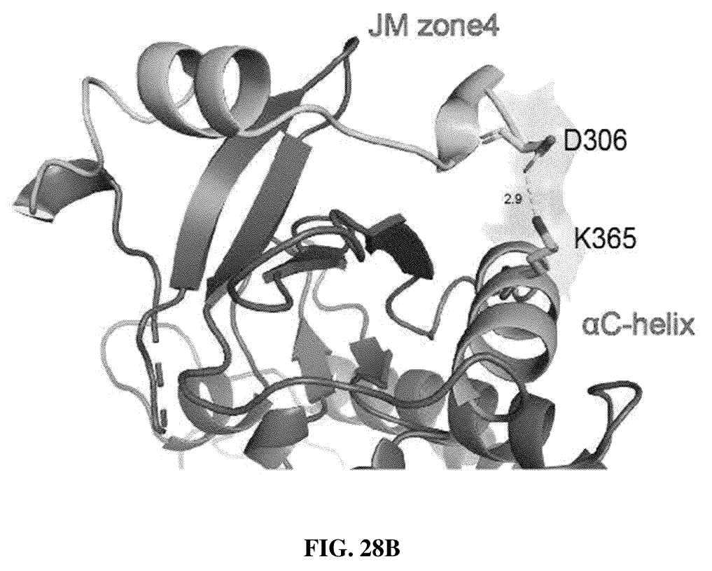

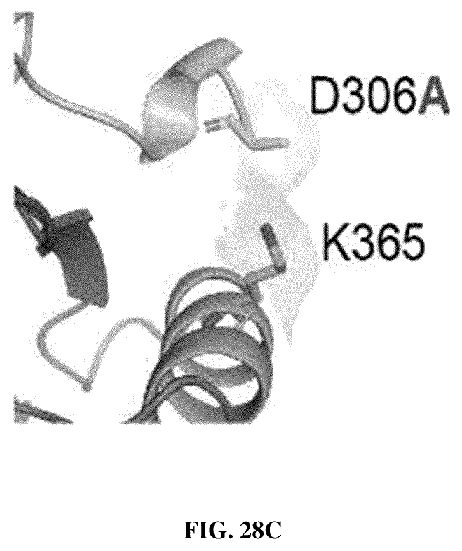

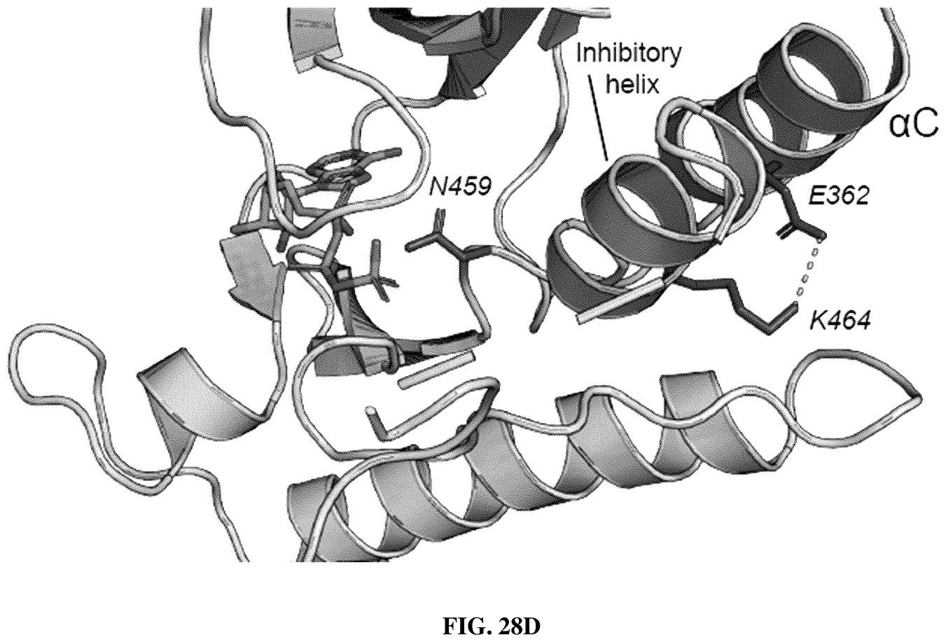

Results from the expression of receptor variants carrying the three A308 substitutions in nfr1-1 pNIN:GUS roots revealed a lower nodulation tendency than that of Chimera A (FIG. 7A). However, only the expression of the chimera carrying the A308D mutation had a significantly reduced nodulation performance (FIG. 28A and FIG. 7B). D306 in CERK6 is located in the N-terminus zone 4 loop that is extended towards the αC-helix of the CERK6 kinase. The Alpha fold structural prediction of a maize CERK6 homologue suggests the presence of a salt bridge between the conserved JM D306 and a conserved arginine in the αC-helix of the kinase. Preliminary analysis of the crystal structure of Lotus CERK6 revealed that D306 creates a 2.9 A salt bridge with the positively charged K365, which is highly conserved across CERK and NFR kinases, that is present in the regulatory αC-helix of the kinase (FIG. 28B). This interaction might play an inhibitory role in the CERK6 kinase function in symbiosis and thus the kinase must be functional when alanine is present, since it fails to form polar contacts with other residues (FIG. 28C). A similar salt bridge was also revealed between E362 and K464 for CERK6 and LYK3, potentially with a similar inhibitory effect (FIG. 28D).

Aspartic acid and glutamic acid are two very similar negatively charged residues, with the only obvious difference being their size, since aspartic acid has one fewer methyl group than glutamic acid. The D-K salt bridge appears to be stronger than the E-K in vitro, as the smaller size of aspartic acid's side chain stabilizes the interaction (Smith and Scholtz (1998) Biochemistry 37(1):33-40). If the stable interaction between zone 4 and the kinase of CERK6 is inhibitory for nodulation, this would explain why the A308E substitution did not have a dramatic impact on nodule formation.

### Example 18: Mutation in the NFR1 Activation Loop Abolishes Nodulation

The following example describes experiments investigating the effects on nodulation of mutations in the NFR1 activation loop (AL).

### Materials and Methods

Materials and methods were as described in the above Examples unless noted otherwise.

Codon mutagenesis on the Activation loop (AL) was performed utilizing the Q5® Site-Directed Mutagenesis Kit with primer pairs designed with the NEBaseChanger online tool following the manufacturer's manual. The mutated clones were transformed into chemical competent E. coli TOP10.

### Results

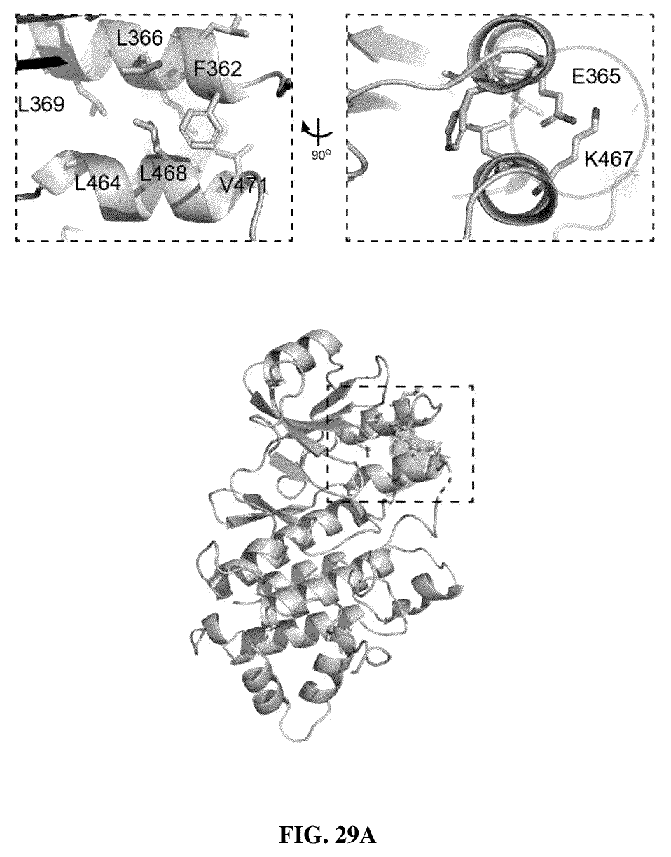

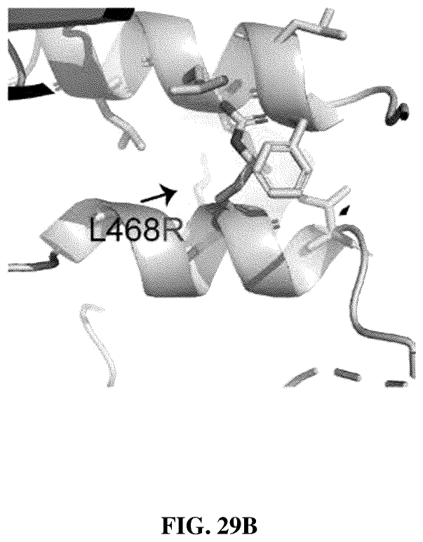

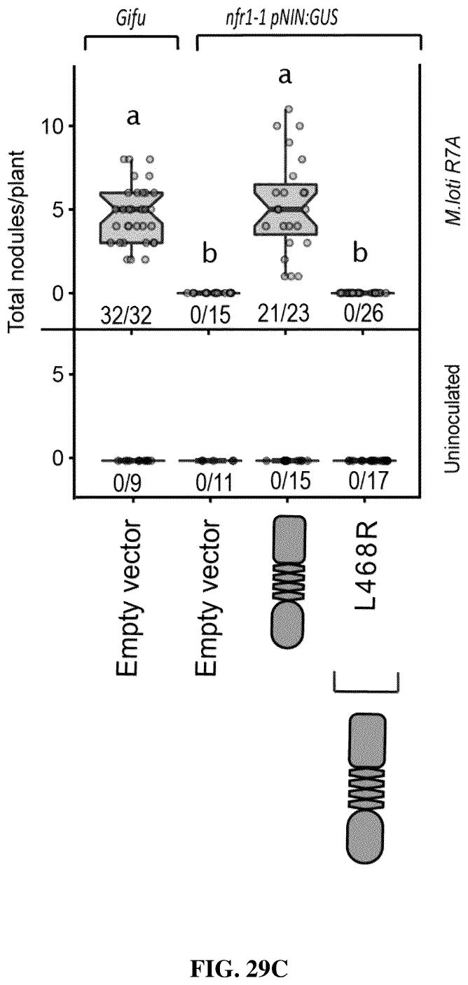

In the MtLYK3 structure, part of the AL appeared to form an α-helix that was parallel to the regulatory αC-helix (FIG. 17C). This conformation has been characterized as an inhibitory mechanism (FIG. 28D) in mammalian tyrosine-kinases that blocks substrate binding and can be reverted by either phosphorylation of the AL or reorientation of the αC-helix (Jeffrey et al. (1995) Nature 376(6538):313-20; Levinson et al. (2006) PLOS Biology 4(5):e144; Wang et al. (2019) JBC 294(12):4511-19). This inhibitory conformation was stabilized by a salt bridge between a glutamic acid located in the AL and an arginine located in the αC-helix. In the NFR1 homology model based on the MtLYK3 crystal structure, K467, in the AL, and E365, in the αC-helix, were found in close proximity to each other, which may allow them to create a salt bridge (FIG. 29A). Moreover, the surface created between the two parallel helixes was non-polar, consisting primarily of leucine and phenylalanine residues, which might promote the formation of the salt bridge mentioned above by keeping a hydrophobic interface between the two helixes (FIGS. 29A-29B).

It was hypothesized that the helical conformation of the NFR1 AL was inhibitory for nodulation and that release of this conformation might lead to a constitutive active kinase. To test this hypothesis, hydrophobic interface between two parallel helixes were disrupted by substituting leucine468, which was located in the AL, with a positive-charged arginine. The mutated NFR1 receptor was then expressed in nfr1 roots, and its efficiency in nodule formation with or without inoculation with M. loti R7A was evaluated. No spontaneous nodules were formed in the uninoculated transformed root, indicating that the L468R mutation did not create a constitutively active kinase form that engages in signaling transduction in absence of NF (FIG. 29C). Surprisingly, none of the transformed roots inoculated with M. loti R7A induced nodule organogenesis, which means that the mutation in the AL created an inactive form of the kinase or a kinase that lacks symbiotic function (FIG. 29C).

### Example 18: The Role of CERK6 Juxtamembrane in Immune Signaling

The following example describes experiments investigating the role of CERK6 juxtamembrane in immune signaling.

### Materials and Methods

Materials and methods were as described in the above Examples unless noted otherwise.

### Results

A previously published study (Bozsoki et al. (2020) Science 369(6504):663-70) utilizing chimera-based approaches to investigate the role of NFR1 and CERK6 intracellular domains identified that the CERK6 core kinase was essential for immune signaling, since its substitution with the corresponding NFR1 kinase failed to initiate the immune pathway that leads to ROS production. In the same study, it was reported that when the transmembrane and juxtamembrane domains of CERK6 are exchanged with the corresponding regions in NFR1, this leads to a low intensity of ROS response (see FIG. 1 of Bozsoki et al. (2020) Science 369(6504):663-70).

These results could be explained by two hypotheses: either the symbiotic NFR1 kinase lost immune-triggering traits during evolution, or it evolved an inhibitory mechanism by a cooperation between the kinase and the TM/JM domain to assure that no robust ROS responses will be induced during NF signaling. To further investigate the role of intracellular CERK6 and NFR1 regions in immunity, additional experiments were performed using chimera-swap approaches.

CERK6 is ubiquitously expressed in all tissues regardless infection from pathogens, and induces ROS production within minutes upon chitin perception (Bozsoki et al. (2017) PNAS 114(38):E8118-27; Lohmann et al. (2010) MPMI 23(4):510-21). This suggests that the receptor is present in the plasma membrane in a resting state that rapidly can be reverted to an active state to induce a robust defense mechanism in response to pathogen invasion. Results from the present study suggest that the existence of an allosteric mechanism between CERK6 zone 4 and the kinase (K) (see FIG. 28B) abolishes nodule organogenesis when coupled with the rest of the NFR1 receptor regions. It was hypothesized that these JM-K interactions inhibit CERK6 kinase's activity in absence of chitin and are released only after chitin perception.

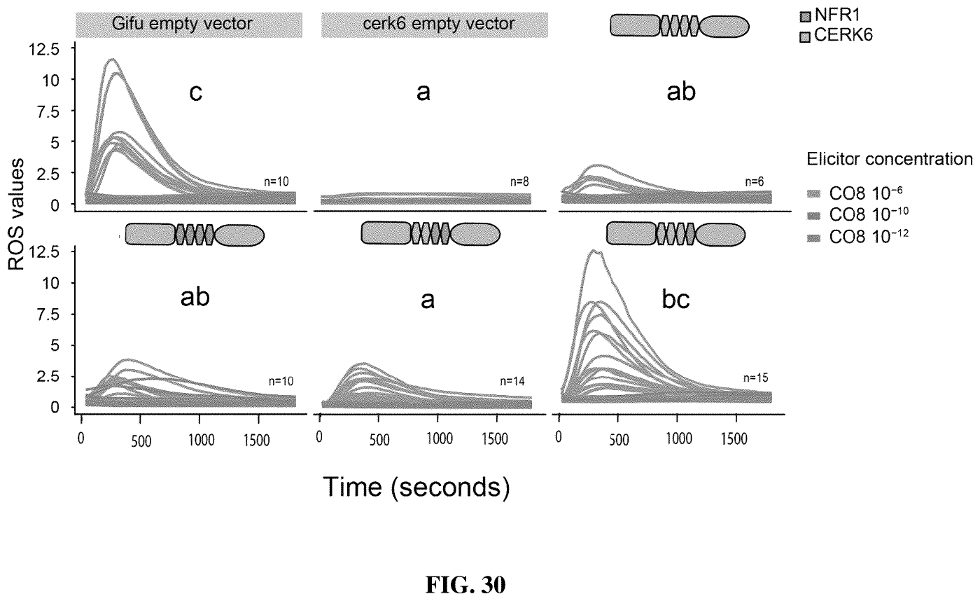

To test if substitution of CERK6 JM regions with the corresponding NFR1 lead to a constitutive active CERK6 or increases the ROS-response intensity upon perception of chitin, a series of swap-domain chimeras between CERK6 and NFR1 TM/JM zones coupled with the EC and K of CERK6 were constructed and expressed in the cerk6 mutant roots. Efficiency of the different constructs was evaluated in a Luminescence-based assay for ROS detection within 30 minutes upon chitin octamer (C08) application in different concentrations. None of the tested constructs or the Gifu wild type and CERK6 complementation were able to induce ROS production with the lower concentrations of C08, (C08-10 and C08-12), (FIG. 30) something that has been reported previously for the native CERK6 receptor (Bozsoki et al. (2017) PNAS 114(38):E8118-27). Also, none of the tested chimeras resulted in a constitutively active receptor, since no cell death was observed in the transformed roots due to ROS overproduction. The constructs containing the entire TM/JM or only zones 3 and 4 of NFR1 behaved similarly to full-length CERK6, while the presence of NFR1 zone 4 alone resulted in an increase in ROS intensity, although it was non-statistically significant compared to the CERK6 complementation, which resembles native Gifu conditions (FIG. 30). It is important to note that in this experiment, the cerk6 roots expressing CERK6 full-length receptor showed a low-intensity ROS response when compared to wild type roots; thus, it was difficult to directly compare the level of ROS production induced by the chimeric constructs. These data indicate that NFR1 zone 4 provided an increased signaling response from the CERK6 kinase when compared to the full-length CERK6.

### Example 19: CERK6 Activation Loop Forms an Inhibitory Helical Conformation in the Absence of the Ligand

The following example describes experiments investigating the structure of the CERK6 activation loop in the absence of the ligand.

### Materials and Methods

Materials and methods were as described in the above Examples unless noted otherwise.

### Results

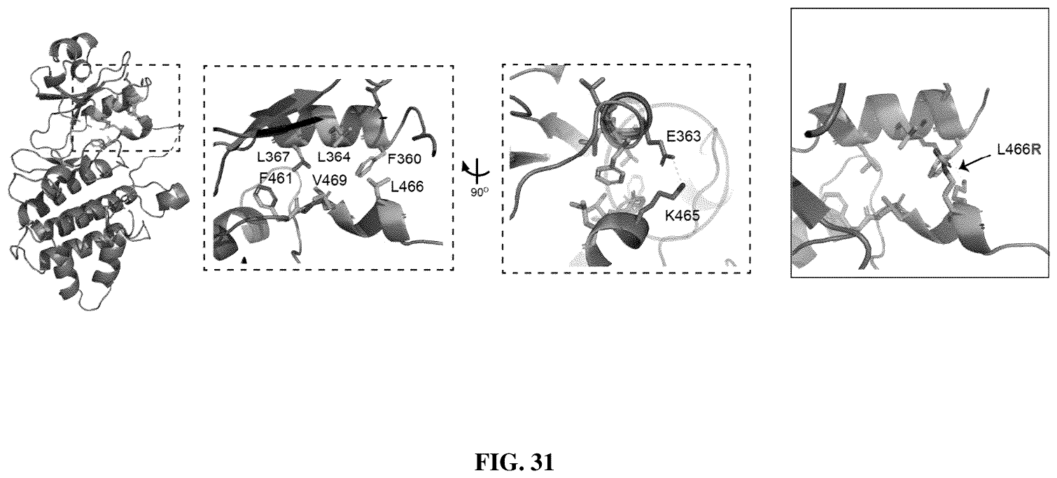

The CERK6 crystal structure revealed that, like in the case of MtLYK3, part of the AL forms an α-helix that was parallel to the regulatory αC-helix (FIG. 31, Table 2). Similarly, the role of the putative inhibitory AL helical conformation in immune signaling was investigated. L466, which was located in the CERK6 AL and was part of the hydrophobic interface between the two helixes, was substituted with the negatively charged arginine, i.e. L466R (FIG. 31). This mutant version of CERK6 was expressed in cerk6 using hairy root transformation, but no transformed roots were developed (Table 2).

TABLE 2
Mutation on the activation loop of CERK6 results in cell death.
Number of Gifu or cerk6 plants used for hairy root transformation
with the indicated constructs. The last column indicates the number
of plants that had hairy roots 21 days post transformation.

| | | Plants used for | Plants with hairy |
|---|---|---|---|
| Genotype | Construct | transformation | roots |
| Gifu | Empty vector | 60 | 40 |
| cerk6 | Empty vector | 45 | 42 |
| cerk6 | CERK6 | 60 | 37 |
| cerk6 | CERK6 (L466R) | 60 | 0 |

This phenotype has been observed before when the CERK6 receptor was overexpressed in cerk6 roots (unpublished data). Overexpression of CERK6 results in cell death, likely due to overactivation of the immune response (Bozsoki et al. (2017) PNAS 114(38):E8118-27). These data suggest that the helical conformation of the CERK6 AL was inhibitory for immune signaling by keeping the kinase in an “off” state in the absence of elicitor, and disruption of this conformation leads to a constitutively active kinase. CERK6 and LYK3 protomers were also captured in the crystals in an inactive state where the αC helix and the inhibitory helix of the activation loops kept each other in an “off” state by the salt bridge established between the glutamate E362 and lysine K464 (FIG. 28D).

1. A modified plant LysM receptor polypeptide comprising a first JM zone 4, wherein the first JM zone 4 has been modified by insertion, deletion, or substitution of one or more amino acids in the first JM zone 4 with the corresponding amino acids from a second JM zone 4 of a second plant LysM receptor polypeptide, wherein the first JM zone 4, the second JM zone 4, or both correspond to amino acids 305 to 327 when aligned to SEQ ID NO: 1 or correspond to amino acids 303 to 325 when aligned to SEQ ID NO: 8.

2. The modified plant LysM receptor polypeptide of claim 1, wherein:
(i) the first JM zone 4 is modified by substituting at least two or at least three amino acid residues in the first JM zone 4 with corresponding amino acid residues that are different in the second JM zone 4, or wherein the first JM zone 4 is modified by substituting three amino acid residues in the first JM zone 4 with corresponding amino acid residues that are different in the second JM zone 4, and/or wherein substitution comprises deletion of an amino acid not found in the second JM zone 4 and insertion of an amino acid found in the second JM zone 4 but not in the first JM zone 4;
(ii) the second JM zone 4 is able to initiate NFR1-mediated root nodule symbiosis signaling, ROS signaling, or different signaling than the first JM zone 4; and/or
(iii) wherein the substituted amino acid residues are selected from amino acid residues corresponding to M306, A308, and K320 of SEQ ID NO: 1 or from amino acid residues corresponding to T304, D306, and T318 of SEQ ID NO: 8.

3. (canceled)

4. The modified plant LysM receptor polypeptide of claim 2,
(i) wherein the second JM zone 4 is able to initiate NFR1-mediated root nodule symbiosis signaling;
(ii) wherein the first JM zone 4 comprises SEQ ID NO: 13, is selected from the group of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40, or comprises SEQ ID NO: 60, or conservative substitutions thereof; and/or wherein the second JM zone 4 comprises SEQ ID NO: 6, or is selected from the group of SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and SEQ ID NO: 52, or conservative substitutions thereof;
(iii) wherein the second JM zone 4 is able to initiate ROS signaling; or
(iv) wherein the first JM zone 4 comprises SEQ ID NO: 6, or is selected from the group of SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and SEQ ID NO: 52, or
conservative substitutions thereof and/or wherein the second JM zone 4 comprises SEQ ID NO: 13, or is selected from the group of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40, or conservative substitutions thereof.

5. (canceled)

6. A modified plant LysM receptor polypeptide comprising:
(1) a modified plant non-NFR1 LysM receptor polypeptide engineered for NFR1-mediated root nodule symbiosis signaling comprising:
(i) a first JM zone 4, wherein the first JM zone 4 was modified by substitution of one or more amino acids in the first JM zone 4 with the corresponding amino acids from a second JM zone 4 from an NFR1 LysM receptor polypeptide with NFR1-mediated root nodule symbiosis signaling; and/or
(ii) a first kinase C-terminus region, wherein the first kinase C-terminus region has been modified by substitution of one or more amino acids in the first kinase C-terminus region with the corresponding amino acids from a second kinase C-terminus region from a NFR1 LysM receptor polypeptide, wherein the first kinase C-terminus region corresponds to amino acids T467, S471, S473, P475, G477, V496, V500, Y510, D517, 1519, V520, S523, S525, 1526, T527, D528, G538, V539, S541, 0542, P543, T546, E547, D548, Q555, D559, V563, R567, M569, A573, K574, Q578, Q582, 1590, T600, D602, W604, V606, G607, F609, N612, N614, V616, M619, and G621 when aligned to SEQ ID NO: 8, and/or wherein the second kinase C-terminus region corresponds to amino acids I469, N473, T475, Q477, I497, I501, F511, N518, V520, L521, G524, L526, V527, A528, E529, E539, A540, N542, K543, S544, C547, D548, A549, P556, E560, E564, L568, 1570, G574, R575, R579, L583, L591, L601, E603, C605, D607, E608, S610, S613, T615, 1617, L620, and V622 when aligned to SEQ ID NO: 1: or
wherein the first kinase C-terminus region comprises amino acids T467, S471, S473, P475, G477, V496, V500, Y510, D517, 1519, V520, S523, S525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, and G621 of SEQ ID NO: 8; or
(2) a modified plant non-CERK6 LysM receptor polypeptide engineered for immune signaling comprising a first kinase N-terminus region, wherein the first kinase N-terminus region has been modified by substitution of one or more amino acids in the first kinase N-terminus region with the corresponding amino acids from a second kinase N-terminus region from a CERK6 LysM receptor polypeptide, wherein the first kinase N-terminus region corresponds to D328, A338, K347, K348, T349, V356, T360, C364, H371, C384, V385, H395, D397, G402, Y404, H406, K410, E411, S416, S417, A425, V445, and L456 when aligned to SEQ ID NO: 1, and/or wherein the second kinase N-terminus region corresponds to A326, S336, E345, R346, A347, M354, K358, A362, R369, S382, 1383, F393, E395, S400, H402, R404, R408, D409, A414, T415, S423, 1443, and Y454 when aligned to SEQ ID NO: 8: or
wherein the first kinase N-terminus region comprises D328, A338, K347, K348, T349, V356, T360, C364, H371, C384, V385, H395, D397, G402, Y404, H406, K410, E411, S416, S417, A425, V445, and L456 of SEQ ID NO: 1.

7. (canceled)

8. (canceled)

9. The modified plant LysM receptor polypeptide of claim 6,
wherein:
(1) the modified plant LysM receptor polypeptide is the non-NFR1 LysM receptor polypeptide, and wherein the first kinase C-terminus region is modified by substituting one or more amino acids of T467, S471, S473, P475, G477, V496, V500, Y510, D517, 1519, V520, S523, S525,1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, or G621 of SEQ ID NO: 8 with one or more amino acids of 1469, N473, T475, Q477, 1497,1501, F511, N518, V520, L521, G524, L526, V527, A528, E529, E539, A540, N542, K543, S544, C547, D548, A549, P556, E560, E564, L568, 1570, G574, R575, R579, L583, L591, L601, E603, C605, D607, E608, S610, S613, T615, 1617, L620, or V622 of SEQ ID NO: 1; or
(2) the modified plant LysM receptor polypeptide is the non-CERK6 LysM receptor polypeptide, and wherein the first kinase N-terminus region is modified by substituting one or more amino acids of D328, A338, K347, K348, T349, V356, T360, C364, H371, C384, V385, H395, D397, G402, Y404, H406, K410, E411, S416, S417, A425, V445, or L456 of SEQ ID NO: 1 with one or more amino acids of A326, S336, E345, R346, A347, M354, K358, A362, R369, S382, 1383, F393, E395, S400, H402, R404, R408, D409, A414, T415, S423, 1443, or Y454 of SEQ ID NO: 8.

10. (canceled)

11. (canceled)

12. (canceled)

13. (canceled)

14. The modified plant LysM receptor polypeptide of claim 1, further comprising a first kinase C-terminus region, wherein the first kinase C-terminus region has been modified by substitution of one or more amino acids in the first kinase C-terminus region with the corresponding amino acids from a second kinase C-terminus region from a NFR1 LysM receptor polypeptide; or
further comprising a first kinase N-terminus region, wherein the first kinase N-terminus region has been modified by substitution of one or more amino acids in the first kinase N-terminus region with the corresponding amino acids from a second kinase N-terminus region from a CERK6 LysM receptor polypeptide.

15. The modified plant LysM receptor polypeptide of claim 14, wherein the first kinase N-terminus region corresponds to D328, A338, K347, K348, T349, V356, T360, C364, H371, C384, V385, H395, D397, G402, Y404, H406, K410, E411, S416, S417, A425, V445, and L456 when aligned to SEQ ID NO: 1, and/or wherein the second kinase N-terminus region corresponds to A326, S336, E345, R346, A347, M354, K358, A362, R369, S382, 1383, F393, E395, S400, H402, R404, R408, D409, A414, T415, S423, 1443, and Y454 when aligned to SEQ ID NO: 8;
wherein the first kinase N-terminus region comprises D328, A338, K347, K348, T349, V356, T360, C364, H371, C384, V385, H395, D397, G402, Y404, H406, K410, E411, S416, S417, A425, V445, and L456 of SEQ ID NO: 1;
wherein the first kinase N-terminus region is modified by substituting one or more amino acids of D328, A338, K347, K348, T349, V356, T360, C364, H371, C384, V385, H395, D397, G402, Y404, H406, K410, E411, S416, S417, A425, V445, or L456 of SEQ ID NO: 1 with one or more amino acids of A326, S336, E345, R346, A347, M354, K358, A362, R369, S382, 1383, F393, E395, S400, H402, R404, R408, D409, A414, T415, S423, 1443, or Y454 of SEQ ID NO: 8; and/or
wherein the modified LysM receptor polypeptide is able to initiate ROS signaling.

16. (canceled)

17. (canceled)

18. The modified plant LysM receptor polypeptide of claim 6,
wherein:
(1) the first kinase C-terminus region of the modified non-NFR1 LysM receptor polypeptide has been modified by substitution of one or more amino acids in the first kinase C-terminus region with the corresponding amino acids from the second kinase C-terminus region from the NFR1 LysM receptor polypeptide, wherein the modified plant non-NFR1 LysM receptor polypeptide is able to initiate NFR1-mediated root nodule symbiosis signaling; or
(2) the first kinase N-terminus region of the modified non-CERK6 LysM receptor polypeptide has been modified by substitution of one or more amino acids in the first kinase N-terminus region with the corresponding amino acids from the second kinase N-terminus region from a CERK6 LysM receptor polypeptide, wherein the modified plant non-CERK6 LysM receptor polypeptide is able to initiate ROS signaling.

19. (canceled)

20. The modified plant LysM receptor polypeptide of claim 1, further comprising a first extracellular domain, wherein the first extracellular domain is modified as compared to the amino acid sequence of the corresponding unmodified plant LysM receptor polypeptide, optionally wherein the first extracellular domain is modified by substituting one or more amino acids of the first extracellular domain with one or more amino acids of a second extracellular domain, and/or wherein the second extracellular domain has a different affinity, selectivity, and/or specificity for oligosaccharides than the first extracellular domain.

21. A genetically modified plant or part thereof comprising a modified plant LysM receptor polypeptide comprising a first JM zone 4 modified by insertion, deletion, or substitution of one or more amino acids in the first JM zone 4 with the corresponding amino acids from a second JM zone 4 of a second plant LysM receptor polypeptide, wherein the first JM zone 4 comprises SEQ ID NO: 13, is selected from the group of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40, or conservative substitutions thereof, or comprises SEQ ID NO: 60, or conservative substitutions thereof, and/or wherein the second JM zone 4 comprises SEQ ID NO: 6, or is selected from the group of SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and SEQ ID NO: 52, conservative substitutions thereof; and
optionally further comprises:
(i) a first kinase C-terminus region, wherein the first kinase C-terminus region has been modified by substitution of one or more amino acids in the first kinase C-terminus region with the corresponding amino acids from a second kinase C-terminus region from a NFR1 LysM receptor polypeptide, wherein the first kinase C-terminus region corresponds to amino acids T467, S471, S473, P475, G477, V496, V500, Y510, D517, 1519, V520, S523, S525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, and G621 when aligned to SEQ ID NO: 8, and/or wherein the second kinase C-terminus region corresponds to amino acids 1469, N473, T475, Q477, 1497, I501, F511, N518, V520, L521, G524, L526, V527, A528, E529, E539, A540, N542, K543, S544, C547, D548, A549, P556, E560, E564, L568, 1570, G574, R575, R579, L583, L591, L601, E603, C605, D607, E608, S610, S613, T615, 1617, L620, and V622 when aligned to SEQ ID NO: 1; and/or
(ii) a first extracellular domain modified by substituting one or more amino acids of the first extracellular domain with one or more amino acids of a second extracellular domain, wherein the second extracellular domain has a different affinity, selectivity, and/or specificity for oligosaccharides than the first extracellular domain,
wherein the modified plant LysM receptor polypeptide is able to initiate NFR1-mediated root nodule symbiosis signaling.

22. (canceled)

23. A genetically modified plant or part thereof comprising a modified plant non-NFR1 LysM receptor polypeptide comprising a first JM zone 4, wherein the first JM zone 4 was modified by substitution of one or more amino acids in the first JM zone 4 with the corresponding amino acids from a second JM zone 4 from an NFR1 LysM receptor polypeptide with NFR1-mediated root nodule symbiosis signaling, wherein the first JM zone 4 comprises SEQ ID NO: 13, is selected from the group of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40, or comprises SEQ ID NO: 60, or conservative substitutions thereof, and/or
wherein the second JM zone 4 comprises SEQ ID NO: 6, or is selected from the group of SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and SEQ ID NO: 52, or conservative substitutions thereof; and optionally further comprises:
(i) a first kinase C-terminus region, wherein the first kinase C-terminus region has been modified by substitution of one or more amino acids in the first kinase C-terminus region with the corresponding amino acids from a second kinase C-terminus region from a NFR1 LysM receptor polypeptide, wherein the first kinase C-terminus region corresponds to amino acids T467, S471, S473, P475, G477, V496, V500, Y510, D517, 1519, V520, S523, S525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, and G621 when aligned to SEQ ID NO: 8, and/or wherein the second kinase C-terminus region corresponds to amino acids 1469, N473, T475, Q477, 1497, I501, F511, N518, V520, L521, G524, L526, V527, A528, E529, E539, A540, N542, K543, S544, C547, D548, A549, P556, E560, E564, L568, 1570, G574, R575, R579, L583, L591, L601, E603, C605, D607, E608, S610, S613, T615, 1617, L620, and V622 when aligned to SEQ ID NO: 1; and/or
(ii) a first extracellular domain modified by substituting one or more amino acids of the first extracellular domain with one or more amino acids of a second extracellular domain, wherein the second extracellular domain has a different affinity, selectivity, and/or specificity for oligosaccharides than the first extracellular domain,
wherein the modified plant non-NFR1 LysM receptor polypeptide is able to initiate NFR1-mediated root nodule symbiosis signaling.

24. (canceled)

25. The genetically modified plant or part thereof of claim 21, wherein the plant is selected from the group consisting of cassava, yam, sweet potato, corn, cowpea, rice, barley, wheat, Trema spp., apple, pear, plum, apricot, peach, almond, walnut, strawberry, raspberry, blackberry, red currant, black currant, melon, cucumber, pumpkin, squash, grape, bean, soybean, pea, chickpea, pigeon pea, lentil, Bambara groundnut, lupin, pulses, Medicago spp., Lotus spp., forage legumes, indigo, legume trees, and hemp.

26. A method of producing the genetically modified plant or part thereof of claim 21, comprising introducing a genetic alteration to the plant comprising a first nucleic acid sequence encoding the modified plant LysM receptor polypeptide or the modified plant non-NFR1 LysM receptor polypeptide.

27. The method of claim 26, wherein the nucleic acid sequence is operably linked to a promoter, wherein the promoter is a root specific promoter, an inducible promoter, a constitutive promoter, or a combination thereof;
optionally wherein the promoter is a root specific promoter, and wherein the promoter is selected from the group consisting of a NFR1 promoter, a NFR5 promoter, a LYK3 promoter, a CERK6 promoter, a NFP promoter, a Lotus japonicus NFR5 promoter (SEQ ID NO: 109), a Lotus japonicus NFR1 promoter (SEQ ID NO: 151), a Lotus japonicus CERK6 promoter (SEQ ID NO: 111), a Medicago truncatula NFP promoter (SEQ ID NO: 110), a Medicago truncatula LYK3 promoter (SEQ ID NO: 112), a maize metallothioneine promoter, a chitinase promoter, a maize ZRP2 promoter, a tomato LeExt1 promoter, a glutamine synthetase soybean root promoter, a RCC3 promoter, a rice antiquitin promoter, a LRR receptor kinase promoter, and an Arabidopsis pCO2 promoter;
optionally wherein the promoter is a constitutive promoter, and wherein the promoter is selected from the group consisting of a CaMV35S promoter, a derivative of the CaMV35S promoter, a maize ubiquitin promoter, a polyubiquitin promoter, a vein mosaic cassava virus promoter, and an Arabidopsis UBQ10 promoter; or
wherein the nucleic acid sequence is inserted into the genome of the plant so that the nucleic acid sequence is operably linked to an endogenous promoter, and wherein the endogenous promoter is a root specific promoter.

28. A method of producing the genetically modified plant or part thereof of claim 21, comprising genetically modifying the plant or part thereof by transforming the plant or part thereof with one or more gene editing components that target an endogenous nuclear genome sequence encoding an endogenous plant LysM receptor polypeptide or plant non-NFR1 LysM receptor polypeptide to genetically modify a first JM zone 4 by insertion, deletion, or substitution of one or more amino acids in the first JM zone 4 with the corresponding amino acids from a second JM zone 4 of a second plant LysM receptor polypeptide, and optionally further comprising:
(i) genetically modifying a first kinase C-terminus region by substitution of one or more amino acids in the first kinase C-terminus region with the corresponding amino acids from a second kinase C-terminus region from a NFR1 LysM receptor polypeptide; and/or
(ii) genetically modifying a first extracellular domain by substituting one or more amino acids of the first extracellular domain with one or more amino acids of a second extracellular domain, wherein the second extracellular domain has a different affinity, selectivity, and/or specificity for oligosaccharides than the first extracellular domain,
wherein the one or more gene editing components comprise a ribonucleoprotein complex that targets the nuclear genome sequence; a vector comprising a TALEN protein encoding sequence, wherein the TALEN protein targets the nuclear genome sequence; a vector comprising a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence.

29. (canceled)

30. (canceled)

31. (canceled)

32. An expression vector, isolated DNA molecule, or recombinant nucleic acid comprising a modified plant LysM receptor polypeptide comprising a modified JM zone 4 domain, a modified kinase C-terminus region, and/or a modified extracellular domain operably linked to at least one expression control sequence, wherein:
(i) the modified JM zone 4 was modified by substitution of one or more amino acids in a first JM zone 4 with the corresponding amino acids from a second JM zone 4 from an NFR1 LysM receptor polypeptide with NFR1-mediated root nodule symbiosis signaling,
wherein the first JM zone 4 comprises SEQ ID NO: 13, is selected from the group of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40, or conservative substitutions thereof, or comprises SEQ ID NO: 60, or conservative substitutions thereof, and/or
wherein the second JM zone 4 comprises SEQ ID NO: 6, or is selected from the group of SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, and SEQ ID NO: 52, or conservative substitutions thereof;
(ii) the modified kinase C-terminus region was modified by substitution of one or more amino acids in a first kinase C-terminus region with the corresponding amino acids from a second kinase C-terminus region from a NFR1 LysM receptor polypeptide, wherein the first kinase C-terminus region corresponds to amino acids T467, S471, S473, P475, G477, V496, V500, Y510, D517, 1519, V520, S523, S525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, and G621 when aligned to SEQ ID NO: 8, and/or wherein the second kinase C-terminus region corresponds to amino acids 1469, N473, T475, Q477, I497, I501, F511, N518, V520, L521, G524, L526, V527, A528, E529, E539, A540, N542, K543, S544, C547, D548, A549, P556, E560, E564, L568, 1570, G574, R575, R579, L583, L591, L601, E603, C605, D607, E608, S610, S613, T615, 1617, L620, and V622 when aligned to SEQ ID NO: 1; and/or
(iii) the modified extracellular domain was modified by substituting one or more amino acids of the first extracellular domain with one or more amino acids of a second extracellular domain, wherein the second extracellular domain has a different affinity, selectivity, and/or specificity for oligosaccharides than the first extracellular domain.

33. (canceled)

34. A bacterial cell or an Agrobacterium cell comprising the expression vector, isolated DNA molecule, or recombinant nucleic acid of claim 32.

35. A genetically modified plant, plant part, plant cell, or seed comprising the expression vector, isolated DNA molecule, or recombinant nucleic acid of claim 32.

36. A composition or kit comprising the expression vector, isolated DNA molecule, or recombinant nucleic acid of claim 32 with a bacterial cell or Agrobacterium cell.

37. A method of initiating NFR1-mediated root nodule symbiosis signaling comprising: introducing a genetic alteration via the expression vector, isolated DNA molecule, or recombinant nucleic acid of claim 32 to a cell, optionally wherein the cell is a plant cell.

38. (canceled)

39. A method of generating a modified plant LysM receptor polypeptide, comprising:
(a) aligning an amino acid sequence of a candidate receptor to SEQ ID NO: 8 to identify a first JM zone 4 corresponding to amino acids 303 to 325 of SEQ ID NO: 8, or aligning an amino acid sequence of a candidate receptor to SEQ ID NO: 1 to identify a first JM zone 4 corresponding to amino acids 305-327 of SEQ ID NO: 1, and optionally further aligning the JM zone 4 of the candidate receptor to SEQ ID NO: 13, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40;
(b) modifying the first JM zone 4 by substituting at least two or at least three amino acid residues in the first JM zone 4 with corresponding amino acid residues that are different in a second JM zone 4; and
(c) generating the modified plant LysM receptor polypeptide wherein the first JM zone 4 has been substituted with corresponding amino acid residues from the second JM zone 4; and
optionally further comprising:
(d-1) aligning an amino acid sequence of a candidate receptor to SEQ ID NO: 8 to identify a first kinase C-terminus region corresponding to amino acids T467, S471, S473, P475, G477, V496, V500, Y510, D517, 1519, V520, S523, S525, 1526, T527, D528, G538, V539, S541, Q542, P543, T546, E547, D548, Q555, D559, V563, R567, M569, A573, K574, Q578, Q582, 1590, T600, D602, W604, V606, G607, F609, N612, N614, V616, M619, and G621 of SEQ ID NO: 8, or aligning an amino acid sequence of a candidate receptor to SEQ ID NO: 1 to identify a first kinase C-terminus region corresponding to amino acids 1469, N473, T475, Q477, I497, I501, F511, N518, V520, L521, G524, L526, V527, A528, E529, E539, A540, N542, K543, S544, C547, D548, A549, P556, E560, E564, L568, 1570, G574, R575, R579, L583, L591, L601, E603, C605, D607, E608, S610, S613, T615, 1617, L620, and V622 of SEQ ID NO: 1;
(e-1) modifying the first kinase C-terminus region by substituting one or more amino acid residues in the first kinase C-terminus region with corresponding amino acid residues that are different in a second kinase C-terminus region; and
(f-1) generating the modified plant LysM receptor polypeptide wherein the first kinase C-terminus region has been substituted with corresponding amino acid residues from the second kinase C-terminus region; and/or
(d-2) aligning an amino acid sequence of a candidate receptor to SEQ ID NO: 1 to identify a first kinase N-terminus region corresponding to amino acids D328, A338, K347, K348, T349, V356, T360, C364, H371, C384, V385, H395, D397, G402, Y404, H406, K410, E411, S416, S417, A425, V445, and L456 of SEQ ID NO: 1, or aligning an amino acid sequence of a candidate receptor to SEQ ID NO: 8 to identify a first kinase N-terminus region corresponding to amino acids A326, S336, E345, R346, A347, M354, K358, A362, R369, S382, 1383, F393, E395, S400, H402, R404, R408, D409, A414, T415, S423,1443, and Y454 of SEQ ID NO: 8;
(e-2) modifying the first kinase N-terminus region by substituting one or more amino acid residues in the first kinase N-terminus region with corresponding amino acid residues that are different in a second kinase N-terminus region; and
(f-2) generating the modified plant LysM receptor polypeptide wherein the first kinase N-terminus region has been substituted with corresponding amino acid residues from the second kinase N-terminus region.

40. (canceled)

41. (canceled)

42. The modified plant LysM receptor polypeptide produced by the method of claim 39.


SEQUENCE LISTING (159 sequences)

SEQ ID NO: 1 - protein, 623 aa, Lotus japonicus
MKLKTGLLLFFILLLGHVCFHVESNCLKGCDLALASYYILPGVFILQNITTFMQSEIVSSNDAITSYNKDKILNDINIQSFQRLNIPFPCDCIGGEFLGHVFEYSASKGDTYETIANLYYANLTTVDLLKRFNSYDPKNIPVNAKVNVTVNCSCGNSQVSKDYGLFITYPIRPGDTLQDIANQSSLDAGLIQSFNPSVNFSKDSGIAFIPGRYKNGVYVPLYHRTAGLASGAAVGISIAGTFVLLLLAFCMYVRYQKKEEEKAKLPTDISMALSTQDGNASSSAEYETSGSSGPGTASATGLTSIMVAKSMEFSYQELAKATNNFSLDNKIGQGGFGAVYYAELRGKKTAIKKMDVQASTEFLCELKVLTHVHHLNLVRLIGYCVEGSLFLVYEHIDNGNLGQYLHGSGKEPLPWSSRVQIALDAARGLEYIHEHTVPVYIHRDVKSANILIDKNLRGKVADFGLTKLIEVGNSTLQTRLVGTFGYMPPEYAQYGDISPKIDVYAFGVVLFELISAKNAVLKTGELVAESKGLVALFEEALNKSDPCDALRKLVDPRLGENYPIDSVLKIAQLGRACTRDNPLLRPSMRSLVVALMTLSSLTEDCDDESSYESQTLINLLSVR

SEQ ID NO: 2 - protein, 226 aa, Lotus japonicus
MKLKTGLLLFFILLLGHVCFHVESNCLKGCDLALASYYILPGVFILQNITTFMQSEIVSSNDAITSYNKDKILNDINIQSFQRLNIPFPCDCIGGEFLGHVFEYSASKGDTYETIANLYYANLTTVDLLKRFNSYDPKNIPVNAKVNVTVNCSCGNSQVSKDYGLFITYPIRPGDTLQDIANQSSLDAGLIQSFNPSVNFSKDSGIAFIPGRYKNGVYVPLYHRTA

SEQ ID NO: 3 - protein, 29 aa, Lotus japonicus
GLASGAAVGISIAGTFVLLLLAFCMYVRY

SEQ ID NO: 4 - protein, 25 aa, Lotus japonicus
QKKEEEKAKLPTDISMALSTQDGNA

SEQ ID NO: 5 - protein, 24 aa, Lotus japonicus
SSSAEYETSGSSGPGTASATGLTS

SEQ ID NO: 6 - protein, 23 aa, Lotus japonicus
IMVAKSMEFSYQELAKATNNFSL

SEQ ID NO: 7 - protein, 296 aa, Lotus japonicus
DNKIGQGGFGAVYYAELRGKKTAIKKMDVQASTEFLCELKVLTHVHHLNLVRLIGYCVEGSLFLVYEHIDNGNLGQYLHGSGKEPLPWSSRVQIALDAARGLEYIHEHTVPVYIHRDVKSANILIDKNLRGKVADFGLTKLIEVGNSTLQTRLVGTFGYMPPEYAQYGDISPKIDVYAFGVVLFELISAKNAVLKTGELVAESKGLVALFEEALNKSDPCDALRKLVDPRLGENYPIDSVLKIAQLGRACTRDNPLLRPSMRSLVVALMTLSSLTEDCDDESSYESQTLINLLSVR

SEQ ID NO: 8 - protein, 622 aa, Lotus japonicus
MEHPRLGFPITLLLFSFILLPSTSQSKCTHGCALAQASYYLLNGSNLTYISEIMQSSLLTKPEDIVSYNQDTIASKDSVQAGQRINVPFPCDCIEGEFLGHTFQYDVQKGDRYDTIAGTNYANLTTVEWLRRFNSYPPDNIPDTGTLNVTVNCSCGDSGVGDYGLFVTYPLRPGETLGSVASNVKLDSALLQKYNPNVNFNQGSGIVYIPAKDQNGSYVLLGSSSGGLAGGAIAGIAAGVAVCLLLLAGFIYVGYFRKKRIQKEELLSQETRAIFPQDGKDENPRSTVNETPGPGGPAAMAGITVDKSVEFSYDELATATDNFSLANKIGQGGFGSVYYAELRGERAAIKKMDMQASKEFLAELKVLTRVHHLNLVRLIGYSIEGSLFLVYEFIENGNLSQHLRGSGRDPLPWATRVQIALDSARGLEYIHEHTVPVYIHRDIKSANILIDKNYRGKVADFGLTKLTEVGSSSLPTGRLVGTFGYMPPEYAQYGDVSPKVDVYAFGVVLYELISAKDAIVKTSESITDSKGLVALFEGVLSQPDPTEDLRKLVDQRLGDNYPVDSVRKMAQLAKACTQDNPQLRPSMRSIVVALMTLSSTTDDWDVGSFYENQNLVNLMSGR

SEQ ID NO: 9 - protein, 226 aa, Lotus japonicus
MEHPRLGFPITLLLFSFILLPSTSQSKCTHGCALAQASYYLLNGSNLTYISEIMQSSLLTKPEDIVSYNQDTIASKDSVQAGQRINVPFPCDCIEGEFLGHTFQYDVQKGDRYDTIAGTNYANLTTVEWLRRFNSYPPDNIPDTGTLNVTVNCSCGDSGVGDYGLFVTYPLRPGETLGSVASNVKLDSALLQKYNPNVNFNQGSGIVYIPAKDQNGSYVLLGSSSG

SEQ ID NO: 10 - protein, 29 aa, Lotus japonicus
GLAGGAIAGIAAGVAVCLLLLAGFIYVGY

SEQ ID NO: 11 - protein, 26 aa, Lotus japonicus
FRKKRIQKEELLSQETRAIFPQDGKD

SEQ ID NO: 12 - protein, 21 aa, Lotus japonicus
ENPRSTVNETPGPGGPAAMAG

SEQ ID NO: 13 - protein, 23 aa, Lotus japonicus
ITVDKSVEFSYDELATATDNFSL

SEQ ID NO: 14 - protein, 297 aa, Lotus japonicus
ANKIGQGGFGSVYYAELRGERAAIKKMDMQASKEFLAELKVLTRVHHLNLVRLIGYSIEGSLFLVYEFIENGNLSQHLRGSGRDPLPWATRVQIALDSARGLEYIHEHTVPVYIHRDIKSANILIDKNYRGKVADFGLTKLTEVGSSSLPTGRLVGTFGYMPPEYAQYGDVSPKVDVYAFGVVLYELISAKDAIVKTSESITDSKGLVALFEGVLSQPDPTEDLRKLVDQRLGDNYPVDSVRKMAQLAKACTQDNPQLRPSMRSIVVALMTLSSTTDDWDVGSFYENQNLVNLMSGR

SEQ ID NO: 15 - protein, 23 aa, Lotus japonicus
ITVDKSVEFSYDELATATDNFSL

SEQ ID NO: 16 - protein, 23 aa, Medicago truncatula
ITVDKSVEFSYDELAAASDNFSM

SEQ ID NO: 17 - protein, 23 aa, Cajanus cajan
ITVDKSVEFTYEELATATDNFSL

SEQ ID NO: 18 - protein, 23 aa, Phaseolus vulgaris
IKVDKSVKFTYEELATATDNFSL

SEQ ID NO: 19 - protein, 23 aa, Glycine max
ITVNKSVEFSYEELATATDNFSL

SEQ ID NO: 20 - protein, 23 aa, Abrus precatorius
IAVEKSLEFSYEELATATNNFNM

SEQ ID NO: 21 - protein, 23 aa, Nissolia schottii
ITVDKSVEFSYEELATATDNFSL

SEQ ID NO: 22 - protein, 23 aa, Arachis sp.
ITVDKSVEFSYEELATATNNFNL

SEQ ID NO: 23 - protein, 23 aa, Arachis sp.
ITVDKSVEFSYEELATATNNFNL

SEQ ID NO: 24 - protein, 23 aa, Casaus castanospermum
ITVDKSVEFSYEELATATNDFSM

SEQ ID NO: 25 - protein, 23 aa, Cicer arietinum
ITVDKSVEFSYDELAAASDNFSL

SEQ ID NO: 26 - protein, 23 aa, Pisum sativum
ITVDKSVEFSYDELAAASDNFSM

SEQ ID NO: 27 - protein, 23 aa, Lupinus angustifolius
ISVDKSVEFSYEELASATDNFSM

SEQ ID NO: 28 - protein, 23 aa, Cercis chinensis
ITVDKSVEFSYEELANATNNFSL

SEQ ID NO: 29 - protein, 23 aa, Chamaecrista fasciculata
ITVDKSVEFSYEELATATNNFNI

SEQ ID NO: 30 - protein, 23 aa, Lupinus angustifolius
ISMDKSVEFSYEELASATNNFSV

SEQ ID NO: 31 - protein, 23 aa, Mimosa pudica
ITVDKSVEFSYEELATATNNFNM

SEQ ID NO: 32 - protein, 23 aa, Mimosa pudica
ITVDKSVEFSYEELATATNNFNM

SEQ ID NO: 33 - protein, 23 aa, Chamaecrista fasciculata
IVVDKSVEFSYEELAAATNNFSM

SEQ ID NO: 34 - protein, 23 aa, Trifilum pratense
ITVDKSVEFSYDELAAASDNFSM

SEQ ID NO: 35 - protein, 23 aa, Manihot esculenta
ITVDKSVEFSYEELSRATDNFNM

SEQ ID NO: 36 - protein, 23 aa, Manihot esculenta
ITVDKSVEFSYEELAQATDNFST

SEQ ID NO: 37 - protein, 23 aa, Parasponia andersonii
ITVDKSVEFSYEELAKATDDFSL

SEQ ID NO: 38 - protein, 23 aa, Parasponia andersonii
ITVDKSVEFSYEELAKATDDFSL

SEQ ID NO: 39 - protein, 23 aa, Manihot esculenta
ITVDKSVEFSYEELAKATHDFSM

SEQ ID NO: 40 - protein, 23 aa, Hordeum vulgare
ITVDKSVEFSYEELFNATEGFNI

SEQ ID NO: 41 - protein, 23 aa, Lotus japonicus
IMVAKSMEFSYQELAKATNNFSL

SEQ ID NO: 42 - protein, 23 aa, Medicago truncatula
IMVAKSTEFTYQELAKATNNFSL

SEQ ID NO: 43 - protein, 23 aa, Glycine max
IMVAKSMEFSYQELAKATNNFSL

SEQ ID NO: 44 - protein, 23 aa, Glycine max
IMVAKSMEFSYQELAKATNNFSL

SEQ ID NO: 45 - protein, 23 aa, Cajanus cajan
IMVAKSMEFSYQELAKATNNFSL

SEQ ID NO: 46 - protein, 23 aa, Phaseolus vulgaris
IMVAKSMEFTYQELAKATNNFSL

SEQ ID NO: 47 - protein, 23 aa, Abrus precatorius
IMVAKSTEFSYQELAKATNNFSL

SEQ ID NO: 48 - protein, 23 aa, Cicer arietinum
IMVAKSTEFSYQELAKATNNFSL

SEQ ID NO: 49 - protein, 23 aa, Lupinus angustifolius
IMVAKSLEFSYQELAKATNNFSL

SEQ ID NO: 50 - protein, 23 aa, Lupinus angustifolius
IMVAKSLEFSYQELAKATNNFSL

SEQ ID NO: 51 - protein, 23 aa, Arachis sp.
IMAAKSMEFSYQELAKATNNFSS

SEQ ID NO: 52 - protein, 23 aa, Arachis sp.
IMAAKSMEFSYQELAKATNNFSS

SEQ ID NO: 53 - protein, 23 aa, Lotus japonicus
IMVAKSMEFSYQELAKATNNFSL

SEQ ID NO: 54 - protein, 23 aa, Lotus japonicus
ITVDKSVEFSYDELATATDNFSL

SEQ ID NO: 55 - skipped

SEQ ID NO: 56 - protein, 626 aa, Hordeum vulgare
MEAPLHSLLLLLLLAAAAGPKTAAAVGDGCSRGCDLALASYYIAPNQNVTYIASLFGFSEYRVLGQYNPGVNNLDYVVAGDRLNVSLTCKCLASLSAPASTFLAASIPYKVATGETYLRIADNYNNLTTADWLVATNTYPANNIPDVATVNATVNCSCGDAGISTDYGLFLTYPLRDRETLASVAANHGFSSPEKMDLLKKYNPGMDGVTGSGIVYIPAKDPNGSYRPLESPGKKSSAGAIAGGVVAGVVALVLGVVLFLFYRRRKAKKDALLPSSEESTRLASAISMQKVTPSTSQADGASPAAGITVDKSVEFSYEELFNATEGFNIIHKIGQGGFGAVYYAELRGEKAAIKKMDMQATQEFLAELKVLTHVHHLNLVRLIGYCTESSLFLVYEFIENGNLSQHLRGTGYEPLSWVERVQIALDSARGLEYIHEHTVPVYIHRDIKSANILIDKNTRAKVADFGLTKLTEVGGGTSLQTRVVGTFGYMPPEYARYGDVSPKVDVYAFGVVLYELISAKDAIVRSAESTSDSKGLVYLFEEALSAPDPKEGIRRLMDPKLGDDYPIDAILKMTHLANACTQEDPKLRPTMRSVVVALMTLSSTSEFWDMNALYENPGLVNLMSGR

SEQ ID NO: 57 - protein, 29 aa, Hordeum vulgare
KKSSAGAIAGGVVAGVVALVLGVVLFLFY

SEQ ID NO: 58 - protein, 23 aa, Hordeum vulgare
RRRKAKKDALLPSSEESTRLASA

SEQ ID NO: 59 - protein, 21 aa, Hordeum vulgare
ISMQKVTPSTSQADGASPAAG

SEQ ID NO: 60 - protein, 23 aa, Hordeum vulgare
ITVDKSVEFSYEELFNATEGFNI

SEQ ID NO: 61 - protein, 297 aa, Hordeum vulgare
IHKIGQGGFGAVYYAELRGEKAAIKKMDMQATQEFLAELKVLTHVHHLNLVRLIGYCTESSLFLVYEFIENGNLSQHLRGTGYEPLSWVERVQIALDSARGLEYIHEHTVPVYIHRDIKSANILIDKNTRAKVADFGLTKLTEVGGGTSLQTRVVGTFGYMPPEYARYGDVSPKVDVYAFGVVLYELISAKDAIVRSAESTSDSKGLVYLFEEALSAPDPKEGIRRLMDPKLGDDYPIDAILKMTHLANACTQEDPKLRPTMRSVVVALMTLSSTSEFWDMNALYENPGLVNLMSGR

SEQ ID NO: 62 - protein, 298 aa, synthetic construct
XNKIGQGGFGAVYYAELRGEKTAIKKMDVQASTEFLCELKVLTHVHHLNLVRLIGYCVEGSLFLVYEXIDNGNLGQYLHGXGKXPLPWSXRVQIALDSARGLEYIHEHTVPVYIHRDVKSANILIDKNXRGKVADFGLTKLIEVGXSTLHTRLVGTFGYMPPEYAQYGDXSPKXDVYAFGVVLYELISAKNAVLKTGEXVAESKGLVXLFEEALNXSXPXEXXRKLVDPRLGENYPIDSVLKXAXLGRACTRDNPLLRPSMRSIVVALMTLSSXTEDCDDXXXSYENQTLINLLSVRX

SEQ ID NO: 63 - protein, 297 aa, synthetic construct
ANKIGQGGFGXVYYAELRGEKAAIKKMDMQAXKEFLAELKVLTXVHHLNLVRLIGYSIEGSLFLVYEXIENGNLSQHLRGSGRDPLPWXTRVQIALDSARGLEYIHEHTVPVYIHRDIKSANILIDKNFRGKVADFGLTKLTEVGSSSLPTGRLVGTFGYMPPEYAQYGDVSPKVDVYAFGVVLYELISAKEAIVKXXESXADSKGLVXLFXGVLXQPDPTEDLXKLVDPRLGDNYPXDSVRKMAQLAKACTQDNPQLRPSMRSIVVALMTLSSTTDDWDVGSFYENQNLVNLMSGR

SEQ ID NO: 64 - protein, 297 aa, synthetic construct
AXKIGQGGFGXVYYAELRGEKAAIKKMDMQASKEFLAELKVLTHVHHLNLVRLIGYCVEGSLFLVYEYIENGNLSQHLRGSGRXPLPWSTRVQIALDSARGLEYIHEHTVPVYIHRDIKSANILIDKNFRXKVADFGLTKLTEVGSXSXPTRLVGTFGYMPPEYAQYGDVSPKVDVYAFGVVLYELISAKEAIVKXNGSVAESKGLVXLFEDVLNEPDPKEDLRKLVDPRLGDNYPXDSVRKMAQLAKACTHENPQLRPSMRSIVVALMTLSSSTEDWDVGSFYENQGLVNLMSGRX

SEQ ID NO: 65 - protein, 296 aa, Lotus japonicus; identical to SEQ ID NO: 7

SEQ ID NO: 66 - protein, 297 aa, Lotus japonicus; identical to SEQ ID NO: 14

SEQ ID NO: 67 - protein, 615 aa, Mimosa pudica
MEPRNISPVFIIIIFFISSVFPITESSCSKSCDLALASYYVWPFSNLTFISDQMKSQILTDPNNIVSYNKDQTISKDYVQSTTRIDIPFPCDCLQDGSLGHLFQYKISEGDTYKTIAETYYSNLTTVESLRKLNGNTIPSSGTINVPVTCYCGDSLVSKLYGLFITWPLRPEDSLASIAQSENLDAELLQRYNPGVNFSQGSGLVFIPGKXXXAGLSGGVIAGISVGVVAGLLLLAVSIYFGCFWKKTDDTRPLSQDFQEFSVNEEKGKNLFSCYNLSSNIESEILGYGGPTGITVDKSVEFSYEELAAATNNFSMANKIGQGGFGAVYYAELRGEVAIKKMDMQATREFLAELKVLTNVHHLNLVRLIGYCTENSLFLVYEFIDNGNLSQHLRGTGAGRDPLPWPTRVHIALDSARGLEYIHEHTVPVYIHRDIKSANILIDNSFRGKVADFGLTKLTEVGNSSLPTGNLVGTFGYMPPEYAQYGDVSPKVDVYAFGVVLFELISAKEAIVKTTQSDVDSKGLVMQFHGVLNQPDPSEDLRKLVDPRLGDDYPTDSVLKMAQLAKACTQENPQLRPTMRSIVVALMTLYSSTADDWDVGAFYNNPNLMNLMSGR

SEQ ID NO: 68 - protein, 550 aa, Chamaecrista fasciculata
MKPLLALALLFLLLETAFLNVESMCIKGCDLALASYYIPLFFTLQNLTILLQSKVLSDYQVIASFNKDSIFNGVNIKSYQSINVPFPCDCIDGDFLGHVFEYTIANGDTYERIASRKYAMLTSVELLKKYNNYSDPNHLPINGKLNVTVNCYCGDRQISKDYGLFVTYPLRDGDSLQTIAEKTNLDLGLLERYNQGMNFSQGSGLVFYPGKGLRGSAITGMSIGGMLGSLLVAIYIYLRYYRKKAVKVALTTEDSTDLSTQDASGSAGYDTSGSGNSAAGLKGIVVSKSVEFSYQEIAKATNNFSFDNKIGQGGFGAVYYAELRGEKTAIKKMDIEASQEFLAELKVLTHVHHLNLVSHLSMKGERQCHGLRGCKLLWIQQEVADFGLSKLIEVGNASLNTRLVGTFGYMSPEYAQYGITSPKIDVYAFGVVLYELISAKNAVLKTGECVGVAESKGLVTLFEEALRWPDPIEGLRKLVDPKLGENYPIDSIYKMAQLARACTQEDPQLRPSMRSIVVALMTLSSYGEDCDHVDSNLYDNQTLINMLSER

SEQ ID NO: 69 - protein, 629 aa, Prosopis alba
MQPKSNSLVLVFFLLLLETSLLNNVESKCLQGCPLALASYYAYKNNLGNITKFLRSDLVSDFGVVSSYNEGKVSNNGMYVQSLTRINIPFPCDCINNGDFLGHVFEYTIAEGDTYDSIATQTYSSLTTVDLLRRFNSYKDPNQLPLNGKLNVTVNCSCGDRQISKDYGLFLTYPLRFGDSLESIVDQTGIDATLLQKYNLRVNFSQESGVVFIPGRDQNGNYVPLDPRSRGLSEVAIAGICVGATFVFLLLSAYVFVKHFRRKTAKVALITENSTDLSSNQDGMIITSTNYETSGSSGSASATGLRGIMVGKSMEFSYQEIAKATNNFSSDNKIGQGGFGAVYYAELRGEKTAIKKMDIEASQEFLAELKVLTHVHHLNLVRLIGYCVEGSLFLVYEYINNGNLSQHLRSSELDALPWCTRVQIALDSARGLEYIHEHTVPVYIHRDVKSLNILIDESFHAKVADFGLSKLIEVGNASLNTRLVGTFGYMSPEYAQYGITSPKIDVYAFGVVLYELISAKNAVLKTGEFVAESKGLVALFEKSLGRPDAVEDLRKLVDPRLGQNYPIDSVFKMAQLARACTQDDPQLRPTMRSIVVALMTLSSNADDEDCDVGSAFSDNHTIINLLSGR

SEQ ID NO: 70 - protein, 629 aa, Arachis duranensis
MKLKRSFLLFFLLLECSCFINLESKCLKGCDLAIASYFVYPGVMLGSIASFMHSNVVPNSNVIISYNKDKMPNNLPVSFSRINIPFPCDCINGEFLGHVFEYSAIEGDTYDLIANLRYSDLTTVEILQRFNNYDPNHVPVNAKVNVTVNCSCGNSNVSKDYGLFVTFPLSEGNTLLQIANQTKLDPKLLQSYNPGVNFNQTRGIVFIPARDQNGVYVPLYPRTGLAKGVAVGISIAGTSGVLLLLICACVRYFQKKEEEKIKLPTEDFISSSTQDASSSGEYETSGSTAASASGLPAIMAAKSMEFSYQELSKATNNFSSDNKIGQGGFGAVYFAELRGQKAAIKKMDVQASTEFLSELKVLTHVHHLNLVRLIGYCVEGSLFLVYEYIENGNLGQYLHGTGREPLAWLTRVQIALDSARGLEYIHEHTVPVYIHRDVKPANILIDKDFRGKVADFGLTKLREVGSSLSLHTRLVGTFGYMPPEYAQYGDVSPKVDVYAFGVVLYELISAKNAVLKTGETVAESSKGLVTLFEEALDKSDPSKAIRKLVDPRLGENYSIDSVLKVAELGRACTRDNPLLRPSMRSVVVALMTLLASSTSIENCDDDDDDDDDDDNSYENRTLIDQLSVR

SEQ ID NO: 71 - protein, 633 aa, Arachis hypogaea
MKLKKSFLLFFLLLECSCFINLESKCLKGCDLAIASYFVYPKVMLGSIASFMHSNVVPNSNVIISYNKDKMPNNLPVSFTRINIPFPCDCINGEFLGHVFEYSAIEGDTYDLIANMRYSDLTTVEILQRFNNYDPNHVPVNAKVNVTVNCSCGNSNVSKDYGLFVTFPLSEGNTLLQIANQTKLDPKLLQGYNPGVNFNQTRGIVFIPGRDQNGVYVPLYPRLAGLAKGVAVGISIAGTSGVLLLLICACVRYFQKKEEEKIKLPTEDFISSSTQVGNASSSGEYETSGSTAASASGLPAIMAAKSMEFSYQELAKATNNFSSDNKIGQGGFGAVYFAELRGQKAAIKKMDVQASTEFLSELKVLTHVHHLNLVRLIGYCVEGSLFLVYEYIENGNLGQYLHGTEREPLAWSTRVQIALDSARGLEYIHEHTVPVYIHRDVKPANILIDKDFRGKVADFGLTKLREVGSSLSLHTRLVGTFGYMPPEYAQYGDVSPKVDVYAFGVVLYELISAKNAVLKTGETVAESSKGLVTLFEEALNKSDPSKAIRKLVDPRLGENYPIDSVLKVAELGRACTRDNPLLRPSMRSVVVALMTLLASSTSIEDCDDNDDDDDDDDDNSYENRTLIDQLSVR

SEQ ID NO: 72 - protein, 630 aa, Arachis ipaensis
MKLKKSFLLFFLLLECSCFINLESKCLKGCDLAIASYFVYPKVMLGSIASFMHSNVVPNSNVIISYNKDKMPNNLPVSFTRINIPFPCDCINGEFLGHVFEYSAIEGDTYDLIANMRYSDLTTVEILQRFNNYDPNHVPVNAKVNVTVNCSCGNSNVSKDYGLFVTFPLSEGNTLLQIANQTKLDPKLLQGYNPGVNFNQTRGIVFIPGRDQNGVYVPLYPRTGLAKGVAVGISIAGTSGVLLLLICACVRYFQKKEEEKIKLPTEDFISSSTQVASSSGEYETSGSTAASASGLPAIMAAKSMEFSYQELAKATNNFSSDNKIGQGGFGAVYFAELRGQKAAIKKMDVQASTEFLSELKVLTHVHHLNLVRLIGYCVEGSLFLVYEYIENGNLGQYLHGTEREPLAWSTRVQIALDSARGLEYIHEHTVPVYIHRDVKPANILIDKDFRGKVADFGLTKLREVGSSLSLHTRLVGTFGYMPPEYAQYGDVSPKVDVYAFGVVLYELISAKNAVLKTGETVAESSKGLVTLFEEALNKSDPSKAIRKLVDPRLGENYPIDSVLKVAELGRACTRDNPLLRPSMRSVVVALMTLLASSTSIEDCDDNDDDDDDDDDNSYENRTLIDQLSVR

SEQ ID NO: 73 - protein, 621 aa, Lupinus angustifolius
MKLKLVFLLLLKYVCFIVESKCIKGCDLALASYYVPVWPIVSLGNITSFMHSNVLTNPNVVTSYNKDKVFNGDVMLALYRTNVPFPCDCIGGEFLGHVFEYSAVEGDTYGLIAMKRYSNLTTVEILKRFNSYDPNHIPVNAKVNVTVKCSCGNSQISKDYGLFITYPLRPGNNLQELSKETKIDAKLLQSYNPGVNFSQENGIVFIPGKDQNGVYVPLYPRTGGVAKGVAVGISIAATCGLVLLVICIYDRYFKKKEGEKAKLSIENSIGFSTQDAYGSGEYETSGSSVHASALTGIMVAKSLEFSYQELAKATNNFSLDNKIGQGGFGAVYYAELRGEKTAIKKMDVQASSEFLAELKVLTHVHHLNLVRLIGYCVEGSLFLVYEYIDNGNLGQYLHGKGKDPLPWSTRLQIALDSARGLEYIHEHTVPLYIHRDVKSANILIDKNLRGKVADFGLTKLIEVGTSSLHTRLVGTFGYMPPEYAQYGDISPKIDVYAFGVVLYELISAKNAVLKTGETVAESKGLVNLFEEALNQINPLEPLTTLVDPRLGDNYPIQSLLKIAELGRACTRDNPLLRPNMKSIVVALMTLSSSNEDNTTSSYDNQTLINLLLDEGFRGITF

SEQ ID NO: 74 - protein, 624 aa, Lupinus angustifolius
MKLKFVFFLLLECVCFIVESKCIKGCDLALASYYVSVWPSISLGNITNFMHSNVLTNSDVIISYNKGKIFNGDVLLSLTRTNVPFPCDCIGGEFLGHVFQYSSVAGDTYDTIAMKSYSNLTTVEFLKRFNSYDPNHIPVNSKVNVTINCSCGNSLISKDYGLFTTYPLRPGNNLQELSKETNIDAKLLQSYNPGANFSQESRIVFIPGRDQNGVYVPLYPRIGGLARGAAVGISIAATCGLVLLIICIYDRYFKKKEGEKTKLSTEDSIGLSTEDGTGSGSGEYEASGSSGHAVGLTSIMVAKSLEFSYQELAKATNNFSLDNKIGQGGFGAVYYAELRGEKTAIKKMDVQASSEFLAELKVLTHIHHSNLVRLIGYCVEGSLFLVYEYIDNGNLGQYLHGKGRDPLPWSTRLQIALDSARGLEYIHEHTVPLYIHRDVKSANILIDKNLRGKVADFGLTKLIEVGTSSFHTRLVGTFGYMPPEYAQYGDISSKIDVYAFGVVLYELISAKSAILKTGETVSESKGLVTLFEGALNQINPLEALPKLVDPRIGDNYPIESVLKIAQLGRACTRDNPLLRPNMKSIVVALMTLSSSAEHSTSYDNQTLINLLLDEGFREIITPTS

SEQ ID NO: 75 - protein, 626 aa, Lupinus alba
MKLKFVFLLLLEYVCFTVESKCIKGCDIALASYYVPVWPTVSLGNITNYMYSNVLTKSNVIISYNKDKVFNGDVMLALYRTNIPIPCDCIGGEFLGHVFEYTAVAGDTYDLIAMKRYSNLTTVEFLKRFNSYDPNHIPVNAKVNVTVNCSCGNSLISKDYGLFITYPLRPGNNLQELSKETNIDAKLLQSYNPSVNFSQENGIVFIPGRDQNGVYVPLYPRTEGLARGVAVGISIAASCGLVLLVICIYDRYFKKKEGEKAKLSIENSLVLSTQDASGSSEYETSGSSVHASGLTGIMVAKSLEFSYQELAKATNNFSLDNKIGQGGFGAVYYAELRGEKTAIKKMDVQASSEFLAELKVLTHVHHLNLVRLIGYCVEGSLFLVYEHIDNGNLGQYLHGKGKDPLPWSTRLQIALDSARGLEYIHEHTVPLYIHRDVKSANILIDKNLRGKVADFGLTKLIEVGTSSLHTRLVGTFGYMPPEYAQYGDISPKIDVYAFGVVLYELISAKNAVLKTGETVAESKGLVSLFEGALNHINPLEALSTLVDPRLGDNYPIESVLKIAQVGRACTRDNPILRPNMKSIVVALMTLSSSTHQDHTSSSSYDHQTLINLLLDEGFRGIITPTF

SEQ ID NO: 76 - protein, 616 aa, Cicer arietinum
MKLKNIFLFFFFMFLECVFFKVESKCVKGCDLALASYYIKPPFEFTNITNFMQSNSDIIISYNKQLVSNNGKLFSFSRINIPFQCECIQGEFLGHMFEYTTNEGDTYDLIANSYYASLTTIDLLQKFNKYDHNQTLPSKVKVNVTVNCSCGNSQISKEYGLFLTYPLRSSDTLQIIAIESKVDEVLIQNYNPNVNFSRGSGIVFIPGRDKNGDYVPLYPRTGLAKGAAVGISMAGIFGFLLFVICIYVKYFQKNEEQNTKLLETSIALSTQSASGSGEYETSGSSGHGTSSAAGLTGIMVAKSTEFSYQELAKATNNFSLDNKIGQGGFGAVYYAELRGEKTAIKKMDVQASTEFLCELKVLTHVHHLNLVRLIGYCVEGSLFLVYEHIDNGNLGQYLHGIGTEPLPWSSRVQIALDSARGLEYIHEHTVPVYIHRDVKSANILIDKNLRGKVADFGLTKLIEVGNSTLHTRLVGTFGYMPPEYAQYGDVSPKIDVYAFGVVIYELISAKNAVLKTGESVAESKGLVALFEEALNKSDPCEGLRKLVDPRLGENYPIDSVLKMAQLGRACTRDNPLLRPSMRSIVVALMTLSSPTEDCDDDTSYENQTLINLLSVR

SEQ ID NO: 77 - protein, 620 aa, Medicago truncatula
MNLKNGLLLFILFLDCVFFKVESKCVKGCDVALASYYIIPSIQLRNISNFMQSKIVLTNSFDVIMSYNRDVVFDKSGLISYTRINVPFPCECIGGEFLGHVFEYTTKEGDDYDLIANTYYASLTTVELLKKFNSYDPNHIPVKAKINVTVICSCGNSQISKDYGLFVTYPLRSDDTLAKIATKAGLDEGLIQNFNQDANFSIGSGIVFIPGRDQNGHFFPLYSRTGIAKGSAVGIAMAGIFGLLLFVIYIYAKYFQKKEEEKTKLPQTSRAFSTQDASGSAEYETSGSSGHATGSAAGLTGIMVAKSTEFTYQELAKATNNFSLDNKIGQGGFGAVYYAELRGEKTAIKKMDVQASSEFLCELKVLTHVHHLNLVRLIGYCVEGSLFLVYEHIDNGNLGQYLHGIGTEPLPWSSRVQIALDSARGLEYIHEHTVPVYIHRDVKSANILIDKNLRGKVADFGLTKLIEVGNSTLHTRLVGTFGYMPPEYAQYGDVSPKIDVYAFGVVLYELITAKNAVLKTGESVAESKGLVQLFEEALHRMDPLEGLRKLVDPRLKENYPIDSVLKMAQLGRACTRDNPLLRPSMRSIVVALMTLSSPTEDCDDDSSYENQSLINLLSTR

SEQ ID NO: 78 - protein, 617 aa, Pisum sativum
MKLKNGLLLFFMFLDCIFFKVESKCVIGCDIALASYYVMPLVELLNITTFMQSKLVTNSSEVIVRYNRDIVFSNDNLFSYFRINIPFPCECIGGEFLGHVFEYTANEGDTYDLIANTYYASLTTVEVLKKFNSYDPNHIPAKAKVNVTVNCSCGNSQISKDYGLFITYPLRPRDTLEKIASHSKLDEGVIQSYNLGVNFSKGSGIVFFPGRDKNGEYVPLYPRAGLGKGAAAGISIAGIFALLLFVICIYIKYFQKKEEEKTKLPQISTALSAQDASGSGEYETSGSSGHGTGSTAGLTGIMVAKSTEFSYQELAKATNNFSLDNKIGQGGFGAVYYAVLRGEKTAIKKMDVQASTEFLCELQVLTHVHHLNLVRLIGYCVEGSLFLVYEHIDNGNLGQYLHGIDKAPLPWPSRVQIALDSARGLEYIHEHTVPVYIHRDVKSANILIDKNLHGKVADFGLTKLIEVGNSTLHTRLVGTFGYMPPEYAQYGDVSPKIDVYAFGVVLYELISAKNAILKTGESAVESKGLVALFEEALNQIDPLEALRILVDPRLKENYPIDSVLKMAQLGRACTRDNPLLRPSMRSLVVALMTLLSHTDDDDTFYENQSLTNLLSVR

SEQ ID NO: 79 - protein, 632 aa, Cajanus cajan
MFLSYPMELKKGLLFLLLLECVFCDVESMCVKECDVALASYYILPGYLTVENVTGWLESSVLSNSDVIKSYNKDKIIKDNLPSFDRINVPFPCDCIHEEFLGHVFEYSAAAGDTYDSIAKVTYANLTTVELLTRFNSYGHDIPQNAKINVTVKCSCGNSQVSKDYGLFITYPLRPGNNLHDIANEVHLDAQLLEKYNPGVNFSKDSGIVFIPGRDQNGDYVPLYPRKAGGIKKKISAKDAAVGVSIAGICGLLLLVVCIYVKYLQKKEGEEVKLPTESSMAFSTQDGTASGSSEYETSGSSGPTTASATGLTGIMVAKSMEFSYQELAKATNNFSLENKIGQGGFGAVYYAELRGEKTAIKKMDVQASTEFLCELKVLTHVHHLNLVRLIGYCVEGSLFLVYEYIDNGNLGQYLHGTGKDPLPWSSRVQIALDSARGLEYIHEHTVPVYIHRDVKSANILIDKNFRGKVADFGLTKLIEVGGSTLHTRLVGTFGYMPPEYAQYGDVSPKVDVYAFGVVLYELISAKNAILRTGESVAETKGLVALFEEALNQSNPGEGIRKLVDPRLGENYPIDSVLKIAQLGRACTRDNPLLRPSMRSIVVALMTLSSPTEDCEDGASYENQTLINLLSVR

SEQ ID NO: 80 - protein, 620 aa, Abrus precatorius
MKPANGFLLFLLLLEYVCCNVESKCMKGCHVALASYYVRPKFLSLDNIMRLMQSKILSTSDVIRSYNKDKILNDNVPSFSRVNIPFPCDCIGDEFLGHVFEYSTAAGDTYDLIAKVKYANLTTVELLQRFNSYDQDDIPANSKLNVTVNCYCGNSQISKDYGMFITYPLRPGNTLQDISNETNLDAHLLQSYNPGVNFSSESGIVFIPGRDQNGDYVPLYPRSGLATGAAVGITIAAICGLILLVIFMYVKYFQKKEGEKAKLPSESSVAFSTQDASGSGEYETSGSTGPANASAAGLTGIMVAKSTEFSYQELAKATNNFSLDNKIGQGGFGAVYYAELRGEKTAIKKMDVQASTEFLCELKVLTHVHHLNLVRLIGYCVEGSLFLVYEYIDNGNLGQYLHGTGKDPLPWSSRVQIALDSARGLEYIHEHTVPVYIHRDVKSANILIDKHFRGKVADFGLTKLIEVGSSTLHTRLVGTFGYMPPEYAQYGDVSPKIDVYAFGVVLYELISAKNAVLKTGESVAESKGLVALFEEALNQSNPLEGIHKLVDPRLGENYPIDSVLKIAQLGRACTRDNPLLRPSMRSIVVALMTLASPTEDCDDDTISYENQTLINLLSVR

SEQ ID NO: 81 - protein, 599 aa, Phaseolus vulgaris
MELKKGLLLLFLLLEYVCCNVEAKCLKGCGAALASYYVSPGISTLDDITHLMKSSVVSNSDDIVSYNKDRIFNKNVLFFYRINIPFPCECIRDEFLGHVFEYSAAAGDTYDSIAKVTYANLTTVELLRRFNSYGQNDIPTNAKVNVTVNCSCGNSQVSQDYGLFITYPLRPGNNLHDIANETQLDAQLLQNYNPGVNFSQESGIVFIPGRGLSRGAAAGISIAGICGLLLLVICIYAKFFQKKEGEKSKLPTMVFSTQYASGSAEYETSGSSGTATATGLTGIMVAKSMEFTYQELAKATNNFSLENKIGQGGFGAVYYAELRGEKTAIKKMDVQASTEFLCELKVLTHVHHLNLVRLIGYCVEGSLFLVYEYIDNGNLGQYLHGIGKDPLPWSSRVQIALDSARGLEYIHEHTVPVYIHRDVKSANILIDKNFRGKVADFGLTKLIEVGGSTLHTRLVGTFGYMPPEYAQYGDVSPKVDVYAFGVVLYELISAKNAVLKTGEYVAESKGLVALFEEALNQSNPSEVIRKLVDPRLGENYPIDSVLKIAQLGRACTRDNPLLRPSMRSIVVALMTLSSPTEDLDDSYENQTLINLLSVR

SEQ ID NO: 82 - protein, 627 aa, Vigna angularis
MFLLLITDPMELRKGLLLLFLLLEYVCCNVEAKCLKGCDLALASYYVSSGLTLDDITHLMKSSVVSNSDDIISYNKDKTFNKNIFLFDRINVPFPCDCIHDEFLGHVFEYSAAEGDTYDSIAKVEYADLTTVELLRRFNSYGQNGIPKNAKVNVTVNCSCGNSQVSQEYGLFITYPLRPDNNLHDIANEAHLDAQLLQNYNPGVNFSKESGIVFIPGRDQFGDYVPLHPRKTGLATGAVAGISIAGICGLLLFIICTYVKFFKKMEGEKPKLPTEKSTAFSIQDASGSADYETSGSSGTATATGLTGIMVAKSMEFTYQELAKATNNFSLENKIGQGGFGAVYYAELRGEKTAIKKMDVQASTEFLCELKVLTHVHHLNLVRLIGYCVEGSLFLVYEHIDNGNLGQYLHGTGKDPLTWSSRVQIALDSARGLEYIHEHTVPVYIHRDVKSANILIDKNFRGKVADFGLTKLIEVGSSTLHTRLVGTFGYMPPEYAQYGDVSPKVDVYAFGVVLYELISAKNAVLKTGEYVAESKGLVALFEEALNQSNPSEAIRKLVDPRLGENYPIDSVLKIAQLGRACTRDNPLLRPSMRSIVVALMTLSSPTEDFDDETASYENQTLINLLSVR

SEQ ID NO: 83 - protein, 627 aa, Vigna unguiculata
MFLLLITNPMELRKGLLLLFLLLEYVCCNVEAKCLKGCNVALGSYYVNTGMSLDVITPLMKSSVVSNSDDIISYNKDKIFNKNVFYFDRINVPFPCDCISDEFLGHVFEYSAAEGDTYDSIAKVEYADLTTVELLRRFNSYGQNGIPKNAKVNVTVNCSCGNSQVSQDYGLFITYPLRPGNNLHDIANEAHLDAQLLQNYNPGVNFSKESGIVFIPGRDQFGEYVPLHPRNTGLAKGDVAGISIAGICGLLLLVICIYVKFFKKKEGEKPKLPTENSTAFSTQDASGSAEYEISGSSGTTTAAGLTGIMVAKSMEFTYQELAKATNNFSLENKIGQGGFGAVYYAELRGEKTAIKKMDVQASTEFLCELKVLTHVHHLNLVRLIGYCVEGSLFLVYEHIDNGNLGQYLHGTGKDPLPWSSRVQIALDSARGLEYIHEHTVPVYIHRDVKSANILIDKNFRGKVADFGLTKLIEVGGSTLHTRLVGTFGYMPPEYAQYGDVSPKVDVYAFGVVLYELISAKNAVLKTGEFVAESKGLVALFEEALNQSNPSEAIRKLVDPRLGENYPIDSVLKIAQLGRACTRDNPLLRPSMRSIVVALMTLSSPTEDFEDETASYENQTLINLLSVR

SEQ ID NO: 84 - protein, 644 aa, Spatholobus suberectus
MFLLFGLRDPMKLKKGLLFFLLLEYVCRSVESKCVKGCDVALASYYVSPGYFPLENITRLMESSVLSNSDVVTSYNKDKIFNDMVQSFVRLNIPFSCGCIGGEFLGHVFEYSAAAGDTYDSIAKVTYANLTTVELLRRFNSYDQNDIPANAKLNVTVNCSCGNSQVSKDYGLFITYPLRPGNNLHDIANETQLDAQLLQSYNPGVNFSQESGIVFIPGRGILSFSHHVHLVHSSLYFDQNGDYVPLYPRKTGLARGAAVGISIAGICGLLLLVIFVYVRYFQNKEGKKDKLPTENSMVFSTQDGTEYETSGSSGLATASAAGLTGIMVAKSMEFSYQELAKATNNFSLENKIGQGGFGAVYYAELGGEKTAIKKMDVQASTEFLCELKVLTHVHHLNLVRLIGYCVEGSLFLVYEYIDNGNLGQYLHGTGKDPLPWSSRVQIALDSARGLEYIHEHTVPVYIHRDVKSANILIDKNLRGKVADFGLTKLIEVGGSTLHTRLVGTFGYMPPEYAQYGDISPKVDVYAFGVVLYELISAKNAVLKTGESVAETKGLVALFEEALSQSNPSEGIRKLVDPRLGENYPVDSVLKIAQLGRACTRDNPLLRPSMRSIVVALMTLSSPTEDCDDDASYENQTLINLLSVR

SEQ ID NO: 85 - protein, 611 aa, Glycine max
MELKKGLLVFFLLLECVCYNVESKCVKGCDVAFASYYVSPDLSLENIARLMESSIEVIISFNEDNISNGYPLSFYRLNIPFPCDCIGGEFLGHVFEYSASAGDTYDSIAKVTYANLTTVELLRRFNGYDQNGIPANARVNVTVNCSCGNSQVSKDYGMFITYPLRPGNNLHDIANEARLDAQLLQRYNPGVNFSKESGTVFIPGRDQHGDYVPLYPRKTGLARGAAVGISIAGICSFLLLVICLYGKYFQKKEGEKTKLPTENSMAFSTQDVSGSAEYETSGSSGTASATGLTGIMVAKSMEFSYQELAKATNNFSLENKIGQGGFGAVYYAELRGEKTAIKKMDVQASTEFLCELKVLTHVHHFNLVRLIGYCVEGSLFLVYEYIDNGNLGQYLHGTGKDPLPWSGRVQIALDSARGLEYIHEHTVPVYIHRDVKSANILIDKNIRGKVADFGLTKLIEVGGSTLHTRLVGTFGYMPPEYAQYGDISPKVDVYAFGVVLYELISAKNAVLKTGESVAESKGLVALFEEALNQSNPSESIRKLVDPRLGENYPIDSVLKIAQLGRACTRDNPLLRPSMRSIVVALMTLSSPTEDCDTSYENQTLINLLSVR

SEQ ID NO: 86 - protein, 618 aa, Glycine max
MELKKWLLFFLLLEYVCCNAESKCVKGCDVALASYYVSPGYLLLENITRLMESIVLSNSDVIIYNKDKIFNENVLAFSRLNIPFPCGCIDGEFLGHVFEYSASAGDTYDSIAKVTYANLTTVELLRRFNSYDQNGIPANATVNVTVNCSCGNSQVSKDYGLFITYLLRPGNNLHDIANEARLDAQLLQSYNPGVNFSKESGDIVFIPGKDQHGDYVPLYPRKTGLATSASVGIPIAGICVLLLVICIYVKYFQKKEGEKAKLATENSMAFSTQDVSGSAEYETSGSSGTASTSATGLTGIMVAKSMEFSYQELAKATNNFSLENKIGQGGFGIVYYAELRGEKTAIKKMDVQASTEFLCELKVLTHVHHLNLVRLIGYCVEGSLFLVYEYIDNGNLGQYLHGTGKDPFLWSSRVQIALDSARGLEYIHEHTVPVYIHRDVKSANILIDKNFRGKVADFGLTKLIEVGGSTLQTRLVGTFGYMPPEYAQYGDISPKVDVYAFGVVLYELISAKNAVLKTVESVAESKGLVALFEEALNQSNPSESIRKLVDPRLGENYPIDSVLKIAQLGRACTRDNPLLRPSMRSIVVALLTLSSPTEDCYDDTSYENQTLINLLSVR

SEQ ID NO: 87 - protein, 620 aa, Medicago truncatula; identical to SEQ ID NO: 77

SEQ ID NO: 88 - protein, 599 aa, Phaseolus vulgaris; identical to SEQ ID NO: 81

SEQ ID NO: 89 - protein, 633 aa, Arachis hypogaea; identical to SEQ ID NO: 71

SEQ ID NO: 90 - protein, 631 aa, Arachis hypogaea
MKLKKSFLLFFLLLECSCFINLESKCLKGCDLAIASYFVYPRVKLGSIASFMHSNVVPNSNVIISYNKDKMPNNLPVSFTTINIPFPCDCINGEFLGHVFEYSAIEGDTYYLIANMRYSDLTTVEILQRFNNYDPNHVPVNAKVNVTVNCSCGNSHVSKDYGLFVTFPLSEGNTLLQIANQTKLDPKLLQSYNPGVNFNQTRGIVFIPGRDQNGVYVPLYPRLAGLAKDVAVGISIAGTSGVLLLLICVCVRYFQKKEEEKIKLPTEDFISSSTQDGNASSSGEYETSGSTAASASGLPAIMAAKSMEFSYQELAKATNNFSSDNKIGQGGFGAVYFAELRGQKAAIKKMDVQASTEFLSELKVLTHVHHLNLVRLIGYCVEGSLFLVYEYIENGNLGQYLHGTGREPLAWLTRVQIALDSARGLEYIHEHTVPVYIHRDVKPANILIDKDFRGKVADFGLTKLREVGSSLSLHTRLVGTFGYMPPEYAQYGDVSPKVDVYAFGVVLYELISAKNAVLKTGETVAESSKGLVTLFEEALNKNDPSKAIRKLVDPRLGENYPIDSVLKVAELGRACTRDNPLLRPSMRSVVVALMTLLASSTSIEDCDDNDDDDDDDNSYENRTLIDQLSVR

SEQ ID NO: 91 - protein, 632 aa, Cajanus cajan; identical to SEQ ID NO: 79

SEQ ID NO: 92 - protein, 616 aa, Cicer arietinum; identical to SEQ ID NO: 76

SEQ ID NO: 93 - protein, 620 aa, Abrus precatorius; identical to SEQ ID NO: 80

SEQ ID NO: 94 - protein, 611 aa, Glycine max; identical to SEQ ID NO: 85

SEQ ID NO: 95 - protein, 618 aa, Glycine max; identical to SEQ ID NO: 86

SEQ ID NO: 96 - protein, 621 aa, Lupinus angustifolius; identical to SEQ ID NO: 73

SEQ ID NO: 97 - protein, 624 aa, Lupinus angustifolius; identical to SEQ ID NO: 74

SEQ ID NO: 98 - protein, 617 aa, Pisum sativum; identical to SEQ ID NO: 78

SEQ ID NO: 99 - protein, 618 aa, Vigna angularis
MELRKGLLLLFLLLEYVCCNVEAKCLKGCDLALASYYVSSGLTLDDITHLMKSSVVSNSDDIISYNKDKTFNKNIFLFDRINVPFPCDCIHDEFLGHVFEYSAAEGDTYDSIAKVEYADLTTVELLRRFNSYGQNGIPKNAKVNVTVNCSCGNSQVSQEYGLFITYPLRPDNNLHDIANEAHLDAQLLQNYNPGVNFSKESGIVFIPGRDQFGDYVPLHPRKTGLATGAVAGISIAGICGLLLFIICTYVKFFKKMEGEKPKLPTEKSTAFSIQDASGSADYETSGSSGTATATGLTGIMVAKSMEFTYQELAKATNNFSLENKIGQGGFGAVYYAELRGEKTAIKKMDVQASTEFLCELKVLTHVHHLNLVRLIGYCVEGSLFLVYEHIDNGNLGQYLHGTGKDPLTWSSRVQIALDSARGLEYIHEHTVPVYIHRDVKSANILIDKNFRGKVADFGLTKLIEVGSSTLHTRLVGTFGYMPPEYAQYGDVSPKVDVYAFGVVLYELISAKNAVLKTGEYVAESKGLVALFEEALNQSNPSEAIRKLVDPRLGENYPIDSVLKIAQLGRACTRDNPLLRPSMRSIVVALMTLSSPTEDFDDETASYENQTLINLLSVR

SEQ ID NO: 100 - protein, 618 aa, Vigna radiata
MELRKGLLLLFLLLEYVCCNVEAKCLKGCDLALASYYVNRGLSLDNITHLMKSSVVSNSDDIISYNKNKIFNKNVFMFNRYNVPFPCDCIRDEFLGHVFEYSAAEGDTYDSIAKVEYADLTTVELLRRFNSYGQNGIPKNAKVNVTVNCSCGNSQVSQEYGLFITYPLRPDNNLHDIANEAHLDAQLLQNYNPGVNFSKESGIVFIPGRDQFGDYVPLHPRKTGLATGAVAGISIAGICGLLLLVICIYVKFFKKMEGEKPKLPTEKSTASSIQDASGSAEYETSGSSGTATATGLTGIMVAKSMEFTYQELAKATNNFSLENKIGQGGFGAVYYAELRGEKTAIKKMDVQASTEFLCELKVLTHVHHLNLVRLIGYCVEGSLFLVYEHIDNGNLGQYLHGTGKDPLTWSSRVQIALDSARGLEYIHEHTVPVYIHRDVKSANILIDKNFRGKVADFGLTKLIEVGSSTLHTRLVGTFGYMPPEYAQYGDVSPKVDVYAFGVVLYELISAKNAVLKTGEFVAESKGLVALFEEALNQSNPSEAIRKLVDPRLGENYPIDSVLKIAQLGRACTRDNPLLRPSMRSIVVALMTLSSPTEDFDDETASYENQTLINLLSVR

SEQ ID NO: 101 - protein, 618 aa, Vigna unguiculata
MELRKGLLLLFLLLEYVCCNVEAKCLKGCNVALGSYYVNTGMSLDVITPLMKSSVVSNSDDIISYNKDKIFNKNVFYFDRINVPFPCDCISDEFLGHVFEYSAAEGDTYDSIAKVEYADLTTVELLRRFNSYGQNGIPKNAKVNVTVNCSCGNSQVSQDYGLFITYPLRPGNNLHDIANEAHLDAQLLQNYNPGVNFSKESGIVFIPGRDQFGEYVPLHPRNTGLAKGDVAGISIAGICGLLLLVICIYVKFFKKKEGEKPKLPTENSTAFSTQDASGSAEYEISGSSGTTTAAGLTGIMVAKSMEFTYQELAKATNNFSLENKIGQGGFGAVYYAELRGEKTAIKKMDVQASTEFLCELKVLTHVHHLNLVRLIGYCVEGSLFLVYEHIDNGNLGQYLHGTGKDPLPWSSRVQIALDSARGLEYIHEHTVPVYIHRDVKSANILIDKNFRGKVADFGLTKLIEVGGSTLHTRLVGTFGYMPPEYAQYGDVSPKVDVYAFGVVLYELISAKNAVLKTGEFVAESKGLVALFEEALNQSNPSEAIRKLVDPRLGENYPIDSVLKIAQLGRACTRDNPLLRPSMRSIVVALMTLSSPTEDFEDETASYENQTLINLLSVR

SEQ ID NO: 102 - protein, 629 aa, Arachis duranensis; identical to SEQ ID NO: 70

SEQ ID NO: 103 - protein, 630 aa, Arachis ipaensis; identical to SEQ ID NO: 72

SEQ ID NO: 104 - protein, 550 aa, Chamaecrista fasciculata; identical to SEQ ID NO: 68

SEQ ID NO: 105 - protein, 594 aa, Mimosa pudica
MQPKSHNALVLAVFFQILLLETTFLINNVESKCHKGCPLALASYLCHQNITLSNITHLLKSNLVSLDFQIISSYNKEGIVPNNIQFFTRINIPFPCECINNDDFLGHVFEYTISKGDTYHSIATQTYSSLTSVDLLRRFNNYSDPNQLPPNGKLNVTVKCSCGNRQISKDYGLFITYPLGFEDSLESIVNQTGIDALLLQSYNPGANFSQQSGVVFIPGRADQTGNYMPLDYIRSGRLRGTAIAGISVAATFVFLLLAAYVFVRYFRKKENAKLALFSENSAELSSQEGSSGVASTTDLRGIMEEKSMEFSYQEIAKATNNFSSDNRIGQGGFGTVYYAELRGQKTAIKKMDIKASQGFLSELKVLTHVHHLNLCFFHYINVNIYNDFDQEKIYNKIYCLNVLPWSTRVQIALDSARGLEYIHEHTVPTYIHRDVKSLNILIDKTYHAKVVADFGLSKLIEVGNASLNTRRLVGTFGYMSPEYAQYGIQSPKIDVYAFGVVLYELISAKNAVLKITNEFVAESKGLVALFEKCLRRPDAVEDLRNLVDPRLGQNYPIESVYKVQKLFEYESGLDSLNFEILKFFIKNSKRKVVT

SEQ ID NO: 106 - protein, 626 aa, Lupinus albus; identical to SEQ ID NO: 75

SEQ ID NO: 107 - protein, 644 aa, Spatholobus suberectus; identical to SEQ ID NO: 84

SEQ ID NO: 108 - protein, 629 aa, Prosopis alba; identical to SEQ ID NO: 69

SEQ ID NO: 109 - DNA, 1317 bp, synthetic construct
  source 1..1317: mol_type = other DNA
ggacatgagattgaagctccaaaattagctcttttttctgatgaatacttaatgctttgttgtattcacttgattaagtgctagaaatcatctttgcatgatcatagattaaatgaatttccagttggtgtgtggagagctattttgttatgctgacatctgcaatttgcagggcatctaatgattgtcatttcttaaattattattggttgtttccgtttctttaattatctgttttaatcttgcaggtcatacaaattaaaatactagccaccacccaagacatactaaatggggtagtagagggaagggtaaggtcgataaggatgactttttattctataaaatttaggagaatttgagcttaagtggcaaggcaaacgacattactatacgaattggctttgtaccagaaacagggaacaaataatattttacaaataagctattatcatgtcagctcatttgttcaactttgatttgattaaaaattaaatgaagttgaatttgttgagctgctttattatatatgccactggatgtttccgcattctaagtgcatgtttgaaaacatttctacaattgattacgaaggaaaaattaatcatggagagaagcttatgtgcgtagcttctgtatttctgaattgattctatctgtacagtagcatttagataatgaatgatcttggttctcgctaagcatcaaaccaatctctacccttttaaaattgcaagaattataagtcatgcattgacccaaatccttctgtggttatgccccttaaaaatccggcaagacatcaagttagttggtcattagggttccaccagctagctgacaccttgtacaacaactggccgtcctaaagttgggtaagcattacaatactaaatgccattttattatattttgcgcatggttatatacctaagtaggatttgtccacagtttctttgattcggaaaggaaaaaatatttagttgacactgacagaagcagattttatatacatatattatgaaatgactcctacatgagatacacgaatctcatccccatgagttgcagtttgacagagtacacacttatcaacttgctggaatataggaaagtctaaccaatgatgtcgatccgtattgccttaattttggtaaatttagtattacatgatcattattgatatactaaaccacaggatattttattgacaatgtgaatgttccatattttcaacaatgctgattccctctgataaagaacaagttccttttctctttccctgttaactatcatttgttccccacttcacaaaca

SEQ ID NO: 110 - DNA, 1135 bp, synthetic construct
  source 1..1135: mol_type = other DNA
cgatccttcgttgtattcacttggtcaagtgcaagaaatcatctttacacaatcatataatgaatggacctttggttgatgtgttgagagctacattcagtcctctgctgacttctgcaacttgaggggaatctaatgattgtcttattgcaattaacaattccttttaattttttgacagaaagagttggttacaaaatctcatctaacatattgttgagttcctcttttttttttttttttttttttttgataaaagggtacatctgttcgcagctctgaagattaatccctcgaatagggttggataacatgaacgacaaatttgtttcttaagagttttaacactgtcatattcttatggcagaattcgaactcgaacccactggttaagctagaagtaagaagtgacccatgccatctcattcaatcgttcttggcttgttttcggtttagatgaatatgaagtacaaaaagctacgctcaagcaaaggagagcaggtaagaagggcatcttatttttttaatatattagatgtcaagcttacgtggcgatgtaaacactgtttctatccgaattagctatgtactagaaatagggaacaaataatataaatggtgagtttgtcaaggtgttttaatatattgaaaccaatgtaactggaagtgtcaacattgaaagctgttcattgcctgtattatattataattgcatatatataatggcattagcctttgttaaacgttaaaccatatcttttgctagtttgccccttaaaaatttggtaaaggcatcaagttagatggtctttaggtaccagccagctagttgacatcgtgtatggacaccaaattgggttataataccatattattcttaccatctttattatattctgcgcatgatttcatacttgtttgggatttgccaacagtgtctaagatttgaaaaagaaaaatagtagaactaatgacagagacatcagcatatatttttaatatcaaaccaaaagatatttgatgtccaaagaagagataaatataaagttagaagtatgacaataagtcttggttgttatttgccataagaaactctcttttctcttcccctcataatttgcatttcctcacaaca

SEQ ID NO: 111 - DNA, 2496 bp, synthetic construct
  source 1..2496: mol_type = other DNA
tcgactatataaggggttcctccatacctaataagacatgtttgtttcatgtcgatctacttactctattttatggttttctcgtgagcaatttacatataaaatacacattaaattgcacccaataactaactagttacaagattagagcacataaatcatggaatgaaacaatagcctatgacttgggtattcggtccacaacatctctagaagttcgtacaacaacgatcaaagagaaagtactgactagtgatatgagaaatttggtcaggaacaatgggctggacacattacgaacttgagcctgaatagtagcagcatatgtctgccttcgcaatgacgaagaaggttggacggagctaaagttaaagctggaagggtgtgtcgtggcatttgaaatttagggttcaactgtagctttgtttgtctaactacccttctagctttaataaaaaaacgtaaatcaattaattaaagagaaatcacgccaacctgtctcaaacccgctctaacttgttggttaaggttatgtgttgtatttcaatttgcttgttgcttcaacttgactccttacctcctttcatgttttttcttgaaaattgtgtaaactcagttttttttttttgagaatgtatgagaatgtgatacaaattaaaatacaaacatacattgaatgagcggggctaaaatgactcaaggtacatatgagttaagtttttaccaacccatctctttgatgagttgggtttaattgggttagctcacgtgttaactatttcgacaattctaactttttggaataatgccatccaaccctaggtaaaactcaacatgacttctcttaaagaaatgaagagtgaaagtctacataaatcttaaacacttaaaacactacaacttggtagatcattaaaatatgctccagaaataccataatacttgaaaataaataatttataaatgtaaaatataaatttgttgctgcacccaagtctgagtgaggaatgaataaagataaataaaatatatatatgcgttgatactaacatatgatatgatgtgatagatgtcatatataatgaacagaatgatagaaattgttgagcgcatggaagcttcttgttcaataattcaagagctgcttgtggagggctgctgcagggttgttatggatgcaattatcaaatatcttgctggaaattgatccaaaagttgactattaccttatcaatgtccggtgtccttcaagccaccattcaattagtcaataaccttttgtttttgtttttttataattttggtgccaaataattttaaccatttatttttatcatatatattatataagatggcaagtctataatcatttaaaattaaattagtctaaatttttctaatatgctgtataaatttattatatataagaataaataatattaaaaatattgtttaattgctaccagttcaatcattaattaactattgactgaaaagaaaaatgtctaacaaattttgttacattctgtttgttttgagaaaaaaaaaattgtttttcattttctgaaaatatttatttttaaatttattaaatttagaaattatgtttgtttttacgctttgttttcatcttcaccgtcaccaccatcgctaccactgtcgtcactattactatataccactaccatcgttgttgccgttgtcattgtcaacaccattgctgtcgtcgttgttattatcgccccacaaccactatcatcatcactgctatcgtcatagctaccacgaacaccaccatcacttttcattgaaaatgaaaaatgtttatttatttattttgattatattttaaaaaatatttttaatcaaccaaatttctttttaactttttttttcttatgaaaaaaaataatatgaaaagaacaaaccaaacagatccttatattcttgtttccattctttatcacccatgagagtagctgtagcgcgttaactttctaatagaggcgggaatttaccgttaaccgttgggtcatcactcagcttactgagtcatcttcttcctactgtaggcggaacccacacgccactgcctctctattattacatcaaattctattttttttacatcatcatcaagataaataaattgcattattcagaaattaattcctgaatctccctatcttaactcaggtgtctctagctattaccacttaagctattattagaagtctacgtcaaattctataataaacacatattcttattcttattattatactaatacaaaccttgatttgaagccaaaaatgataaaacaattgcagactcgttcaaacaacagacaacatgtaccattaaattccaccccaaattctcaaagcaaattctgaattctccaactagtttccattctcctagtcaactcaactcaaaacccagatgagaatgagataattccaactttaacaaacacacacaattcatcattca

SEQ ID NO: 112 - DNA, 2000 bp, synthetic construct
  source 1..2000: mol_type = other DNA
aaataatgtgtctaaaattaacaaagatattttatggttaatttattgagcttagttaaagtcattttctaaaacgagacattgtagttgttagataaataatttcctttaattttcatcaaagtttgtaaataattatagggcaggacacatatgtccatcaattgatatatatatatcttttgtaataacgatcttatagaagaatcaatatctacaaagaaagttatcacttgtctcaaattatgctttatgcaactggtattgtcatgtagattccattttggtttatatgatattgaagtcaacgaatcacagctgcttaggcgtggttcataatttaaaattgaaactatggtgtttgttggaattatgagaggacagactgtgaagtggttgcacagctaccgacagggttgccattactgatgcaaaggattaaacaatagaacattttcaatcaacttaatctatgtttgacatcacgctcgatgtgtcagaatcacgacgacccactgtaatttttaaaaaaattacattttataatttttgtagaatcacggtgtatcacagtgattttgacatacccactgtaataccaaacatgcacttgcattctttcttccgtttgatgccaaaaatggtattgtttataatacacttaaaataattcatatccaagttgttgtataaaaaaataaacaatgtgatttcccaaaaaaaaaaaataataagtgattggcatgcaacatcagattttgttgtccaagtggtcacatttgaaatatttgagtttacatttttctcactaatatctttatattgaacttcaatctgtctagctataatcaaatggttgactaaaatggttaataagcttcaattaaattcaaattatttgaaagaacttgaatttaaatcttaacttgaacataaccccgaagtggatgatttacccaaaaaaattacctaactactctaataatcattttaacaataagagtattttactcaaagaaatttatataatcacaaaatcctagtatactcaatgattttcttaaaaaactattataaccagaaatgacaatatgagacaaatataattattttaataaaaatatctaaattagaattatttgaaaatgtaaaactatattattttttgtagttttccaaactttattaagcatcgtttaattgcacacaaaatttactccaattttttttagtctctgaaagttgctttttggtccatgttttagttttggtaattataattaagtttgttcatattaattagttctaatgtgtgtactatatagatcatactaattttgttcccaaactcatcttagttaggatctttgtatctactataagattaatccttgtgccgatataagaatatttgtcatgttatcaactatcacaatattgtattggtgagatcatataagatttgatgtcaacatcttcgtaaaggtgtcagattcgattctccccggtatcaatttaagtgagctaatttagcttcttaaaaaataaaatcaaacaacttttacataaactcagtgaaaacttggatataaagtatccttatactactctttagtcttgattagtctctgcaaagatatttatatgtactttgtattatcataagaacattcattgacattttaagttaatgaattactaacatgtcaactcttattctagccaacagttactttgttccctccacattctctttgaaatagtcaaacgtatccaatcatgcatgtctgttctgatcataacagcaaaagcatgtgtatagaaaattgatagttgaattagagtcattttccataaaaaaatattcaataagtgtgacattatttttcgtatgaattaatccattttttgctgatttgagattctttctttctttgcttcttgctttccttcatcagccattttttttgttttctctttctctctctcttcttgattca

SEQ ID NO: 113 - protein, 23 aa, synthetic construct
  VARIANT 2: note = X=T, K, A, S, V, or M
  VARIANT 3: note = X=V, M, or A
  VARIANT 4: note = X=D,N,E, or A
  VARIANT 7: note = X=V,L,M, or T
  VARIANT 8: note = X=E or K
  VARIANT 10: note = X=S or T
  VARIANT 12: note = X=D,E, or Q
  VARIANT 15: note = X=A,S, or F
  VARIANT 16: note = X=T,A,S,N,R,Q, or K
  VARIANT 18: note = X=T or S
  VARIANT 19: note = X=D,N,H, or E
  VARIANT 20: note = X=N,D, or G
  VARIANT 22: note = X=S or N
  VARIANT 23: note = X=L,M,I,V,T, or S
IXXXKSXXFXYXELXXAXXXFXX

SEQ ID NO: 114 - protein, 679 aa, Medicago truncatula
MNLKNGLLLFILFLDCVFFKVESKCVKGCDVALASYYIIPSIQLRNISNFMQSKIVLTNSFDVIMSYNRDVVFDKSGLISYTRINVPFPCECIGGEFLGHVFEYTTKEGDDYDLIANTYYASLTTVELLKKFNSYDPNHIPVKAKINVTVICSCGNSQISKDYGLFVTYDYDLIANTYYASLTTVELLKKFNSYDPNHIPVKAKINVTVICSCGNSQISKDYGLFVTYPLRSDDTLAKIATKAGLDEGLIQNFNQDANFSIGSGIVFIPGRDQNGHFFPLYSRTGIAKGSAVGIAMAGIFGLLLFVIYIYAKYFQKKEEEKTKLPQTSRAFSTQDASGSAEYETSGSSGHATGSAAGLTGIMVAKSTEFTYQELAKATNNFSLDNKIGQGGFGAVYYAELRGEKTAIKKMDVQASSEFLCELKVLTHVHHLNLVRLIGYCVEGSLFLVYEHIDNGNLGQYLHGIGTEPLPWSSRVQIALDSARGLEYIHEHTVPVYIHRDVKSANILIDKNLRGKVADFGLTKLIEVGNSTLHTRLVGTFGYMPPEYAQYGDVSPKIDVYAFGVVLYELITAKNAVLKTGESVAESKGLVQLFEEALHRMDPLEGLRKLVDPRLKENYPIDSVLKMAQLGRACTRDNPLLRPSMRSIVVALMTLSSPTEDCDDDSSYENQSLINLLSTR

SEQ ID NO: 115 - protein, 618 aa, Glycine max; identical to SEQ ID NO: 86

SEQ ID NO: 116 - protein, 613 aa, Glycine max
MELKKGLLVFFLLLECVCYNVESKCVKGCDVAFASYYVSPDLSLENIARLMESSIEVIISFNEDNISNGYPLSFYRLNIPFPCDCIGGEFLGHVFEYSASAGDTYDSIAKVTYANLTTVELLRRFNGYDQNGIPANARVNVTVNCSCGNSQVSKDYGMFITYPLRPGNNLHDIANEARLDAQLLQRYNPGVNFSKESGTVFIPGRDQHGDYVPLYPRKTGLARGAAVGISIAGICSFLLLVICLYGKYFQKKEGEKTKLPTENSMAFSTQDGTVSGSAEYETSGSSGTASATGLTGIMVAKSMEFSYQELAKATNNFSLENKIGQGGFGAVYYAELRGEKTAIKKMDVQASTEFLCELKVLTHVHHFNLVRLIGYCVEGSLFLVYEYIDNGNLGQYLHGTGKDPLPWSGRVQIALDSARGLEYIHEHTVPVYIHRDVKSANILIDKNIRGKVADFGLTKLIEVGGSTLHTRLVGTFGYMPPEYAQYGDISPKVDVYAFGVVLYELISAKNAVLKTGESVAESKGLVALFEEALNQSNPSESIRKLVDPRLGENYPIDSVLKIAQLGRACTRDNPLLRPSMRSIVVALMTLSSPTEDCDTSYENQTLINLLSVR

SEQ ID NO: 117 - protein, 632 aa, Cajanus cajan; identical to SEQ ID NO: 79

SEQ ID NO: 118 - protein, 599 aa, Phaseolus vulgaris; identical to SEQ ID NO: 81

SEQ ID NO: 119 - protein, 620 aa, Abrus precatorius; identical to SEQ ID NO: 80

SEQ ID NO: 120 - protein, 616 aa, Cicer arietinum; identical to SEQ ID NO: 76

SEQ ID NO: 121 - protein, 624 aa, Lupinus angustifolius; identical to SEQ ID NO: 74

SEQ ID NO: 122 - protein, 621 aa, Lupinus angustifolius; identical to SEQ ID NO: 73

SEQ ID NO: 123 - protein, 588 aa, Trifolium sp.
MQSKIVKNSPKVIMSYNKDIVFSNDNLFSYFRINVPFPCECIGGEFLGHVFEYSSSEGDTYDLIANTYYASLTTVEVLKKFNSYDPNHIPVKGKVNVTVNCSCGNSQIXXXXXXXXXXXXXXXXXXXXXRVSILVSILAKGVGLCSFQEEIKMEIMFPYILGLAKGAAIGITIAGIFGLVLIVICIYVKYFQKKEEEKVKLPQTSIALSTQDGNGIFFLIICLLIFVSDAGEASGSGEYETSGSSVPGTGSTAGLTGIMVAKSTEFSYQELAKATNNFSLEHKIGQGGFGAVYYAELRGEKTAIKKMDVQASTEFLCELKVLTHVHHLNLVRLIGYCVEGSLFLVYEHIDNGNLGEYLHGIGKIRTKKVLIFFCREPLPWSSRVQIALDSARGLEYIHEHTVPVYIHRDVKSANILIDKNLRGKVADFGLTKLIEVGNSTLHTRLVGTFGYMPPEYAQYGDVSPKIDVYAFGVVLYELISAKNAILKTGESVAESKGLVVLFEEALNQIDPLEGIRKLVDPRLKENYPIDSVLKMVELARGCTRDNPLLRPSMRSIVVALMTLSSPTEDCYYDTSYENQSLINILSLR

SEQ ID NO: 124 - protein, 633 aa, Arachis hypogaea; identical to SEQ ID NO: 71

SEQ ID NO: 125 - protein, 631 aa, Arachis hypogaea; identical to SEQ ID NO: 90

SEQ ID NO: 126 - protein, 621 aa, Medicago truncatula
MEHQPRFTSFISLPLFSIFLASIPFITESKCTKGCSLALANFYVSQGSNLTYISSIMRSNIQTRPEDIVEYSREIIPSKDSVQAGQRLNVPFPCDCIDGQFLGHKFSYDVETGDTYETVATNNYANLTNVEWLRRFNTYPPNDIPDTGTLNVTVNCSCGDADVGNYALFVTYPLRPGETLVSVANSSKVDSSLLQRYNPGVNFNQGSGIVFVPGKDQNGSFVFLGSSSGLGGGAIGGIAVGIVVVLLLVAAAIYFGYFRKKKIQKEELFSRDSTALFSQDGKDENSHGAANVTQRPGVMTGITVDKSVEFSYDELAAASDNFSMANKIGQGGFGSVYYAELRGEKAAIKKMDMQATKEFLAELKVLTRVHHLNLVRLIGYSIEGSLFLVYEYIENGNLSQHLRGSGRDPLPWATRVQIALDSARGLEYIHEHTVPVYIHRDIKPANILIDKNFRGKVADFGLTKLTEVGSSSLPTGRLVGTFGYMPPEYAQYGDVSPKVDVYAFGVVLYELISAKEAIVKSSESVADSKGLVGLFEGVLSQPDPTEDLRKIVDPRLGDNYPADSVRKMAQLAKACTQENPQLRPSMRSIVVALMTLSSTTDDWDVGSFYENQNLVNLMSGR

SEQ ID NO: 127 - protein, 624 aa, Cajanus cajan
MEPRFRFLLILVLLVLVLVIVPLEVESACKQGCPVALGSYYMWSGSKLTYISQIMPSALLTKPEDIVAYNKDTVPNKDSVQAFIRVNVPFPCDCVDQQFLAHTFQYDVQSQDTYDYVARTVFANLTDVAWLRRFNSYAPDNIPDTGTLNVTVNCSCGNSDVGGYGLFVTYPLRPGDTLGSVASSVGLDSGLLQRYNPGVNFNQGTGLVYIPGKDQNGSYVFLSSSSGAGLPGGAIAGIAVGVVAGLLLLGVCLYVGYFRKKKIRKVELPLDSSALFAQDGKDETSRSSANETSGPGGPAAITGITVDKSVEFTYEELATATDNFSLANKIGQGGFGSVYYAELRGEKAAIKKMDMQASKEFLAELKVLTRVHHLNLVRLIGYSIEGSLFLVYEYIENGNLSQHLRGAGSREPLPWATRVQIALDSARGLEYIHEYTVPVYIHRDIKSANILIDKNFRGKVADFGLTKLTEVGSSSLPTGRLVGTFGYMPPEYAQYGDVSPKVDVYAFGVVLYELISAKEAIVKTSESEADSKGLVALFDGVLSQPDPTEDLCKLVDPRLGDNYPIDSVRKMAQLGKACTQDNPQLRPSMRSIVVALMTLSSTTDDWDVGSFYENQNLVNLMSGR

SEQ ID NO: 128 - protein, 618 aa, Phaseolus vulgaris
MEGGLGCVLLPLLLFCVVLTAESACKEGCPLALGSYYMWQNSNLTYISQIMASSLLTTADDIVLYNKDTIPNKDSVQAFIRVNVPFPCDCIDGQFLAHTFKYDVQSQDSYEYVARTVYSNLTDVAWLRNFNSYEPDNIPDTATLNVTVNCSCGNSDVADYGLFITYPLRTGETLGSVAAAVSLDSGLLQRYNPGVNFNQGSGLVYIPGKDQNGSYVFLASSSGGLAVGAIAGIVVGVVAVLLLLGVFIYFRRFRKKIQKDELPRDSSALFAQHGKDEASRSSANETLGPGGPAAITGIKVDKSVKFTYEELATATDNFSLANKIGQGGFGSVYYAELRGEKAAIKKMDMQATKEFHAELNVLTRVHHLNLVRLIGYSMEGSLFLVYEYIENGNLSQHLRGSGSREPLPWATRVQIALDSARGLEYIHEHTVPLYIHRDIKSANILIDKNFRGKVADFGLTKLTEVGSSSLPTGRLVGTFGYMPPEYAQYGDVSPKVDVYAFGVVLYELISAKEAIVKTNDSAVDSKGLVALFDGVFGQPDPTEDLCKLVDPRLGDNYPIDSVRKMAQLAKACTQDNPQLRPSMRSIVVALMTLSSTTDEWDVGSFYENQNLVNLMSGR

SEQ ID NO: 129 - protein, 618 aa, Glycine max
MEALRLAYLLLPWWLVFSTAESACKEGCGVALGSYYLWRGSNLTYISSIMASSLLTTPDDIVNYNKDTVPSKDIIIADQRVNVPFPCDCIDGQFLGHTFRYDVQSQDTYETVARSWFANLTDVAWLRRFNTYPPDNIPDTGTLNVTVNCSCGNTDVANYGLFVTYPLRIGDTLGSVAANLSLDSALLQRYNPDVNFNQGTGLVYVPGKDQNGSFVRLPSSSGGLTGRAIAGIAVGIVAALLLLGVCIYVGYFRKKIQKDEFLPRDSTALFAQDGKDETSRSSANETSGPGGPAIITDITVNKSVEFSYEELATATDNFSLANKIGQGGFGSVYYAELRGEKAAIKKMDMQASKEFLAELNVLTRVHHLNLVRLIGYSIEGSLFLVYEYIENGNLSQHLRGSGSREPLPWATRVQIALDSARGLEYIHEHTVPVYIHRDIKSANILIDKNFRGKVADFGLTKLTEVGSSSLPTGRLVGTFGYMPPEYAQYGDVSPKVDVYAFGVVLYELISAKEAIVKTNDSVADSKGLVALFDGVLSQPDPTEELCKLVDPRLGDNYPIDSVRKMAQLAKACTQDNPQLRPSMRSIVVALMTLSSTTDDWDVGSFYENQNLVNLMSGR

SEQ ID NO: 130 - protein, 627 aa, Abrus precatorius
MGSRFGFLILLPMFFFIMLLLFFSLPFTVESKCTEGCPLALASYFLWRGSNLTYVSQIMASNVLTRPEDIVSYNKDTVPNKDSIQALSRVNVPFPCDCINGEFLGYTFKYDVQTGDTYETVAGTNYANLTNVAWLRRFNSYPPNNIPDTGTLNVTVNCSCGMSEVSNYGLFITYPLRPGETLGTVASSVGLDSALLQSYNPSVNFNQGSGLVFIPGKDQNGSYVFLSSSSGGLAGGAVAGIAVGVVVGLLFLGVCIYVGYFRKKKIQKEELLPQESSALFAQNGKDETSRSSGNETAGAGGPAAMTGIAVEKSLEFSYEELATATNNFNMANKIGQGGFGAVYYAELRGEKAAIKKMDMQASKEFLAELKVLTRVHHLNLVRLIGYSIEGSLFLVYEYIENGNLSQHLRGSGRDPLPWATRVQIALDSARGLEYIHEHTVPVYIHRDIKSANILIDRNFRGKVADFGLTKLTEVGSSSLPTGRLVGTFGYMPPEYAQYGDVSPKVDVYAFGVVLYELISAKEAIVKTSESDADSKGLVALFEGVLSQPDPTEGLCKLVDPRLGDNYPIDSVRKMAQLAKACTQDNPQLRPSMRSIVVALMTLSSTTDDWDVGSFYENQNLVNLMSGR

SEQ ID NO: 131 - protein, 624 aa, Nissolia schottii
MEPKFGFRDLLLLVLLCSATFSAESKCSPSQGCPRALGSYYVWRGSNLTYISQIMASNLLKTADDIVSYNKDTVPNKDSVQADIRVNVPFPCDCIDGEFLGHTFKYDVQKGDTYTLVAGTNYANLTTVDWLRRFNSYPPNNIPDTGTLNVTVNCSCGNSQVSNYGLFVTYPLRPGETLGTVARDVGLDSGLLQRYNPSVNFNQGSGLVFIPGKDQNGSFVFLGSSSGAGLASGAIAGIAVGIVAGLLLLAVGIYFGYFRKKKMQTEEFLPQDSKALFAQDGKDETSRRAAYETSGPGGTTAITGITVDKSVEFSYEELATATDNFSLANKIGQGGFGAVYYAELRGEKAAIKKMDMQASKEFLAELKVLTRVHHLNLVRLIGYSIEGSLFLVYEFIENGNLGQHLRGSGRDPLPWATRVQIALDSARGLEYIHEHTVPVYIHRDIKSANILIDKNFRGKVADFGLTKLTEVGSSSLPTGRLVGTFGYMPPEYAQYGDVSPKVDVYAFGVVLYELISAKEAIVKTGEYDADSKGLVALFEGALSQPDPTEDLCKLVDPRLGDNYPIDSVRKMAQLAKACTQDNPQLRPSMRSIVVALMTLSSTTEDWDVGSFYENQNLVNLMSGR

SEQ ID NO: 132 - protein, 622 aa, Arachis ipaensis
MEPRYGFASVLLQLLLLGCVLYGAESQCSKGCPLALASYYMWTGSNLTYVSQIMKSNVLSDPNGIVNYNKDTIPNKDSVQAFIRVNVPFPCDCINGEFLGHTFKYDIQSGDTYEHVATNNYANLTNVNWLRKFNTYPPNNIPNTGTLNVTVNCSCGNRQVANYGLFITYPLRPGDTLQAVAKNQSVDAFLLQKYNPSVNFNQGSGLVYIPGKDQNGSFVFLSSSSGGLAAGAIAGIVVGIVAGLLLVAVGIYFGYFRKKKMQREELLSQDSKPMFRQDGKDETSRSAAYEAAGPAGPGTITGITVDKSVEFSYEELATATNNFNLANKIGQGGFGAVYYAELRGEKAAIKKMDMQASKEFLAELKVLTRVHHLNLVRLIGYSIEGSLFLVYEYIENGNLSQHLRGSGREPLPWATRVQIALDSARGLEYIHEHTVPVYIHRDIKSANILIDKNYRGKVADFGLTKLTEVGSSSLPTGRLVGTFGYMPPEYAQYGDVSPKVDVYAFGVVLFELISAKEAIVKTSESVADSKGLVALFEGVLSQPDPTEDLCKLVDPRLGENYPIDSVRKMAHLAKACTQDNPQLRPSMRSIVVALMTLSSTTDDWDVGSFYENQNLVNLMSGR

SEQ ID NO: 133 - protein, 622 aa, Arachis duranensis
MEPRHRFVSVLLQLLLLGCVLYGAESQCSKGCPLALASYYMWTGSNLTYVSEIMKSNVLSDPNDIVNYNKDTIPNKDSVQASIRVNVPFPCDCINGEFLGHTFQYDIQSGDTYEHVATNNYANLTNVNWLRKFNSYPPNNIPDTGTLNVTVNCSCGNRQVANYGLFITYPLRPGDTLQAVAKNQSVDAFLLQKYNPSVNFNQGSGLVYIPGKDQNGSFVFLSSSSGGLAAGAIAGIAIGIVAGLLLVAVGIYFGYFRKKKMQREELLSQDSKPMFRQDGKDETSRSAAYEAAGPAGPGTITGITVDKSVEFSYEELATATNNFNLANKIGQGGFGAVYYAELRGEKAAIKKMDMQASKEFLAELKVLTRVHHLNLVRLIGYSIEGSLFLVYEYIENGNLSQHLRGSGREPLPWATRVQIALDSARGLEYIHEHTVPVYIHRDIKSANILIDKNYRGKVADFGLTKLTEVGSSSLPTGRLVGTFGYMPPEYAQYGDVSPKVDVYAFGVVLFELISAKEAIVKTSESVADSKGLVALFEGVLSQPDPTEDLCKLVDPRLGENYPIDSVRKMAHLAKACTQDNPQLRPSMRSIVVALMTLSSTTDDWDVGSFYENQNLVNLMSGR

SEQ ID NO: 134 - protein, 623 aa, Casaus castanospermum
MEPRFGFLVSMLFLLLLGSFLYSAESLCSQGCPLALGSYYFWPGANLTYVYQIMKSQVLTSPDDIVSYNKDVIPNKDSVQSFIRVNVPFPCDCINGEFLGHTFQYDVNTGDTYSFVAGTNYANLTTVDWLRHFNSYPPDRIPDTGTLNVTINCSCGNSQVSNYGLFITYPLRPGETLGSVARNMTLDAGLLQRYNPTVNFNQGSGLVYIPGKDQNGSYVFLGSSSGGLAGGAIAGISVGVVAGLLLLAFSVYVVYFQKKKREKVELLTQDSKALFAQDGKDGASRNAEYETATSVSPAPVITGITVDKSVEFSYEELATATNDFSMANRIGRGGFGSVYFAELRGEKAAIKKMDMQASKEFLAELKVLTRVHHLNLVRLIGYSIERSLFLVYEFIENGDLSQHLRGSERDPLPWATRVQIALDSARGLEYIHEHTVPVYIHRDIKSANLLIDKNFRGKVADFGLTKLTEVGSSSLPTGRLVGTFGYMPPEYAQYGDVSPKVDVYAFGVVLYELISAKEAIVKTSESVADSQGLVALFDGVLNQPDPTADLCKLVDPRLGDSYPIDSVRKMAQLAKACTQDNPQLRPSMRSIVVALMTLSSTTDEWDVGSFYENQNLVNRMSGR

SEQ ID NO: 135 - protein, 622 aa, Cicer arietinum
MEQLRFTSFLFFSSLLFSIPFISESKCTKGCSPALASYYLNSGANLTHISGIFSSSILTKPEDIVDYNQDTIANKDTIIAGKRINIPFPCDCIAGEFLAHTFSYDVQTGDTYETVATNNYANLTNVQWLQRFNSYPANNIPDTGTLNVTVNCSCGNSDVANYGLFLTYPLRPGETLGSVANSSNIDSSLLRSYNPGVNFNQGSGLVFIPGKDQKGNFVFLSSSSGGLGSGVIAGIAVGIVVVLLSLAVAIYFGIFRKKKIQKEELLSRDSTALFSRGRMDENSHGAANVTTRHGVPAAITGITVDKSVEFSYDELAAASDNFSLANKIGQGGFGSVYYAELRGEKAAIKKMDMQASKEFLAELKVLTRVHHLNLVRLIGYSIEGSLFLVYEFIENGNLSQHLRGSGRDPLPWATRVQIALDSARGLEYIHEHTVPVYIHRDIKPANILIDKNFRGKVADFGLTKLTEVGSSSLPTGRLVGTFGYMPPEYAQYGDVSPKVDVYAFGVVLYELISAKEAIVKTNESVAADSKGLVALFEGVLSQPDPTEDLHKLVDPRLGDTYPVDSVRKMAQLAKACTQENPQLRPSMRSIVVALMTLSSSTDDWDVGSFYENQNLVNLMSGR

SEQ ID NO: 136 - protein, 614 aa, Pisum sativum
MKQQHRFTFFISLPLFISLLASIPTTTQSKCTKGCSLALANFYLASGTNLTYVAGILKSPILTKPEDIVDYNRDTVPNKDIILGGERVNIPFPCDCINGDFLAHNFSYDVQTGDTYASVAGSNYANLTNVQWLRNFNTYPPNSIPDTGTLNVMINCSCGDREIADYGLFVTYPLRPGETLGSVANSTKLDSALLQRYNPGVNFNQGSGLVFIPGKDKNGSYVFLDTSSGGLAGGAIAGIAVGIVVLLLLAAGVYFGYFRKKKIQKENSLSRDSTSLFPQDGKDETSRNAVITGITVDKSVEFSYDELAAASDNFSMANKIGQGGFGSVYYAELRGEKAAIKKMDMQATKEFLAELNVLTRVHHLNLVRLIGYSIEGSLFLVYEYIENGNLSQHLRGSGRDPLPWATRVQIALDSARGLEYIHEHTVPVYIHRDIKPANILIDKNFRGKVADFGLTKLTEVGSSSLPTGRLVGTFGYMPPEYAQYGDVSPKVDVYAFGVVLYEIISAKEAIVKTSESVAADSKGLVSMFEGVLSQPDPSEDLRKIVDPRLGDNYPVDSVRKMAQLAKACTQENPQLRPSMRSIVVALMTLSSTTDDWDIGSFYENQNLVNLMSGR

SEQ ID NO: 137 - protein, 608 aa, Lupinus angustifolius
MKPILMFLIMFLLWLLLLSSAESKCTQGCSLALASYYMYSGSTLTSISQVMSSQLLQIPEDIVTYNKDTIPNKDSVQAFIRVNVPFPCDCIDGEFLGHMFQYDVKTGDTYQLVAETEYANLTNIDWLMKFNSYPANNIPDTGTLNVTVNCSCGEKNVSNYGLFITYPLRPGDTLDSVSKSVDLDSGLLQRYNPGVNFNQGSGLVYIPGKDQNGSYVFLNSSSEGLAGGVIAGIVIGVLAGILLLVAGIYVGYFRKKKIQKEELLEQDSKSLFVQNGMDETARTAATTGISVDKSVEFSYEELASATDNFSMANKIGQGGFGVVYYAELRGEKAAIKKMDMQASKEFLAELKVLTHVHHLNLVRLIGYSIEGSLFLVYEFIENGNLSQHLRGSGRDPLPWPARVQIALDSARGLEYIHEHTVPVYIHRDIKSANILIDKNFRGKVADFGLTKLTEVGSSSLPTGRLVGTFGYMPPEYAQYGDVSPKVDVYAFGVVLYELISAKEAIIQANESIADSKGLVALFEGVLNQPDSTEDLCKVVDPRLGDNYPIDSVRKLAQLAKACTQDNPQLRPSMRSIVVALMTLSSTTDDWDVGSFYENQNLVNLMSGR

SEQ ID NO: 138 - protein, 637 aa, Cercis sp.
METRFGFSFFFVFFASILLLAESKCSKGCDLALASYYVWQGSNLTYIAGIMKSQKVSSPDDIVSYNKDTVPNKDSVQAFIRVNVPFPCDCIDDEFLGHNFQYDVNTGDTYETIAKTNFANLTTVDWLQNFNSYPPNSIPDTSTLNVTINCSCGNSQVSKDYGLFITYPLRPGETLQSVADNVSLDTGLLQRYNPSVNFNQGSGLVYIPGKDQNGSYVPLGSRFRRNATLSLYFSSIQAGLAGGAIAGISVGIIAGLLLLAVSGYIGYFRKKKIEEMKLLSQDSKVLSVHNRSDEASGNAQYETSGSGGGGGGALITGITVDKSVEFSYEELANATNNFSLANKIGQGGFGAVYYAELRGEKAAIKKMDMQASREFLAELKVLTHVHHLNLVRLIGYCVESSLFLVYEYIENGNLSQHLRGSGIDPLPWSSRVQIALDSARGLEYIHEHTVPVYIHRDIKSANILIDRNFHGKVADFGLAKLTEVGSSSLPTGRLVGTFGYMPPEYAQYGDVSPKVDVYAFGVVLYELISAKEAIVRIDESDADSKGLIALFEGVLNQPDPAEDLRKLVDPRLGDTYPIDSVRKMAQLAKACTQDNPQLRPSMRSIVVALMTLSSNTEDWDVGSFYDNQALVNLMSGR

SEQ ID NO: 139 - protein, 618 aa, Chamaecrista fasciculata
MEPRFGFPVFFFFFFFSLTSIFLGAESKCSETCNLALASYYIQDGSNLTYISTILKSQLVSSPDDIVSYNKDKIPNKDSVPSDIRLNVPFPCDCIEDEFLGYNFLYDVQTGDTYERIARTNFANLTTVDWLQKFNSYPPNNIPDTGTLNVTINCSCGNRDVSKDYGLFITYPLMPGQTLQSVAQEVNLDTGLLQRYNPSVNFNQGSGLVYIPGKXXXGLGVGAIVGISIGVVAGVLLLAGYIYIRYFQKKKKEEVQLLSIYPNGHSSQPRRDATSGNAEYGTSGSVSPAVGGVAGITGITVDKSVEFSYEELATATNNFNIANKIGQGGFGAVYYAELRGEKAAIKKMDMQATREFLAELKVLTHVHHLNLVRLIGYCVESSLFLVYEYIENGNLSQHLRSAGRDPLPWSNRVQIALDSARGLEYIHEHTVPVYIHRDIKSANILIDKNFHGKVADFGLTKLTEVGSSTNPTGRLVGTFGYMPPEYAQYGEVSPKVDVYAFGVVLYELISAKEAIIKTAESITDSKGLVALFEGVLNLPDPTEDLRKLVDPRLGDDYPIDSVRKMAILAKACTQDNPQLRPTMRSIVVALMTLSSTTEDWDVGSFYENPALLNLMSGR

SEQ ID NO: 140 - protein, 610 aa, Lupinus angustifolius
MEPIFWFLIKLSPLFFLCSNAESKCTQGCPIALASYYMLSGSNLTYISQIMSSHVLHSPEDIVSYNKDKVQPFTRVNVPFPCDCIKGEFLGHMFQYVVQTGDTYETVAGTNYANLTNVEWLRRFNTYLPDNISSTGMLNVTVNCSCGNSDVSDYELFITYPLRPGETLGSVAKSVKLDSGLLQRYNPSVNFNQGSGLVYIPGKDQNGSYVFLSSSSGGLAGGAIAGIAVGVVAGILLLVVCIYVGCFRKKKIQKEEVVRPDSKSHSVPDGMDEISLVAAYETSRPRGSAAIAGISMDKSVEFSYEELASATNNFSVANKIGQGGFAVVYYAELRGEKAAIKKMDMQASKEFLAELNVLTHVHHLNLVRLIGYSIKGSLCLVYEFIENGNLSQHLHGSGREPLPWTIRVQIALDSARGLEYIHEHTMPIYIHRDIKSANILIDKNFRGKVADFGLAKLAEVGSSLRPTVRLVGTFGYMPPEYAQYGDVSPKVDVYAFGVVLYELISAKEAIIQSIVDSKGLVHWFKEVLSQPHSTEDLCKLVDPKLGDNYPIDSVLKLAQLAKACTQHNPQLRPSMRSIVVALMTLSSTTDDWDVGSFYENQNLVNLMSGK

SEQ ID NO: 141 - protein, 627 aa, Mimosa pudica
MEPRFGFLLFMLASIFVSSKSQCSESCDTALASYYVKNGTNLTFISKLMTSKLVSTPDDIVSYNKDKIPNKDSLSASIRINIPFPCDCINGQFLGHMFQYDVATGDTYDKIASTDFANLTTVEWLQKFNSYAADNIPDTATLNVTVNCSCGNRDVSDDYGLFITYPLLPGETLQSVASEVGFNDTGLLQRYNPSVNFNQGSGLVFLPGKDQNGTYVPLHIRXXXGLSTGAIVGICIGVVAGLLLLAVGIYFGYFKKKKTSKVKVLSQGLTSQDRRGKASDTTTGNDEYGTSVSGGPVSSIAGMTNITVDKSVEFSYEELATATNNFNMANMIGQGGFGAVYYAELRGEKAAIKKMDMQATKEFLAELKVLTNVHHLNLVRLIGYCVEDSLFLVYEYIENGNLSQHLRGSGPGRDPLPWSTRVQIALDSARGLEYIHEHTVPVYIHRDIKPANILIDKNFRGKVADFGLAKLTEVGSGTLPTGRLVGTFGYMPPEYAQYGDVSPKIDVYAFGVVLYELISAKEAIIKINESITDSKGLVSMVNGVLNQPNPMEDLRKLVDPRLGDDYSMDSVFKVAQLAKACTQDNPQLRPSMRSIVVALMTLSSATEDWDVGSFYQNPALMNLMSGR

SEQ ID NO: 142 - protein, 606 aa, Chamaecrista fasciculata
MEPRFVLLILLFIFLASCFRSTESNCSKGCDLTLGSYYVWPNLNLTFLSTVMKSQVLTDTNDIVSYNSGKVITNDYVQSGERINIPFPCDCIDGEFLGHKFDYSISKGDTYKTIAEEYYSNLTTVEWLKKFNTDQIPDNGTLTVTINCSCGNSQVSNLYGLFITYPLRPGETLKSVANDTGFDSELLQKYNPGVNFSQGSGLVYIPGKDQNGAYVPLESSSGSGGLAGGVIAGISVGVVAVLLLLAFWIYATFFRKKIDDEELLSQDLEIAALSGKGSGGPAGITGIVVDKSVEFSYEELAAATNNFSMANKIGQGGFGAVYYAELRGEKAAIKKMDMQATREFLAELKVLTHVHHLNLVRLIGYCVESSLFLVYEFIENGDLSEHLRGSGRDPLPWSTRVQIALDSARGLEYIHEHTVPVYIHRDIKSANILIDKSFHGKVADFGLAKLTEVGSSSLPTGHLVGTFGYMPPEYAQYGDVSPKVDVYAFGVVLFELLSAKEAIVKTSQSTADSKGLVTMFEGILNQRDPTKDICNLVDPRLGDNYPIDSVLKMAQLAKACTMENPQLRPSMRSIVVALMALSSTTVDWDAGYLYENRTVLNPMSGR

SEQ ID NO: 143 - protein, 564 aa, Trifolium sp.
MEKPQPRFIISFISLSFFFTLLSSIPSTTESKCTKGCPLALASFYAAKGSNLTYISSIMKSSILTRPEDIVDYNDDIIPNKDMFIGDQRINLPFPCDCINNDFLGHTFSYDVQKADTYETVAVTNYANLTDVQWLRKFNSYGPNNIPDTGTLNVTVNCSCGDSDVANYGLFVTYPLRPGETLGSVANSTKLDSSLLQRYNPGVNFNQGSGLVFIPGKDQNGSFVFLGSSSGGLAGGAIAGIAVGIVVVLLLAAGIYFGYFRKKKKEELLSRDSTALFSQDGKDENSRRSANGPGVPGVMTGITVDKSVEFSYDELAAASDNFSMANKIGQGGFGSVYYAELRGEKAAIKKMDMQATKEFLAELKVLTRVHHLNLVRLIGYSIEGSLFLVYEDKNFRGKVADFGLTKLTEVGSSSLPTGRLVGTFGYMPPEYAQYGDVSPKVDVYAFGVVLYELISAKEAIVKSSESITADSKGLVGLFEGVLSQPDPTEDLRKLVDPRLGDNYPADSVRKMAQLAKACTQENPQLRPSMRSIVVALMTLSSSTDDWDVGSFYENQNLVNLMSGR

SEQ ID NO: 144 - protein, 623 aa, Manihot esculenta
MSRKLGLALGFFLLLSHFYSTECKCSRTCDLALASYYVSSGDTLSVIAQKFKSNVLQSEDTIVNYNRDKTNNKDFIDAFIRINIPFPCDCIRDESLGLEFLGYKFQYNVESQDTYTTVANKTYSNLTTVSWLEQFNSYPATNIPDTGVLNVTVNCSCGDSSVSNDYGLFITYPLRPGDTLESIARESNISADLLQRYNVGADFSAGTGLVYVPGKDENNSYRPLTSSTGGIAGGAIAGIAIGTVAVVLLVAVCVYFGLYRKKKVKEAISLSGSQELSQALQAPGSNSDKPVESSGLRPSPGLAGITVDKSVEFSYEELSRATDNFNMANKIGQGGFGSVYYAELRGEKAAIKKMDMQASKEFFAELKVLTHVHHLNLVRLIGYCVEGSLFLVYEYIENGNLSQHLRGSGRDPLPWSARLQIALDSARGLEYIHEHTVPVYIHRDIKSANILIDKNFRGKVADFGLTKLTEVGGSSLPTRLVGTFGYMPPEYAQYGDVSPKVDVYAFGVVLYELISAKDAIVKTNDSSAETKGLVSLFEDALDEADAREELPKLVDPRLGDNYPIDSVEKLAQLAKACTHENPQLRPSMRSIVVALMTLSSSTEDWDVGSFYENKPLANLLSGR

SEQ ID NO: 145 - protein, 617 aa, Manihot esculenta
MNSLSEFACGFFLLLCFGCFAESKCSRGCDLALGSYYVWQKTNLTFISDVMKSYIIESPDTIVSYNRDQVPSKDSLTASIRINVPFPCDCINGEFLGHVFQYNVKSGDTYDVVSSVYYSNLTTVNWLKQFNSYPENNVPDTGVLNVTVNCSCGDSSISKDYGLFITYPLRPIDTLDSIASASNISADLLQRYNVGANFSQGSGLVYIPGKDENNSYRPLTSSTGIAGGLIAVISIGAVAGVLIVGVCVYFGFYRKKLKKATLLSGSHDLSAQTLQAPGSNSDKPVESTGLAPSPVLTGITVDKSVEFSYEELAQATDNFSTANKIGEGGFGSVYYAELRSEKAAIKKMDMQASKEFFAELKVLTHVHHLNLVRLIGYCVEGSLFLVYEYIENGNLSQHLRGSGRDPLPWSARVQIALDSARGLEYIHEHTVPVYIHRDIKSANILIDKNFRGKVADFGLTKLTEVGSGSLPTRLVGTFGYMPPEYAQYGDVSPKVDVYAFGVVLYELISAKEAIVKINGSSSETRGLVALFEEVLNEPDPKEELCKLIDPRLGDNYPFDSVRKMAQLAKACTHENPHLRPSMRSIVVALMTLSSSTEDWDVGSFYENQALVNLMSGR

SEQ ID NO: 146 - protein, 620 aa, Parasponia andersonii
MKAQAGAGGLCMFLVFISLCFRVESKCSKGCDLALASFYVWPFSNLTLISDLFDKIGQDGLDEIISYNRDKIPTKDFIESTIRINIPFPCDICVNNQYLGHVFEYNVNSGDTYTTVVSNYSSLTTVQSLTESNPEYNPNNIPDDGKLNVTVNCSCGNSLISKAYGLFITYPLRSEDSLDKLAAEHNISADLIKSYNPNVNFSQGSGLVFIPGKDRNGSYRPLTSSSGLAGGVIAGIIVAAVVGMLLLAVCVYFTFYRQKKVDGKLIPAVSEDPSSDYLNGPESSSDKAAAAPGDAGAGLAGITVDKSVEFSYEELAKATDDFSLAHKIGQGGFGAVYYAELRGEKAAIKKMDMQASREFLAELKVLTRVHHLNLVRLIGYCVEGSLFLVYEYIENGNLSQHLRGSEREPLPWSTRVQIALDSARGLEYIHEHTVPVYIHRDIKSANILIDKNFHAKVADFGLTKLTEVGSISIPTRLVGTFGYMPPEYAQYGDVSPKVDVYAFGVVIYELISAKEAIVKPDGSVAESKGLVGLFEDVLNQPDSKDDLRKLVDPRLGENYPVDSVRKMAQLAKACTHENPQLRPSMRSIVVALMTLSSSTEDWDVGSFYENQGLVNLMSGR

SEQ ID NO: 147 - protein, 614 aa, Parasponia andersonii
MKARAGLWFLVLISLCFRVKSKCGKGCDLAFASFYVYEGSNLTFISDLFNKNSPDEIVSYNTDKIPNKDSIQSTIRINIPFPCDVCVNDQYLGHLLEYNVVSGDTYEKVVSKYSNLTTVQSLTESNPQYDPNNIPDVNGKLNVTVNCSCGNSQISKAYGLFITYPLRWDDTLDKIATAQNISADLISKYNPGVNFSQGSGLVFIPEKDRNGSYRPLTSSSGLAGGVIAGIIVAAVVGMLLLAVCVYFTFYRQKKVDGKLIPAVSEDPSSDYLNGPESSSDKAAAAPGDAGAGLAGITVDKSVEFSYEELAKATDDFSLAHKIGQGGFGAVYYAELRGEKAAIKKMDMQASREFLAELKVLTRVHHLNLVRLIGYCVEGSLFLVYEYIENGNLSQHLRGSEREPLPWSTRVQIALDSARGLEYIHEHTVPVYIHRDIKSANILIDKNFHAKVADFGLTKLTEVGSISIPTRLVGTFGYMPPEYAQYGDVSPKVDVYAFGVVIYELISAKEAIVKPDGSVAESKGLVGLFEDVLNQPDSKDDLRKLVDPRLGENYPVDSVRKMAQLAKACTHENPQLRPSMRSIVVALMTLSSSTEDWDVGSFYENQGLVNLMSGR

SEQ ID NO: 148 - protein, 583 aa, Manihot esculenta
MIRLFHALLFLSLKLKAEANCKTGCDLALASYNVWAGVNLTYISNLFSKSITEILPYNPHVPNQDSILTDTRINVPFSCDCLNGDFLGHTFTYNTQPGDTYNKIATIAFANLTTEDWVHRVNIYDPTRIPDHASINVTVNCSCGDKRVSKNYGLFTTYPLRPGENLSFGLPADLLQRYNPGMNFSAGTGIIYVPAKDSTGNYPPLKISAAGISSRVIAGISIAGVAGALLFASFIYFRFYKKNKVHKSLLSETTEHHYSQHGRGITVDKSVEFSYEELAKATHDFSMANKIGQGGFGSVYYAELRGEKAAIKKMDMQASKEFLAELKVLTHVHHLNLVRLIGYCVESSLFLVYEFIENGNLSQHLRGSERDPLPWVTRVQIALDSARGLEYIHEHTVPVYIHRDVKSANILIDKNFRGKVADFGLTKLTEYGSASVHTRLVGTFGYMPPEYAQYGDVSPKIDVYAFGVVLYELISAKEAVVKTNEIVTESKGLVALFEDVLIEPDPKEDIRELVDPRLGDSYPLDSVCKMAQLAKACTQENPQLRPSMRSIVVALMTLSSSTEDWDVGSLYENQALVNLMSGR

SEQ ID NO: 149 - protein, 626 aa, Hordeum vulgare; identical to SEQ ID NO: 56

SEQ ID NO: 150 - protein, 233 aa, Hordeum vulgare
MEAPLHSLLLLLLLAAAAGPKTAAAVGDGCSRGCDLALASYYIAPNQNVTYIASLFGFSEYRVLGQYNPGVNNLDYVVAGDRLNVSLTCKCLASLSAPASTFLAASIPYKVATGETYLRIADNYNNLTTADWLVATNTYPANNIPDVATVNATVNCSCGDAGISTDYGLFLTYPLRDRETLASVAANHGFSSPEKMDLLKKYNPGMDGVTGSGIVYIPAKDPNGSYRPLESPG

SEQ ID NO: 151 - DNA, 4171 bp, synthetic construct
  source 1..4171: mol_type = other DNA
gcatgcatatagctctatttctttagtaatgttacacctgcacgatgtccataataatagaagtcataatacatatacagattaaaattaaataaacaatttctaatcaaatttaaaaatgtcaacttaatttcattattaaaatataacaatatgaataaccaaaaataaattaagacattcacccccccccccccgaaaagaaatttaagacaattacaattttttggtatatatattaaagacttccaattatggacataggatctcaacttagtaatcttcactttaggaaagtcttttccccacaagtcacaaccatctattaatatcaatacaaaatgaagtcaactcaataaaaagatccttttataggaaattgatgaataaaactgatatatatttcagttaaaattgttcaaacattagtgcaatggacagaagtatcctttgtgccctcatttgccaacaactggctcatcaagcaataaattaattcgccatttccaaacttttgcagttttaagtagaagatatccattcgttgaaactttcttcacaccaccaatttcctcctaaatgggttaacaaatgtgcaatgatcgaaatatatagttgaaacgatcaagatcctctcaatggtaaaagaatttgaccacgctaagttttattatctcactagctattaatttaattatcatttatcttttcaattattaaacacacaaataatcaatcctaaaatgatgaaacttgacatggtctatttttacaataacttaaccaaaaacttataagttagcaactttcaaaaacaggttttcccttgttaagaatagacaaatcaaatggagtgtgttaaatattgtgtttaaaatagtgtgttgcaagcatttctcttataaaaaatcagtataaatatgtttggaactgtttattttagtttatcttatattataaatacaaaacaagtgtttggtaaagctaatgaaaataacttaaaacatacctatggtttgtcggatgtgtcgcgggtggagctccttgccattttgtgtggcctttgtatgtgttgagatatggccatgggttattgtagaatccttcttcttttggattcttctttgttcctgtctctcattcaacgctcatgttacccatttcatccatgccaagttttttttatacatgcatctaaattttttccgccatatcttaattttgtttttattaaaattaaaaagaatatgattgaatgtcaatgtaaattttttttacacagacaatgcatatccattaaggtttgttagaattacactccaccccatttttatctaaaatctacatcccaccccattttatatagaggcaaatttagtgacgaaaaatattcttcattaataattagttattatttaaattgttaatcaataatttcaaaaaaaaattcaatataatccaataaatttaaaaatgaaaacatcacaatcctcctcattctctcaatcgcgttttacctccgtaaatttacaatgcaaattatgcaatagcacacctgcccgatttacaaaccatatttcgaacatagtgaaacatgcttgtgttttcatatttggtgataattcaattttaatcaaaataatctctttatacctccaattttcaaaattgggttgtaggccaaaaaagcaacacaaatgggtgaagaaaatagagaaacaaaattatgaaaatatgaagtggatctgaggttattagagcccaacgaggcggtggagcatcgtttttaacaaaatccaacaatatctttaggggtgaaatccaccaaccgagcgttcgctcacaacttaaaggggtgaaattcacaagaagtagtttaaaggggtgaaattcacaagaagtagttgaacaagtgactttaggaatgtgcgattcacgttctggggttcaggtcgcaacaaaactttgaggcatggtggtgccatgtggtttcacattgtcggacagtggagccgtgttaaagggagtaaaggcttggtggtggccgtttgtggtgaaaaatatgatttggagatagatgggacgtggacttaacagacagagtggatggtttttttttaaatttaattcagtaaaattatttttataaattaatgtatgatagtgtatatgcattaaatttatttaaatttttactaattagtaatttgtttttagtagtgacgaatttgtttttgtcactaaattttgcctttataaaaaatggggtggagtgtagatttttaaaaagaatggggtggagtgtcattcttgcaaatcttgaggggggtgagtgtattttactcaactcttaaaaaaattaggaattaattagttgtaaattataaaagtttatttcattgaataacataacaaattaaaggcaaaaaaatacaaaacttcattttatatgtatttcagaaaaattgcctactttcaattatgagaaactaaaattatgtttagtttaaaatgagcatagattcaaaaattaataaataatatatatagcaggatacatgcctatcaattaacatatcgtttgtccacgatgatgatcttattggaggatcaatatcttcaaattaacaaagttatcacttggctcttattggtcataatgcaataaaaaaattgcaattagtatcaaatcaaactgaaatttgcaactatatgctgctggtgttgtcgcgcagattcctttttgatttttatgggaatgaagtcaatgaagcaacagtttcacaggcgtgcttaaaaataaaaaaattggaaatttgatgtttgttaggattatgagaggacacaatgggaggatgtttcacaagctgcagacagggttgccacttcagatgcaaaggattaaataaacaaagccaaggtttgcaatcaacaagattccatcgtcgttttgcttcctttaatcgtattaatcaaaagcacaccaagtaaagcatcaatatataacatccaagaaatcacaacatgatagttgctcgtgtcgtctattaactatgatgtcaggagttcgatccccgctcatgtgaatggaagtcatttcgttgttagatgtttaccgtttaatgcaaatactcgcggtgagataataagtcattgttgtgggcgaataccctaaaataagaataaaattaaatatagcatccaagttattgcccaaatatataaacaatggtattgttgacattattaggcataaaagcagtaggtaagtgtattatatttatttaattttttaaaattttgaaattaattaataattgttaacataagtaaaccatttttagcaaaaactctacacttctattaccttaacaagtacatttttgatggtacaccttaacaattaacaagtcatatgattgacaaacatattttatatgctttacaatttattctaaaatcaaagtttatgggaagaagctcataaaagtagttcctgggtgttttttagaatagagaagttgatcatgttagaaattaagttaaaaatgagttgaaagtgatttatgtttgattatatttatgagaaaaatgaattgtctgatgtaatattgtaaaatctaacaattaattaagtaccacagaaactagaatttatagcttcaccttagaattgattttggagttaaaatcaattattaaaggagcaattattaaaggagacatccaaatacactagttaattttgacaatcaattctaacacttgcaaatgtgtaaccaaacttactatcagtaagtgaactaatgattcccaagtcaacttttgttctagctagccaaccgttactatgttccctccacaatacattctccttgaaactgtcaagtgtcaactgcacccaaacatccttgtttgtgatgaaaagatcgaaaacgtgtgcttatgaatttacatgtttacattcaccaaaaatcaaaagttacacctctatacttatcacatatgtttgagtcactttccatataaaatcccatagtctattaattatcagagtaagtgtgacattagtttcaagagaaaaataaatgatcaaaacctggtagagagtcctagaaattcaatgttctgatttctttcattcatctctgctgccattttgatttgcaca

SEQ ID NO: 152 - protein, 72 aa, Lotus japonicus
QKKEEEKAKLPTDISMALSTQDGNASSSAEYETSGSSGPGTASATGLTSIMVAKSMEFSYQELAKATNNFSL

SEQ ID NO: 153 - protein, 69 aa, Lotus japonicus
RKKRIQKEELLSQETRAIFPQDGKDENPRSTVNETPGPGGPAAMAGITVDKSVEFSYDELATATDNFSL

SEQ ID NO: 154 - protein, 23 aa, Lotus japonicus
IMVAKSMEFSYQELAKATNNFSL

SEQ ID NO: 155 - protein, 23 aa, Lotus japonicus
ITVDKSVEFSYDELATATDNFSL

SEQ ID NO: 156 - protein, 23 aa, Trifolium pratense
IMVAKSTEFSYQELAKATNNFSL

SEQ ID NO: 157 - protein, 23 aa, Trifolium pratense
IMVAKSTEFSYQELAKATNNFNL

SEQ ID NO: 158 - protein, 23 aa, Arabidopsis thaliana
ISVDKSVEFSLEELAKATDNFNL

SEQ ID NO: 159 - protein, 23 aa, Oryza sativum
ITVDKSVEFSYEELSNATQGFSI